(19) United States
(12) Patent Application Publication
(10) Pub. No.: US 2022/0378829 A1
(43) Pub. Date: Dec. 1, 2022

(54) GENETICALLY ENGINEERED IMMUNE CELLS TARGETING CD70 FOR USE IN TREATING SOLID TUMORS

(71) Applicant: CRISPR THERAPEUTICS AG, Zug (CH)

(72) Inventors: Jonathan Alexander TERRETT, Cambridge, MA (US); Mary-Lee DEQUÉANT, Cambridge, MA (US); Matthias WILL, Cambridge, MA (US)

(21) Appl. No.: 17/742,947

(22) Filed: May 12, 2022

Related U.S. Application Data
(60) Provisional application No. 63/187,625, filed on May 12, 2021.

(51) Int. Cl. A61K 35/17; A61P 35/00; C07K 14/725; C07K 16/28; C12N 15/62; C12N 5/0783

(52) U.S. Cl. CPC A61K 35/17; A61P 35/00; C07K 14/7051; C07K 16/2803; C07K 16/2866; C12N 15/625; C12N 5/0636; A61K 2039/505

(57) ABSTRACT

A method for treating a solid tumor (e.g., a CD70+ solid tumor) comprising one or more cycles of treatment, each cycle comprising administering to a human patient in need thereof an effective amount of a population of genetically engineered T cells after a lymphodepleting therapy, and optionally a treatment comprising an anti-CD38 antibody. The population of genetically engineered T cells comprises T cells expressing a chimeric antigen receptor (CAR) that binds CD70.

### CROSS REFERENCE TO RELATED APPLICATIONS

This application claims the benefit of the filing date of U.S. Provisional Application No. 63/187,625, filed May 12, 2021, the entire contents of which are incorporated by reference herein.

### SEQUENCE LISTING

The instant application contains a Sequence Listing which has been filed electronically in ASCII format and is hereby incorporated by reference in its entirety. Said ASCII copy, created on May 4, 2022, is named 095136-0676-048US1_SEQ.txt and is 72,908 bytes in size.

### BACKGROUND

Chimeric antigen receptor (CAR) T-cell therapy uses genetically-modified T cells to more specifically and efficiently target and kill cancer cells. After T cells have been collected from the blood, the cells are engineered to include CARs on their surface. The CARs may be introduced into the T cells using CRISPR/Cas9 gene editing technology. When these allogeneic CAR T cells are injected into a patient, the receptors enable the T cells to kill cancer cells.

### SUMMARY

The present disclosure is based, at least in part, on the surprising discovery that anti-CD70 CAR+ T cells reduced tumor burden in various subcutaneous solid tumor xenograft models. It has also been demonstrated that the anti-CD70 CAR T cells described herein displayed long-term in vivo efficacy that prevented tumor growth after re-exposure to tumor cells. Significant reductions in tumor burden were also observed after redosing of anti-CD70 CAR T cells. Further, CTX130 cell distribution, expansion, and persistence were observed in human subjects receiving the CAR-T cells. Superior treatment efficacy was also observed in human patients having RCC (a representative CD70+ solid tumor) who received the CTX130 cell treatment regimen disclosed herein.

In some aspects, the present disclosure features a method for treating a solid tumor, the method comprising multiple cycles of treatment, wherein each cycle of treatment comprises: (i) performing a lymphodepletion treatment to a human patient having a solid tumor (e.g., renal cell carcinoma), which optionally is a CD70+ solid tumor; and (ii) administering to the human patient an effective amount of a population of genetically engineered T cells after step (i). The population of genetically engineered T cells comprises T cells expressing a chimeric antigen receptor (CAR) that binds CD70, optionally a disrupted TRAC gene, a disrupted β2M gene, a disrupted CD70 gene, or a combination thereof. In some instances, a nucleotide sequence encoding the CAR is inserted into a generic site of the genetically engineered T cells, for example, the disrupted TRAC gene.

In some embodiments, the lymphodepletion treatment in step (i) may comprise co-administering to the human patient fludarabine at 30 mg/m² and cyclophosphamide at 500 mg/m² intravenously per day for three days. In some instances, step (i) can be performed about 2-7 days prior to step (ii).

In some embodiments, the effective amount of the genetically engineered T cells in step (ii) may range from about 1×10⁶ CAR+ cells to about 9×10⁸ CAR+ cells. For example, the effective amount of the genetically engineered T cells in step (ii) may range from about 3×10⁷ CAR+ cells to about 1×10⁸ CAR+ cells. In other examples, the effective amount of the genetically engineered T cells in step (ii) may range from about 1×10⁸ CAR+ cells to about 3×10⁸ CAR+ cells. In yet other examples, the effective amount of the genetically engineered T cells in step (ii) may range from about 3×10⁸ CAR+ cells to about 4.5×10⁸ CAR+ cells. Alternatively, the effective amount of the genetically engineered T cells in step (ii) may range from about 4.5×10⁸ CAR+ cells to about 6×10⁸ CAR+ cells. In other examples, the effective amount of the genetically engineered T cells in step (ii) may range from about 6×10⁸ CAR+ cells to about 7.5×10⁸ CAR+ cells. In other examples, the effective amount of the genetically engineered T cells in step (ii) may range from or about 7.5×10⁸ CAR+ cells to about 9×10⁸ CAR+ cells. In specific examples, the effective amount of the genetically engineered T cells in step (ii) may be one of the following: about 3×10⁷ CAR+ T cells, about 1×10⁸ CAR+ T cells, about 3×10⁸ CAR+ T cells, about 4.5×10⁸ CAR+ T cells, about 6×10⁸ CAR+ T cells, about 7.5×10⁸ CAR+ T cells, or about 9×10⁸ CAR+ T cells.

In any of the methods disclosed herein, prior to step (ii) and after step (i), the human patient does not show one or more of the following features: (a) active uncontrolled infection, (b) worsening of clinical status compared to the clinical status prior to step (i), and (c) Grade≥2 acute neurological toxicity.

In some instances, the method disclosed herein may comprise two cycles of the treatment disclosed herein. In other instances, the disclosed herein may comprise three cycles of the treatment disclosed herein. In some examples, the human patient may show partial response or complete response after a cycle of treatment and loss of response within 2 years after administration of the genetically engineered T cells. Alternatively or in addition, the human patient may show stable disease or progressive disease with significant clinical benefit about 6 weeks after administration of the genetically engineered T cells. In some instances, administration of the genetically engineered T cells in two consecutive cycles is about 8 weeks apart.

In any methods disclosed herein, the human patient may not show one or more of the following prior to a subsequent cycle of the treatment: (a) dose-limiting toxicity (DLT), (b) Grade≥3 CRS that does not resolve to ≤Grade 2 within 72 hours after the immediate preceding cycle of the treatment, (c) Grade>1 GvHD, and (d) Grade≥2 ICANS. In some instances, the method may further comprise, between two consecutive cycles of the treatment, confirming presence of CD70+ tumor cells in the human patient.

In some embodiments, each cycle of the treatment may further comprise: (iii) administering to the human patient a first dose of an anti-CD38 antibody; and (iv) administering to the human patient a second dose of the anti-CD38 antibody after step (iii). In one specific example, the anti-CD38 antibody is daratumumab. The first dose of the anti-CD38 antibody may be administered to the human patient at least 12 hours prior to the lymphodepletion treatment in step (i). Alternatively or in addition, the first dose of the anti-CD38 antibody may be administered to the human patient within 10 days of administration of the genetically engineered T cells in step (ii). In some embodiments, the second dose of the anti-CD38 antibody in step (iv) may be administered to the human patient three weeks after administration of the genetically engineered T cells in step (iii).

In some instances, a third dose of the anti-CD38 antibody may be given to a suitable human patient, e.g., a human patient who achieves stable disease or a better response. In some examples, the third dose of the anti-CD38 antibody may be administered to the human patient about 6-7 weeks after administration of the genetically engineered T cells in step (ii).

In some instances, the first dose, the second dose, and/or the third dose of the anti-CD38 antibody can be 16 mg/kg by intraveneous infusion. In some examples, the first dose, the second dose, and/or the third dose of the anti-CD38 antibody can be split evenly into two portions (8 mg/kg each), which can be administered to the human patient in two consecutive days via intravenous infusion. Alternatively, the first dose, the second dose, and/or the third dose of the anti-CD38 antibody can be 8 mg/kg via intravenous infusion. In some examples, the second dose, and/or the third dose of the anti-CD38 antibody can be 1800 mg via subcutaneous injection.

In some embodiments, the method may comprise administering to the human patient one or more additional doses of the anti-CD38 antibody.

Prior to administration of a subsequent dose of the anti-CD38 antibody, the human patient may be free of one or more of the following: (a) severe or unmanageable toxicity with prior doses of the anti-CD38 antibody, (b) disease progression, (c) ongoing uncontrolled infection, (d) grade≥3 thrombocytopenia; (e) ≥3 neutropenia; and (f) CD4+ T cell count<100/μl.

In any of the methods disclosed herein, prior to the lymphodepletion treatment of each cycle, the human patient may not show one or more of the following features: (a) significant worsening of clinical status, (b) requirement for supplemental oxygen to maintain a saturation level of greater than 92%, (c) uncontrolled cardiac arrhythmia, (d) hypotension requiring vasopressor support, (e) active infection, (f) platelet count≤100,000/mm³, absolute neutrophil count≤1500/mm³, and/or hemoglobin≤9 g/dL without prior blood cell transfusion; and (g) Grade≥2 acute neurological toxicity.

In other aspects, provided herein is a method for treating a solid tumor (e.g., renal cell carcinoma), the method comprising: (i) administering to a human patient having a solid tumor, which optionally is a CD70+ solid tumor, one or more doses of an anti-CD38 antibody, (ii) performing a lymphodepletion treatment to the human patient after the first dose of the anti-CD38 antibody; and (iii) administering to the human patient an effective amount of a population of genetically engineered T cells, which expresses a chimeric antigen receptor (CAR) that binds CD70 and is deficient in MHC Class I expression (e.g., have a substantially reduced level of MHC Class I expression as relative to a wild-type counterpart or no detectable level of MHC Class I expression).

In some instances, the population of genetically engineered T cells comprises a disrupted β2M gene. In some examples, the population of genetically engineered T cells comprises T cells having a disrupted TRAC gene, and a disrupted β2M gene. In some examples, the population of genetically engineered T cells comprises T cells having a disrupted TRAC gene, a disrupted β2M gene, and a disrupted CD70 gene. In some examples, a nucleotide sequence encoding the CAR that binds CD70 is inserted into a suitable genetic site of the genetically engineered T cells. In one specific example, the nucleotide sequence encoding the CAR that binds CD70 is inserted into the disrupted TRAC gene.

In some embodiments, step (i) may comprise administering to the human patient a first dose of the anti-CD38 antibody at least 12 hours prior to the lymphodepletion treatment and within 10 days of the administration of the genetically engineered T cells. In some examples, step (i) may further comprise administering to the human patient a second dose of the anti-CD38 antibody about three weeks after administration of the genetically engineered T cells. In one specific example, the anti-CD38 antibody is daratumumab. In some embodiments, step (i) may further comprise administering to the human patient a third dose of the anti-CD38 antibody about 6-7 weeks after administration of the genetically engineered T cells.

The first dose, the second dose, and/or the third dose of the anti-CD38 antibody (e.g., daratumumab) may be about 16 mg/kg via intravenous infusion. In some examples, the first dose, the second dose, the third dose, or a combination thereof of the anti-CD38 antibody are split evenly into two portions (8 mg/kg each), which can be administered to the human patient in two consecutive days. In other examples, the first dose, the second dose, and/or the third dose of the anti-CD38 antibody is 8 mg/kg via intravenous infusion. In some examples, the second dose, and/or the third dose of the anti-CD38 antibody can be 1800 mg via subcutaneous injection.

In some embodiments, the lymphodepletion treatment in step (ii) may comprise co-administering to the human patient fludarabine at 30 mg/m² and cyclophosphamide at 500 mg/m² intravenously per day for three days. In some examples, step (ii) can be performed about 2-7 days prior to step (iii).

In some embodiments, the effective amount of the genetically engineered T cells in step (ii) may range from about 1×10⁶ CAR+ cells to about 9×10⁸ CAR+ cells. For example, the effective amount of the genetically engineered T cells in step (ii) may range from about 3×10⁷ CAR+ cells to about 1×10⁸ CAR+ cells. In other examples, the effective amount of the genetically engineered T cells in step (ii) may range from about 1×10⁸ CAR+ cells to about 3×10⁸ CAR+ cells. In yet other examples, the effective amount of the genetically engineered T cells in step (ii) may range from about 3×10⁸ CAR+ cells to about 4.5×10⁸ CAR+ cells. Alternatively, the effective amount of the genetically engineered T cells in step (ii) may range from about 4.5×10⁸ CAR+ cells to about 6×10⁸ CAR+ cells. In other examples, the effective amount of the genetically engineered T cells in step (ii) may range from about 6×10⁸ CAR+ cells to about 7.5×10⁸ CAR+ cells. In other examples, the effective amount of the genetically engineered T cells in step (ii) may range from or about 7.5×10⁸ CAR+ cells to about 9×10⁸ CAR+ cells. In specific examples, the effective amount of the genetically engineered T cells in step (ii) may be one of the following: about 3×10⁷ CAR+ T cells, about 1×10⁸ CAR+ T cells, about 3×10⁸ CAR+ T cells, about 4.5×10⁸ CAR+ T cells, about 6×10⁸ CAR+ T cells, about 7.5×10⁸ CAR+ T cells, or about 9×10⁸ CAR+ T cells.

In some embodiments, prior to step (iii) and after step (ii), the human patient does not show one or more of the following features: (a) active uncontrolled infection, (b) worsening of clinical status compared to the clinical status prior to step (ii), and (c) Grade≥2 acute neurological toxicity.

In some embodiments, prior to a subsequent dose of the anti-CD38 antibody, the human patient is free of one or more of the following: (a) severe or unmanageable toxicity with prior doses of the anti-CD38 antibody, (b) disease progression, (c) ongoing uncontrolled infection, (d) grade≥3 thrombocytopenia; (e) ≥3 neutropenia, and (f) CD4+ T cell count<100/μl.

In some embodiments, prior to the lymphodepletion treatment of step (ii), the human patient may not show one or more of the following features: (a) significant worsening of clinical status, (b) requirement for supplemental oxygen to maintain a saturation level of greater than 92%, (c) uncontrolled cardiac arrhythmia, (d) hypotension requiring vasopressor support, (e) active infection, (f) platelet count≤100,000/mm³, absolute neutrophil count≤1500/mm³, and/or hemoglobin≤9 g/dL without prior blood cell transfusion; and (g) Grade≥2 acute neurological toxicity.

Any of the methods disclosed herein may further comprise monitoring the human patient for development of acute toxicity after administration of the genetically engineered T cells. Exemplary acute toxicity comprises cytokine release syndrome (CRS), neurotoxicity, tumor lysis syndrome, GvHD, viral encephalitis, on target off-tumor toxicity, and uncontrolled T cell proliferation. In some examples, the neurotoxicity is immune effector cell-associated neurotoxicity (ICANS). In some examples, the on target off-tumor toxicity comprises activity of the population of genetically engineered T cells against activated T lymphocytes, B lymphocytes, dentritic cells, osteoblasts and/or renal tubular-like epithelium.

In some embodiments, the human patient for treatment by any of the methods disclosed herein can have unresectable or metastatic RCC. In some instances, the human patient has relapsed or refractory RCC. For example, the human patient has clear cell differentiation. In some examples, the human patient has undergone a prior anti-cancer therapy. Examples include, but are not limited to, a checkpoint inhibitor, a tyrosine kinase inhibitor, a vascular growth factor inhibitor, or a combination thereof. In some examples, the human patient is subject to an additional anti-cancer therapy after treatment with the population of genetically engineered T cells.

In some embodiments, the human patient for treatment by any of the methods disclosed herein may have one or more of the following features: (a) Karnofsky performance status (KPS)≥80%, (b) adequate organ function, (c) free of treatment with prior anti-CD70 or adoptive T cell or NK cell therapy, (d) free of contraindications to lymphodepletion therapy, (e) free of central nervous system (CNS) manifestation of malignancy, (f) free of prior central nervous system disorders, (g) free of pleural effusion or ascites or pericardial infusion, (h) free of unstable angina, arrhythmia, and/or myocardial infarction, (i) free of diabetes mellitus, (j) free of uncontrolled infections, (k) free of immunodeficiency disorders or autoimmune disorders that require immunosuppressive therapy, (l) free of liver vaccine or herbal medicines, and (m) free of solid organ transplantation or bone marrow transplant.

In some embodiments, the human patient is an adult.

In some embodiments, the genetically engineered T cells for use in any of the methods discloses herein express a CAR that binds CD70 (anti-CD70 CAR), which may comprise an extracellular domain, a CD8 transmembrane domain, a 4-1BB co-stimulatory domain, and a CD3ζ cytoplasmic signaling domain. In some examples, the extracellular domain is a single-chain antibody fragment (scFv) that binds CD70. Such an scFv may comprise a heavy chain variable domain (V_{H}) comprising SEQ ID NO: 49, and a light chain variable domain (V_{L}) comprising SEQ ID NO: 50. In one example, the scFv comprises SEQ ID NO: 48. In one specific example, the CAR comprises SEQ ID NO: 46 or SEQ ID NO: 81.

In some embodiments, the genetically engineered T cells for use in any of the methods discloses herein comprises a disrupted TRAC gene, which may be produced by a CRISPR/Cas9 gene editing system. In some examples, the CRISPR/Cas9 gene editing system may comprise a guide RNA comprising a spacer sequence of SEQ ID NO: 8 or 9. In some examples, the disrupted TRAC gene has a deletion of the region targeted by the spacer sequence of SEQ ID NO: 8 or 9, or a portion thereof.

In some embodiments, the genetically engineered T cells for use in any of the methods discloses herein comprises a disrupted β2M gene, which can be produced by a CRISPR/Cas9 gene editing system. In some examples, the CRISPR/Cas9 gene editing system may comprise a guide RNA comprising a spacer sequence of SEQ ID NO: 12 or 13.

In some embodiments, the genetically engineered T cells for use in any of the methods discloses herein comprises a disrupted CD70 gene, which may be produced by a CRISPR/Cas9 gene editing system. In some examples, the CRISPR/Cas9 gene editing system may comprise a guide RNA comprising a spacer sequence of SEQ ID NO: 4 or 5.

Also within the scope of the present disclosure are any of the anti-CD70 CAR T cells (e.g., the CTX130 cells), optionally in combination with an NK cell inhibitor such as an anti-CD38 antibody (e.g., daratumumab), for use in treating a CD70 positive solid tumor such as RCC, e.g., using a treatment regimen as disclosed herein. Further, provided herein are uses of the anti-CD70 CAR T cells, optionally in combination with an NK cell inhibitor such as an anti-CD38 antibody (e.g., daratumumab), for manufacturing a medicament for use in treating the CD70 positive solid tumor such as RCC, following a treatment regimen as disclosed herein.

The details of one or more embodiments of the invention are set forth in the description below. Other features or advantages of the present invention will be apparent from the following drawings and detailed description of several embodiments, and also from the appended claims.

### BRIEF DESCRIPTION OF THE DRAWINGS

FIG. 1 includes graphs showing efficient multiple gene editing in TRAC⁻/β2M⁻/CD70⁻/anti-CD70 CAR⁺ (i.e., 3×KO, CD70 CAR⁺) T cells.

FIG. 2 includes a graph showing that normal proportions of CD4+ and CD8+ T cells are maintained among the TRAC⁻/β2M⁻/CD70⁻/anti-CD70 CAR⁺ T cell population.

FIG. 3 includes a graph showing robust cell expansion in TRAC⁻/β2M⁻/CD70⁻/anti-CD70 CAR⁺ T cells. The total number of viable cells was quantified in 3×KO (TRAC−/β2M−/CD70−) and 2×KO (TRAC−/132M−) anti-CD70 CAR T cells. 3×KO cells were generated with either CD70 sgRNA T7 or T8.

FIG. 4 includes a graph showing robust cell killing of A498 cells by 3×KO (TRAC⁻/β2M⁻/CD70⁻) anti-CD70 CAR⁺ T cells compared to 2×KO (TRAC⁻/β2M⁻) anti-CD70 CAR⁺ T cells.

FIG. 5 includes a graph showing A498 cell killing by anti-CD70 CAR T cells after serial rechallenge. 3×KO (TRAC⁻/β2M⁻/CD70⁻) and the development lot of CTX130 cells (CTX130) anti-CD70 CAR+ T cells were utilized.

FIGS. 6A-6C include graphs showing results from testing of the development lot of CTX130 cells (lot 01) for cytokine secretion in the presence of CD70+ renal cell carcinoma cells. CTX130 cells were co-cultured with CD70+(A498; FIG. 6A or ACHN; FIG. 6B) or CD70− (MCF7; FIG. 6C) target cells at the indicated ratios. Unedited T cells were used as control T cells. IFN-γ (left) and IL-2 (right) levels were determined. Mean of biological triplicates±the standard deviation are shown.

FIGS. 7A-7C include graphs showing results from testing of the development lot of CTX130 cells (lot 01) for cell killing activity against CD70 high (A498; FIG. 7A), CD70 low (ACHN; FIG. 7B), and CD70 negative (MCF7; FIG. 7C) cells lines at multiple T cell to target cell ratios. Each data point represents data from triplicates±the standard deviation. Negative values are shown as zero.

FIGS. 8A-8H includes graphs showing expression of CD70 on various types of cancer cells and cytotoxicity of anti-CD70 CAR-T cells against such. FIG. 8A: relative CD70 expression in five different cancer cell lines as indicated. FIG. 8B: relative CD70 expression in three different cancer cell lines as indicated. FIG. 8C is a graph showing relative CD70 expression in nine different cancer cell lines. FIG. 8D is a graph showing cell kill activity using triple knockout TRAC⁻/β2M⁻/CD70⁻/anti-CD70 CAR⁺ T cells against additional solid tumor cell lines with varying levels of CD70 expression (4:1, 1:1, or 0.25:1 effector:target cell ratio). FIG. 8E is a graph showing cell kill activity using the triple knockout TRAC⁻/β2M⁻/CD70⁻/anti-CD70 CAR⁺ T cells against solid tumor cell lines after a co-culture period of 24 hours or 96 hours. FIGS. 8F-8H include graphs showing cell kill activity using the triple knockout TRAC⁻/β2M/CD70⁻/anti-CD70 CAR⁺ T cells (3KO (CD70), CD70 CAR+) against CD70-deficient chronic myelogenous leukemia (K562) cells (FIG. 8F), CD70-expressing multiple myeloma (MM.1S) cells (FIG. 8G), and CD70-expressing T cell lymphoma (HuT78) cells (FIG. 8H) at various effector:target ratios.

FIGS. 9A-9D includes graphs showing results from testing CTX130 cells in various subcutaneous renal cell carcinoma tumor xenograft models. FIG. 9A: a subcutaneous A498-NOG model. FIG. 9B: a subcutaneous 786-O-NSG model. FIG. 9C: a subcutaneous Caki-2-NSG model. FIG. 9D: a subcutaneous Caki-1-NSG model. Tumor volumes were measured twice weekly for the duration of the study. Each point represents the mean tumor volume±standard error.

FIG. 10 includes a graph showing results from testing the efficacy of CTX130 cells in a subcutaneous A498 xenograft model with tumor re-challenge. Tumors were allowed to grow to an average size of approximately 51 mm³ after which the tumor-bearing mice were randomized in two groups (N=5/group). Group 1 was left untreated while Group 2 received 7×10⁶ CAR+CTX130 cells and Group 3 received 8×10⁶ CAR+TRAC−B2M−Anti-CD70 CAR T cells. On Day 25, a tumor re-challenge was initiated whereby 5×10⁶ A498 cells were injected into the left flank of treated mice and into a new control group (Group 4). Tumor volume was measured twice weekly for the duration of the study. Each point represents the mean tumor volume±standard error.

FIG. 11 includes a graph showing results from testing the efficacy of CTX130 cells in a subcutaneous A498 xenograft model with redosing of CTX130 cells. When mean tumor size reached an average size of approximately 453 mm³, mice were either left untreated or injected intravenously (N=5) with 8.6×10⁶ CAR+CTX130 cells per mouse. Group 2 mice were treated with a second and third dose of 8.6×10⁶ CAR+CTX130 cells per mouse on day 17 and 36, respectively. Group 3 mice were treated with a second dose of 8.6×10⁶ CAR+CTX130 cells per mouse on day 36. Tumor volumes were measured twice weekly for the duration of the study. Each point represents the mean tumor volume±standard error.

FIG. 12A includes a graph showing results from an experiment designed to assess tumor volume reduction in a human ovarian tumor xenograft model (e.g., SKOV-3 tumor cells) exposed to 3×KO (TRAC−/B2M−/CD70−) anti-CD70 CAR T cells.

FIG. 12B includes a graph showing results from an experiment designed to assess tumor volume reduction in a human non-small cell lung tumor xenograft model (e.g., NCI-H1975 tumor cells) exposed to 3×KO (TRAC−/B2M−/CD70−) anti-CD70 CAR T cells.

FIG. 12C includes a graph showing results from an experiment designed to assess tumor volume reduction in a human pancreatic tumor xenograft model (e.g., Hs766T tumor cells) exposed to 3×KO (TRAC−/B2M−/CD70−) anti-CD70 CAR T cells.

FIG. 12D includes a graph showing results from an experiment designed to assess tumor volume reduction in a human gastric tumor xenograft model (e.g., SNU-1 tumor cells) exposed to 3×KO (TRAC−/B2M−/CD70−) anti-CD70 CAR T cells.

FIGS. 13A-13D include schematic illustrations depicting exemplary clinical study designs to evaluate CTX130 cells administration to adult subjects with a CD70+ solid tumor, either taken alone or in combination with daratumumab. FIG. 3A: an exemplary clinical study designs of single dose escalation to evaluate CTX130 cells administration to adult subjects with a CD70+ solid tumor such as RCC. D; Day; DLT: dose-limiting toxicity; M: month; max: maximum; min: minimum. The DLT evaluation period is the first 28 days after CTX130 infusion. FIG. 13B: an exemplary clinical study design of a multiple dose regimen to evaluate CTX130 cells administration to adult subjects with a CD70+ solid tumor such as RCC. ICF: informed consent form. LD chemo: lymphodepleting chemotherapy. Pre-LD chemo assessments are required prior to Cycles 2 and 3 only and must be completed before initiation of LD chemo. FIG. 13C: an exemplary clinical study design of single dose escalation with Daratumumab added to the lymphodepletion regimen to evaluate CTX130 cells administration to adult subjects with a CD70+ solid tumor such as RCC. Subjects receive an infusion of daratumumab (single dose of 16 mg/kg IV or 1800 mg SC) followed by LD chemotherapy (co-administration of fludarabine 30 mg/m² and cyclophosphamide 500 mg/m² IV daily for 3 days). Daratumumab infusion is administered at least 12 h prior to starting LD chemotherapy and within 10 days of CTX130 infusion. CTX130 will be administered 48 hours to 7 days after LD chemotherapy. Daratumumab administration at 16 mg/kg IV or 1800 mg SC can be repeated at Day 22 (and at Day 42+7 days in subjects who achieve SD or better). D: day; Dara: daratumumab; DLT: dose-limiting toxicity; h: hours; LD chemo: lymphodepleting chemotherapy; M: month; SD: stable disease. Note: DLT evaluation period=28 days. FIG. 13D: an exemplary clinical study design of a multiple dose regimen with Daratumumab added to the lymphodepletion regimen to evaluate CTX130 cells administration to adult subjects with a CD70+ solid tumor such as RCC. In each cycle, the initial administration of daratumumab (single dose 16 mg/kg IV or 1800 mg SC) is followed by LD chemotherapy (co-administration of fludarabine 30 mg/m² and cyclophosphamide 500 mg/m² IV daily for 3 days). Daratumumab administration (16 mg/kg IV or 1800 mg SC) may be repeated at Day 22 of each cycle. Pre-LD chemo assessments are required prior to Cycles 2 and 3 only and can be completed before initial infusion of daratumumab in each of these 2 cycles. Daratumumab is administered at least 12 h prior to starting LD chemotherapy and within 10 days prior to CTX130 infusion. CTX130 is administered 48 h to 7 days after LD chemotherapy. Dara: daratumumab; ICF: informed consent form; LD chemo: lymphodepleting chemotherapy.

The details of one or more embodiments of the invention are set forth in the description below. Other features or advantages of the present invention will be apparent from the following drawings and detailed description of several embodiments, and also from the appended claims.

### DETAILED DESCRIPTION

CD70 is a type II membrane protein and ligand for the tumor necrosis factor receptor (TNFR) superfamily member CD27 (Goodwin, (1993) Cell, 73, 447-456) with a healthy tissue expression distribution limited to activated lymphocytes and subsets of dendritic and thymic epithelial cells and in both humans and mice (Hintzen, (1994) The Journal of Immunology, 152, 1762-1773; Grewal, (2008) Expert Opin Ther Targets, 12, 341-51; Coquet et al. (2013) J Exp Med, 210, 715-728; Tesselaar et al., (2003) J Immunol, 170, 33-40). Ligation of CD70 expressed on the surface of dendritic cells with T cell expressed CD27 generates a costimulatory signal that contributes to T cell activation and proliferation characteristic of TNF/TNFR pairs (Watts, (2005) Immunol, 23, 23-68). In addition, CD70 is itself a signaling molecule that is upregulated on activated lymphocytes and may act as a checkpoint limiting uncontrolled T cell expansion (O'Neill et al., (2017) J Immunol, 199, 3700-3710). CD27 is a constitutively expressed T cell surface receptor, and CD27-CD70 mediated stimulation of lymphocytes is controlled mainly by the restricted spatial and temporal expression pattern of CD70. Typically CD70 remains on the surface of activated lymphocytes for a maximum of a few days (Hintzen, (1994) The Journal of Immunology, 152, 1762-1773; Lens, (1999) British Journal of Hematology, 106, 491-503; Nolte, (2009) Immunological Reviews, 229, 216-31).

In contrast to its tightly controlled normal tissue expression, CD70 is commonly expressed at elevated levels in many solid tumors (Flieswasser et al., Cancers, 11 1161, 1-13, 2019; Grewal, (2008) Expert Opin Ther Targets, 12, 341-51; Wajant, 2016 Expert Opin Ther Targets, 20, 959-73). The restricted expression pattern of CD70 in normal tissues and its widespread expression in various malignancies makes it an attractive target for antibody-based therapeutics.

Surprisingly, the anti-CD70 CAR+ T cells as disclosed herein successfully reduced tumor burden in various subcutaneous CD70 positive solid tumor xenograft models and displayed long-term in vivo efficacy that prevented tumor growth after re-exposure to tumor cells. Specifically, the anti-CD70 CAR+ T cells have significantly reduced tumor burden in ovarian, lung, pancreatic, and gastric xenograft models. Significant reductions in tumor burden were also observed after redosing of anti-CD70 CAR T cells. See also International Patent Application Nos. PCT/IB2020/060719, filed Nov. 13, 2020 and PCT/IB2020/060720, filed Nov. 13, 2020, the relevant disclosures of each of which are incorporated by reference for the subject matter and purpose referenced herein.

Without wishing to be bound by theory, it is believed that CAR T cells with disrupted MHC Class I are not able to provide the required WIC Class I-NK KIR receptor binding that prevents NK-cells from eliminating WIC-Class I sufficient cells, i.e., self-cells. Thus, allogeneic CAR T cells with disrupted WIC Class I are susceptible to elimination by NK cell-mediated immune surveillance. It was discovered that the administration of an NK cell inhibitor, such as anti-CD38 monoclonal antibody daratumumab, resulted in a reduction of NK cell numbers. The depletion of NK cells, in turn, protects the allogeneic CAR T cell from host NK-mediated cell lysis. The combination of CAR T cell therapy and NK cell inhibitors such as daratumumab thus presents an improvement over the existing CAR T cell therapy.

It was demonstrated that T cells isolated from PBMCs also express CD38 protein on the cell surface. Surprisingly, the addition of an anti-CD38 monoclonal antibody at doses that depleted NK cells did not affect T cell numbers, even after multi-day culture with an anti-CD38 monoclonal antibody. Nor does the addition of anti-CD38 monoclonal antibody at doses that depleted NK cell numbers induce CAR T cell activation. Accordingly, without wishing to be bound by theory, it is believed that anti-CD38 monoclonal antibody treatment is NK cell-specific and induces reduction of NK cells without causing undesirable non-specific CAR T cell activation or elimination. The addition of an NK cell inhibitor, such as an anti-CD38 monoclonal antibody (e.g., daratumumab), could suppress specific T cells, B cells, and/or NK cells to mitigate potential host immune responses to the allogenic CAR T cells. The NK cell inhibitor may also allow increased expansion and persistence of the CAT T cells. It therefore represents an improvement to existing CAR T cell therapy. See also WO2020/261219, the relevant discloses of which are incorporated by reference for the subject matter and purpose referenced herein.

Accordingly, the present disclosure provides, in some aspects, therapeutic uses of anti-CD70 CAR+ T cells (e.g., CTX130 cells), either taken alone or in combination with an NK cell inhibitor such as an inhibitor of CD38 (e.g., anti-CD38 antibody such as Daratumumab) for treating solid tumors such as renal cell carcinoma (RCC). The anti-CD70 CAR+ T cells may be given to a patient as a single dose. Alternatively, multiple doses (e.g., up to 3 doses) may be given to a patient, either taken alone or in combination with the NK cell inhibitor (e.g., Daratumumab). The anti-CD70 CAR T cells, methods of producing such (e.g., via the CRISPR approach), as well as components and processes (e.g., the CRISPR approach for gene editing and components used therein) for making the anti-CD70 CAR+ T cells disclosed herein are also within the scope of the present disclosure.

### I. Anti-CD70 Allogeneic CAR T Cells

Disclosed herein are anti-CD70 CAR T cells (e.g., CTX130 cells) for use in treating a hematopoietic cell malignancy, such as a T cell malignancy, a B cell malignancy, or a myeloid cell malignancy. In some embodiments, the anti-CD70 CAR T cells are allogeneic T cells having a disrupted TRAC gene, a disrupted B2M gene, a disrupted CD70 gene, or a combination thereof. In specific examples, the anti-CD70 CAR T cells express an anti-CD70 CAR and have endogenous TRAC, B2M, and CD70 genes disrupted. Any suitable gene editing methods known in the art can be used for making the anti-CD70 CAR T cells disclosed herein, for example, nuclease-dependent targeted editing using zinc-finger nucleases (ZFNs), transcription activator-like effector nucleases (TALENs), or RNA-guided CRISPR-Cas9 nucleases (CRISPR/Cas9; Clustered Regular Interspaced Short Palindromic Repeats Associated 9).

Exemplary genetic modifications of the anti-CD70 CAR T cells include targeted disruption of T cell receptor alpha constant (TRAC), β2M, CD70, or a combination thereof. The disruption of the TRAC locus results in loss of expression of the T cell receptor (TCR) and is intended to reduce the probability of Graft versus Host Disease (GvHD), while the disruption of the β2M locus results in lack of expression of the major histocompatibility complex type I (MHC I) proteins and is intended to improve persistence by reducing the probability of host rejection. The disruption of CD70 results in loss of expression of CD70, which prevents possible cell-to-cell fratricide prior to insertion of the CD70 CAR. The addition of the anti-CD70 CAR directs the modified T cells towards CD70-expressing tumor cells.

The anti-CD70 CAR may comprise an anti-CD70 single-chain variable fragment (scFv) specific for CD70, followed by hinge domain and transmembrane domain (e.g., a CD8 hinge and transmembrane domain) that is fused to an intracellular co-signaling domain (e.g., a 4-1BB co-stimulatory domain) and a CD3ζ signaling domain.

### (i) Chimeric Antigen Receptor (CAR)

A chimeric antigen receptor (CAR) refers to an artificial immune cell receptor that is engineered to recognize and bind to an antigen expressed by undesired cells, for example, disease cells such as cancer cells. A T cell that expresses a CAR polypeptide is referred to as a CAR T cell. CARs have the ability to redirect T-cell specificity and reactivity toward a selected target in a non-MHC-restricted manner. The non-MHC-restricted antigen recognition gives CAR-T cells the ability to recognize an antigen independent of antigen processing, thus bypassing a major mechanism of tumor escape. Moreover, when expressed on T-cells, CARs advantageously do not dimerize with endogenous T-cell receptor (TCR) alpha and beta chains.

There are various generations of CARs, each of which contains different components. First generation CARs join an antibody-derived scFv to the CD3zeta (ζ or z) intracellular signaling domain of the T-cell receptor through hinge and transmembrane domains. Second generation CARs incorporate an additional co-stimulatory domain, e.g., CD28, 4-1BB (41BB), or ICOS, to supply a costimulatory signal. Third-generation CARs contain two costimulatory domains (e.g., a combination of CD27, CD28, 4-1BB, ICOS, or OX40) fused with the TCR CD3ζ chain. Maude et al., Blood. 2015; 125(26):4017-4023; Kakarla and Gottschalk, Cancer J. 2014; 20(2):151-155). Any of the various generations of CAR constructs is within the scope of the present disclosure.

Generally, a CAR is a fusion polypeptide comprising an extracellular domain that recognizes a target antigen (e.g., a single chain fragment (scFv) of an antibody or other antibody fragment) and an intracellular domain comprising a signaling domain of the T-cell receptor (TCR) complex (e.g., CD3C) and, in most cases, a co-stimulatory domain. (Enblad et al., Human Gene Therapy. 2015; 26(8):498-505). A CAR construct may further comprise a hinge and transmembrane domain between the extracellular domain and the intracellular domain, as well as a signal peptide at the N-terminus for surface expression. Examples of signal peptides include MLLLVTSLLLCELPHPAFLLIP (SEQ ID NO: 52) and MALPVTALLLPLALLLHAARP (SEQ ID NO: 53). Other signal peptides may be used.

(a) Antigen Binding Extracellular Domain

The antigen-binding extracellular domain is the region of a CAR polypeptide that is exposed to the extracellular fluid when the CAR is expressed on cell surface. In some instances, a signal peptide may be located at the N-terminus to facilitate cell surface expression. In some embodiments, the antigen binding domain can be a single-chain variable fragment (scFv, which may include an antibody heavy chain variable region (V_{H}) and an antibody light chain variable region (V_{L}) (in either orientation). In some instances, the V_{H} and V_{L} fragment may be linked via a peptide linker. The linker, in some embodiments, includes hydrophilic residues with stretches of glycine and serine for flexibility as well as stretches of glutamate and lysine for added solubility. The scFv fragment retains the antigen-binding specificity of the parent antibody, from which the scFv fragment is derived. In some embodiments, the scFv may comprise humanized V_{H} and/or V_{L} domains. In other embodiments, the V_{H} and/or V_{L} domains of the scFv are fully human.

The antigen-binding extracellular domain may be specific to a target antigen of interest, for example, a pathologic antigen such as a tumor antigen. In some embodiments, a tumor antigen is a “tumor associated antigen,” referring to an immunogenic molecule, such as a protein, that is generally expressed at a higher level in tumor cells than in non-tumor cells, in which it may not be expressed at all, or only at low levels. In some embodiments, tumor-associated structures, which are recognized by the immune system of the tumor-harboring host, are referred to as tumor-associated antigens. In some embodiments, a tumor-associated antigen is a universal tumor antigen, if it is broadly expressed by most types of tumors. In some embodiments, tumor-associated antigens are differentiation antigens, mutational antigens, overexpressed cellular antigens or viral antigens. In some embodiments, a tumor antigen is a “tumor specific antigen” or “TSA,” referring to an immunogenic molecule, such as a protein, that is unique to a tumor cell. Tumor specific antigens are exclusively expressed in tumor cells, for example, in a specific type of tumor cells.

In some examples, the CAR constructs disclosed herein comprise a scFv extracellular domain capable of binding to CD70. An example of an anti-CD70 CAR is provided in Examples below.

(b) Transmembrane Domain

The CAR polypeptide disclosed herein may contain a transmembrane domain, which can be a hydrophobic alpha helix that spans the membrane. As used herein, a “transmembrane domain” refers to any protein structure that is thermodynamically stable in a cell membrane, preferably a eukaryotic cell membrane. The transmembrane domain can provide stability of the CAR containing such.

In some embodiments, the transmembrane domain of a CAR as provided herein can be a CD8 transmembrane domain. In other embodiments, the transmembrane domain can be a CD28 transmembrane domain. In yet other embodiments, the transmembrane domain is a chimera of a CD8 and CD28 transmembrane domain. Other transmembrane domains may be used as provided herein. In some embodiments, the transmembrane domain is a CD8a transmembrane domain containing the sequence of FVPVFLPAKPTTTPAPRPPTPAPTIAS QPLSLRPEACRPAAGG AVHTRGLDFACDIYIWAPLAGTCGVLLLSLVITLYCNHRNR (SEQ ID NO: 54) or IYIWAPLAGTCGVLLLSLVITLY (SEQ ID NO: 55). Other transmembrane domains may be used.

(c) Hinge Domain

In some embodiments, a hinge domain may be located between an extracellular domain (comprising the antigen binding domain) and a transmembrane domain of a CAR, or between a cytoplasmic domain and a transmembrane domain of the CAR. A hinge domain can be any oligopeptide or polypeptide that functions to link the transmembrane domain to the extracellular domain and/or the cytoplasmic domain in the polypeptide chain. A hinge domain may function to provide flexibility to the CAR, or domains thereof, or to prevent steric hindrance of the CAR, or domains thereof.

In some embodiments, a hinge domain may comprise up to 300 amino acids (e.g., 10 to 100 amino acids, or 5 to 20 amino acids). In some embodiments, one or more hinge domain(s) may be included in other regions of a CAR. In some embodiments, the hinge domain may be a CD8 hinge domain. Other hinge domains may be used.

(d) Intracellular Signaling Domains

Any of the CAR constructs contain one or more intracellular signaling domains (e.g., CD3ζ, and optionally one or more co-stimulatory domains), which are the functional end of the receptor. Following antigen recognition, receptors cluster and a signal is transmitted to the cell.

CD3ζ is the cytoplasmic signaling domain of the T cell receptor complex. CD3ζ contains three (3) immunoreceptor tyrosine-based activation motif (ITAM)s, which transmit an activation signal to the T cell after the T cell is engaged with a cognate antigen. In many cases, CD3C provides a primary T cell activation signal but not a fully competent activation signal, which requires a co-stimulatory signaling.

In some embodiments, the CAR polypeptides disclosed herein may further comprise one or more co-stimulatory signaling domains. For example, the co-stimulatory domains of CD28 and/or 4-1BB may be used to transmit a full proliferative/survival signal, together with the primary signaling mediated by CD3ζ. In some examples, the CAR disclosed herein comprises a CD28 co-stimulatory molecule. In other examples, the CAR disclosed herein comprises a 4-1BB co-stimulatory molecule. In some embodiments, a CAR includes a CD3ζ signaling domain and a CD28 co-stimulatory domain. In other embodiments, a CAR includes a CD3ζ signaling domain and 4-1BB co-stimulatory domain. In still other embodiments, a CAR includes a CD3ζ signaling domain, a CD28 co-stimulatory domain, and a 4-1BB co-stimulatory domain.

It should be understood that methods described herein encompasses more than one suitable CAR that can be used to produce genetically engineered T cells expressing the CAR, for example, those known in the art or disclosed herein. Examples can be found in, e.g., WO 2019/097305A2, and WO2019/215500, the relevant disclosures of each of the prior applications are incorporated by reference herein for the purpose and subject matter referenced herein.

For example, the CAR binds CD70 (also known as a “CD70 CAR” or an “anti-CD70 CAR”). The amino acid sequence of an exemplary CAR that binds CD70 is provided in SEQ ID NO: 46 or SEQ ID NO: 81. See also amino acid sequences and coding nucleotide sequences of components in an exemplary anti-CD70 CAR construct in Table 1 below.

TABLE 1
Sequences of Exemplary Anti-CD70 CAR Construct Components.

| | | SEQ ID |
|---|---|---|
| Description | Sequence | NO: |
| CD70 | CCTGCAGGCAGCTGCGCGCTCGCTCGCTCACTGAGGCCGCCCGGGCGTCGG | 43 |
| rAAV | GCGACCTTTGGTCGCCCGGCCTCAGTGAGCGAGCGAGCGCGCAGAGAGGGA | |
| (CD70B scFV | GTGGCCAACTCCATCACTAGGGGTTCCTGCGGCCGCACGCGTGAGATGTAA | |
| with 41BB) | GGAGCTGCTGTGACTTGCTCAAGGCCTTATATCGAGTAAACGGTAGTGCTG | |
| | GGGCTTAGACGCAGGTGTTCTGATTTATAGTTCAAAACCTCTATCAATGAG | |
| | AGAGCAATCTCCTGGTAATGTGATAGATTTCCCAACTTAATGCCAACATAC | |
| | CATAAACCTCCCATTCTGCTAATGCCCAGCCTAAGTTGGGGAGACCACTCC | |
| | AGATTCCAAGATGTACAGTTTGCTTTGCTGGGCCTTTTTCCCATGCCTGCC | |
| | TTTACTCTGCCAGAGTTATATTGCTGGGGTTTTGAAGAAGATCCTATTAAA | |
| | TAAAAGAATAAGCAGTATTATTAAGTAGCCCTGCATTTCAGGTTTCCTTGA | |
| | GTGGCAGGCCAGGCCTGGCCGTGAACGTTCACTGAAATCATGGCCTCTTGG | |
| | CCAAGATTGATAGCTTGTGCCTGTCCCTGAGTCCCAGTCCATCACGAGCAG | |
| | CTGGTTTCTAAGATGCTATTTCCCGTATAAAGCATGAGACCGTGACTTGCC | |
| | AGCCCCACAGAGCCCCGCCCTTGTCCATCACTGGCATCTGGACTCCAGCCT | |
| | GGGTTGGGGCAAAGAGGGAAATGAGATCATGTCCTAACCCTGATCCTCTTG | |
| | TCCCACAGATATCCAGAACCCTGACCCTGCCGTGTACCAGCTGAGAGACTC | |
| | TAAATCCAGTGACAAGTCTGTCTGCCTATTCACCGATTTTGATTCTCAAAC | |
| | AAATGTGTCACAAAGTAAGGATTCTGATGTGTATATCACAGACAAAACTGT | |
| | GCTAGACATGAGGTCTATGGACTTCAGGCTCCGGTGCCCGTCAGTGGGCAG | |
| | AGCGCACATCGCCCACAGTCCCCGAGAAGTTGGGGGGAGGGGTCGGCAATT | |
| | GAACCGGTGCCTAGAGAAGGTGGCGCGGGGTAAACTGGGAAAGTGATGTCG | |
| | TGTACTGGCTCCGCCTTTTTCCCGAGGGTGGGGGAGAACCGTATATAAGTG | |
| | CAGTAGTCGCCGTGAACGTTCTTTTTCGCAACGGGTTTGCCGCCAGAACAC | |
| | AGGTAAGTGCCGTGTGTGGTTCCCGCGGGCCTGGCCTCTTTACGGGTTATG | |
| | GCCCTTGCGTGCCTTGAATTACTTCCACTGGCTGCAGTACGTGATTCTTGA | |
| | TCCCGAGCTTCGGGTTGGAAGTGGGTGGGAGAGTTCGAGGCCTTGCGCTTA | |
| | AGGAGCCCCTTCGCCTCGTGCTTGAGTTGAGGCCTGGCCTGGGCGCTGGGG | |
| | CCGCCGCGTGCGAATCTGGTGGCACCTTCGCGCCTGTCTCGCTGCTTTCGA | |
| | TAAGTCTCTAGCCATTTAAAATTTTTGATGACCTGCTGCGACGCTTTTTTT | |
| | CTGGCAAGATAGTCTTGTAAATGCGGGCCAAGATCTGCACACTGGTATTTC | |
| | GGTTTTTGGGGCCGCGGGCGGCGACGGGGCCCGTGCGTCCCAGCGCACATG | |
| | TTCGGCGAGGCGGGGCCTGCGAGCGCGGCCACCGAGAATCGGACGGGGGTA | |
| | GTCTCAAGCTGGCCGGCCTGCTCTGGTGCCTGGCCTCGCGCCGCCGTGTAT | |
| | CGCCCCGCCCTGGGCGGCAAGGCTGGCCCGGTCGGCACCAGTTGCGTGAGC | |
| | GGAAAGATGGCCGCTTCCCGGCCCTGCTGCAGGGAGCTCAAAATGGAGGAC | |
| | GCGGCGCTCGGGAGAGCGGGCGGGTGAGTCACCCACACAAAGGAAAAGGGC | |
| | CTTTCCGTCCTCAGCCGTCGCTTCATGTGACTCCACGGAGTACCGGGCGCC | |
| | GTCCAGGCACCTCGATTAGTTCTCGAGCTTTTGGAGTACGTCGTCTTTAGG | |
| | TTGGGGGGAGGGGTTTTATGCGATGGAGTTTCCCCACACTGAGTGGGTGGA | |
| | GACTGAAGTTAGGCCAGCTTGGCACTTGATGTAATTCTCCTTGGAATTTGC | |
| | CCTTTTTGAGTTTGGATCTTGGTTCATTCTCAAGCCTCAGACAGTGGTTCA | |
| | AAGTTTTTTTCTTCCATTTCAGGTGTCGTGACCACCATGGCGCTTCCGGTG | |
| | ACAGCACTGCTCCTCCCCTTGGCGCTGTTGCTCCACGCAGCAAGGCCGCAG | |
| | GTCCAGTTGGTGCAAAGCGGGGCGGAGGTGAAAAAACCCGGCGCTTCCGTG | |
| | AAGGTGTCCTGTAAGGCGTCCGGTTATACGTTCACGAACTACGGGATGAAT | |
| | TGGGTTCGCCAAGCGCCGGGGCAGGGACTGAAATGGATGGGGTGGATAAAT | |
| | ACCTACACCGGCGAACCTACATACGCCGACGCTTTTAAAGGGCGAGTCACT | |
| | ATGACGCGCGATACCAGCATATCCACCGCATACATGGAGCTGTCCCGACTC | |
| | CGGTCAGACGACACGGCTGTCTACTATTGTGCTCGGGACTATGGCGATTAT | |
| | GGCATGGACTACTGGGGTCAGGGTACGACTGTAACAGTTAGTAGTGGTGGA | |
| | GGCGGCAGTGGCGGGGGGGGAAGCGGAGGAGGGGGTTCTGGTGACATAGTT | |
| | ATGACCCAATCCCCAGATAGTTTGGCGGTTTCTCTGGGCGAGAGGGCAACG | |
| | ATTAATTGTCGCGCATCAAAGAGCGTTTCAACGAGCGGATATTCTTTTATG | |
| | CATTGGTACCAGCAAAAACCCGGACAACCGCCGAAGCTGCTGATCTACTTG | |
| | GCTTCAAATCTTGAGTCTGGGGTGCCGGACCGATTTTCTGGTAGTGGAAGC | |
| | GGAACTGACTTTACGCTCACGATCAGTTCACTGCAGGCTGAGGATGTAGCG | |
| | GTCTATTATTGCCAGCACAGTAGAGAAGTCCCCTGGACCTTCGGTCAAGGC | |
| | ACGAAAGTAGAAATTAAAAGTGCTGCTGCCTTTGTCCCGGTATTTCTCCCA | |
| | GCCAAACCGACCACGACTCCCGCCCCGCGCCCTCCGACACCCGCTCCCACC | |
| | ATCGCCTCTCAACCTCTTAGTCTTCGCCCCGAGGCATGCCGACCCGCCGCC | |
| | GGGGGTGCTGTTCATACGAGGGGCTTGGACTTCGCTTGTGATATTTACATT | |
| | TGGGCTCCGTTGGCGGGTACGTGCGGCGTCCTTTTGTTGTCACTCGTTATT | |
| | ACTTTGTATTGTAATCACAGGAATCGCAAACGGGGCAGAAAGAAACTCCTG | |
| | TATATATTCAAACAACCATTTATGAGACCAGTACAAACTACTCAAGAGGAA | |
| | GATGGCTGTAGCTGCCGATTTCCAGAAGAAGAAGAAGGAGGATGTGAACTG | |
| | CGAGTGAAGTTTTCCCGAAGCGCAGACGCTCCGGCATATCAGCAAGGACAG | |
| | AATCAGCTGTATAACGAACTGAATTTGGGACGCCGCGAGGAGTATGACGTG | |
| | CTTGATAAACGCCGGGGGAGAGACCCGGAAATGGGGGGTAAACCCCGAAGA | |
| | AAGAATCCCCAAGAAGGACTCTACAATGAACTCCAGAAGGATAAGATGGCG | |
| | GAGGCCTACTCAGAAATAGGTATGAAGGGCGAACGACGACGGGGAAAAGGT | |
| | CACGATGGCCTCTACCAAGGGTTGAGTACGGCAACCAAAGATACGTACGAT | |
| | GCACTGCATATGCAGGCCCTGCCTCCCAGATAATAATAAAATCGCTATCCA | |
| | TCGAAGATGGATGTGTGTTGGTTTTTTGTGTGTGGAGCAACAAATCTGACT | |
| | TTGCATGTGCAAACGCCTTCAACAACAGCATTATTCCAGAAGACACCTTCT | |
| | TCCCCAGCCCAGGTAAGGGCAGCTTTGGTGCCTTCGCAGGCTGTTTCCTTG | |
| | CTTCAGGAATGGCCAGGTTCTGCCCAGAGCTCTGGTCAATGATGTCTAAAA | |
| | CTCCTCTGATTGGTGGTCTCGGCCTTATCCATTGCCACCAAAACCCTCTTT | |
| | TTACTAAGAAACAGTGAGCCTTGTTCTGGCAGTCCAGAGAATGACACGGGA | |
| | AAAAAGCAGATGAAGAGAAGGTGGCAGGAGAGGGCACGTGGCCCAGCCTCA | |
| | GTCTCTCCAACTGAGTTCCTGCCTGCCTGCCTTTGCTCAGACTGTTTGCCC | |
| | CTTACTGCTCTTCTAGGCCTCATTCTAAGCCCCTTCTCCAAGTTGCCTCTC | |
| | CTTATTTCTCCCTGTCTGCCAAAAAATCTTTCCCAGCTCACTAAGTCAGTC | |
| | TCACGCAGTCACTCATTAACCCACCAATCACTGATTGTGCCGGCACATGAA | |
| | TGCACCAGGTGTTGAAGTGGAGGAATTAAAAAGTCAGATGAGGGGTGTGCC | |
| | CAGAGGAAGCACCATTCTAGTTGGGGGAGCCCATCTGTCAGCTGGGAAAAG | |
| | TCCAAATAACTTCAGATTGGAATGTGTTTTAACTCAGGGTTGAGAAAACAG | |
| | CTACCTTCAGGACAAAAGTCAGGGAAGGGCTCTCTGAAGAAATGCTACTTG | |
| | AAGATACCAGCCCTACCAAGGGCAGGGAGAGGACCCTATAGAGGCCTGGGA | |
| | CAGGAGCTCAATGAGAAAGGTAACCACGTGCGGACCGAGGCTGCAGCGTCG | |
| | TCCTCCCTAGGAACCCCTAGTGATGGAGTTGGCCACTCCCTCTCTGCGCGC | |
| | TCGCTCGCTCACTGAGGCCGGGCGACCAAAGGTCGCCCGACGCCCGGGCTT | |
| | TGCCCGGGCGGCCTCAGTGAGCGAGCGAGCGCGCAGCTGCCTGCAGG | |
| CD70 | GAGATGTAAGGAGCTGCTGTGACTTGCTCAAGGCCTTATATCGAGTAAACG | 44 |
| LHA to RHA | GTAGTGCTGGGGCTTAGACGCAGGTGTTCTGATTTATAGTTCAAAACCTCT | |
| (CD70B scFV | ATCAATGAGAGAGCAATCTCCTGGTAATGTGATAGATTTCCCAACTTAATG | |
| with 41BB) | CCAACATACCATAAACCTCCCATTCTGCTAATGCCCAGCCTAAGTTGGGGA | |
| | GACCACTCCAGATTCCAAGATGTACAGTTTGCTTTGCTGGGCCTTTTTCCC | |
| | ATGCCTGCCTTTACTCTGCCAGAGTTATATTGCTGGGGTTTTGAAGAAGAT | |
| | CCTATTAAATAAAAGAATAAGCAGTATTATTAAGTAGCCCTGCATTTCAGG | |
| | TTTCCTTGAGTGGCAGGCCAGGCCTGGCCGTGAACGTTCACTGAAATCATG | |
| | GCCTCTTGGCCAAGATTGATAGCTTGTGCCTGTCCCTGAGTCCCAGTCCAT | |
| | CACGAGCAGCTGGTTTCTAAGATGCTATTTCCCGTATAAAGCATGAGACCG | |
| | TGACTTGCCAGCCCCACAGAGCCCCGCCCTTGTCCATCACTGGCATCTGGA | |
| | CTCCAGCCTGGGTTGGGGCAAAGAGGGAAATGAGATCATGTCCTAACCCTG | |
| | ATCCTCTTGTCCCACAGATATCCAGAACCCTGACCCTGCCGTGTACCAGCT | |
| | GAGAGACTCTAAATCCAGTGACAAGTCTGTCTGCCTATTCACCGATTTTGA | |
| | TTCTCAAACAAATGTGTCACAAAGTAAGGATTCTGATGTGTATATCACAGA | |
| | CAAAACTGTGCTAGACATGAGGTCTATGGACTTCAGGCTCCGGTGCCCGTC | |
| | AGTGGGCAGAGCGCACATCGCCCACAGTCCCCGAGAAGTTGGGGGGAGGGG | |
| | TCGGCAATTGAACCGGTGCCTAGAGAAGGTGGCGCGGGGTAAACTGGGAAA | |
| | GTGATGTCGTGTACTGGCTCCGCCTTTTTCCCGAGGGTGGGGGAGAACCGT | |
| | ATATAAGTGCAGTAGTCGCCGTGAACGTTCTTTTTCGCAACGGGTTTGCCG | |
| | CCAGAACACAGGTAAGTGCCGTGTGTGGTTCCCGCGGGCCTGGCCTCTTTA | |
| | CGGGTTATGGCCCTTGCGTGCCTTGAATTACTTCCACTGGCTGCAGTACGT | |
| | GATTCTTGATCCCGAGCTTCGGGTTGGAAGTGGGTGGGAGAGTTCGAGGCC | |
| | TTGCGCTTAAGGAGCCCCTTCGCCTCGTGCTTGAGTTGAGGCCTGGCCTGG | |
| | GCGCTGGGGCCGCCGCGTGCGAATCTGGTGGCACCTTCGCGCCTGTCTCGC | |
| | TGCTTTCGATAAGTCTCTAGCCATTTAAAATTTTTGATGACCTGCTGCGAC | |
| | GCTTTTTTTCTGGCAAGATAGTCTTGTAAATGCGGGCCAAGATCTGCACAC | |
| | TGGTATTTCGGTTTTTGGGGCCGCGGGCGGCGACGGGGCCCGTGCGTCCCA | |
| | GCGCACATGTTCGGCGAGGCGGGGCCTGCGAGCGCGGCCACCGAGAATCGG | |
| | ACGGGGGTAGTCTCAAGCTGGCCGGCCTGCTCTGGTGCCTGGCCTCGCGCC | |
| | GCCGTGTATCGCCCCGCCCTGGGCGGCAAGGCTGGCCCGGTCGGCACCAGT | |
| | TGCGTGAGCGGAAAGATGGCCGCTTCCCGGCCCTGCTGCAGGGAGCTCAAA | |
| | ATGGAGGACGCGGCGCTCGGGAGAGCGGGCGGGTGAGTCACCCACACAAAG | |
| | GAAAAGGGCCTTTCCGTCCTCAGCCGTCGCTTCATGTGACTCCACGGAGTA | |
| | CCGGGCGCCGTCCAGGCACCTCGATTAGTTCTCGAGCTTTTGGAGTACGTC | |
| | GTCTTTAGGTTGGGGGGAGGGGTTTTATGCGATGGAGTTTCCCCACACTGA | |
| | GTGGGTGGAGACTGAAGTTAGGCCAGCTTGGCACTTGATGTAATTCTCCTT | |
| | GGAATTTGCCCTTTTTGAGTTTGGATCTTGGTTCATTCTCAAGCCTCAGAC | |
| | AGTGGTTCAAAGTTTTTTTCTTCCATTTCAGGTGTCGTGACCACCATGGCG | |
| | CTTCCGGTGACAGCACTGCTCCTCCCCTTGGCGCTGTTGCTCCACGCAGCA | |
| | AGGCCGCAGGTCCAGTTGGTGCAAAGCGGGGCGGAGGTGAAAAAACCCGGC | |
| | GCTTCCGTGAAGGTGTCCTGTAAGGCGTCCGGTTATACGTTCACGAACTAC | |
| | GGGATGAATTGGGTTCGCCAAGCGCCGGGGCAGGGACTGAAATGGATGGGG | |
| | TGGATAAATACCTACACCGGCGAACCTACATACGCCGACGCTTTTAAAGGG | |
| | CGAGTCACTATGACGCGCGATACCAGCATATCCACCGCATACATGGAGCTG | |
| | TCCCGACTCCGGTCAGACGACACGGCTGTCTACTATTGTGCTCGGGACTAT | |
| | GGCGATTATGGCATGGACTACTGGGGTCAGGGTACGACTGTAACAGTTAGT | |
| | AGTGGTGGAGGCGGCAGTGGCGGGGGGGGAAGCGGAGGAGGGGGTTCTGGT | |
| | GACATAGTTATGACCCAATCCCCAGATAGTTTGGCGGTTTCTCTGGGCGAG | |
| | AGGGCAACGATTAATTGTCGCGCATCAAAGAGCGTTTCAACGAGCGGATAT | |
| | TCTTTTATGCATTGGTACCAGCAAAAACCCGGACAACCGCCGAAGCTGCTG | |
| | ATCTACTTGGCTTCAAATCTTGAGTCTGGGGTGCCGGACCGATTTTCTGGT | |
| | AGTGGAAGCGGAACTGACTTTACGCTCACGATCAGTTCACTGCAGGCTGAG | |
| | GATGTAGCGGTCTATTATTGCCAGCACAGTAGAGAAGTCCCCTGGACCTTC | |
| | GGTCAAGGCACGAAAGTAGAAATTAAAAGTGCTGCTGCCTTTGTCCCGGTA | |
| | TTTCTCCCAGCCAAACCGACCACGACTCCCGCCCCGCGCCCTCCGACACCC | |
| | GCTCCCACCATCGCCTCTCAACCTCTTAGTCTTCGCCCCGAGGCATGCCGA | |
| | CCCGCCGCCGGGGGTGCTGTTCATACGAGGGGCTTGGACTTCGCTTGTGAT | |
| | ATTTACATTTGGGCTCCGTTGGCGGGTACGTGCGGCGTCCTTTTGTTGTCA | |
| | CTCGTTATTACTTTGTATTGTAATCACAGGAATCGCAAACGGGGCAGAAAG | |
| | AAACTCCTGTATATATTCAAACAACCATTTATGAGACCAGTACAAACTACT | |
| | CAAGAGGAAGATGGCTGTAGCTGCCGATTTCCAGAAGAAGAAGAAGGAGGA | |
| | TGTGAACTGCGAGTGAAGTTTTCCCGAAGCGCAGACGCTCCGGCATATCAG | |
| | CAAGGACAGAATCAGCTGTATAACGAACTGAATTTGGGACGCCGCGAGGAG | |
| | TATGACGTGCTTGATAAACGCCGGGGGAGAGACCCGGAAATGGGGGGTAAA | |
| | CCCCGAAGAAAGAATCCCCAAGAAGGACTCTACAATGAACTCCAGAAGGAT | |
| | AAGATGGCGGAGGCCTACTCAGAAATAGGTATGAAGGGCGAACGACGACGG | |
| | GGAAAAGGTCACGATGGCCTCTACCAAGGGTTGAGTACGGCAACCAAAGAT | |
| | ACGTACGATGCACTGCATATGCAGGCCCTGCCTCCCAGATAATAATAAAAT | |
| | CGCTATCCATCGAAGATGGATGTGTGTTGGTTTTTTGTGTGTGGAGCAACA | |
| | AATCTGACTTTGCATGTGCAAACGCCTTCAACAACAGCATTATTCCAGAAG | |
| | ACACCTTCTTCCCCAGCCCAGGTAAGGGCAGCTTTGGTGCCTTCGCAGGCT | |
| | GTTTCCTTGCTTCAGGAATGGCCAGGTTCTGCCCAGAGCTCTGGTCAATGA | |
| | TGTCTAAAACTCCTCTGATTGGTGGTCTCGGCCTTATCCATTGCCACCAAA | |
| | ACCCTCTTTTTACTAAGAAACAGTGAGCCTTGTTCTGGCAGTCCAGAGAAT | |
| | GACACGGGAAAAAAGCAGATGAAGAGAAGGTGGCAGGAGAGGGCACGTGGC | |
| | CCAGCCTCAGTCTCTCCAACTGAGTTCCTGCCTGCCTGCCTTTGCTCAGAC | |
| | TGTTTGCCCCTTACTGCTCTTCTAGGCCTCATTCTAAGCCCCTTCTCCAAG | |
| | TTGCCTCTCCTTATTTCTCCCTGTCTGCCAAAAAATCTTTCCCAGCTCACT | |
| | AAGTCAGTCTCACGCAGTCACTCATTAACCCACCAATCACTGATTGTGCCG | |
| | GCACATGAATGCACCAGGTGTTGAAGTGGAGGAATTAAAAAGTCAGATGAG | |
| | GGGTGTGCCCAGAGGAAGCACCATTCTAGTTGGGGGAGCCCATCTGTCAGC | |
| | TGGGAAAAGTCCAAATAACTTCAGATTGGAATGTGTTTTAACTCAGGGTTG | |
| | AGAAAACAGCTACCTTCAGGACAAAAGTCAGGGAAGGGCTCTCTGAAGAAA | |
| | TGCTACTTGAAGATACCAGCCCTACCAAGGGCAGGGAGAGGACCCTATAGA | |
| | GGCCTGGGACAGGAGCTCAATGAGAAAGG | |
| CD70 CAR | ATGGCGCTTCCGGTGACAGCACTGCTCCTCCCCTTGGCGCTGTTGCTCCACG | 45 |
| nucleotide | CAGCAAGGCCGCAGGTCCAGTTGGTGCAAAGCGGGGCGGAGGTGAAAAAACC | |
| sequence | CGGCGCTTCCGTGAAGGTGTCCTGTAAGGCGTCCGGTTATACGTTCACGAAC | |
| (CD70B scFv | TACGGGATGAATTGGGTTCGCCAAGCGCCGGGGCAGGGACTGAAATGGATGG | |
| with 41BB) | GGTGGATAAATACCTACACCGGCGAACCTACATACGCCGACGCTTTTAAAGG | |
| | GCGAGTCACTATGACGCGCGATACCAGCATATCCACCGCATACATGGAGCTG | |
| | TCCCGACTCCGGTCAGACGACACGGCTGTCTACTATTGTGCTCGGGACTATG | |
| | GCGATTATGGCATGGACTACTGGGGTCAGGGTACGACTGTAACAGTTAGTAG | |
| | TGGTGGAGGCGGCAGTGGCGGGGGGGGAAGCGGAGGAGGGGGTTCTGGTGAC | |
| | ATAGTTATGACCCAATCCCCAGATAGTTTGGCGGTTTCTCTGGGCGAGAGGG | |
| | CAACGATTAATTGTCGCGCATCAAAGAGCGTTTCAACGAGCGGATATTCTTT | |
| | TATGCATTGGTACCAGCAAAAACCCGGACAACCGCCGAAGCTGCTGATCTAC | |
| | TTGGCTTCAAATCTTGAGTCTGGGGTGCCGGACCGATTTTCTGGTAGTGGAA | |
| | GCGGAACTGACTTTACGCTCACGATCAGTTCACTGCAGGCTGAGGATGTAGC | |
| | GGTCTATTATTGCCAGCACAGTAGAGAAGTCCCCTGGACCTTCGGTCAAGGC | |
| | ACGAAAGTAGAAATTAAAAGTGCTGCTGCCTTTGTCCCGGTATTTCTCCCAG | |
| | CCAAACCGACCACGACTCCCGCCCCGCGCCCTCCGACACCCGCTCCCACCAT | |
| | CGCCTCTCAACCTCTTAGTCTTCGCCCCGAGGCATGCCGACCCGCCGCCGGG | |
| | GGTGCTGTTCATACGAGGGGCTTGGACTTCGCTTGTGATATTTACATTTGGG | |
| | CTCCGTTGGCGGGTACGTGCGGCGTCCTTTTGTTGTCACTCGTTATTACTTT | |
| | GTATTGTAATCACAGGAATCGCAAACGGGGCAGAAAGAAACTCCTGTATATA | |
| | TTCAAACAACCATTTATGAGACCAGTACAAACTACTCAAGAGGAAGATGGCT | |
| | GTAGCTGCCGATTTCCAGAAGAAGAAGAAGGAGGATGTGAACTGCGAGTGAA | |
| | GTTTTCCCGAAGCGCAGACGCTCCGGCATATCAGCAAGGACAGAATCAGCTG | |
| | TATAACGAACTGAATTTGGGACGCCGCGAGGAGTATGACGTGCTTGATAAAC | |
| | GCCGGGGGAGAGACCCGGAAATGGGGGGTAAACCCCGAAGAAAGAATCCCCA | |
| | AGAAGGACTCTACAATGAACTCCAGAAGGATAAGATGGCGGAGGCCTACTCA | |
| | GAAATAGGTATGAAGGGCGAACGACGACGGGGAAAAGGTCACGATGGCCTCT | |
| | ACCAAGGGTTGAGTACGGCAACCAAAGATACGTACGATGCACTGCATATGCA | |
| | GGCCCTGCCTCCCAGATAA | |
| CD70 CAR | MALPVTALLLPLALLLHAARPQVQLVQSGAEVKKPGASVKVSCKASGYTFTN | 46 |
| amino acid | YGMNWVRQAPGQGLKWMGWINTYTGEPTYADAFKGRVTMTRDTSISTAYMEL | |
| sequence | SRLRSDDTAVYYCARDYGDYGMDYWGQGTTVTVSSGGGGSGGGGSGGGGSGD | |
| (CD70B scFv | IVMTQSPDSLAVSLGERATINCRASKSVSTSGYSFMHWYQQKPGQPPKLLIY | |
| with 41BB) | LASNLESGVPDRFSGSGSGTDFTLTISSLQAEDVAVYYCQHSREVPWTFGQG | |
| | TKVEIKSAAAFVPVFLPAKPTTTPAPRPPTPAPTIASQPLSLRPEACRPAAG | |
| | GAVHTRGLDFACDIYIWAPLAGTCGVLLLSLVITLYCNHRNRKRGRKKLLYI | |
| | FKQPFMRPVQTTQEEDGCSCRFPEEEEGGCELRVKFSRSADAPAYQQGQNQL | |
| | YNELNLGRREEYDVLDKRRGRDPEMGGKPRRKNPQEGLYNELQKDKMAEAYS | |
| | EIGMKGERRRGKGHDGLYQGLSTATKDTYDALHMQALPPR | |
| CD70 CAR | QVQLVQSGAEVKKPGASVKVSCKASGYTFTNYGMNWVRQAPGQGLKWMGWIN | 81 |
| amino acid | TYTGEPTYADAFKGRVTMTRDTSISTAYMELSRLRSDDTAVYYCARDYGDYG | |
| sequence | MDYWGQGTTVTVSSGGGGSGGGGSGGGGSGDIVMTQSPDSLAVSLGERATIN | |
| (without signal | CRASKSVSTSGYSFMHWYQQKPGQPPKLLIYLASNLESGVPDRFSGSGSGTD | |
| peptide) | FTLTISSLQAEDVAVYYCQHSREVPWTFGQGTKVEIKSAAAFVPVFLPAKPT | |
| (CD70B scFv | TTPAPRPPTPAPTIASQPLSLRPEACRPAAGGAVHTRGLDFACDIYIWAPLA | |
| with 41BB) | GTCGVLLLSLVITLYCNHRNRKRGRKKLLYIFKQPFMRPVQTTQEEDGCSCR | |
| | FPEEEEGGCELRVKFSRSADAPAYQQGQNQLYNELNLGRREEYDVLDKRRGR | |
| | DPEMGGKPRRKNPQEGLYNELQKDKMAEAYSEIGMKGERRRGKGHDGLYQGL | |
| | STATKDTYDALHMQALPPR | |
| CD70B | CAGGTCCAGTTGGTGCAAAGCGGGGCGGAGGTGAAAAAACCCGGCGCTTCCG | 47 |
| scFv nucleotide | TGAAGGTGTCCTGTAAGGCGTCCGGTTATACGTTCACGAACTACGGGATGAA | |
| sequence | TTGGGTTCGCCAAGCGCCGGGGCAGGGACTGAAATGGATGGGGTGGATAAAT | |
| | ACCTACACCGGCGAACCTACATACGCCGACGCTTTTAAAGGGCGAGTCACTA | |
| | TGACGCGCGATACCAGCATATCCACCGCATACATGGAGCTGTCCCGACTCCG | |
| | GTCAGACGACACGGCTGTCTACTATTGTGCTCGGGACTATGGCGATTATGGC | |
| | ATGGACTACTGGGGTCAGGGTACGACTGTAACAGTTAGTAGTGGTGGAGGCG | |
| | GCAGTGGCGGGGGGGGAAGCGGAGGAGGGGGTTCTGGTGACATAGTTATGAC | |
| | CCAATCCCCAGATAGTTTGGCGGTTTCTCTGGGCGAGAGGGCAACGATTAAT | |
| | TGTCGCGCATCAAAGAGCGTTTCAACGAGCGGATATTCTTTTATGCATTGGT | |
| | ACCAGCAAAAACCCGGACAACCGCCGAAGCTGCTGATCTACTTGGCTTCAAA | |
| | TCTTGAGTCTGGGGTGCCGGACCGATTTTCTGGTAGTGGAAGCGGAACTGAC | |
| | TTTACGCTCACGATCAGTTCACTGCAGGCTGAGGATGTAGCGGTCTATTATT | |
| | GCCAGCACAGTAGAGAAGTCCCCTGGACCTTCGGTCAAGGCACGAAAGTAGA | |
| | AATTAAA | |
| CD70B | QVQLVQSGAEVKKPGASVKVSCKASGYTFTNYGMNWVRQAPGQGLKWMGWIN | 48 |
| scFv amino acid | TYTGEPTYADAFKGRVTMTRDTSISTAYMELSRLRSDDTAVYYCARDYGDYG | |
| sequence | MDYWGQGTTVTVSSGGGGSGGGGSGGGGSGDIVMTQSPDSLAVSLGERATIN | |
| (linker | CRASKSVSTSGYSFMHWYQQKPGQPPKLLIYLASNLESGVPDRFSGSGSGTD | |
| underlined) | FTLTISSLQAEDVAVYYCQHSREVPWTFGQGTKVEIK | |
| CD70 V_{H} | QVQLVQSGAEVKKPGASVKVSCKASGYTFTNYGMNWVRQAPGQGLKWMGWIN | 49 |
| | TYTGEPTYADAFKGRVTMTRDTSISTAYMELSRLRSDDTAVYYCARDYGDYG | |
| | MDYWGQGTTVTVSS | |
| CD70 V_{L} | DIVMTQSPDSLAVSLGERATINCRASKSVSTSGYSFMHWYQQKPGQPPKLLI | 50 |
| | YLASNLESGVPDRFSGSGSGTDFTLTISSLQAEDVAVYYCQHSREVPWTFGQ | |
| | GTKVEIK | |
| Linker | GGGGSGGGGSGGGGSG | 51 |
| signal peptide | MLLLVTSLLLCELPHPAFLLIP | 52 |
| signal peptide | MALPVTALLLPLALLLHAARP | 53 |
| CD8a | FVPVFLPAKPTTTPAPRPPTPAPTIASQPLSLRPEACRPAAGGAVHTRGLDF | 54 |
| transmembrane | ACDIYIWAPLAGTCGVLLLSLVITLYCNHRNR | |
| domain | | |
| CD8a | IYIWAPLAGTCGVLLLSLVITLY | 55 |
| transmembrane | | |
| 4-1BB | AAACGGGGCAGAAAGAAACTCCTGTATATATTCAAACAACCATTTATGAGAC | 56 |
| nucleotide | CAGTACAAACTACTCAAGAGGAAGATGGCTGTAGCTGCCGATTTCCAGAAGA | |
| sequence | AGAAGAAGGAGGATGTGAACTG | |
| 4-1BB amino | KRGRKKLLYIFKQPFMRPVQTTQEEDGCSCRFPEEEEGGCEL | 57 |
| acid sequence | | |
| CD28 nucleotide | TCAAAGCGGAGTAGGTTGTTGCATTCCGATTACATGAATATGACTCCTCGCC | 58 |
| sequence | GGCCTGGGCCGACAAGAAAACATTACCAACCCTATGCCCCCCCACGAGACTT | |
| | CGCTGCGTACAGGTCC | |
| CD28 amino | SKRSRLLHSDYMNMTPRRPGPTRKHYQPYAPPRDFAAYRS | 59 |
| acid sequence | | |
| CD3ζ nucleotide | CGAGTGAAGTTTTCCCGAAGCGCAGACGCTCCGGCATATCAGCAAGGACAGA | 60 |
| sequence | ATCAGCTGTATAACGAACTGAATTTGGGACGCCGCGAGGAGTATGACGTGCT | |
| | TGATAAACGCCGGGGGAGAGACCCGGAAATGGGGGGTAAACCCCGAAGAAAG | |
| | AATCCCCAAGAAGGACTCTACAATGAACTCCAGAAGGATAAGATGGCGGAGG | |
| | CCTACTCAGAAATAGGTATGAAGGGCGAACGACGACGGGGAAAAGGTCACGA | |
| | TGGCCTCTACCAAGGGTTGAGTACGGCAACCAAAGATACGTACGATGCACTG | |
| | CATATGCAGGCCCTGCCTCCCAGA | |
| CD3ζ amino acid | RVKFSRSADAPAYQQGQNQLYNELNLGRREEYDVLDKRRGRDPEMGGKPRRK | 61 |
| sequence | NPQEGLYNELQKDKMAEAYSEIGMKGERRRGKGHDGLYQGLSTATKDTYDAL | |
| | HMQALPPR | |
| TRAC-LHA | GAGATGTAAGGAGCTGCTGTGACTTGCTCAAGGCCTTATATCGAGTAAACGG | 62 |
| | TAGTGCTGGGGCTTAGACGCAGGTGTTCTGATTTATAGTTCAAAACCTCTAT | |
| | CAATGAGAGAGCAATCTCCTGGTAATGTGATAGATTTCCCAACTTAATGCCA | |
| | ACATACCATAAACCTCCCATTCTGCTAATGCCCAGCCTAAGTTGGGGAGACC | |
| | ACTCCAGATTCCAAGATGTACAGTTTGCTTTGCTGGGCCTTTTTCCCATGCC | |
| | TGCCTTTACTCTGCCAGAGTTATATTGCTGGGGTTTTGAAGAAGATCCTATT | |
| | AAATAAAAGAATAAGCAGTATTATTAAGTAGCCCTGCATTTCAGGTTTCCTT | |
| | GAGTGGCAGGCCAGGCCTGGCCGTGAACGTTCACTGAAATCATGGCCTCTTG | |
| | GCCAAGATTGATAGCTTGTGCCTGTCCCTGAGTCCCAGTCCATCACGAGCAG | |
| | CTGGTTTCTAAGATGCTATTTCCCGTATAAAGCATGAGACCGTGACTTGCCA | |
| | GCCCCACAGAGCCCCGCCCTTGTCCATCACTGGCATCTGGACTCCAGCCTGG | |
| | GTTGGGGCAAAGAGGGAAATGAGATCATGTCCTAACCCTGATCCTCTTGTCC | |
| | CACAGATATCCAGAACCCTGACCCTGCCGTGTACCAGCTGAGAGACTCTAAA | |
| | TCCAGTGACAAGTCTGTCTGCCTATTCACCGATTTTGATTCTCAAACAAATG | |
| | TGTCACAAAGTAAGGATTCTGATGTGTATATCACAGACAAAACTGTGCTAGA | |
| | CATGAGGTCTATGGACTTCA | |
| EF1α promoter | GGCTCCGGTGCCCGTCAGTGGGCAGAGCGCACATCGCCCACAGTCCCCGAGA | 63 |
| | AGTTGGGGGGAGGGGTCGGCAATTGAACCGGTGCCTAGAGAAGGTGGCGCGG | |
| | GGTAAACTGGGAAAGTGATGTCGTGTACTGGCTCCGCCTTTTTCCCGAGGGT | |
| | GGGGGAGAACCGTATATAAGTGCAGTAGTCGCCGTGAACGTTCTTTTTCGCA | |
| | ACGGGTTTGCCGCCAGAACACAGGTAAGTGCCGTGTGTGGTTCCCGCGGGCC | |
| | TGGCCTCTTTACGGGTTATGGCCCTTGCGTGCCTTGAATTACTTCCACTGGC | |
| | TGCAGTACGTGATTCTTGATCCCGAGCTTCGGGTTGGAAGTGGGTGGGAGAG | |
| | TTCGAGGCCTTGCGCTTAAGGAGCCCCTTCGCCTCGTGCTTGAGTTGAGGCC | |
| | TGGCCTGGGCGCTGGGGCCGCCGCGTGCGAATCTGGTGGCACCTTCGCGCCT | |
| | GTCTCGCTGCTTTCGATAAGTCTCTAGCCATTTAAAATTTTTGATGACCTGC | |
| | TGCGACGCTTTTTTTCTGGCAAGATAGTCTTGTAAATGCGGGCCAAGATCTG | |
| | CACACTGGTATTTCGGTTTTTGGGGCCGCGGGCGGCGACGGGGCCCGTGCGT | |
| | CCCAGCGCACATGTTCGGCGAGGCGGGGCCTGCGAGCGCGGCCACCGAGAAT | |
| | CGGACGGGGGTAGTCTCAAGCTGGCCGGCCTGCTCTGGTGCCTGGCCTCGCG | |
| | CCGCCGTGTATCGCCCCGCCCTGGGCGGCAAGGCTGGCCCGGTCGGCACCAG | |
| | TTGCGTGAGCGGAAAGATGGCCGCTTCCCGGCCCTGCTGCAGGGAGCTCAAA | |
| | ATGGAGGACGCGGCGCTCGGGAGAGCGGGCGGGTGAGTCACCCACACAAAGG | |
| | AAAAGGGCCTTTCCGTCCTCAGCCGTCGCTTCATGTGACTCCACGGAGTACC | |
| | GGGCGCCGTCCAGGCACCTCGATTAGTTCTCGAGCTTTTGGAGTACGTCGTC | |
| | TTTAGGTTGGGGGGAGGGGTTTTATGCGATGGAGTTTCCCCACACTGAGTGG | |
| | GTGGAGACTGAAGTTAGGCCAGCTTGGCACTTGATGTAATTCTCCTTGGAAT | |
| | TTGCCCTTTTTGAGTTTGGATCTTGGTTCATTCTCAAGCCTCAGACAGTGGT | |
| | TCAAAGTTTTTTTCTTCCATTTCAGGTGTCGTGA | |
| Synthetic | AATAAAATCGCTATCCATCGAAGATGGATGTGTGTTGGTTTTTTGTGTG | 64 |
| poly(A) signal | | |
| TRAC-RHA | TGGAGCAACAAATCTGACTTTGCATGTGCAAACGCCTTCAACAACAGCATTA | 65 |
| | TTCCAGAAGACACCTTCTTCCCCAGCCCAGGTAAGGGCAGCTTTGGTGCCTT | |
| | CGCAGGCTGTTTCCTTGCTTCAGGAATGGCCAGGTTCTGCCCAGAGCTCTGG | |
| | TCAATGATGTCTAAAACTCCTCTGATTGGTGGTCTCGGCCTTATCCATTGCC | |
| | ACCAAAACCCTCTTTTTACTAAGAAACAGTGAGCCTTGTTCTGGCAGTCCAG | |
| | AGAATGACACGGGAAAAAAGCAGATGAAGAGAAGGTGGCAGGAGAGGGCACG | |
| | TGGCCCAGCCTCAGTCTCTCCAACTGAGTTCCTGCCTGCCTGCCTTTGCTCA | |
| | GACTGTTTGCCCCTTACTGCTCTTCTAGGCCTCATTCTAAGCCCCTTCTCCA | |
| | AGTTGCCTCTCCTTATTTCTCCCTGTCTGCCAAAAAATCTTTCCCAGCTCAC | |
| | TAAGTCAGTCTCACGCAGTCACTCATTAACCCACCAATCACTGATTGTGCCG | |
| | GCACATGAATGCACCAGGTGTTGAAGTGGAGGAATTAAAAAGTCAGATGAGG | |
| | GGTGTGCCCAGAGGAAGCACCATTCTAGTTGGGGGAGCCCATCTGTCAGCTG | |
| | GGAAAAGTCCAAATAACTTCAGATTGGAATGTGTTTTAACTCAGGGTTGAGA | |
| | AAACAGCTACCTTCAGGACAAAAGTCAGGGAAGGGCTCTCTGAAGAAATGCT | |
| | ACTTGAAGATACCAGCCCTACCAAGGGCAGGGAGAGGACCCTATAGAGGCCT | |
| | GGGACAGGAGCTCAATGAGAAAGG | |

(ii) Knock-Out of TRAC, B2M, and/or CD70 Genes

The anti-CD70 CAR-T cells disclosed herein may further have a disrupted TRAC gene, a disrupted B2M gene, a disrupted CD70 gene, or a combination thereof. The disruption of the TRAC locus results in loss of expression of the T cell receptor (TCR) and is intended to reduce the probability of Graft versus Host Disease (GvHD), while the disruption of the β2M locus results in lack of expression of the major histocompatibility complex type I (MHC I) proteins and is intended to improve persistence by reducing the probability of host rejection. The disruption of the CD70 gene would minimize the fratricide effect in producing the anti-CD70 CAR-T cells. Further, disruption of the CD70 gene unexpectedly increased healthy and activity of the resultant engineered T cells. The addition of the anti-CD70 CAR directs the modified T cells towards CD70-expressing tumor cells.

As used herein, the term “a disrupted gene” refers to a gene containing one or more mutations (e.g., insertion, deletion, or nucleotide substitution, etc.) relative to the wild-type counterpart so as to substantially reduce or completely eliminate the activity of the encoded gene product. The one or more mutations may be located in a non-coding region, for example, a promoter region, a regulatory region that regulates transcription or translation; or an intron region. Alternatively, the one or more mutations may be located in a coding region (e.g., in an exon). In some instances, the disrupted gene does not express or expresses a substantially reduced level of the encoded protein. In other instances, the disrupted gene expresses the encoded protein in a mutated form, which is either not functional or has substantially reduced activity. In some embodiments, a disrupted gene is a gene that does not encode functional protein. In some embodiments, a cell that comprises a disrupted gene does not express (e.g., at the cell surface) a detectable level (e.g. by antibody, e.g., by flow cytometry) of the protein encoded by the gene. A cell that does not express a detectable level of the protein may be referred to as a knockout cell. For example, a cell having a β2M gene edit may be considered a β2M knockout cell if β2M protein cannot be detected at the cell surface using an antibody that specifically binds β2M protein.

In some embodiments, a disrupted gene may be described as comprising a mutated fragment relative to the wild-type counterpart. The mutated fragment may comprise a deletion, a nucleotide substitution, an addition, or a combination thereof. In other embodiments, a disrupted gene may be described as having a deletion of a fragment that is present in the wild-type counterpart. In some instances, the 5′ end of the deleted fragment may be located within the gene region targeted by a designed guide RNA such as those disclosed herein (known as on-target sequence) and the 3′ end of the deleted fragment may go beyond the targeted region. Alternatively, the 3′ end of the deleted fragment may be located within the targeted region and the 5′ end of the deleted fragment may go beyond the targeted region.

In some instances, the disrupted TRAC gene in the anti-CD70 CAR-T cells disclosed herein may comprise a deletion, for example, a deletion of a fragment in Exon 1 of the TRAC gene locus. In some examples, the disrupted TRAC gene comprises a deletion of a fragment comprising the nucleotide sequence of SEQ ID NO: 17, which is the target site of TRAC guide RNA TA-1. See sequence tables below. In some examples, the fragment of SEQ ID NO: 17 may be replaced by a nucleic acid encoding the anti-CD70 CAR. Such a disrupted TRAC gene may comprise the nucleotide sequence of SEQ ID NO: 44.

The disrupted B2M gene in the anti-CD70 CAR-T cells disclosed herein may be generated using the CRISPR/Cas technology. In some examples, a B2M gRNA provided in the sequence table below can be used. The disrupted B2M gene may comprise a nucleotide sequence of any one of SEQ ID Nos: 31-36. See Table 4 below.

Alternatively or in addition, the disrupted CD70 gene in the anti-CD70 CAR-T cells disclosed herein may be generated using the CRISPR/Cas technology. In some examples, a CD70 gRNA provided in the sequence table below can be used. The disrupted CD70 gene may comprise a nucleotide sequence of any one of SEQ ID NOs:37-42. See Table 5 below.

(iii) Exemplary Anti-CD70 CAR T Cells

In some examples, the anti-CD70 CAR T cells are CTX130 cells, which are CD70-directed T cells having disrupted TRAC gene, B2M gene, and CD70 gene. CTX130 cells can be produced via ex vivo genetic modification using CRISPR/Cas9 (Clustered Regularly Interspaced Short Palindromic Repeats/CRISPR associated protein 9) gene editing components (sgRNAs and Cas9 nuclease).

Also within the scope of the present disclosure are populations of anti-CD70 CAR T cells (e.g., a population of CTX130 cells), which comprises genetically engineered cells (e.g., CRISPR-Cas9-mediated gene edited) expressing the anti-CD70 CAR disclosed herein and disrupted TRAC, B2M, and CD70 genes; and the nucleotide sequence encoding the anti-CD70 CAR is inserted into the TRAC locus.

It should be understood that gene disruption encompasses gene modification through gene editing (e.g., using CRISPR/Cas gene editing to insert or delete one or more nucleotides). As used herein, the term “a disrupted gene” refers to a gene containing one or more mutations (e.g., insertion, deletion, or nucleotide substitution, etc.) relative to the wild-type counterpart so as to substantially reduce or completely eliminate the activity of the encoded gene product. The one or more mutations may be located in a non-coding region, for example, a promoter region, a regulatory region that regulates transcription or translation; or an intron region. Alternatively, the one or more mutations may be located in a coding region (e.g., in an exon). In some instances, the disrupted gene does not express or expresses a substantially reduced level of the encoded protein. In other instances, the disrupted gene expresses the encoded protein in a mutated form, which is either not functional or has substantially reduced activity. In some embodiments, a disrupted gene is a gene that does not encode functional protein. In some embodiments, a cell that comprises a disrupted gene does not express (e.g., at the cell surface) a detectable level (e.g., by antibody, e.g., by flow cytometry) of the protein encoded by the gene. A cell that does not express a detectable level of the protein may be referred to as a knockout cell. For example, a cell having a β2M gene edit may be considered a β2M knockout cell if β2M protein cannot be detected at the cell surface using an antibody that specifically binds β2M protein.

In specific instances, the anti-CD70 CAR+ T cells are CTX130 cells, which are produced using CRISPR technology to disrupt targeted genes, and adeno-associated virus (AAV) transduction to deliver the CAR construct. CRISPR-Cas9-mediated gene editing involves three guide RNAs (sgRNAs): CD70-7 sgRNA (SEQ ID NO: 2) which targets the CD70 locus, TA-1 sgRNA (SEQ ID NO: 6) which targets the TRAC locus, and B2M-1 sgRNA (SEQ ID NO: 10) which targets the β2M locus. The anti-CD70 CAR of CTX130 cells is composed of an anti-CD70 single-chain antibody fragment (scFv) specific for CD70, followed by a CD8 hinge and transmembrane domain that is fused to an intracellular co-signaling domain of 4-1BB and a CD3t signaling domain. As such, CTX130 is a CD70-directed T cell immunotherapy comprised of allogeneic T cells that are genetically modified ex vivo using CRISPR/Cas9 gene editing components (sgRNA and Cas9 nuclease).

In some embodiments, at least 50% of a population of CTX130 cells may not express a detectable level of 132M surface protein. For example, at least 55%, at least 60%, at least 70%, at least 75%, at least 80%, at least 85%, at least 90%, or at least 95% of the engineered T cells of a population may not express a detectable level of β2M surface protein. In some embodiments, 50%-100%, 50%-90%, 50%-80%, 50%-70%, 50%-60%, 60%-100%, 60%-90%, 60%-80%, 60%-70%, 70%-100%, 70%-90%, 70%-80%, 80%-100%, 80%-90%, or 90%-100% of the engineered T cells of a population does not express a detectable level of β2M surface protein.

Alternatively or in addition, at least 50% of a population of CTX130 cells may not express a detectable level of TRAC surface protein. For example, at least 55%, at least 60%, at least 70%, at least 75%, at least 80%, at least 85%, at least 90%, or at least 95% of the engineered T cells of a population may not express a detectable level of TRAC surface protein. In some embodiments, 50%-100%, 50%-90%, 50%-80%, 50%-70%, 50%-60%, 60%-100%, 60%-90%, 60%-80%, 60%-70%, 70%-100%, 70%-90%, 70%-80%, 80%-100%, 80%-90%, or 90%-100% of the engineered T cells of a population does not express a detectable level of TRAC surface protein.

In some embodiments, at least 50% of a population of CTX130 cells may not express a detectable level of CD70 surface protein. For example, at least 55%, at least 60%, at least 70%, at least 75%, at least 80%, at least 85%, at least 90%, at least 95%, or at least 98% of the engineered T cells of a population may not express a detectable level of CD70 surface protein. In some embodiments, 50%-100%, 50%-90%, 50%-80%, 50%-70%, 50%-60%, 60%-100%, 60%-90%, 60%-80%, 60%-70%, 70%-100%, 70%-90%, 70%-80%, 80%-100%, 80%-90%, 90%-100%, or 95%-100% of the engineered T cells of a population does not express a detectable level of CD70 surface protein.

In some embodiments, a substantial percentage of the population of CTX130 cells may comprise more than one gene edit, which results in a certain percentage of cells not expressing more than one gene and/or protein.

For example, at least 50% of a population of CTX130 cells may not express a detectable level of two surface proteins, e.g., does not express a detectable level of β2M and TRAC proteins, β2M and CD70 proteins, or TRAC and CD70 proteins. In some embodiments, 50%-100%, 50%-90%, 50%-80%, 50%-70%, 50%-60%, 60%-100%, 60%-90%, 60%-80%, 60%-70%, 70%-100%, 70%-90%, 70%-80%, 80%-100%, 80%-90%, or 90%-100% of the engineered T cells of a population does not express a detectable level of two surface proteins. In another example, at least 50% of a population of the CTX130 cells may not express a detectable level of all of the three target surface proteins β2M, TRAC, and CD70 proteins. In some embodiments, 50%-100%, 50%-90%, 50%-80%, 50%-70%, 50%-60%, 60%-100%, 60%-90%, 60%-80%, 60%-70%, 70%-100%, 70%-90%, 70%-80%, 80%-100%, 80%-90%, or 90%-100% of the engineered T cells of a population does not express a detectable level of β2M, TRAC, and CD70 surface proteins.

In some embodiments, the population of CTX130 cells may comprise more than one gene edit (e.g., in more than one gene), which may be an edit described herein. For example, the population of CTX130 cells may comprise a disrupted TRAC gene via the CRISPR/Cas technology using guide RNA TA-1 (see also Table 2, SEQ ID NOS: 6-7). Alternatively or in addition, the population of CTX130 cells may comprise a disrupted β2M gene via CRISPR/Cas9 technology using the guide RNA of B2M-1 (see also Table 2, SEQ ID NOS: 10-11). Such CTX130 cells may comprise Indels in the β2M gene, which comprise one or more of the nucleotide sequences listed in Table 4. For example, the population of CTX130 cells may comprise a disrupted CD70 gene via the CRISPR/Cas technology using guide RNA CD70-7 (see also Table 2, SEQ ID NOS: 2-3). Further, the population of the CTX130 cells may comprise Indels in the CD70 gene, which may comprise one or more nucleotide sequences listed in Table 5.

In some embodiments, the CTX130 cells may comprise a deletion in the TRAC gene relative to unmodified T cells. For example, the CTX130 cells may comprise a deletion of the fragment AGAGCAACAGTGCTGTGGCC (SEQ ID NO: 17) in the TRAC gene, or a portion of thereof, e.g., a fragment of SEQ ID NO: 17 comprising 1, 2, 3, 4, 5, 6, 7, 8, 9, 10, 11, 12, 13, 14, 15, 16, 17, 18, or 19 consecutive base pairs. In some embodiments, the CTX130 cells include a deletion comprising the fragment of SEQ ID NO: 17 in the TRAC gene. In some embodiments, an engineered T cell comprises a deletion of SEQ ID NO: 17 in the TRAC gene relative to unmodified T cells. In some embodiments, an engineered T cell comprises a deletion comprising SEQ ID NO: 17 in the TRAC gene relative to unmodified T cells.

Further, the population of CTX130 cells may comprise cells expressing an anti-CD70 CAR such as those disclosed herein (e.g., SEQ ID NO: 46 or SEQ ID NO: 81). The coding sequence of the anti-CD70 CAR may be inserted into the TRAC locus, e.g., at the region targeted by guide RNA TA-1 (see also Table 2, SEQ ID NOS: 6-7). In such instances, the amino acid sequence of the exemplary anti-CD70 CAR comprises the amino acid sequence of SEQ ID NO:46.

In some embodiments, at least 30% at least 35%, at least 40%, at least 45%, at least 50%, at least 55%, at least 60%, at least 65%, at least 70%, at least 75%, at least 80%, at least 85%, at least 90%, at least 95%, at least 98%, at least 99%, or 100% of the CTX130 cells are CAR+ cells, which express the anti-CD70 CAR. See also WO 2019/097305A2, and WO2019/215500, the relevant disclosures of each of which are incorporated by reference for the subject matter and purpose referenced herein.

In specific examples, the anti-CD70 CAR-T cells disclosed herein (e.g., CTX130 cells) is a population of T cells having ≥30% CAR+ T cells, ≤0.4% TCR+ T cells, ≤30% B2M+ T cells, and ≤2% CD70+ T cells.

### (v) Pharmaceutical Compositions

In some aspects, the present disclosure provides pharmaceutical compositions comprising any of the populations of genetically engineered anti-CD70 CAR T cells as disclosed herein, for example, CTX130 cells, and a pharmaceutically acceptable carrier. Such pharmaceutical compositions can be used in cancer treatment in human patients, which is also disclosed herein.

As used herein, the term “pharmaceutically acceptable” refers to those compounds, materials, compositions, and/or dosage forms which are, within the scope of sound medical judgment, suitable for use in contact with the tissues, organs, and/or bodily fluids of the subject without excessive toxicity, irritation, allergic response, or other problems or complications commensurate with a reasonable benefit/risk ratio. As used herein, the term “pharmaceutically acceptable carrier” refers to solvents, dispersion media, coatings, antibacterial agents, antifungal agents, isotonic and absorption delaying agents, or the like that are physiologically compatible. The compositions can include a pharmaceutically acceptable salt, e.g., an acid addition salt or a base addition salt. See, e.g., Berge et al., (1977) J Pharm Sci 66:1-19.

In some embodiments, the pharmaceutical composition further comprises a pharmaceutically acceptable salt. Non-limiting examples of pharmaceutically acceptable salts include acid addition salts (formed from a free amino group of a polypeptide with an inorganic acid (e.g., hydrochloric or phosphoric acids), or an organic acid such as acetic, tartaric, mandelic, or the like). In some embodiments, the salt formed with the free carboxyl groups is derived from an inorganic base (e.g., sodium, potassium, ammonium, calcium or ferric hydroxides), or an organic base such as isopropylamine, trimethylamine, 2-ethylamino ethanol, histidine, procaine, or the like).

In some embodiments, the pharmaceutical composition disclosed herein comprises a population of the genetically engineered anti-CD70 CAR-T cells (e.g., CTX130 cells) suspended in a cryopreservation solution (e.g., CryoStor® C55). The cryopreservation solution for use in the present disclosure may also comprise adenosine, dextrose, dextran-40, lactobionic acid, sucrose, mannitol, a buffer agent such as N-)2-hydroxethyl) piperazine-N′-(2-ethanesulfonic acid) (HEPES), one or more salts (e.g., calcium chloride, magnesium chloride, potassium chloride, potassium bicarbonate, potassium phosphate, etc.), one or more base (e.g., sodium hydroxide, potassium hydroxide, etc.), or a combination thereof. Components of a cryopreservation solution may be dissolved in sterile water (injection quality). Any of the cryopreservation solution may be substantially free of serum (undetectable by routine methods).

In some instances, a pharmaceutical composition comprising a population of genetically engineered anti-CD70 CAR-T cells such as the CTX130 cells suspended in a cryopreservation solution (e.g., substantially free of serum) may be placed in storage vials.

Any of the pharmaceutical compositions disclosed herein, comprising a population of genetically engineered anti-CD70 CAR T cells as also disclosed herein (e.g., CTX130 cells), which optionally may be suspended in a cryopreservation solution as disclosed herein may be stored in an environment that does not substantially affect viability and bioactivity of the T cells for future use, e.g., under conditions commonly applied for storage of cells and tissues. In some examples, the pharmaceutical composition may be stored in the vapor phase of liquid nitrogen at ≤−135° C. No significant changes were observed with respect to appearance, cell count, viability, % CAR⁺ T cells, % TCR⁺ T cells, % B2M⁺ T cells, and % CD70⁺ T cells after the cells have been stored under such conditions for a period of time.

In some embodiments, the pharmaceutical composition disclosed herein can be a suspension for infusion, comprising the anti-CD70 CAR T cells disclosed herein such as the CTX130 cells. In some examples, the suspension may comprise about 25-85×10⁶ cells/ml (e.g., 50×10⁶ cells/ml) with ≥30% CAR+ T cells, ≤0.4% TCR+ T cells, ≤30% B2M+ T cells, and ≤2% CD70+ T cells. In some examples, the suspension may comprise about 25×10⁶ CAR+ cells/mL. In specific examples, the pharmaceutical composition may be placed in a vial, each comprising about 1.5×10⁸ CAR+ T cells such as CTX130 cells (e.g., viable cells). In other examples, the pharmaceutical composition may be placed in a vial, each comprising about 3×10⁸ CAR+ T cells such as CTX130 cells (e.g., viable cells).

### II. Preparation of Anti-CD70 CAR T Cells

Any suitable gene editing methods known in the art can be used for making the genetically engineered immune cells (e.g., T cells such as CTX130 cells) disclosed herein, for example, nuclease-dependent targeted editing using zinc-finger nucleases (ZFNs), transcription activator-like effector nucleases (TALENs), or RNA-guided CRISPR-Cas9 nucleases (CRISPR/Cas9; Clustered Regular Interspaced Short Palindromic Repeats Associated 9). In specific examples, the genetically engineered immune cells such as CTX130 cells are produced by the CRISPR technology in combination with homologous recombination using an adeno-associated viral vector (AAV) as a donor template.

(i) Sources of T Cells

In some embodiments, primary T cells isolated from one or more donors may be used for making the genetically engineered anti-CD70 CAR-T cells. For example, primary T cells may be isolated from a suitable tissue of one or more healthy human donors, e.g., peripheral blood mononuclear cells (PBMCs), bone marrow, lymph nodes tissue, cord blood, thymus issue, tissue from a site of infection, ascites, pleural effusion, spleen tissue, or a combination thereof. In some embodiments, a subpopulation of primary T cells expressing TCRαβ, CD3, CD4, CD8, CD27 CD28, CD38, CD45RA, CD45RO, CD62L, CD127, CD122, CD95, CD197, CCR7, KLRG1, MHC-I proteins, MHC-II proteins, or a combination thereof may be further enriched, using a positive or negative selection technique, which is known in the art. In some embodiments, the T cell subpopulation express TCRαβ, CD4, CD8, or a combination thereof. In some embodiments, the T cell subpopulation express CD3, CD4, CD8, or a combination thereof. In some embodiments, the primary T cells for use in making the genetic edits disclosed herein may comprise at least 40%, at least 50%, or at least 60% CD27+CD45RO− T cells.

In other embodiments, the T cells for use in generating the genetically engineered T cells disclosed herein may be derived from a T cell bank. A T cell bank may comprise T cells with genetic editing of certain genes (e.g., genes involved in cell self renewal, apoptosis, and/or T cell exhaustion or replicative senescence) to improve T cell persistence in cell culture. A T cell bank may be produced from bona fide T cells, for example, non-transformed T cells, terminally differentiated T cells, T cells having stable genome, and/or T cells that depend on cytokines and growth factors for proliferation and expansion. Alternatively, such a T cell bank may be produced from precursor cells such as hematopoietic stem cells (e.g., iPSCs), e.g., in vitro culture. In some examples, the T cells in the T cell bank may comprise genetic editing of one or more genes involved in cell self-renewal, one or more genes involved in apoptosis, and/or one or more genes involved in T cell exhaustion, so as to disrupt or reduce expression of such genes, leading to improved persistence in culture. Examples of the edited genes in a T cell bank include, but are not limited to, Tet2, Fas, CD70, Reg1, or a combination thereof. Compared with the non-edited T counterpart, T cells in a T cell bank may have enhanced expansion capacity in culture, enhanced proliferation capacity, greater T cell activation, and/or reduced apoptosis levels. Additional information of T cell bank may be found in International Application No. PCT/IB2020/058280, the relevant disclosures of which are incorporated by reference for the subject matter and purpose referenced herein.

In some embodiments, parent T cells for use in making the genetically engineered CAR T cells (e.g., any of the T cells derived from primary T cell sources) may be undergone one or more rounds of stimulation, activation, expansion, or a combination thereof. In some embodiments, the parent T cells are activated and stimulated to proliferate in vitro before gene editing. In some embodiments, the T cells are activated, expanded, or both, before or after gene editing. In some embodiments, the T cells are activated and expanded at the same time as gene editing. In some embodiments, the T cells are activated and expanded for about 1-4 days, e.g., about 1-3 days, about 1-2 days, about 2-3 days, about 2-4 days, about 3-4 days, about 1 day, about 2 days, about 3 days, or about 4 days. In some embodiments, the allogeneic T cells are activated and expanded for about 4 hours, about 6 hours, about 12 hours, about 18 hours, about 24 hours, about 36 hours, about 48 hours, about 60 hours, or about 72 hours. Non-limiting examples of methods to activate and/or expand T cells are described in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; and 6,867,041.

(ii) CRISPR-Cas9-Mediated Gene Editing System for Genetic Engineering of T Cells

The CRISPR-Cas9 system is a naturally-occurring defense mechanism in prokaryotes that has been repurposed as an RNA-guided DNA-targeting platform used for gene editing. It relies on the DNA nuclease Cas9, and two noncoding RNAs, crisprRNA (crRNA) and trans-activating RNA (tracrRNA), to target the cleavage of DNA. CRISPR is an abbreviation for Clustered Regularly Interspaced Short Palindromic Repeats, a family of DNA sequences found in the genomes of bacteria and archaea that contain fragments of DNA (spacer DNA) with similarity to foreign DNA previously exposed to the cell, for example, by viruses that have infected or attacked the prokaryote. These fragments of DNA are used by the prokaryote to detect and destroy similar foreign DNA upon re-introduction, for example, from similar viruses during subsequent attacks. Transcription of the CRISPR locus results in the formation of an RNA molecule comprising the spacer sequence, which associates with and targets Cas (CRISPR-associated) proteins able to recognize and cut the foreign, exogenous DNA. Numerous types and classes of CRISPR/Cas systems have been described (see, e.g., Koonin et al., (2017) Curr Opin Microbiol 37:67-78).

crRNA drives sequence recognition and specificity of the CRISPR-Cas9 complex through Watson-Crick base pairing typically with a 20 nucleotide (nt) sequence in the target DNA. Changing the sequence of the 5′ 20 nt in the crRNA allows targeting of the CRISPR-Cas9 complex to specific loci. The CRISPR-Cas9 complex only binds DNA sequences that contain a sequence match to the first 20 nt of the crRNA, if the target sequence is followed by a specific short DNA motif (with the sequence NGG) referred to as a protospacer adjacent motif (PAM).

TracrRNA hybridizes with the 3′ end of crRNA to form an RNA-duplex structure that is bound by the Cas9 endonuclease to form the catalytically active CRISPR-Cas9 complex, which can then cleave the target DNA.

Once the CRISPR-Cas9 complex is bound to DNA at a target site, two independent nuclease domains within the Cas9 enzyme each cleave one of the DNA strands upstream of the PAM site, leaving a double-strand break (DSB) where both strands of the DNA terminate in a base pair (a blunt end).

After binding of CRISPR-Cas9 complex to DNA at a specific target site and formation of the site-specific DSB, the next key step is repair of the DSB. Cells use two main DNA repair pathways to repair the DSB: non-homologous end joining (NHEJ) and homology-directed repair (HDR).

NHEJ is a robust repair mechanism that appears highly active in the majority of cell types, including non-dividing cells. NHEJ is error-prone and can often result in the removal or addition of between one and several hundred nucleotides at the site of the DSB, though such modifications are typically <20 nt. The resulting insertions and deletions (indels) can disrupt coding or noncoding regions of genes. Alternatively, HDR uses a long stretch of homologous donor DNA, provided endogenously or exogenously, to repair the DSB with high fidelity. HDR is active only in dividing cells, and occurs at a relatively low frequency in most cell types. In many embodiments of the present disclosure, NHEJ is utilized as the repair operant.

(a) Cas9

In some embodiments, the Cas9 (CRISPR associated protein 9) endonuclease is used in a CRISPR method for making the genetically engineered T cells as disclosed herein. The Cas9 enzyme may be one from Streptococcus pyogenes, although other Cas9 homologs may also be used. It should be understood, that wild-type Cas9 may be used or modified versions of Cas9 may be used (e.g., evolved versions of Cas9, or Cas9 orthologues or variants), as provided herein. In some embodiments, Cas9 comprises a Streptococcus pyogenes-derived Cas9 nuclease protein that has been engineered to include C- and N-terminal SV40 large T antigen nuclear localization sequences (NLS). The resulting Cas9 nuclease (sNLS-spCas9-sNLS) is a 162 kDa protein that is produced by recombinant E. coli fermentation and purified by chromatography. The spCas9 amino acid sequence can be found as UniProt Accession No. Q99ZW2, which is provided herein as SEQ ID NO: 1.

Amino acid sequence of Cas9 nuclease
(SEQ ID NO: 1):
MDKKYSIGLDIGTNSVGWAVITDEYKVPSKKFKVLGNTDRHSIKKNLIGAL
LFDSGETAEATRLKRTARRRYTRRKNRICYLQEIFSNEMAKVDDSFFHRLE
ESFLVEEDKKHERHPIFGNIVDEVAYHEKYPTIYHLRKKLVDSTDKADLRL
IYLALAHMIKFRGHFLIEGDLNPDNSDVDKLFIQLVQTYNQLFEENPINAS
GVDAKAILSARLSKSRRLENLIAQLPGEKKNGLFGNLIALSLGLTPNFKSN
FDLAEDAKLQLSKDTYDDDLDNLLAQIGDQYADLFLAAKNLSDAILLSDIL
RVNTEITKAPLSASMIKRYDEHHQDLTLLKALVRQQLPEKYKEIFFDQSKN
GYAGYIDGGASQEEFYKFIKPILEKMDGTEELLVKLNREDLLRKQRTFDNG
SIPHQIHLGELHAILRRQEDFYPFLKDNREKIEKILTFRIPYYVGPLARGN
SRFAWMTRKSEETITPWNFEEVVDKGASAQSFIERMTNFDKNLPNEKVLPK
HSLLYEYFTVYNELTKVKYVTEGMRKPAFLSGEQKKAIVDLLFKTNRKVTV
KQLKEDYFKKIECFDSVEISGVEDRFNASLGTYHDLLKIIKDKDFLDNEEN
EDILEDIVLTLTLFEDREMIEERLKTYAHLFDDKVMKQLKRRRYTGWGRLS
RKLINGIRDKQSGKTILDFLKSDGFANRNFMQLIHDDSLTFKEDIQKAQVS
GQGDSLHEHIANLAGSPAIKKGILQTVKVVDELVKVMGRHKPENIVIEMAR
ENQTTQKGQKNSRERMKRIEEGIKELGSQILKEHPVENTQLQNEKLYLYYL
QNGRDMYVDQELDINRLSDYDVDHIVPQSFLKDDSIDNKVLTRSDKNRGKS
DNVPSEEVVKKMKNYWRQLLNAKLITQRKFDNLTKAERGGLSELDKAGFIK
RQLVETRQITKHVAQILDSRMNTKYDENDKLIREVKVITLKSKLVSDFRKD
FQFYKVREINNYHHAHDAYLNAVVGTALIKKYPKLESEFVYGDYKVYDVRK
MIAKSEQEIGKATAKYFFYSNIMNFFKTEITLANGEIRKRPLIETNGETGE
IVWDKGRDFATVRKVLSMPQVNIVKKTEVQTGGFSKESILPKRNSDKLIAR
KKDWDPKKYGGFDSPTVAYSVLVVAKVEKGKSKKLKSVKELLGITIMERSS
FEKNPIDFLEAKGYKEVKKDLIIKLPKYSLFELENGRKRMLASAGELQKGN
ELALPSKYVNFLYLASHYEKLKGSPEDNEQKQLFVEQHKHYLDEIIEQISE
FSKRVILADANLDKVLSAYNKHRDKPIREQAENIIHLFTLTNLGAPAAFKY
FDTTIDRKRYTSTKEVLDATLIHQSITGLYETRIDLSQLGGD

(b) Guide RNAs (gRNAs)

CRISPR-Cas9-mediated gene editing as described herein includes the use of a guide RNA or a gRNA. As used herein, a “gRNA” refers to a genome-targeting nucleic acid that can direct the Cas9 to a specific target sequence within a CD70 gene or a TRAC gene or a β2M gene for gene editing at the specific target sequence. A guide RNA comprises at least a spacer sequence that hybridizes to a target nucleic acid sequence within a target gene for editing, and a CRISPR repeat sequence.

An exemplary gRNA targeting a CD70 gene is provided in SEQ ID NO: 2. See also WO2019/215500, the relevant disclosures of which are incorporated by reference herein for the subject matter and purpose referenced herein. Other gRNA sequences may be designed using the CD70 gene sequence located on chromosome 19 (GRCh38: chromosome 19: 6,583,183-6,604,103; Ensembl; ENSG00000125726). In some embodiments, gRNAs targeting the CD70 genomic region and Cas9 create breaks in the CD70 genomic region resulting Indels in the CD70 gene disrupting expression of the mRNA or protein.

An exemplary gRNA targeting a TRAC gene is provided in SEQ ID NO: 6. See WO2019/097305A2, the relevant disclosures of which are incorporated by reference herein for the subject matter and purpose referenced herein. Other gRNA sequences may be designed using the TRAC gene sequence located on chromosome 14 (GRCh38: chromosome 14: 22,547,506-22,552,154; Ensembl; ENSG00000277734). In some embodiments, gRNAs targeting the TRAC genomic region and Cas9 create breaks in the TRAC genomic region resulting Indels in the TRAC gene disrupting expression of the mRNA or protein.

An exemplary gRNA targeting a β2M gene is provided in SEQ ID NO: 10. See also WO 2019/097305A2, the relevant disclosures of which are incorporated by reference herein for the purpose and subject matter referenced herein. Other gRNA sequences may be designed using the β2M gene sequence located on Chromosome 15 (GRCh38 coordinates: Chromosome 15: 44,711,477-44,718,877; Ensembl: ENSG00000166710). In some embodiments, gRNAs targeting the β2M genomic region and RNA-guided nuclease create breaks in the β2M genomic region resulting in Indels in the β2M gene disrupting expression of the mRNA or protein.

TABLE 2
sgRNA Sequences and Target Gene Sequences.

| | | | SEQ ID |
|---|---|---|---|
| sgRNA Sequences | | | NO: |
| CD70 | Modified | G*C*U*UUGGUCCCAUUGGUCGCguuuuagagcuagaaauagc | 2 |
| sgRNA | | aaguuaaaauaaggcuaguccguuaucaacuugaaaaaguggc | |
| (CD70-7) | | accgagucggugcU*U*U*U | |
| | Unmodified | GCUUUGGUCCCAUUGGUCGCguuuuagagcuagaaauagcaag | 3 |
| | | uuaaaauaaggcuaguccguuaucaacuugaaaaaguggcacc | |
| | | gagucggugcUUUU | |
| CD70 | Modified | G*C*U*UUGGUCCCAUUGGUCGC | 4 |
| sgRNA | Unmodified | GCUUUGGUCCCAUUGGUCGC | 5 |
| spacer | | | |
| TRAC | Modified | A*G*A*GCAACAGUGCUGUGGCCguuuuagagcuagaaauagc | 6 |
| sgRNA | | aaguuaaaauaaggcuaguccguuaucaacuugaaaaaguggc | |
| (TA-1) | | accgagucggugcU*U*U*U | |
| | Unmodified | AGAGCAACAGUGCUGUGGCCguuuuagagcuagaaauagcaag | 7 |
| | | uuaaaauaaggcuaguccguuaucaacuugaaaaaguggcacc | |
| | | gagucggugcUUUU | |
| TRAC | Modified | A*G*A*GCAACAGUGCUGUGGCC | 8 |
| sgRNA | Unmodified | AGAGCAACAGUGCUGUGGCC | 9 |
| spacer | | | |
| β2M | Modified | G*C*U*ACUCUCUCUUUCUGGCCguuuuagagcuagaaauagc | 10 |
| sgRNA | | aaguuaaaauaaggcuaguccguuaucaacuugaaaaaguggc | |
| (B2M-1) | | accgagucggugcU*U*U*U | |
| | Unmodified | GCUACUCUCUCUUUCUGGCCguuuuagagcuagaaauagcaag | 11 |
| | | uuaaaauaaggcuaguccguuaucaacuugaaaaaguggcacc | |
| | | gagucggugcUUUU | |
| β2M | Modified | G*C*U*ACUCUCUCUUUCUGGCC | 12 |
| spacer | Unmodified | GCUACUCUCUCUUUCUGGCC | 13 |
| sgRNA | | | |

Target Sequences (PAM)

| | | |
|---|---|---|
| CD70 | GCTTTGGTCCCATTGGTCGC (GGG) | 14 |
| target | | |
| sequence | | |
| with | | |
| (PAM) | | |
| CD70 | GCTTTGGTCCCATTGGTCGC | 15 |
| target | | |
| sequence | | |
| TRAC | AGAGCAACAGTGCTGTGGCC (TGG) | 16 |
| target | | |
| sequence | | |
| with | | |
| (PAM) | | |
| TRAC | AGAGCAACAGTGCTGTGGCC | 17 |
| target | | |
| sequence | | |
| β2M target | GCTACTCTCTCTTTCTGGCC (TGG) | 18 |
| sequence | | |
| with | | |
| (PAM) | | |
| β2M target | GCTACTCTCTCTTTCTGGCC | 19 |
| sequence | | |

Exemplary sgRNA Formulas

| | | |
|---|---|---|
| sgRNA | nnnnnnnnnnnnnnnnnnnnguuuuagagcuagaaauagcaaguuaaaauaagg | 20 |
| sequence | cuaguccguuaucaacuugaaaaguggcaccgagucggugcuuuu | |
| sgRNA | nnnnnnnnnnnnnnnnnnnnguuuuagagcuagaaauagcaaguuaaaauaagg | 21 |
| sequence | cuaguccguuaucaacuugaaaaaguggcaccgagucggugc | |
| sgRNA | n(17-30)guuuuagagcuagaaauagcaaguuaaaauaaggcuaguccguuaucaacu | 22 |
| sequence | ugaaa aaguggcaccgagucggugcu(1-8 ) | |

* indicates a nucleotide with a 2′-O-methyl phosphorothioate modification.
“n” refers to the spacer sequence at the 5′ end.

In Type II systems, the gRNA also comprises a second RNA called the tracrRNA sequence. In the Type II gRNA, the CRISPR repeat sequence and tracrRNA sequence hybridize to each other to form a duplex. In the Type V gRNA, the crRNA forms a duplex. In both systems, the duplex binds a site-directed polypeptide, such that the guide RNA and site-direct polypeptide form a complex. In some embodiments, the genome-targeting nucleic acid provides target specificity to the complex by virtue of its association with the site-directed polypeptide. The genome-targeting nucleic acid thus directs the activity of the site-directed polypeptide.

As is understood by the person of ordinary skill in the art, each guide RNA is designed to include a spacer sequence complementary to its genomic target sequence. See Jinek et al., Science, 337, 816-821 (2012) and Deltcheva et al., Nature, 471, 602-607 (2011).

In some embodiments, the genome-targeting nucleic acid (e.g., gRNA) is a double-molecule guide RNA. In some embodiments, the genome-targeting nucleic acid (e.g., gRNA) is a single-molecule guide RNA.

A double-molecule guide RNA comprises two strands of RNA molecules. The first strand comprises in the 5′ to 3′ direction, an optional spacer extension sequence, a spacer sequence and a minimum CRISPR repeat sequence. The second strand comprises a minimum tracrRNA sequence (complementary to the minimum CRISPR repeat sequence), a 3′ tracrRNA sequence and an optional tracrRNA extension sequence.

A single-molecule guide RNA (referred to as a “sgRNA”) in a Type II system comprises, in the 5′ to 3′ direction, an optional spacer extension sequence, a spacer sequence, a minimum CRISPR repeat sequence, a single-molecule guide linker, a minimum tracrRNA sequence, a 3′ tracrRNA sequence and an optional tracrRNA extension sequence. The optional tracrRNA extension may comprise elements that contribute additional functionality (e.g., stability) to the guide RNA. The single-molecule guide linker links the minimum CRISPR repeat and the minimum tracrRNA sequence to form a hairpin structure. The optional tracrRNA extension comprises one or more hairpins. A single-molecule guide RNA in a Type V system comprises, in the 5′ to 3′ direction, a minimum CRISPR repeat sequence and a spacer sequence.

The “target sequence” is in a target gene that is adjacent to a PAM sequence and is the sequence to be modified by Cas9. The “target sequence” is on the so-called PAM-strand in a “target nucleic acid,” which is a double-stranded molecule containing the PAM-strand and a complementary non-PAM strand. One of skill in the art recognizes that the gRNA spacer sequence hybridizes to the complementary sequence located in the non-PAM strand of the target nucleic acid of interest. Thus, the gRNA spacer sequence is the RNA equivalent of the target sequence.

For example, if the CD70 target sequence is 5′-GCTTTGGTCCCATTGGTCGC-3′ (SEQ ID NO: 15), then the gRNA spacer sequence is 5′-GCUUUGGUCCCAUUGGUCGC-3′ (SEQ ID NO: 5). In another example, if the TRAC target sequence is 5′-AGAGCAACAGTGCTGTGGCC-3′ (SEQ ID NO: 17), then the gRNA spacer sequence is 5′-AGAGCAACAGUGCUGUGGCC-3′ (SEQ ID NO: 9). In yet another example, if the β2M target sequence is 5′-GCTACTCTCTCTTTCTGGCC-3′ (SEQ ID NO: 19), then the gRNA spacer sequence is 5′-GCUACUCUCUCUUUCUGGCC-3′ (SEQ ID NO: 13). The spacer of a gRNA interacts with a target nucleic acid of interest in a sequence-specific manner via hybridization (i.e., base pairing). The nucleotide sequence of the spacer thus varies depending on the target sequence of the target nucleic acid of interest.

In a CRISPR/Cas system herein, the spacer sequence is designed to hybridize to a region of the target nucleic acid that is located 5′ of a PAM recognizable by a Cas9 enzyme used in the system. The spacer may perfectly match the target sequence or may have mismatches. Each Cas9 enzyme has a particular PAM sequence that it recognizes in a target DNA. For example, S. pyogenes recognizes in a target nucleic acid a PAM that comprises the sequence 5′-NRG-3′, where R comprises either A or G, where N is any nucleotide and N is immediately 3′ of the target nucleic acid sequence targeted by the spacer sequence.

In some embodiments, the target nucleic acid sequence has 20 nucleotides in length. In some embodiments, the target nucleic acid has less than 20 nucleotides in length. In some embodiments, the target nucleic acid has more than 20 nucleotides in length. In some embodiments, the target nucleic acid has at least: 5, 10, 15, 16, 17, 18, 19, 20, 21, 22, 23, 24, 25, 30 or more nucleotides in length. In some embodiments, the target nucleic acid has at most: 5, 10, 15, 16, 17, 18, 19, 20, 21, 22, 23, 24, 25, 30 or more nucleotides in length. In some embodiments, the target nucleic acid sequence has 20 bases immediately 5′ of the first nucleotide of the PAM. For example, in a sequence comprising 5′-NNNNNNNNNNNNNNNNNNNNNRG-3′, the target nucleic acid can be the sequence that corresponds to the Ns, wherein N can be any nucleotide, and the underlined NRG sequence is the S. pyogenes PAM.

A spacer sequence in a gRNA is a sequence (e.g., a 20 nucleotide sequence) that defines the target sequence (e.g., a DNA target sequences, such as a genomic target sequence) of a target gene of interest. An exemplary spacer sequence of a gRNA targeting a CD70 gene is provided in SEQ ID NO: 4. An exemplary spacer sequence of a gRNA targeting a TRAC gene is provided in SEQ ID NO: 8. An exemplary spacer sequence of a gRNA targeting a β2M gene is provided in SEQ ID NO: 12.

The guide RNA disclosed herein may target any sequence of interest via the spacer sequence in the crRNA. In some embodiments, the degree of complementarity between the spacer sequence of the guide RNA and the target sequence in the target gene can be about 60%, 65%, 70%, 75%, 80%, 85%, 90%, 95%, 97%, 98%, 99%, or 100%. In some embodiments, the spacer sequence of the guide RNA and the target sequence in the target gene is 100% complementary. In other embodiments, the spacer sequence of the guide RNA and the target sequence in the target gene may contain up to 10 mismatches, e.g., up to 9, up to 8, up to 7, up to 6, up to 5, up to 4, up to 3, up to 2, or up to 1 mismatch.

Non-limiting examples of gRNAs that may be used as provided herein are provided in WO 2019/097305A2, and WO2019/215500, the relevant disclosures of each of the prior applications are herein incorporated by reference for the purposes and subject matter referenced herein. For any of the gRNA sequences provided herein, those that do not explicitly indicate modifications are meant to encompass both unmodified sequences and sequences having any suitable modifications.

The length of the spacer sequence in any of the gRNAs disclosed herein may depend on the CRISPR/Cas9 system and components used for editing any of the target genes also disclosed herein. For example, different Cas9 proteins from different bacterial species have varying optimal spacer sequence lengths. Accordingly, the spacer sequence may have 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, or more than 50 nucleotides in length. In some embodiments, the spacer sequence may have 18-24 nucleotides in length. In some embodiments, the targeting sequence may have 19-21 nucleotides in length. In some embodiments, the spacer sequence may comprise 20 nucleotides in length.

In some embodiments, the gRNA can be a sgRNA, which may comprise a 20 nucleotide spacer sequence at the 5′ end of the sgRNA sequence. In some embodiments, the sgRNA may comprise a less than 20 nucleotide spacer sequence at the 5′ end of the sgRNA sequence. In some embodiments, the sgRNA may comprise a more than 20 nucleotide spacer sequence at the 5′ end of the sgRNA sequence. In some embodiments, the sgRNA comprises a variable length spacer sequence with 17-30 nucleotides at the 5′ end of the sgRNA sequence.

In some embodiments, the sgRNA comprises no uracil at the 3′ end of the sgRNA sequence. In other embodiments, the sgRNA may comprise one or more uracil at the 3′ end of the sgRNA sequence. For example, the sgRNA can comprise 1-8 uracil residues, at the 3′ end of the sgRNA sequence, e.g., 1, 2, 3, 4, 5, 6, 7, or 8 uracil residues at the 3′ end of the sgRNA sequence.

Any of the gRNAs disclosed herein, including any of the sgRNAs, may be unmodified. Alternatively, it may contain one or more modified nucleotides and/or modified backbones. For example, a modified gRNA such as an sgRNA can comprise one or more 2′-O-methyl phosphorothioate nucleotides, which may be located at either the 5′ end, the 3′ end, or both.

In certain embodiments, more than one guide RNAs can be used with a CRISPR/Cas nuclease system. Each guide RNA may contain a different targeting sequence, such that the CRISPR/Cas system cleaves more than one target nucleic acid. In some embodiments, one or more guide RNAs may have the same or differing properties such as activity or stability within the Cas9 RNP complex. Where more than one guide RNA is used, each guide RNA can be encoded on the same or on different vectors. The promoters used to drive expression of the more than one guide RNA is the same or different.

It should be understood that more than one suitable Cas9 and more than one suitable gRNA can be used in methods described herein, for example, those known in the art or disclosed herein. In some embodiments, methods comprise a Cas9 enzyme and/or a gRNA known in the art. Examples can be found in, e.g., WO 2019/097305A2, and WO2019/215500, the relevant disclosures of each of the prior applications are herein incorporated by reference for the purposes and subject matter referenced herein.

In some embodiments, gRNAs targeting the TRAC genomic region create Indels in the TRAC gene comprising at least one nucleotide sequence selected from the sequences in Table 3. In some embodiments, the gRNA (e.g., SEQ ID NO: 6) targeting the TRAC genomic region creates Indels in the TRAC gene comprising at least one nucleotide sequence selected from the sequences in Table 3.

TABLE 3
Edited TRAC Gene Sequence.

| | Sequence (Deletions indicated by dashes (-); | |
|---|---|---|
| Description | insertions indicated by bold) | SEQ ID NO: |
| TRAC gene edit | AA---------------------GAGCAACAAATCTGACT | 23 |
| TRAC gene edit | AAGAGCAACAGTGCTGT-GCCTGGAGCAACAAATCTGACT | 24 |
| TRAC gene edit | AAGAGCAACAGTG-------CTGGAGCAACAAATCTGACT | 25 |
| TRAC gene edit | AAGAGCAACAGT------GCCTGGAGCAACAAATCTGACT | 26 |
| TRAC gene edit | AAGAGCAACAGTG---------------------CTGACT | 27 |
| TRAC gene edit | AAGAGCAACAGTGCTGTGGGCCTGGAGCAACAAATCTGACT | 28 |
| TRAC gene edit | AAGAGCAACAGTGC--TGGCCTGGAGCAACAAATCTGACT | 29 |
| TRAC gene edit | AAGAGCAACAGTGCTGTGTGCCTGGAGCAACAAATCTGACT | 30 |

In some embodiments, gRNAs targeting the β2M genomic region create Indels in the β2M gene comprising at least one nucleotide sequence selected from the sequences in Table 4. In some embodiments, the gRNA (e.g., SEQ ID NO: 10) targeting the β2M genomic region creates Indels in the β2M gene comprising at least one nucleotide sequence selected from the sequences in Table 4.

TABLE 4
Edited β2M Gene Sequence.

| | Sequence (Deletions indicated by dashes (-); | SEQ ID |
|---|---|---|
| Description | insertions indicated by bold) | NO: |
| β2M gene-edit | CGTGGCCTTAGCTGTGCTCGCGCTACTCTCTCTTTCT- | 31 |
| | GCCTGGAGGCTATCCAGCGTGAGTCTCTCCTACCCTCCCGCT | |
| β2M gene-edit | CGTGGCCTTAGCTGTGCTCGCGCTACTCTCTCTTTC-- | 32 |
| | GCCTGGAGGCTATCCAGCGTGAGTCTCTCCTACCCTCCCGCT | |
| β2M gene-edit | CGTGGCCTTAGCTGTGCTCGCGCTACTCTCTCTTT---- | 33 |
| | CTGGAGGCTATCCAGCGTGAGTCTCTCCTACCCTCCCGCT | |
| β2M gene-edit | CGTGGCCTTAGCTGTGCTCGCGCTACTCTCTCTTTCTGGATAGCCTG | 34 |
| | GAGGCTATCCAGCGTGAGTCTCTCCTACCCTCCCGCT | |
| β2M gene-edit | CGTGGCCTTAGCTGTGCTCGC------------------------- | 35 |
| | GCTATCCAGCGTGAGTCTCTCCTACCCTCCCGCT | |
| β2M gene-edit | CGTGGCCTTAGCTGTGCTCGCGCTACTCTCTCTTTCTGTGGCCTGGA | 36 |
| | GGCTATCCAGCGTGAGTCTCTCCTACCCTCCCGCT | |

In some embodiments, gRNAs targeting the CD70 genomic region create Indels in the CD70 gene comprising at least one nucleotide sequence selected from the sequences in Table 5. In some embodiments, the gRNA (e.g., SEQ ID NO: 2) targeting the CD70 genomic region creates Indels in the CD70 gene comprising at least one nucleotide sequence selected from the sequences in Table 5.

TABLE 5
Edited CD70 Gene Sequence.

| | Sequence (Deletions indicated by dashes (-); | |
|---|---|---|
| Description | insertions indicated by bold) | SEQ ID NO: |
| CD70 gene-edit | CACACCACGAGGCAGATCACCAAGCCCGCG-- | 37 |
| | CAATGGGACCAAAGCAGCCCGCAGGACG | |
| CD70 gene-edit | CACACCACGAGGCAGATCACCAAGCCCGCGAACCAATGGGACCAAAG | 38 |
| | CAGCCCGCAGGACG | |
| CD70 gene-edit | CACACCACGAGGCAGATC------------ | 39 |
| | ACCAATGGGACCAAAGCAGCCCGCAGGACG | |
| CD70 gene-edit | CACACCACGAGGCAGATCACCAAGCCCGCG- | 40 |
| | CCAATGGGACCAAAGCAGCCCGCAGGACG | |
| CD70 gene-edit | CACACCACGAGGCAGATCACCAAGCCCGC- | 41 |
| | ACCAATGGGACCAAAGCAGCCCGCAGGACG | |
| CD70 gene-edit | CACACCACGAGGCAGATCACCA------------------------- | 42 |
| | AGCCCGCAGGACG | |

(iii) AAV Vectors for Delivery of CAR Constructs to T Cells

A nucleic acid encoding a CAR construct can be delivered to a cell using an adeno-associated virus (AAV). AAVs are small viruses which integrate site-specifically into the host genome and can therefore deliver a transgene, such as CAR. Inverted terminal repeats (ITRs) are present flanking the AAV genome and/or the transgene of interest and serve as origins of replication. Also present in the AAV genome are rep and cap proteins which, when transcribed, form capsids which encapsulate the AAV genome for delivery into target cells. Surface receptors on these capsids, which confer AAV serotype, which determines which target organs the capsids primarily binds and thus what cells the AAV most efficiently infects. There are twelve currently known human AAV serotypes. In some embodiments, the AAV for use in delivering the CAR-coding nucleic acid is AAV serotype 6 (AAV6).

Adeno-associated viruses are among the most frequently used viruses for gene therapy for several reasons. First, AAVs do not provoke an immune response upon administration to mammals, including humans. Second, AAVs are effectively delivered to target cells, particularly when consideration is given to selecting the appropriate AAV serotype. Finally, AAVs have the ability to infect both dividing and non-dividing cells because the genome can persist in the host cell without integration. This trait makes them an ideal candidate for gene therapy.

A nucleic acid encoding a CAR can be designed to insert into a genomic site of interest in the host T cells. In some embodiments, the target genomic site can be in a safe harbor locus.

In some embodiments, a nucleic acid encoding a CAR (e.g., via a donor template, which can be carried by a viral vector such as an adeno-associated viral (AAV) vector) can be designed such that it can insert into a location within a TRAC gene to disrupt the TRAC gene in the genetically engineered T cells and express the CAR polypeptide. Disruption of TRAC leads to loss of function of the endogenous TCR. For example, a disruption in the TRAC gene can be created with an endonuclease such as those described herein and one or more gRNAs targeting one or more TRAC genomic regions. Any of the gRNAs specific to a TRAC gene and the target regions can be used for this purpose, e.g., those disclosed herein.

In some examples, a genomic deletion in the TRAC gene and replacement by a CAR coding segment can be created by homology directed repair or HDR (e.g., using a donor template, which may be part of a viral vector such as an adeno-associated viral (AAV) vector). In some embodiments, a disruption in the TRAC gene can be created with an endonuclease as those disclosed herein and one or more gRNAs targeting one or more TRAC genomic regions, and inserting a CAR coding segment into the TRAC gene.

A donor template as disclosed herein can contain a coding sequence for a CAR. In some examples, the CAR-coding sequence may be flanked by two regions of homology to allow for efficient HDR at a genomic location of interest, for example, at a TRAC gene using CRISPR-Cas9 gene editing technology. In this case, both strands of the DNA at the target locus can be cut by a CRISPR Cas9 enzyme guided by gRNAs specific to the target locus. HDR then occurs to repair the double-strand break (DSB) and insert the donor DNA coding for the CAR. For this to occur correctly, the donor sequence is designed with flanking residues which are complementary to the sequence surrounding the DSB site in the target gene (hereinafter “homology arms”), such as the TRAC gene. These homology arms serve as the template for DSB repair and allow HDR to be an essentially error-free mechanism. The rate of homology directed repair (HDR) is a function of the distance between the mutation and the cut site so choosing overlapping or nearby target sites is important. Templates can include extra sequences flanked by the homologous regions or can contain a sequence that differs from the genomic sequence, thus allowing sequence editing.

Alternatively, a donor template may have no regions of homology to the targeted location in the DNA and may be integrated by NHEJ-dependent end joining following cleavage at the target site.

A donor template can be DNA or RNA, single-stranded and/or double-stranded, and can be introduced into a cell in linear or circular form. If introduced in linear form, the ends of the donor sequence can be protected (e.g., from exonucleolytic degradation) by methods known to those of skill in the art. For example, one or more dideoxynucleotide residues are added to the 3′ terminus of a linear molecule and/or self-complementary oligonucleotides are ligated to one or both ends. See, for example, Chang et al., (1987) Proc. Natl. Acad. Sci. USA 84:4959-4963; Nehls et al., (1996) Science 272:886-889. Additional methods for protecting exogenous polynucleotides from degradation include, but are not limited to, addition of terminal amino group(s) and the use of modified internucleotide linkages such as, for example, phosphorothioates, phosphoramidates, and O-methyl ribose or deoxyribose residues.

A donor template can be introduced into a cell as part of a vector molecule having additional sequences such as, for example, replication origins, promoters and genes encoding antibiotic resistance. Moreover, a donor template can be introduced into a cell as naked nucleic acid, as nucleic acid complexed with an agent such as a liposome or poloxamer, or can be delivered by viruses (e.g., adenovirus, AAV, herpesvirus, retrovirus, lentivirus and integrase defective lentivirus (IDLV)).

A donor template, in some embodiments, can be inserted at a site nearby an endogenous promoter (e.g., downstream or upstream) so that its expression can be driven by the endogenous promoter. In other embodiments, the donor template may comprise an exogenous promoter and/or enhancer, for example, a constitutive promoter, an inducible promoter, or tissue-specific promoter to control the expression of the CAR gene. In some embodiments, the exogenous promoter is an EF1α promoter. Other promoters may be used.

Furthermore, exogenous sequences may also include transcriptional or translational regulatory sequences, for example, promoters, enhancers, insulators, internal ribosome entry sites, sequences encoding 2A peptides and/or polyadenylation signals.

### III. NK Cell Inhibitors

NK cells play an important role in both innate and adaptive immunity—including mediating anti-tumor and anti-viral responses. Because NK cells do not require prior sensitization or priming to mediate its cytotoxic function, they are the first line of defense against virus-infected and malignant cells that have missing or nonfunctioning MHC class I (e.g., disrupted MHC class I, or disrupted MCH Class I subunits). NK cells recognize “non-self” cells without the need for antibodies and antigen-priming. MHC class I-specific inhibitory receptors on NK cells negatively regulate NK cell function. Engagement of NK cell inhibitory receptors with their MHC class I ligand checks NK cell-mediated lysis. When MHC class I-disrupted cells fail to bind inhibitory NK receptors (e.g., KIRs), the cells become susceptible to NK cell-mediated lysis. This phenomenon is also referred to as the “missing self recognition.” See e.g., Malmberg K J et al., Immunogenetics (2017), 69:547-556; Cruz-Munoz M E et al., J. Leukoc. Biol. (2019), 105:955-971.

Therefore, engineered human CAR T cells comprising disrupted MHC class I as described herein are susceptible to NK cell-mediated lysis, thus reducing the persistence and subsequent efficacy of the engineered human CAR T cells. Accordingly, in some embodiments the present disclosure provides NK cell inhibitors for use in combination with CAR T cell therapy comprising a population of engineered human CAR T cells as described herein.

The NK cell inhibitor to be used in the methods described herein can be a molecule that blocks, suppresses, or reduces the activity or number of NK cells, either directly or indirectly. The term “inhibitor” implies no specific mechanism of biological action whatsoever, and is deemed to expressly include and encompass all possible pharmacological, physiological, and biochemical interactions with NK cells whether direct or indirect. For the purpose of the present disclosure, it will be explicitly understood that the term “inhibitor” encompasses all the previously identified terms, titles, and functional states and characteristics whereby the NK cell itself, a biological activity of the NK cell (including but not limited to its ability to mediate cell killing), or the consequences of the biological activity, are substantially nullified, decreased, or neutralized in any meaningful degree, e.g., by at least 20%, 50%, 70%, 85%, 90%, 100%, 150%, 200%, 300%, or 500%, or by 10-fold, 20-fold, 50-fold, 100-fold, 1000-fold, or 10⁴-fold.

NK cell inhibitors may be a small molecule compound, a peptide or polypeptide, a nucleic acid, etc. Such NK cell inhibitors may be found in, for example, in International Patent Application No. PCT/IB2020/056085, the relevant discloses of which are incorporated by reference for the subject matter and purpose referenced herein. In some embodiments, the NK cell inhibitor disclosed herein is an antibody specific to CD38.

A. Antibodies that bind CD38 (Anti-CD38 Antibodies)

In some embodiments, the present disclosure provides antibodies that specifically bind CD38 (anti-CD38 antibodies) for use in the methods described herein. CD38, also known as cyclic ADP ribose hydrolase, is a 46-kDa type II transmembrane glycoprotein that synthesizes and hydrolyzes cyclic adenosine 5′-diphosphate-ribose, an intracellular calcium ion mobilizing messenger. A multifunctional protein, CD38 is also involved in receptor-mediated cell adhesion and signaling. An amino acid sequence of an exemplary human CD38 protein is provided in SEQ ID NO: 70 (NCBI Reference Sequence: NP001766.2). See Table 6 below. Methods for generating antibodies that specifically bind human CD38 are known to those of ordinary skill in the art.

An antibody (interchangeably used in plural form) as used herein is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term “antibody” encompasses not only intact (i.e., full-length) monoclonal antibodies, but also antigen-binding fragments (such as Fab, Fab′, F(ab′)2, Fv, single chain variable fragment (scFv)), mutants thereof, fusion proteins comprising an antibody portion, humanized antibodies, chimeric antibodies, diabodies, linear antibodies, single chain antibodies, single domain antibodies (e.g., camel or llama VHH antibodies), multi-specific antibodies (e.g., bispecific antibodies) and any other modified configuration of the immunoglobulin molecule that comprises an antigen recognition site of the required specificity, including glycosylation variants of antibodies, amino acid sequence variants of antibodies, and covalently modified antibodies.

A typical antibody molecule comprises a heavy chain variable region (VH) and a light chain variable region (VL), which are usually involved in antigen binding. These regions/residues that are responsible for antigen-binding can be identified from amino acid sequences of the VH/VL sequences of a reference antibody (e.g., an anti-CD38 antibody as described herein) by methods known in the art. The VH and VL regions can be further subdivided into regions of hypervariability, also known as “complementarity determining regions” (“CDR”), interspersed with regions that are more conserved, which are known as “framework regions” (“FR”). Each VH and VL is typically composed of three CDRs and four FRs, arranged from amino-terminus to carboxy-terminus in the following order: FR1, CDR1, FR2, CDR2, FR3, CDR3, FR4. The extent of the framework region and CDRs can be precisely identified using methodology known in the art, for example, by the Kabat definition, the Chothia definition, the AbM definition, and/or the contact definition, all of which are well known in the art. As used herein, a CDR may refer to the CDR defined by any method known in the art. Two antibodies having the same CDR means that the two antibodies have the same amino acid sequence of that CDR as determined by the same method. See, e.g., Kabat, E. A., et al. (1991) Sequences of Proteins of Immunological Interest, Fifth Edition, U.S. Department of Health and Human Services, NIH Publication No. 91-3242, Chothia et al., (1989) Nature 342:877; Chothia, C. et al. (1987) J. Mol. Biol. 196:901-917, Al-lazikani et al (1997) J. Molec. Biol. 273:927-948; and Almagro, J. Mol. Recognit. 17:132-143 (2004). See also hgmp.mrc.ac.uk and bioinforg.uk/abs.

An antibody includes an antibody of any class, such as IgD, IgE, IgG, IgA, or IgM (or sub-class thereof), and the antibody need not be of any particular class. Depending on the antibody amino acid sequence of the constant domain of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known.

The antibodies to be used as provided herein can be murine, rat, human, or any other origin (including chimeric or humanized antibodies). In some examples, the antibody comprises a modified constant region, such as a constant region that is immunologically inert, e.g., does not trigger complement mediated lysis, or does not stimulate antibody-dependent cell mediated cytotoxicity (ADCC).

In some embodiments, an antibody of the present disclosure is a humanized antibody. Humanized antibodies refer to forms of non-human (e.g., murine) antibodies that are specific chimeric immunoglobulins, immunoglobulin chains, or antigen-binding fragments thereof that contain minimal sequence derived from non-human immunoglobulin. For the most part, humanized antibodies are human immunoglobulins (recipient antibody) in which residues from a complementary determining region (CDR) of the recipient are replaced by residues from a CDR of a non-human species (donor antibody) such as mouse, rat, or rabbit having the desired specificity, affinity, and capacity. In some instances, Fv framework region (FR) residues of the human immunoglobulin are replaced by corresponding non-human residues. Furthermore, the humanized antibody may comprise residues that are found neither in the recipient antibody nor in the imported CDR or framework sequences, but are included to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. A humanized antibody optimally also will comprise at least a portion of an immunoglobulin constant region or domain (Fc), typically that of a human immunoglobulin. Other forms of humanized antibodies have one or more CDRs (one, two, three, four, five, and/or six) which are altered with respect to the original antibody, which are also termed one or more CDRs “derived from” one or more CDRs from the original antibody. Humanized antibodies may also involve affinity maturation.

In some embodiments, an antibody of the present disclosure is a chimeric antibody, which can include a heavy constant region and a light constant region from a human antibody. Chimeric antibodies refer to antibodies having a variable region or part of variable region from a first species and a constant region from a second species. Typically, in these chimeric antibodies, the variable region of both light and heavy chains mimics the variable regions of antibodies derived from one species of mammals (e.g., a non-human mammal such as mouse, rabbit, and rat), while the constant portions are homologous to the sequences in antibodies derived from another mammal such as human. In some embodiments, amino acid modifications can be made in the variable region and/or the constant region.

In some embodiments, an antibody of the present disclosure specifically binds a target antigen (e.g., human CD38). An antibody that “specifically binds” (used interchangeably herein) to a target or an epitope is a term well understood in the art, and methods to determine such specific binding are also well known in the art. A molecule is said to exhibit “specific binding” if it reacts or associates more frequently, more rapidly, with greater duration and/or with greater affinity with a particular target antigen than it does with alternative targets. An antibody “specifically binds” to a target antigen if it binds with greater affinity, avidity, more readily, and/or with greater duration than it binds to other substances. For example, an antibody that specifically (or preferentially) binds to a CD38 epitope, or is an antibody that binds this epitope with greater affinity, avidity, more readily, and/or with greater duration than it binds to other epitopes of the same antigen or a different antigen. It is also understood by reading this definition that, for example, an antibody that specifically binds to a first target antigen may or may not specifically or preferentially bind to a second target antigen. As such, “specific binding” or “preferential binding” does not necessarily require (although it can include) exclusive binding. Generally, but not necessarily, reference to binding means preferential binding.

Also within the scope of the present disclosure are functional variants of any of the exemplary antibodies as disclosed herein. A functional variant may contain one or more amino acid residue variations in the VH and/or VL, or in one or more of the HC CDRs and/or one or more of the VL CDRs as relative to a reference antibody, while retaining substantially similar binding and biological activities (e.g., substantially similar binding affinity, binding specificity, inhibitory activity, anti-tumor activity, or a combination thereof) as the reference antibody.

In some instances, the amino acid residue variations can be conservative amino acid residue substitutions. As used herein, a “conservative amino acid substitution” refers to an amino acid substitution that does not alter the relative charge or size characteristics of the protein in which the amino acid substitution is made. Variants can be prepared according to methods for altering polypeptide sequence known to one of ordinary skill in the art such as are found in references which compile such methods, e.g., Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York. Conservative substitutions of amino acids include substitutions made amongst amino acids within the following groups: (a) A→G, S; (b) R→K, H; (c) N→Q, H; (d) D→E, N; (e) C→S, A; (f) Q→N; (g) E→D, Q; (h) G→A; (i) H→N, Q; (j) I→L, V; (k) L→I, V; (l) K→R, H; (m) M→L, I, Y; (n) F→Y, M, L; (o) P→A; (p) S→T; (q) T→S; (r) W→Y, F; (s) Y→W, F; and (t) V→I, L.

Anti-CD38 antibodies have been tested in various pre-clinical and clinical studies, e.g., for NK/T cell lymphoma, or T-cell acute lymphoblastic leukemia. Exemplary anti-CD38 antibodies tested for anti-tumor properties include SAR650984 (also referred to as isatuximab, chimeric mAb), which is in phase I clinical trials in patients with CD38+ B-cell malignancies (Deckert J. et al., Clin. Cancer. Res. (2014): 20(17):4574-83), MOR202 (also referred to as MOR03087, fully human mAb), and TAK-079 (fully human mAb).

In some embodiments, an anti-CD38 antibody for use in the present disclosure includes SAR650984 (Isatuximab), MOR202, Ab79, Ab10, HM-025, HM-028, HM-034; as well as antibodies disclosed in U.S. Pat. Nos. 9,944,711, 7,829,673, WO2006/099875, WO 2008/047242, WO2012/092612, and EP 1 720 907 B1, herein incorporated by reference. In some embodiments, the anti-CD38 antibody disclosed herein may be a functional variant of any of the reference antibodies disclosed herein. Such a functional variant may comprise the same heavy chain and light chain complementary determining regions as the reference antibody. In some examples, the functional variant may comprise the same heavy chain variable region and the same light chain variable region as the reference antibody.

In some embodiments, the anti-CD38 antibody for use in the present disclosure is daratumumab. Daratumumab (also referred to as Darzalex®, HuMax-CD38, or IgG1-005) is a fully human IgGκ monoclonal antibody that targets CD38 and has been approved for treating multiple myeloma. It is used as a monotherapy or as a combination therapy for treating newly diagnosed or previously treated multiple myeloma patients. Daratumumab is described in U.S. Pat. No. 7,829,673 and WO2006/099875.

Daratumumab binds an epitope on CD38 that comprises two β-strands located at amino acids 233-246 and 267-280 of CD38. Experiments with CD38 mutant polypeptides show that the 5274 amino acid residue is important for daratumumab binding. (van de Donk NWCJ et al., Immunol. Rev. (2016) 270:95-112). Daratumumab's binding orientation to CD38 allows for Fc-receptor mediated downstream immune processes.

Mechanisms of action attributed to Daratumumab as a lymphoma and multiple myeloma therapy includes Fc-dependent effector mechanisms such as complement-dependent cytotoxicity (CDC), natural killer (NK)-cell mediated antibody-dependent cellular cytotoxicity (ADCC) (De Weers M, et al., J. Immunol. (2011) 186:1840-8), antibody-mediated cellular phagocytosis (ADCP) (Overdijk M B et al., MAbs (2015), 7(2):311-21), and apoptosis after cross-linking (van de Donk NWCJ and Usmani S Z, Front. Immunol. (2018), 9:2134).

The full heavy chain amino acid sequence of daratumumab is set forth in SEQ ID NO: 71 and the full light chain amino acid sequence of daratumumab is set forth in SEQ ID NO: 73. The amino acid sequence of the heavy chain variable region of daratumumab is set forth in SEQ ID NO: 64 and the amino acid sequence of the light chain variable region of daratumumab is set forth in SEQ ID NO: 74. Daratumumab includes the heavy chain complementary determining regions (HCDRs) 1, 2, and 3 (SEQ ID NOs: 75, 76, and 77, respectively), and the light chain CDRs (LCDRs) 1, 2, and 3 (SEQ ID NOs. 78, 79, and 80, respectively). See Table 6 below. In some embodiments, these sequences can be used to produce a monoclonal antibody that binds CD38. For example, methods for making daratumumab are described in U.S. Pat. No. 7,829,673 (incorporated herein by reference for the purpose and subject matter referenced herein).

TABLE 6
Amino Acid Sequences of Daratumumab and CD38

| Name | | SEQ |
|---|---|---|
| Description | Amino Acid Sequences | ID NO |
| CD38 | MANCEFSPVSGDKPCCRLSRRAQLCLGVSILVLILVVVLAVVVPRWR | 70 |
| | QQWSGPGTTKRFPETVLARCVKYTEIHPEMRHVDCQSVWDAFKGAFI | |
| | SKHPCNITEEDYQPLMKLGTQTVPCNKILLWSRIKDLAHQFTQVQRD | |
| | MFTLEDTLLGYLADDLTWCGEFNTSKINYQSCPDWRKDCSNNPVSVF | |
| | WKTVSRRFAEAACDVVHVMLNGSRSKIFDKNSTFGSVEVHNLQPEK | |
| | VQTLEAWVIHGGREDSRDLCQDPTIKELESIISKRNIQFSCKNIYRPDKF | |
| | LQCVKNPEDSSCTSEI | |
| Daratumumab | EVQLLESGGGLVQPGGSLRLSCAVSGFTFNSFAMSWVRQAPGKGLE | 71 |
| heavy chain full | WVSAISGSGGGTYYADSVKGRFTISRDNSKNTLY LQMNSLRAEDTA | |
| sequence | VYFCAKDKILWFGEPVFDYWGQGTLVTVSSASTKGPSVFPLAPSSKST | |
| | SGGTAALGCLVKDYFPEPVTVSWNSGALTSGVHTFPAVLQSSGLYSL | |
| | SSVVTVPSSSLGTQTYICNVNHK | |
| | PSNTKVDKRVEPKSCDKTHTCPPCP | |
| | APELLGGPSVFLFPPKPKDTLMISRTPEVTCVVVDVSHEDPEVKFNWY | |
| | V | |
| | DGVEVHNAKTKPREEQYNSTYRVVSVLTVLHQDWLNGKEYKCKVS | |
| | N | |
| | KALPAPIEKTISKAKGQPREPQVYTLPPSREEMTKNQVSLTCLVKGFY | |
| | PS | |
| | DIAVEWESNGQPENNYKTTPPVLDSDGSFFLYSKLTVDKSRWQQGNV | |
| | FSC SVMHEALHNHYTQKSLSLSP GK | |
| Daratumumab | EVQLLESGGGLVQPGGSLRLSCAVSGFTFNSFAMSWVRQAPGKGLE | 72 |
| heavy chain | WVSAISGSGGGTYYADSVKGRFTISRDNSKNTLYLQMNSLRAEDTAV | |
| variable region | YFCAKDKILWFGEPVFDYWGQGTLVTVSSAS | |
| Daratumumab | EIVLTQSPAT LSLSPGERAT LSCRASQSVS SYLAWYQQKPQAPRLLIY | 73 |
| light chain full | DASNRATGIPARFSGSGSGTDFTLTISSLEPEDFAVYYCQQRSNWPPTF | |
| sequence | G | |
| | QGTKVEIKRTVAAPSVFIFPPSDEQLKSGTASVVCLLNNFYPREAKVQ | |
| | W | |
| | KVDNALQSGNSQESVTEQDSKDSTYSLSSTLTLSKADYEKHKVYACE | |
| | VTHQGLSSPVTKSFNRGEC | |
| Daratumumab | EIVLTQSPATLSLSPGERATLSCRASQSVSSYLAWYQQKPGQAPRLLIY | 74 |
| light chain | DASNRATGIPARFSGSGSGTDFTLTISSLEPEDFAVYYCQQRSNWPPTF | |
| variable region | GQGTKVEIK | |
| Daratumumab | SFAMS | 75 |
| heavy chain | | |
| CDRI | | |
| Daratumumab | AISGSGGGTY YADSVKG | 76 |
| heavy chain | | |
| CDR2 | | |
| Daratumumab | DKILWFGEPV FDY | 77 |
| heavy chain | | |
| CDR3 | | |
| Daratumumab | RASQSVSSYL A | 78 |
| light chain | | |
| CDR1 | | |
| Daratumumab | DASNRAT | 79 |
| light chain | | |
| CDR2 | | |
| Daratumumab | QQRSNWPPT | 80 |
| light chain | | |
| CDR3 | | |

In some embodiments, an anti-CD38 antibody for use in the present disclosure is daratumumab, an antibody having the same functional features as daratumumab, or an antibody which binds to the same epitope as daratumumab or competes against daratumumab from binding to CD38.

In some embodiments, the anti-CD38 antibody comprises: (a) an immunoglobulin heavy chain variable region and (b) an immunoglobulin light variable region, wherein the heavy chain variable region and the light chain variable region defines a binding site (paratope) for CD38. In some embodiments, the heavy chain variable region comprises an HCDR1 comprising the amino acid sequence set forth in SEQ ID NO: 75, an HCDR2 comprising the amino acid sequence set forth in SEQ ID NO: 76; and an HCDR3 comprising the amino acid sequence in SEQ ID NO: 77. The HCDR1, HCDR2, and HCDR3 sequences are separated by the immunoglobulin framework (FR) sequences.

In some embodiments, the anti-CD38 antibody comprises: (a) an immunoglobulin light chain variable region and (b) an immunoglobulin heavy chain variable region, wherein the light chain variable region and the heavy chain variable region defines a binding site (paratope) for CD38. In some embodiments, the light chain variable region comprises an LCDR1 comprising the amino acid sequence set forth in SEQ ID NO: 78, an LCDR2 comprising the amino acid sequence set forth in SEQ ID NO: 79; and an LCDR3 comprising the amino acid sequence in SEQ ID NO: 80. The LCDR1, LCDR2, and LCDR3 sequences are separated by the immunoglobulin framework (FR) sequences.

In some embodiments, the anti-CD38 antibody comprises an immunoglobulin heavy chain variable region (VH) comprising the amino acid sequence set forth in SEQ ID NO: 72, and an immunoglobulin light chain variable region (VL). In some embodiments, the anti-CD38 antibody comprises an immunoglobulin light chain variable region (VL) comprising the amino acid sequence set forth in SEQ ID NO: 74, and an immunoglobulin heavy chain variable region (VH). In some embodiments, the anti-CD38 antibody comprises a VH comprising an amino acid sequence that is at least 70%, 75%, 70%, 85%, 90%, 95%, 96%, 97%, 98%, and 99% identical to the amino acid sequence set forth in SEQ ID NO: 72, and comprises an VL comprising an amino acid sequence that is at least 70%, 75%, 70%, 85%, 90%, 95%, 96%, 97%, 98%, and 99% identical to the amino acid sequence set forth in SEQ ID NO: 74.

The “percent identity” of two amino acid sequences is determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST and XBLAST programs (version 2.0) of Altschul, et al. J. Mol. Biol. 215:403-10, 1990. BLAST protein searches can be performed with the XBLAST program, score=50, wordlength=3 to obtain amino acid sequences homologous to the protein molecules of the invention. Where gaps exist between two sequences, Gapped BLAST can be utilized as described in Altschul et al., Nucleic Acids Res. 25(17):3389-3402, 1997. When utilizing BLAST and Gapped BLAST programs, the default parameters of the respective programs (e.g., XBLAST and NBLAST) can be used.

CD38 is expressed on NK cells and infusion of daratumumab results in a reduction of NK cells in peripheral blood and bone marrow. The reduction of NK cells is due to NK-cell killing via ADCC, in which NK cells mediate cytotoxic killing of neighboring NK cells. Administration of daratumumab has also been shown to decrease cell numbers of myeloid derived suppressor cells, regulatory T cells, and regulatory B cells. The elimination of regulatory immune cells results in increased T cell responses and increased T cell numbers (J Krejcik et al., Blood (2016), 128(3):384-394.

Accordingly, in some embodiments, the anti-CD38 antibody (e.g., daratumumab) reduces absolute NK cell numbers. In some embodiments, the anti-CD38 antibody reduces NK cell percentage in PBMCs. In some embodiments, the anti-CD38 antibody inhibits NK cell activity through Fc-mediated mechanisms. In other embodiments, the anti-CD38 antibody mediates the killing of NK cells through CDC. In other embodiments, the anti-CD38 antibody mediates the killing of NK cells through ADCC. In other embodiments, the anti-CD38 antibody enhances phagocytosis of NK cells. In other embodiments, the anti-CD38 antibody enhances apoptosis induction after FcγR-mediated cross-linking.

In some embodiments, the anti-CD38 antibody is daratumumab or an antibody having the same functional features as daratumumab, for example, a functional variant of daratumumab. In some examples, a functional variant comprises substantially the same V_{H} and V_{L} CDRs as daratumumab. For example, it may comprise only up to 8 (e.g., 8, 7, 6, 5, 4, 3, 2, or 1) amino acid residue variations in the total CDR regions of the antibody and binds the same epitope of CD38 with substantially similar affinity (e.g., having a KD value in the same order) as daratumumab. In some instances, the functional variants may have the same heavy chain CDR3 as daratumumab, and optionally the same light chain CDR3 as daratumumab. Alternatively or in addition, the functional variants may have the same heavy chain CDR2 as daratumumab. Such an anti-CD38 antibody may comprise a V_{H} fragment having CDR amino acid residue variations in only the heavy chain CDR1 as compared with the V_{H} of daratumumab. In some examples, the anti-CD38 antibody may further comprise a V_{L} fragment having the same V_{L} CDR3, and optionally same V_{L} CDR1 or V_{L} CDR2 as daratumumab. Alternatively or in addition, the amino acid residue variations can be conservative amino acid residue substitutions (see above disclosures).

In some embodiments, the anti-CD38 antibody may comprise heavy chain CDRs that are at least 80% (e.g., 85%, 90%, 95%, or 98%) sequence identity, individually or collectively, as compared with the V_{H} CDRs of daratumumab. Alternatively or in addition, the anti-CD38 antibody may comprise light chain CDRs that are at least 80% (e.g., 85%, 90%, 95%, or 98%) sequence identity, individually or collectively, as compared with the V_{L} CDRs as daratumumab. As used herein, “individually” means that one CDR of an antibody shares the indicated sequence identity relative to the corresponding CDR of daratumumab. “Collectively” means that three V_{H} or V_{L} CDRs of an antibody in combination share the indicated sequence identity relative the corresponding three V_{H} or V_{L} CDRs of daratumumab.

In some embodiments, the anti-CD38 antibody binds to the same epitope bound by daratumumab on human CD38. In some embodiments, the anti-CD38 antibody competes with daratumumab for binding to human CD38.

Competition assays for determining whether an antibody binds to the same epitope as daratumumab, or competes with daratumumab for binding to CD38, are known in the art. Exemplary competition assays include immunoassays (e.g., ELISA assay, RIA assays), surface plasmon resonance, (e.g., BIAcore analysis), bio-layer interferometry, and flow cytometry.

A competition assay typically involves an immobilized antigen (e.g., CD38), a test antibody (e.g., CD38-binding antibody) and a reference antibody (e.g., daratumumab). Either one of the reference or test antibody is labeled, and the other unlabeled. In some embodiments, competitive binding is determined by the amount of a reference antibody bound to the immobilized antigen in increasing concentrations of the test antibody. Antibodies that compete with a reference antibody include antibodies that bind the same or overlapping epitopes as the reference antibody. In some embodiments, the test antibodies bind to adjacent, non-overlapping epitopes such that the proximity of the antibodies causes a steric hindrance sufficient to affect the binding of the reference antibody to the antigen.

A competition assay can be conducted in both directions to ensure that the presence of the label or steric hindrance does not interfere or inhibit binding to the epitope. For example, in the first direction, the reference antibody is labeled and the test antibody is unlabeled. In the second direction, the test antibody is labeled, and the reference antibody is unlabeled. In another embodiment, in the first direction, the reference antibody is bound to the immobilized antigen, and increasing concentrations of the test antibody are added to measure competitive binding. In the second direction, the test antibody is bound to the immobilized antigen, and increasing concentrations of the reference antibody are added to measure competitive binding.

In some embodiments, two antibodies can be determined to bind to the same epitope if essentially all amino acid mutations in the antigen that reduce or eliminate the binding of one antibody reduce or eliminate binding of the other. Two antibodies can be determined to bind to overlapping epitopes if only a subset of the mutations that reduce or eliminate the binding of one antibody reduces or eliminates the binding of the other.

In some embodiments, the heavy chain of any of the anti-CD38 antibodies as described herein (e.g., daratumumab) may further comprise a heavy chain constant region (CH) or a portion thereof (e.g., CH1, CH2, CH3, or a combination thereof). The heavy chain constant region can of any suitable origin, e.g., human, mouse, rat, or rabbit. Alternatively or in addition, the light chain of the anti-CD38 antibody may further comprise a light chain constant region (CL), which can be any CL known in the art. In some examples, the CL is a kappa light chain. In other examples, the CL is a lambda light chain. Antibody heavy and light chain constant regions are well known in the art, e.g., those provided in the IMGT database (www.imgt.org) or at www.vbase2.org/vbstat.php., both of which are incorporated by reference herein.

Any of the anti-CD38 antibodies, including human antibodies or humanized antibodies, can be prepared by conventional approaches, for example, hybridoma technology, antibody library screening, or recombinant technology. See, for example, Harlow and Lane, (1998) Antibodies: A Laboratory Manual, Cold Spring Harbor Laboratory, New York, WO 87/04462, Morrison et al., (1984) Proc. Nat. Acad. Sci. 81:6851, and Queen et al., Proc. Natl. Acad. Sci. USA, 86:10029-10033 (1989).

It should be understood that the described antibodies are only exemplary and that any anti-CD38 antibodies can be used in the compositions and methods disclosed herein. Methods for producing antibodies are known to those of skill in the art.

### III. Treatment of CD70 Positive Solid Tumors

In some embodiments, the T cells of the present disclosure (e.g., CTX130 cells) are engineered with a chimeric antigen receptor (CAR) designed to target CD70. CD70 was initially identified as the ligand for CD27, a co-stimulatory receptor involved in T cell proliferation and survival. CD70 is only found on a small percentage of activated T cells and antigen presenting cells in draining lymph nodes during viral infection. Many human tumors also express CD70, including, but not limited to, solid cancers such as breast cancer, gastric cancer, ovarian cancer, and glioblastoma. Due to its restricted expression pattern (Flieswasser et al., Cancers, (2019) 11:1611) on normal tissues and overexpression in numerous cancers, CD70 is an attractive therapeutic target. Non-limiting examples of cancers (e.g., solid tumors) that may be treated as provided herein include pancreatic cancer, gastric cancer, ovarian cancer, cervical cancer, breast cancer, thyroid cancer, nasopharyngeal cancer, non-small cell lung (NSCLC), glioblastoma, lymphoma, and/or melanoma.

In some aspects, provided herein are methods for treating a human patient having a CD70 positive tumor (e.g., CD70+ solid tumor) using a population of any of the anti-CD70 CAR T cells such as the CTX130 cells as disclosed herein, either taken alone or in combination with an NK cel inhibitor such as an anti-CD38 antibody (e.g., Daratumumab), either by a single dose (a single cycle of treatment) or multiple doses (multiple cycles of treatment).

Such an allogeneic anti-CD70 CAR T cell therapy may comprise two stages of treatment (i) a conditioning regimen (lymphodepleting treatment), which comprises giving one or more doses of one or more lymphodepleting agents to a suitable human patient, and (ii) a treatment regimen (anti-CD70 CAR T cell therapy), which comprises administration of the population of anti-CD70 CART cells such as the CTX130 cells as disclosed herein to the human patient. When applicable, multiple doses of the anti-CD70 CAR T cells may be given to the human patient and a lymphodepletion treatment can be applied to the human patient prior to each dose of the anti-CD70 CAR T cells. Alternatively, the treatment regimen may further comprise administering to the human patient one or more doses of an NK cell inhibitor such as daratumumab.

(i) Patient Population

A human patient may be any human subject for whom diagnosis, treatment, or therapy is desired. A human patient may be of any age. In some embodiments, the human patient is an adult (e.g., a person who is at least 18 years old). In some embodiments, the human patient is a child. In some embodiments, the human patient has a body weight>42 kg (e.g., 60 kg).

A human patient to be treated by the methods described herein can be a human patient having, suspected of having, or a risk for having a CD70+ solid tumor (e.g., a lung cancer, a gastric cancer, an ovarian cancer, a pancreatic cancer, a prostate cancer, or a RCC). A subject suspected of having a CD70+ solid tumor might show one or more symptoms of cancer, e.g., fatigue, lump or area of thickening that can be felt under the skin, weight changes including unexplained weight loss or weight gain, skin changes (e.g., yellowing, darkening or redness of the skin, sores that won't heal, or changes to existing moles), changes in bowel or bladder habits, persistent cough or trouble breathing, difficulty swallowing, hoarseness, persistent indigestion or discomfort after eating, persistent, unexplained muscle or joint pain, persistent, unexplained fevers or night sweats, or unexplained bleeding or bruising.

A subject at risk for a CD70+ solid tumor can be a subject having one or more of the risk factors for a CD70+ solid tumor, e.g., age, smoking, obesity, high blood pressure, excessive exposure to the sun, exposure to chemicals and/or viruses, family history, or genetic conditions. A human patient who needs the anti-CD70 CAR T cell (e.g., CTX130 cell) treatment may be identified by routine medical examination, e.g., laboratory tests, biopsy, imaging tests (e.g., magnetic resonance imaging (MRI) scans, a computerized tomography (CT) scan, bone scan, ultrasound exams, positron emission tomography (PET) scan, and X-ray).

Examples of CD70+ solid tumors that may be treated as provided herein include pancreatic cancer, gastric cancer, ovarian cancer, cervical cancer, breast cancer, thyroid cancer, nasopharyngeal cancer, non-small cell lung (NSCLC), glioblastoma, RCC, and/or melanoma.

In some embodiments, the human patient to be treated by the methods described herein can be a human patient having a tumor comprising CD70-expressing tumor cells (CD70-expressing tumor), which may be identified by any method known in the art, for example, by an immune assay such as immunohistochemistry (IHC) or flow cytometry.

Any of the methods disclosed herein may further comprise a step of identifying a human patient suitable for the allogeneic anti-CD70 CAR T therapy based on presence and/or level of CD70+ tumor cells in the patient.

A human patient to be treated by methods described herein may be a human patient having an advanced solid tumor, for example, unresectable or metastatic solid tumor. In some embodiments, the human patient may have a solid tumor that has relapsed following a treatment and/or that has been become resistant to a treatment and/or that has been non-responsive to a treatment. A human patient to be treated by methods described herein may be a human patient that has had recent prior treatment. Alternatively, the human patient may be free of prior treatment.

In some embodiments, the human patient has a relapsed or refractory CD70+ solid tumor. As used herein, “a refractory CD70+ solid tumor” refers to a CD70+ solid tumor that does not respond to or becomes resistant to a treatment. As used herein, “a relapsed CD70+ solid tumor” refers to a CD70+ solid tumor that returns following a period of complete response. In some embodiments, relapse occurs after the treatment. In other embodiments, relapse occurs during the treatment. A lack of response may be determined by routine medical practice. In some embodiments, the human patient has a relapsed CD70+ solid tumor. In some embodiments, the human patient has a refractory CD70+ solid tumor.

In some instances, a human patient to be treated by a method described herein can be a human patient having, suspected of having, or a risk for having renal cell carcinoma (RCC). A subject suspected of having RCC might show one or more symptoms of RCC, e.g., unexplained weight loss, anemia, abdominal pain, blood in the urine, or lumps in the abdomen. A subject at risk for RCC can be a subject having one or more of the risk factors for RCC, e.g., smoking, obesity, high blood pressure, family history of RCC, or genetic conditions such as von Hippel-Lindau disease. A human patient who needs the anti-CD70 CAR T cell (e.g., CTX130 cell) treatment may be identified by routine medical examination, e.g., laboratory tests, biopsy, magnetic resonance imaging (MRI) scans, or ultrasound exams.

Examples of renal cell carcinomas (RCCs) that may be treated using methods described herein include, but are not limited to, clear cell renal carcinomas (ccRCC), papillary renal cell carcinomas (pRCC), and chromophobe renal cell carcinomas (crRCC). These three subtypes account for more than 90% of all RCCs.

In some embodiments, the human patient has unresectable or metastatic RCC. In some embodiments, the human patient has relapsed or refractory RCC. As used herein, “refractory RCC” refers to RCC that does not respond to or becomes resistant to a treatment. As used herein, “relapsed RCC” refers to RCC that returns following a period of complete response. In some embodiments, relapse occurs after the treatment. In other embodiments, relapse occurs during the treatment. A lack of response may be determined by routine medical practice. In some embodiments, the human patient has predominantly clear cell RCC (ccRCC). In some embodiments, the human patient has advanced (e.g., unresectable or metastatic) RCC with clear cell differentiation (e.g., predominantly). In some embodiments, the human patient has relapsed or refractory RCC with clear cell differentiation (e.g., predominantly).

A human patient may be screened to determine whether the patient is eligible to undergo a conditioning regimen (lymphodepleting treatment) and/or a treatment regimen (anti-CD70 CAR T cell therapy, either taken alone or in combination with daratumumab). For example, a human patient who is eligible for lymphodepletion treatment does not show one or more of the following features: (a) worsening of clinical status, (b) requirement for supplemental oxygen to maintain a saturation level of greater than 90% (e.g., greater than 92%), (c) uncontrolled cardiac arrhythmia, (d) hypotension requiring vasopressor support, (e) active infection, (f) platelet count≤100,000/mm³, absolute neutrophil count≤1500/mm³, and/or hemoglobin≤9 g/dL without prior blood cell transfusion; and (g) grade≥2 acute neurological toxicity. In another example, a human patient who is eligible for a treatment regimen does not show one or more of the following features: (a) active uncontrolled infection, (b) worsening of clinical status compared to the clinical status prior to lymphodepletion treatment, and (c) grade≥2 acute neurological toxicity (e.g., ICANS).

A human patient may be screened and excluded from the conditioning regimen and/or treatment regimen based on such screening results. For example, a human patient may be excluded from a conditioning regimen and/or a treatment regimen if the patient meets any of the following exclusion criteria: (a) prior treatment with any anti-CD70 targeting agents, (b) prior treatment with any CAR T cells or any other modified T or natural killer (NK) cells, (c) prior anaphylactic reaction to any lymphodepletion treatment or any of the excipients of any treatment regimen, (d) detectable malignant cells from cerebrospinal fluid (CSF) or magnetic resonance imaging (MRI) indicating brain metastases, (e) history or presence of clinically relevant CNS pathology, (f) unstable angina, arrhythmia, or myocardial infarction within 6 months prior to screening, and (g) uncontrolled, acute life-threatening bacterial, viral, or fungal infection. In some instances, the human patient may be free of diabetes mellitus with an HBA1c level of 6.5% or 48 mmol/ml.

A human patient subjected to lymphodepletion treatment may be screened for eligibility to receive one or more doses of the anti-CD70 CAR T cells disclosed herein such as the CTX130 cells. For example, a human patient subjected to lymphodepletion treatment that is eligible for an anti-CD70 CAR T cell treatment does not show one or more of the following features: (a) active uncontrolled infection, (b) worsening of clinical status, and (c) grade≥2 acute neurological toxicity (e.g., ICANS).

Following each dosing of anti-CD70 CAR T cells, a human patient may be monitored for acute toxicities such as cytokine release syndrome (CRS), tumor lysis syndrome (TLS), neurotoxicity (e.g., ICANS), graft versus host disease (GvHD), viral encephalitis, on target off-tumor toxicity, and/or uncontrolled T cell proliferation. The on target off-tumor toxicity may comprises activity of the population of genetically engineered T cells against activated T lymphocytes, B lymphocytes, dentritic cells, osteoblasts and/or renal tubular-like epithelium. One or more of the following potential toxicity may also be monitored: hytotension, renal insufficiency, hemophagocytic lymphohistiocytosis (HLH), prolonged cytopenias, and/or drug-induced liver injury. After each dose of anti-CD70 CAR T cells, a human patient may be monitored for at least 28 days for development of toxicity.

When a human patient exhibits one or more symptoms of acute toxicity, the human patient may be subjected to toxicity management. Treatments for patients exhibiting one or more symptoms of acute toxicity are known in the art. For example, a human patient exhibiting a symptom of CRS (e.g., cardiac, respiratory, and/or neurological abnormalities) may be administered an anti-cytokine therapy. In addition, a human patient that does not exhibit a symptom of CRS may be administered an anti-cytokine therapy to promote proliferation of anti-CD70 CART cells.

Alternatively, or in addition to, when a human patient exhibits one or more symptoms of acute toxicity, treatment of the human patient may be terminated. Patient treatment may also be terminated if the patient exhibits one or more signs of an adverse event (AE), e.g., the patient has an abnormal laboratory finding and/or the patient shows signs of disease progression.

Any of the human patients treated using a method disclosed herein may receive subsequent treatment. For example, the human patient is subject to an anti-cytokine therapy. In another example, the human patient is subject to autologous or allogeneic hematopoietic stem cell transplantation after treatment with the population of genetically engineered T cells.

(ii) Conditioning Regimen (Lymphodepleting Therapy)

Any human patients suitable for the treatment methods disclosed herein may receive a lymphodepleting therapy to reduce or deplete the endogenous lymphocyte of the subject.

Lymphodepletion refers to the destruction of endogenous lymphocytes and/or T cells, which is commonly used prior to immunotransplantation and immunotherapy. Lymphodepletion can be achieved by irradiation and/or chemotherapy. A “lymphodepleting agent” can be any molecule capable of reducing, depleting, or eliminating endogenous lymphocytes and/or T cells when administered to a subject. In some embodiments, the lymphodepleting agents are administered in an amount effective in reducing the number of lymphocytes by at least 10%, 20%, 30%, 40%, 50%, 60%, 70%, 80%, 90%, 95%, 96%, 96%, 97%, 98%, or at least 99% as compared to the number of lymphocytes prior to administration of the agents. In some embodiments, the lymphodepleting agents are administered in an amount effective in reducing the number of lymphocytes such that the number of lymphocytes in the subject is below the limits of detection. In some embodiments, the subject is administered at least one (e.g., 2, 3, 4, 5 or more) lymphodepleting agents.

In some embodiments, the lymphodepleting agents are cytotoxic agents that specifically kill lymphocytes. Examples of lymphodepleting agents include, without limitation, fludarabine, cyclophosphamide, bendamustin, 5-fluorouracil, gemcitabine, methotrexate, dacarbazine, melphalan, doxorubicin, vinblastine, cisplatin, oxaliplatin, paclitaxel, docetaxel, irinotecan, etopside phosphate, mitoxantrone, cladribine, denileukin diftitox, or DAB-IL2. In some instances, the lymphodepleting agent may be accompanied with low-dose irradiation. The lymphodepletion effect of the conditioning regimen can be monitored via routine practice.

In some embodiments, the method described herein involves a conditioning regimen that comprises one or more lymphodepleting agents, for example, fludarabine and cyclophosphamide. A human patient to be treated by the method described herein may receive multiple doses of the one or more lymphodepleting agents for a suitable period (e.g., 1-5 days) in the conditioning stage. The patient may receive one or more of the lymphodepleting agents once per day during the lymphodepleting period. In one example, the human patient receives fludarabine at about 20-50 mg/m² (e.g., 30 mg/m²) per day for 2-4 days (e.g., 3 days) and cyclophosphamide at about 300-600 mg/m² (e.g., 500 mg/m²) per day for 2-4 days (e.g., 3 days). In one example, the human patient receives fludarabine at about 20-50 mg/m² (e.g., 20 mg/m² or 30 mg/m²) per day for 2-4 days (e.g., 3 days) and cyclophosphamide at about 300-600 mg/m² (e.g., 500 mg/m²) per day for 2-4 days (e.g., 3 days). In another example, the human patient receives fludarabine at about 20-30 mg/m² (e.g., 25 mg/m²) per day for 2-4 days (e.g., 3 days) and cyclophosphamide at about 300-600 mg/m² (e.g., 300 mg/m² or 400 mg/m²) per day for 2-4 days (e.g., 3 days). If needed, the dose of cyclophosphamide may be increased, for example, to up to 1,000 mg/m².

The human patient may then be administered any of the anti-CD70 CAR T cells such as CTX130 cells within a suitable period after the lymphodepleting therapy as disclosed herein. For example, a human patient may be subject to one or more lymphodepleting agent about 2-7 days (e.g., for example, 2, 3, 4, 5, 6, 7 days) before administration of the anti-CD70 CAR+ T cells (e.g., CTX130 cells).

Since the allogeneic anti-CD70 CAR-T cells such as CTX130 cells can be prepared in advance, the lymphodepleting therapy as disclosed herein may be applied to a human patient having a CD70+ tumor within a short time window (e.g., within 2 weeks) after the human patient is identified as suitable for the allogeneic anti-CD70 CAR-T cell therapy disclosed herein.

Methods described herein encompass redosing a human patient with anti-CD70 CAR+ T cells. In such instances, the human patient is subjected to lymphodepletion treatment prior to redosing. For example, a human patient may be subject to a first lymphodepletion treatment and a first dose of CTX130 followed by a second lymphodepletion treatment and a second dose of CTX130. In another example, a human patient may be subject to a first lymphodepletion treatment and a first dose of CTX130, a second lymphodepletion treatment and a second dose of CTX130, and a third lymphodepletion treatment and a third dose of CTX130.

Prior to any of the lymphodepletion steps (e.g., prior to the initial lymphodepletion step or prior to any follow-on lymphodepletion step in association with a re-dosing of the anti-CD70 CAR T cells such as CTX130 cells), a human patient may be screened for one or more features to determine whether the patient is eligible for lymphodepletion treatment. For example, prior to lymphodepletion, a human patient eligible for lymphodepletion treatment does not show one or more of the following features: (a) significant worsening of clinical status, (b) requirement for supplemental oxygen to maintain a saturation level of greater than 90%, (c) uncontrolled cardiac arrhythmia, (d) hypotension requiring vasopressor support, (e) active infection, (f) platelet count≤100,000/mm³, absolute neutrophil count≤1500/mm³, and/or hemoglobin≤9 g/dL without prior blood cell transfusion; and (g) grade≥2 acute neurological toxicity (e.g., ICANs).

Following lymphodepletion, a human patient may be screened for one or more features to determine whether the patient is eligible for treatment with anti-CD70 CAR T cells. For example, prior to anti-CD70 CAR T cell treatment and after lymphodepletion treatment, a human patient eligible for anti-CD70 CAR T cells treatment does not show one or more of the following features: (a) active uncontrolled infection, (b) worsening of clinical status, and (c) grade≥2 acute neurological toxicity.

(iii) Administration of Anti-CD70 CAR T Cells

Aspects of the present disclosure provide methods of treating a CD70+ solid tumor comprising subjecting a human patient to lymphodepletion treatment and administering to the human patient a dose of a population of genetically engineered T cells described herein (e.g., CTX130 cells).

Administering anti-CD70 CAR T cells may include placement (e.g., transplantation) of a genetically engineered T cell population into a human patient by a method or route that results in at least partial localization of the genetically engineered T cell population at a desired site, such as a tumor site, such that a desired effect(s) can be produced. The genetically engineered T cell population can be administered by any appropriate route that results in delivery to a desired location in the subject where at least a portion of the implanted cells or components of the cells remain viable. The period of viability of the cells after administration to a subject can be as short as a few hours, e.g., twenty-four hours, to a few days, to as long as several years, or even the life time of the subject, i.e., long-term engraftment. For example, in some aspects described herein, an effective amount of the genetically engineered T cell population can be administered via a systemic route of administration, such as an intraperitoneal or intravenous route.

In some embodiments, the genetically engineered T cell population is administered systemically, which refers to the administration of a population of cells other than directly into a target site, tissue, or organ, such that it enters, instead, the subject's circulatory system and, thus, is subject to metabolism and other like processes. Suitable modes of administration include injection, infusion, instillation, or ingestion. Injection includes, without limitation, intravenous, intramuscular, intra-arterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. In some embodiments, the route is intravenous.

An effective amount refers to the amount of a genetically engineered T cell population needed to prevent or alleviate at least one or more signs or symptoms of a medical condition (e.g., cancer), and relates to a sufficient amount of a genetically engineered T cell population to provide the desired effect, e.g., to treat a subject having a medical condition. An effective amount also includes an amount sufficient to prevent or delay the development of a symptom of the disease, alter the course of a symptom of the disease (for example but not limited to, slow the progression of a symptom of the disease), or reverse a symptom of the disease. It is understood that for any given case, an appropriate effective amount can be determined by one of ordinary skill in the art using routine experimentation.

An effective amount of a genetically engineered T cell population may comprise about 1×10⁶ cells to about 1.0×10⁹ CAR+ cells, e.g., about 3.0×10⁷ cells to about 1.0×10⁹ cells that express an anti-CD70 CAR (CAR⁺ cells), for example, CAR⁺ CTX130 cells.

In some instances, an effective amount of a genetically engineered T cell population may comprise about 3.0×10⁷ cells to about 9×10⁸ cells that express an anti-CD70 CAR (CAR⁺ cells), for example, CAR⁺ CTX130 cells. In some instances, an effective amount of a genetically engineered T cell population may comprise about 3.0×10⁷ cells to about 7.5×10⁸ cells that express an anti-CD70 CAR (CAR⁺ cells), for example, CAR⁺ CTX130 cells. In some instances, an effective amount of a genetically engineered T cell population may comprise about 3.0×10⁷ cells to about 6×10⁸ cells that express an anti-CD70 CAR (CAR⁺ cells), for example, CAR⁺ CTX130 cells. In some instances, an effective amount of a genetically engineered T cell population may comprise about 3.0×10⁷ cells to about 4.5×10⁸ cells that express an anti-CD70 CAR (CAR⁺ cells), for example, CAR⁺ CTX130 cells. In some instances, an effective amount of a genetically engineered T cell population may comprise about 3.0×10⁷ cells to about 1×10⁸ cells that express an anti-CD70 CAR (CAR⁺ cells), for example, CAR⁺ CTX130 cells. In some instances, an effective amount of a genetically engineered T cell population may comprise about 1.0×10⁸ cells to about 9×10⁸ cells that express an anti-CD70 CAR (CAR⁺ cells), for example, CAR⁺ CTX130 cells. In some instances, an effective amount of a genetically engineered T cell population may comprise about 3.0×10⁸ cells to about 9×10⁸ cells that express an anti-CD70 CAR (CAR⁺ cells), for example, CAR⁺ CTX130 cells. In some instances, an effective amount of a genetically engineered T cell population may comprise about 4.5×10⁸ cells to about 9×10⁸ cells that express an anti-CD70 CAR (CAR⁺ cells), for example, CAR⁺ CTX130 cells. In some instances, an effective amount of a genetically engineered T cell population may comprise about 6.0×10⁸ cells to about 9×10⁸ cells that express an anti-CD70 CAR (CAR⁺ cells), for example, CAR⁺ CTX130 cells. In some instances, an effective amount of a genetically engineered T cell population may comprise about 7.5×10⁸ cells to about 9×10⁸ cells that express an anti-CD70 CAR (CAR⁺ cells), for example, CAR⁺ CTX130 cells. In some instances, an effective amount of a genetically engineered T cell population may comprise about 3.0×10⁸ cells to about 4.5×10⁸ cells that express an anti-CD70 CAR (CAR⁺ cells), for example, CAR⁺ CTX130 cells. In some instances, an effective amount of a genetically engineered T cell population may comprise about 4.5×10⁸ cells to about 6×10⁸ cells that express an anti-CD70 CAR (CAR⁺ cells), for example, CAR⁺ CTX130 cells. In some instances, an effective amount of a genetically engineered T cell population may comprise about 6×10⁸ cells to about 7.5×10⁸ cells that express an anti-CD70 CAR (CAR⁺ cells), for example, CAR⁺ CTX130 cells.

In some embodiments, an effective amount of a genetically engineered T cell population may comprise at least 3.0×10⁷ CAR⁺ CTX130 cells, at least 1×10⁸ CAR⁺ CTX130 cells, at least 3.0×10⁸ CAR⁺ CTX130 cells, at least 4×10⁸ CAR⁺ CTX130 cells, at least 4.5×10⁸ CAR⁺ CTX130 cells, at least 5×10⁸ CAR⁺ CTX130 cells, at least 5.5×10⁸ CAR⁺ CTX130 cells, at least 6×10⁸ CAR⁺ CTX130 cells, at least 6.5×10⁸ CAR⁺ CTX130 cells, at least 7×10⁸ CAR⁺ CTX130 cells, at least 7.5×10⁸ CAR⁺ CTX130 cells, at least 8×10⁸ CAR⁺ CTX130 cells, at least 8.5×10⁸ CAR⁺ CTX130 cells, or at least 9×10⁸ CAR⁺ CTX130 cells. In some examples, the amount of the CAR⁺ CTX130 cells may not exceed 1×10⁹ cells.

In some examples, an effective amount of the genetically engineered T cell population as disclosed herein (e.g., the CTX130 cells) may range from about 3.0×10⁷ CAR+CTX130 cells. In some examples, an effective amount of the genetically engineered T cell population as disclosed herein (e.g., the CTX130 cells) may range from about 1.0×10⁸ CAR+CTX130 cells. In some examples, an effective amount of the genetically engineered T cell population as disclosed herein (e.g., the CTX130 cells) may range from about 3.0×10⁸ CAR+CTX130 cells. In some examples, an effective amount of the genetically engineered T cell population as disclosed herein (e.g., the CTX130 cells) may range from about 4.5×10⁸ CAR+CTX130 cells. In some examples, an effective amount of the genetically engineered T cell population as disclosed herein (e.g., the CTX130 cells) may range from about 6.0×10⁸ CAR+CTX130 cells. In some examples, an effective amount of the genetically engineered T cell population as disclosed herein (e.g., the CTX130 cells) may range from about 7.5×10⁸ CAR+CTX130 cells. In some examples, an effective amount of the genetically engineered T cell population as disclosed herein (e.g., the CTX130 cells) may range from about 9.0×10⁸ CAR+CTX130 cells.

In some embodiments, the amount of the anti-CD70 CAR T cells such as CTX130 cells administered to a human patient does not exceed 1×10⁵ TCR⁺ cells/kg. In other embodiments, the amount of the anti-CD70 CAR T cells such as CTX130 cells administered to a human patient does not exceed 7×10⁴ TCR⁺ cells/kg.

In some embodiments, a suitable dose of CTX130 cells administered from one or more vials of the pharmaceutical composition, each comprising about 1.5×10⁸ CAR+CTX130 cells. In some embodiments, a suitable dose of CTX130 cells is administered from one or more vials of the pharmaceutical composition, each comprising about 3×10⁸ CAR+CTX130 cells. In some embodiments, a suitable dose of CTX130 cells administered to a subject is one or more folds of 1.5×10⁸ CAR+CTX130 cells, for example, 1-fold, 2-fold, 3-fold, 4-fold, 5-fold, or 6-fold of CAR+CTX130 cells. In some embodiments a suitable dose of CTX130 cells is administered from one or more full or partial vials of the pharmaceutical composition.

The efficacy of anti-CD70 CAR T cell therapy described herein can be determined by the skilled clinician. An anti-CD70 CART cell therapy is considered “effective”, if any one or all of the signs or symptoms of, as but one example, levels of CD70 are altered in a beneficial manner (e.g., decreased by at least 10%), or other clinically accepted symptoms or markers of a CD70+ solid tumor are improved or ameliorated. Efficacy can also be measured by failure of a subject to worsen as assessed by hospitalization or need for medical interventions (e.g., progression of the CD70+ solid tumor is halted or at least slowed). Methods of measuring these indicators are known to those of skill in the art and/or described herein. Treatment includes any treatment of a CD70+ solid tumor in a human patient and includes: (1) inhibiting the disease, e.g., arresting, or slowing the progression of symptoms; or (2) relieving the disease, e.g., causing regression of symptoms; and (3) preventing or reducing the likelihood of the development of symptoms.

Treatment methods described herein encompass repeating lymphodepletion and redosing of anti-CD70 CAR T cells. Prior to each redosing of anti-CD70 CAR T cells, the patient is subjected to another lymphodepletion treatment. The doses of anti-CD70 CAR T cells may be the same for the first, second, and third doses. For example, each of the first, second, and third doses is 1×10⁶ CAR+ cells, 1×10⁷ CAR+ cells, 3×10⁷ CAR+ cells, 1×10⁸ CAR+ cells, 1.5×10⁸ CAR+ cells, 4.5×10⁸ CAR⁺ cells, 6×10⁸ CAR⁺ cells, 7.5×10⁸ CAR⁺ cells, 9.8×10⁸, or 1×10⁹ CAR+ cells. In other instances, the doses of anti-CD70 CART cells may increase in number of CAR+ cells as the number of doses increases. For example, the first dose is 1×10⁶ CAR+ cells, the second dose is 1×10⁷ CAR+ cells, and the third dose is 1×10⁸ CAR+ cells. Alternatively, the first dose of CAR+ cells is lower than the second and/or third dose of CAR+ cells, e.g., the first dose is 1×10⁶ CAR+ cells and the second and the third doses are 1×10⁸ CAR+ cells. In some examples, the dose of anti-CD70 CART cells may increase by 1.5×10⁸ CAR+ cells for each subsequent dose.

Patients may be assessed for redosing following each administration of anti-CD70 CAR T cells. For example, following a first dose of anti-CD70 CAR T cells, a human patient may be eligible for receiving a second dose of anti-CD70 CART cells if the patient does not show one or more of the following: (a) dose-limiting toxicity (DLT), (b) grade 4 CRS that does not resolve to grade 2 within 72 hours, (c) grade>1 GvHD, (d) grade≥3 neurotoxicity, (e) active infection, (f) hemodynamically unstable, and (g) organ dysfunction. In another example, following a second dose of anti-CD70 CART cells, a human patient may be eligible for receiving a third dose of CTX130 if that patient does not show one or more of the following: (a) dose-limiting toxicity (DLT), (b) grade 4 CRS that does not resolve to grade 2 within 72 hours, (c) grade>1 GvHD, (d) grade≥3 neurotoxicity, (e) active infection, (f) hemodynamically unstable, and (g) organ dysfunction.

In some embodiments, a human patient as disclosed herein may be given multiple doses of the anti-CD70 CAR T cells (e.g., the CTX130 cells as disclosed herein), i.e., re-dosing. The human patient may be given up to three doses in total (i.e., re-dosing for no more than 2 times). The interval between two consecutive doses may be about 8 weeks to about 2 years. In some examples, a human patient may be re-dosed if the patient achieved a partial response (PR) or complete response (CR) after a first dose (or a second dose) and subsequently progressed within 2 years of last dose. In other examples, a human patient may be re-dosed when the patient achieved PR (but not CR) or stable disease (SD) after the most recent dose. In yet other examples, the human patient may be re-dosed about 8 weeks after the immediate proceeding dose, regardless of the patient's response to the treatment. See also Example 10 below.

Redosing of anti-CD70 CART cells such as CTX130 cells may take place about 8 weeks to about 2 years after the first dose of the anti-CD70 CAR T cells. For example, redosing of anti-CD70 CART cells may take place about 8-10 weeks after the first dose of anti-CD70 CART cells. In other examples, redosing of anti-CD70 CART cells may take place about 14-18 weeks after the first dose of the anti-CD70 CAR T cells. When a patient is administered two doses, the second dose may be administered 8 weeks to two years (e.g., 8-10 weeks or 14-18 weeks) after the preceding dose. In some examples, a patient can be administered three doses. The third dose may be administered 14-18 weeks after the first dose, and the second dose may be administered 6-10 weeks after the first dose. In some instances, the interval between two consecutive doses may be about 6-10 weeks.

In some examples, a human patient may be given up to 2 additional doses of the anti-CD70 CAR T cells (e.g., the CTX130 cells), each accompanied with an LD therapy, when the human patient shows loss of response within the first 2 years after the last dose of the anti-CD70 CAR T cells. Alternatively, a human patient may be given up to 2 additional doses of the anti-CD70 CAR T cells (e.g., the CTX130 cells), each accompanied with an LD therapy, when the human patient shows stable disease or progressive disease with significant clinical benefit after the last treatment with the anti-CD70 CAR T cells (e.g., about 6 weeks after the last treatment) as determined by a medical practioner.

Following each dosing of anti-CD70 CAR T cells, a human patient may be monitored for acute toxicities such as cytokine release syndrome (CRS), tumor lysis syndrome (TLS), neurotoxicity (e.g., ICANS), graft versus host disease (GvHD), viral encephalitis, on target off-tumor toxicity, and/or uncontrolled T cell proliferation. The on target off-tumor toxicity may comprises activity of the population of genetically engineered T cells against activated T lymphocytes, B lymphocytes, dentritic cells, osteoblasts and/or renal tubular-like epithelium. One or more of the following potential toxicity may also be monitored: hytotension, renal insufficiency, hemophagocytic lymphohistiocytosis (HLH), prolonged cytopenias, and/or drug-induced liver injury. After each dose of anti-CD70 CAR T cells, a human patient may be monitored for at least 28 days for development of toxicity. If development of toxicity is observed, the human patient may be subjected to toxicity management. Treatments for patients exhibiting one or more symptoms of acute toxicity are known in the art. For example, a human patient exhibiting a symptom of CRS (e.g., cardiac, respiratory, and/or neurological abnormalities) may be administered an anti-cytokine therapy. In addition, a human patient that does not exhibit a symptom of CRS may be administered an anti-cytokine therapy to promote proliferation of anti-CD70 CART cells.

Anti-CD70 CAR T cell treatment methods described herein may be used on a human patient that has undergone a prior anti-cancer therapy. For example, anti-CD70 CAR T cells as described herein may be administered to a patient that has been previously treated with a checkpoint inhibitor, a tyrosine kinase inhibitor, a vascular endothelial growth factor inhibitoror, or a combination thereof.

Anti-CD70 CAR T cells treatment methods described herein may also be used in combination therapies. For example, anti-CD70 CAR T cells treatment methods described herein may be co-used with other therapeutic agents, for treating a CD70+ solid tumor, or for enhancing efficacy of the genetically engineered T cell population and/or reducing side effects of the genetically engineered T cell population.

(iv) NK Cell Inhibitor Treatment

In some embodiments, any of the anti-CD70 CAR T cells such as the CTX130 cells disclosed herein are used in combination with an NK cell inhibitor, such as a CD38 inhibitor. In some instances, the CD38 inhibitor is an anti-CD38 antibody. In one specific example, the anti-CD38 antibody is daratumumab.

An NK cell inhibitor such as daratumumab may be formulated in a pharmaceutical composition and given to a suitable subject as disclosed herein at a suitable time point relative to the LD and/or allogeneic anti-CD70 CAR-T cell (e.g., CTX130) therapy. A pharmaceutical composition comprising daratumumab and one or more pharmaceutically acceptable carriers may be administered to the subject via a suitable route, for example, orally, parenterally, by inhalation spray, rectally, nasally, buccally, vaginally or via an implanted reservoir.

In some embodiments, the pharmaceutical composition comprising daratumumab is to be administered by injection, for example, intravenous infusion or subcutaneous injection. A sterile injectable composition, e.g., a sterile injectable aqueous or oleaginous suspension, can be formulated according to techniques known in the art using suitable dispersing or wetting agents (such as Tween® 80) and suspending agents. The sterile injectable preparation can also be a sterile injectable solution or suspension in a non-toxic parenterally acceptable diluent or solvent, for example, as a solution in 1,3-butanediol. Among the acceptable vehicles and solvents that can be employed are mannitol, water, Ringer's solution and isotonic sodium chloride solution. In addition, sterile, fixed oils are conventionally employed as a solvent or suspending medium (e.g., synthetic mono- or diglycerides). Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions can also contain a long-chain alcohol diluent or dispersant, or carboxymethyl cellulose or similar dispersing agents. Other commonly used surfactants such as Tweens or Spans or other similar emulsifying agents or bioavailability enhancers which are commonly used in the manufacture of pharmaceutically acceptable solid, liquid, or other dosage forms can also be used for the purposes of formulation.

The pharmaceutical compositions as described herein can comprise pharmaceutically acceptable carriers, excipients, or stabilizers in the form of lyophilized formulations or aqueous solutions. Remington: The Science and Practice of Pharmacy 20th Ed. (2000) Lippincott Williams and Wilkins, Ed. K. E. Hoover. Such carriers, excipients or stabilizers may enhance one or more properties of the active ingredients in the compositions described herein, e.g., bioactivity, stability, bioavailability, and other pharmacokinetics and/or bioactivities.

Acceptable carriers, excipients, or stabilizers are nontoxic to recipients at the dosages and concentrations used, and may comprise buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; benzoates, sorbate and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, serine, alanine or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™ (polysorbate), PLURONICS™ (nonionic surfactants), or polyethylene glycol (PEG).

In some embodiments, an effective amount of daratumumab (e.g., about 10-20 mg/kg such as about 16 mg/kg, e.g., via intravenous infusion) may be given to the subject via a suitable route (e.g., intravenous infusion). The effective amount of daratumumab may split into two parts (e.g., equally) and be administered to the subject on two consecutive days. In some embodiments, a reduced dose of daratumumab (e.g., 8 mg/kg via intravenous infusion) may be administered to a patient. In other embodiments, an effective amount of daratumumab may be about 1500-2000 mg (e.g., 1800 mg) by subcutaneous injection.

In some examples, administration of daratumumab may be performed prior to the LD therapy. In specific examples, administration of daratumumab may be performed within 3 days (e.g., at least 12 hours) prior to the LD therapy. Alternatively or in addition, administration of daratumumab may be performed no more than 10 days prior to the treatment with the anti-CD70 CAR-T cells such as CTX130 cells. In one example, administration of daratumumab may be performed at least 12 hours prior to starting the LD treatment and within 10 days of the administration of the anti-CD70 CAR-T cells such as CTX130 cells.

In some instances, daratumumab treatment may be repeated once every 2-4 weeks. In some examples, daratumumab treatment may be repeated once every 3 weeks. For example, a patient may be given a second dose of daratumumab about 3 weeks after administration of the anti-CD70 CAR-T cells. In some instances (e.g., a patient achieves stable disease or a better response), a third dose of darabumumab may be given to the patient, e.g., about 6-7 weeks after administration of the anti-CD70 CAR-T cells. A subsequent dose of daratumumab may be the same as the preceding dose of daratumumab given to the patient, for example, 16 mg/kg, via intravenous infusion, which may split into two parts as disclosed herein. Alternatively, the subsequent doses of daratumumab may be lower than that of the preceding dose. The additional doses of daratumumab may vary as determined by a medical practitioner. If the subject exhibits disease progress or severe toxicity, the additional daratumumab treatment may be terminated. In some embodiments, a lower daratumumab dose, for example, 8 mg/kg, may be used.

NK cell inhibitors such as anti-CD38 antibodies (e.g., daratumumab) were found to suppress potential host immune responses to allogenic CAR T cells, for example, immune responses mediated by NK cells against allogenic CAR T cells that are deficient in MHC Class I expression. The NK cell inhibitor may also allow increased expansion and persistence of the CAR T cells. Accordingly, the NK cell inhibitors as disclosed herein, such as anti-CD38 antibodies (e.g., daratumumab), could be co-used with CAR T cells that express an anti-CD70 CAR and are deficient in MHC Class I expression. In some instances, the anti-CD70 CAR T cells that are deficient in MHC Class I expression may have a level of MHC Class I expression at least 50% (e.g., at least 60%, at least 70%, at least 80%, or at least 90%) lower than the anti-CD70 CAR T cell counterpart that is not deficient in MHC Class I expression (i.e., having the same genetic editings except for MHC Class I). In some examples, the anti-CD70 CAR T cells that are deficient in MHC Class I expression may have no detectable level of MHC Class I expression as measured by a conventional assay.

In some embodiments, the deficience in MHC Class I expression may be caused by gene editing of one or more genes coding for components of the MHC Class I complex to disrupte the expression thereof. Such gene editing may be achieved by a conventional method. In some examples, the one or more genes coding for MHC Class I components may be disrupted by a CRISPR/Cas gene editing system. In some examples, the β2M gene can be disrupted via a gene editing method, for example, CRISPR. More details for disrupting the β2M gene via a CRISPR/Cas gene editing system are provided elsewhere herein.

The combined therapy of an NK cell inhibitor such as an anti-CD38 antibody (e.g., daratumumab) and anti-CD70 CAR T cells deficient in MHC Class I expression for treating a target CD70+ solid tumor such as RCC is also within the scope of the present disclosure. Such a combined therapy may involve any of the treatment regimens as also disclosed herein.

(v) Exemplary Treatment Regimens

In some embodiments, a treatment method as provided herein may be performed as follows. A suitable human patient having one of the target CD70+ solid tumor (e.g., RCC) as disclosed herein may be identified via routine medical practice or as disclosed herein (see Example 10 below). Such a human patient may meet the inclusion and/or exclusion criteria disclosed in Example 10 below. An LD chemotherapy can be performed to the human patient. Such an LD chemotherapy may comprise co-administration (e.g., intravenous injection) of fludarabine at 30 mg/m² and cyclophosphamide at 500 mg/m² daily for three days. Within about 2-7 days, the human patient can be administered the anti-CD70 CAR T cells disclosed herein (e.g., CTX130) via intravenous infusion at one of the following doses: 3.0×10⁷ CAR+ cells, 1×10⁸ CAR+ cells, 3.0×10⁸ CAR+ cells, 4.5×10⁸ CAR+ cells, 6.0×10⁸ CAR+ cells, 7.5×10⁸ CAR+ cells, or 9.0×10⁸ CAR+ cells. Optionally, the human patient may be administered up to two additional doses of the anti-CD70 CAR T cells, each accompanied with the LD therapy, when the patient shows 1) loss of response within the first 2 years after the last dose of the anti-CD70 CAR T cells, or 2) stable disease or progressive disease with significant clinical benefit after the last dose of the anti-CD70 CAR T cells (e.g., at least 6 weeks after the treatment). Significant clinical benefit can be assessed by a medical practioner. The additional dose(s) may be the same as the initial dose. Alternatively, the subsequent dose(s) may be adjusted according to the patient's response to the initial dose, which can be determined by a medical practioner.

In some embodiments, a treatment method as provided herein may be performed as follows. A suitable human patient having one of the target CD70+ solid tumor (e.g., RCC) as disclosed herein may be identified via routine medical practice or as disclosed herein (see Example 10 below). Such a human patient may meet the inclusion and/or exclusion criteria disclosed in Example 10 below. Multiple cycles of the treatment regimen (e.g., up to 3) may be performed to the human patient as disclosed below. In each treatment cycle, an LD chemotherapy can be performed to the human patient. Such an LD chemotherapy may comprise co-administration (e.g., intravenous injection) of fludarabine at 30 mg/m² and cyclophosphamide at 500 mg/m² daily for three days. Within about 2-7 days, the human patient can be administered the anti-CD70 CAR T cells disclosed herein (e.g., CTX130) via intravenous infusion at one of the following doses: 3.0×10⁷ CAR+ cells, 1×10⁸ CAR+ cells, 3.0×10⁸ CAR+ cells, 4.5×10⁸ CAR+ cells, 6.0×10⁸ CAR+ cells, 7.5×10⁸ CAR+ cells, or 9.0×10⁸ CAR+ cells. This treatment regimen can be repeated multiple times, for example, up to three times, i.e., the human patient receives up to three doses of the anti-CD70 CAR T cells. In some examples, multiple doses of the anti-CD70 CAR T cells may be the same. In other examples, a subsequent dose of the anti-CD70 CAR T cells may be higher than the preceding one. Alternatively, a subsequent dose of the anti-CD70 CAR T cells may be lower than the preceding one. In some instances, two consecutive doses of the anti-CD70 CAR T cells may be about 8 weeks apart (e.g., regardless of disease response).

In some embodiments, a treatment method as provided herein may be performed as follows. A suitable human patient having one of the target CD70+ solid tumor (e.g., RCC) as disclosed herein may be identified via routine medical practice or as disclosed herein (see Example 10 below). Such a human patient may meet the inclusion and/or exclusion criteria disclosed in Example 10 below. A first dose of darabumumab (e.g., 16 mg/kg IV or 1800 mg SC) may be administered to the human patient via intravenous infusion. In some instances, the dose of daratumumab may be split into two parts evenly (e.g., 8 mg/kg i.v. each), which can be administered to the patient on two consecutive days. An LD chemotherapy can be performed to the human patient at a suitable time point after the daratumumab treatment, for example, at least 12 hours after the daratumumab treatment. Such an LD chemotherapy may comprise co-administration (e.g., intravenous injection) of fludarabine at 30 mg/m² and cyclophosphamide at 500 mg/m² daily for three days. Within about 2-7 days after the LD therapy and within 10 days after the daratumumab treatment, the human patient can be administered the anti-CD70 CAR T cells disclosed herein (e.g., CTX130) via intravenous infusion at one of the following doses: 3.0×10⁷ CAR+ cells, 1×10⁸ CAR+ cells, 3.0×10⁸ CAR+ cells, 4.5×10⁸ CAR+ cells, 6.0×10⁸ CAR+ cells, 7.5×10⁸ CAR+ cells, or 9.0×10⁸ CAR+ cells. Following the anti-CD70 CAR T cell therapy, a second dose of daratumumab may be administered to the human patient, for example, about three weeks after administration of the genetically engineered T cells. The second dose of daratumumab may be the same as the first dose of daratumumab. Alternatively, the second dose of daratumumab may be lower than the first dose. In some instances, a third dose of daratumumab may be administered to the patient about 6-7 weeks after administration of the genetically engineered T cells, for example, when the patient achieves stable disease or a better disease response.

Optionally, the human patient may be administered up to two additional doses of the anti-CD70 CAR T cells, each accompanied with the LD therapy and optionally the daratumumab treatment, when the patient shows 1) loss of response within the first 2 years after the last dose of the anti-CD70 CAR T cells, or 2) stable disease or progressive disease with significant clinical benefit after the last dose of the anti-CD70 CAR T cells (e.g., at least 6 weeks after the treatment). Significant clinical benefit can be assessed by a medical practioner. The additional dose(s) may be the same as the initial dose. Alternatively, the subsequent dose(s) may be adjusted according to the patient's response to the initial dose, which can be determined by a medical practioner. Alternatively or in addition, additional doses of daratumumab may be performed to the human patient, e.g., at a lower dose such as 8 mg/kg (i.v.).

In some embodiments, a treatment method as provided herein may be performed as follows. A suitable human patient having one of the target CD70+ solid tumor (e.g., RCC) as disclosed herein may be identified via routine medical practice or as disclosed herein (see Example 10 below). Such a human patient may meet the inclusion and/or exclusion criteria disclosed in Example 10 below. Multiple cycles of the treatment regimen (e.g., up to 3) may be performed to the human patient as disclosed below. In each treatment cycle, a first dose of darabumumab (e.g., 16 mg/kg i.v., or 1800 mg s.c.) may be administered to the human patient via intravenous infusion. In some instances, the dose of daratumumab may be split into two parts evenly (e.g., 8 mg/kg i.v. each), which can be administered to the patient on two consecutive days. An LD chemotherapy can be performed to the human patient at a suitable time point after the daratumumab treatment, for example, at least 12 hours after the daratumumab treatment. Such an LD chemotherapy may comprise co-administration (e.g., intravenous injection) of fludarabine at 30 mg/m² and cyclophosphamide at 500 mg/m² daily for three days. Within about 2-7 days, the human patient can be administered the anti-CD70 CAR T cells disclosed herein (e.g., CTX130) via intravenous infusion at one of the following doses: 3.0×10⁷ CAR+ cells, 1×10⁸ CAR+ cells, 3.0×10⁸ CAR+ cells, 4.5×10⁸ CAR+ cells, 6.0×10⁸ CAR+ cells, 7.5×10⁸ CAR+ cells, or 9.0×10⁸ CAR+ cells. Following the anti-CD70 CAR T cell therapy, a second dose of daratumumab may be administered to the human patient, for example, about three weeks after administration of the anti-CD70 CAR-T cells such as CTX130 cells. The second dose of daratumumab may be the same as the first dose of daratumumab. Alternatively, the second dose of daratumumab may be lower than the first dose. In some instances, a third dose of daratumumab may be administered to the patient about 6-7 weeks after administration of the genetically engineered T cells, for example, when the patient achieves stable disease or a better disease response.

This treatment regimen can be repeated multiple times, for example, up to three times, i.e., the human patient receives up to three doses of the anti-CD70 CAR T cells. In some examples, multiple doses of the anti-CD70 CAR T cells may be the same. In other examples, a subsequent dose of the anti-CD70 CAR T cells may be higher than the preceding one. Alternatively, a subsequent dose of the anti-CD70 CAR T cells may be lower than the preceding one. In some instances, two consecutive doses of the anti-CD70 CAR T cells may be about 8 weeks apart (e.g., regardless of disease response).

### V. Kit for Treating CD70 Positive Solid Tumors

The present disclosure also provides kits for use of a population of anti-CD70 CAR T cells such as CTX130 T cells and optionally an NK cell inhibitor such as an anti-CD38 antibody (e.g., daratumumab) as described herein in methods for treating C, such as those disclosed herein (e.g., RCC). Such kits may include a first container comprising a first pharmaceutical composition that comprises any of the populations of genetically engineered anti-CD70 CAR T cells (e.g., those described herein such as CTX130 cells), and a pharmaceutically acceptable carrier, and optionally a second container comprising a second pharmaceutical composition comprising the NK cell inhibitor such as daratumumab. The anti-CD70 CAR-T cells may be suspended in a cryopreservation solution such as those disclosed herein. Optionally, the kit may further comprise a third container comprising a third pharmaceutical composition that comprises one or more lymphodepleting agents.

In some embodiments, the kit can comprise instructions for use in any of the methods described herein. The included instructions can comprise a description of administration of the anti-CD70 CAR T cells, and optionally daratumumab and any additional therapeutic agents to a subject to achieve the intended activity in a human patient having a CD70 positive solid tumor such as those disclosed herein (e.g., RCC). The kit may further comprise a description of selecting a human patient suitable for treatment based on identifying whether the human patient is in need of the treatment.

The instructions relating to the use of a population of anti-CD70 CAR-T cells such as CTX130 T cells described herein generally include information as to dosage, dosing schedule, and route of administration for the intended treatment. The instructions may also include information relating to the use of daratumumab, for example, dosage, dosing schedule, and route of administration for the intended treatment. The containers may be unit doses, bulk packages (e.g., multi-dose packages) or sub-unit doses. Instructions supplied in the kits of the disclosure are typically written instructions on a label or package insert. The label or package insert indicates that the population of genetically engineered T cells is used for treating, delaying the onset, and/or alleviating a symptom of the hematopoietic maligancy in a subject.

The kits provided herein are in suitable packaging. Suitable packaging includes, but is not limited to, vials, bottles, jars, flexible packaging, and the like. Also contemplated are packages for use in combination with a specific device, such as an inhaler, nasal administration device, or an infusion device. A kit may have a sterile access port (for example, the container may be an intravenous solution bag or a vial having a stopper pierceable by a hypodermic injection needle). The container may also have a sterile access port. At least one active agent in the pharmaceutical composition is a population of the anti-CD70 CAR-T cells such as the CTX130 cells as disclosed herein.

Kits optionally may provide additional components such as buffers and interpretive information. Normally, the kit comprises a container and a label or package insert(s) on or associated with the container. In some embodiment, the disclosure provides articles of manufacture comprising contents of the kits described above.

### General Techniques

The practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook, et al., 1989) Cold Spring Harbor Press; Oligonucleotide Synthesis (M. J. Gait, ed. 1984); Methods in Molecular Biology, Humana Press; Cell Biology: A Laboratory Notebook (J. E. Cellis, ed., 1989) Academic Press; Animal Cell Culture (R. I. Freshney, ed. 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds. 1993-8) J. Wiley and Sons; Methods in Enzymology (Academic Press, Inc.); Handbook of Experimental Immunology (D. M. Weir and C. C. Blackwell, eds.): Gene Transfer Vectors for Mammalian Cells (J. M. Miller and M. P. Calos, eds., 1987); Current Protocols in Molecular Biology (F. M. Ausubel, et al. eds. 1987); PCR: The Polymerase Chain Reaction, (Mullis, et al., eds. 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practice approach (D. Catty, ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds. Harwood Academic Publishers, 1995); DNA Cloning: A practical Approach, Volumes I and II (D. N. Glover ed. 1985); Nucleic Acid Hybridization (B. D. Hames & S. J. Higgins eds. (1985; Transcription and Translation (B. D. Hames & S. J. Higgins, eds. (1984; Animal Cell Culture (R.I. Freshney, ed. (1986»; Immobilized Cells and Enzymes (IRL Press, (1986; and B. Perbal, A practical Guide To Molecular Cloning (1984); F. M. Ausubel et al. (eds.).

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

In order that the invention described may be more fully understood, the following examples are set forth. The examples described in this application are offered to illustrate the methods and compositions provided herein and are not to be construed in any way as limiting their scope.

### Example 1: Generation of T Cells with Multiple Gene Knockouts

This example describes the use of CRISPR/Cas9 gene editing technology to produce human T cells that lack expression of two or three genes simultaneously. Specifically, the T cell receptor (TCR) gene (gene edited in the TCR Alpha Constant (TRAC) region), the β2-microglobulin (β2M) gene, and the Cluster of Differentiation 70 (CD70) gene were edited by CRISPR/Cas9 gene editing to produce T cells deficient in two or more of the listed genes. The following abbreviations are used in for brevity and clarity:

2×KO: TRAC⁻/β2M⁻

3×KO (CD70): TRAC⁻/β2M⁻/CD70⁻

Activated primary human T cells were electroporated with Cas9:gRNA RNP complexes. The nucleofection mix contained the Nucleofector™ Solution, 5×10⁶ cells, 1 μM Cas9, and 5 μM gRNA (as described in Hendel et al., Nat Biotechnol. 2015; 33(9):985-989, PMID: 26121415). For the generation of double knockout T cells (2×KO), the cells were electroporated with two different RNP complexes, each containing Cas9 protein and one of the following sgRNAs: TRAC (SEQ ID NO: 6) and β2M (SEQ ID NO: 10) at the concentrations indicated above. For the generation of triple knockout T cells (3×KO), the cells were electroporated with three different RNP complexes, each RNA complex containing Cas protein and one of the following sgRNAs: (a) TRAC (SEQ ID NO: 6), β2M (SEQ ID NO: 10), and CD70 (SEQ ID NO: 2 or 66). The unmodified versions (or other modified versions) of the gRNAs may also be used (e.g., SEQ ID NOS: 3, 7, 11, and/or 67). See also sequences in Table 7.

TABLE 7
gRNA Sequences/Target Sequences.

| Name | Unmodified Sequence | Modified Sequence |
|---|---|---|
| TRAC sgRNA | AGAGCAACAGUGCUGUG | A*G*A*GCAACAGUGCUGUGGC |
| | GCCguuuuagagcuagaaauagcaa | Cguuuuagagcuagaaauagcaaguuaaaaua |
| | guuaaaauaaggcuaguccguuauca | aggcuaguccguuaucaacuugaaaaaguggc |
| | acuugaaaaaguggcaccgagucggu | accgagucggugcU*U*U*U (SEQ ID |
| | gcUUUU | NO: 6) |
| | (SEQ ID NO: 7) | |
| TRAC sgRNA spacer | AGAGCAACAGUGCUGUG | A* G* A* GCAACAGUGCUGUGGC |
| | GCC (SEQ ID NO: 9) | C (SEQ ID NO: 8) |
| β2M sgRNA | GCUACUCUCUCUUUCUGG | G*C*U*ACUCUCUCUUUCUGGC |
| | CCguuuuagagcuagaaauagcaag | Cguuuuagagcuagaaauagcaaguuaaaaua |
| | uuaaaauaaggcuaguccguuaucaac | aggcuaguccguuaucaacuugaaaaaguggc |
| | uugaaaaaguggcaccgagucggugc | accgagucggugcU*U*U*U |
| | UUUU | (SEQ ID NO: 10) |
| | (SEQ ID NO: 11) | |
| β2M sgRNA spacer | GCUACUCUCUCUUUCUGG | G*C*U*ACUCUCUCUUUCUGGC |
| | CC (SEQ ID NO: 13) | C (SEQ ID NO: 12) |
| CD70 sgRNA; also referred | GCUUUGGUCCCAUUGGU | G*C*U*UUGGUCCCAUUGGUCG |
| to as: T7 | CGCguuuuagagcuagaaauagcaa | Cguuuuagagcuagaaauagcaaguuaaaaua |
| | guuaaaauaaggcuaguccguuauca | aggcuaguccguuaucaacuugaaaaaguggc |
| | acuugaaaaaguggcaccgagucggu | accgagucggugcU*U*U*U (SEQ ID |
| | gcUUUU | NO: 2) |
| | (SEQ ID NO: 3) | |
| CD70 sgRNA spacer; also | GCUUUGGUCCCAUUGGU | G*C*U*Uuggucccauuggucg |
| referred to as: T7 | CGC (SEQ ID NO: 5) | C (SEQ ID NO: 4) |
| CD70 sgRNA; also referred | GCCCGCAGGACGCACCCA | G*C*C*CGCAGGACGCACCCAUA |
| to as: T8 | UAguuuuagagcuagaaauagcaag | guuuuagagcuagaaauagcaaguuaaaauaa |
| | uuaaaauaaggcuaguccguuaucaac | ggcuaguccguuaucaacuugaaaaaguggca |
| | uugaaaaaguggcaccgagucggugc | ccgagucggugcU*U*U*U (SEQ ID |
| | UUUU | NO: 66) |
| | (SEQ ID NO: 67) | |
| CD70 sgRNA spacer; also | GCCCGCAGGACGCACCCA | G*C*C*CGCAGGACGCACCCAUA |
| referred to as: T8 | UA (SEQ ID NO: 69) | (SEQ ID NO: 68) |

About one (1) week post electroporation, cells were either left untreated or treated with phorbol myristate acetate (PMA)/ionomycin overnight. The next day cells were processed for flow cytometry (see, e.g., Kalaitzidis D et al., J Clin Invest 2017; 127(4): 1405-1413) to assess TRAC, β2M, and CD70 expression levels at the cell surface of the edited cell population. The following primary antibodies were used (Table 8):

TABLE 8
Antibodies.

| Antibody | Clone | Fluor | Catalogue # | Dilution | For 1 |
|---|---|---|---|---|---|
| TCR | BW242/412 | PE | 130-091-236 | 1:100 | 1 μL |
| | | | (Miltenyi) | | |
| β2M | 2M2 | PE-Cy7 | 316318 (Biolegend) | 1:100 | 1 μL |
| CD70 | 113-16 | FITC | 355105 (Biolegend) | 1:100 | 1 μL |

Table 9 shows highly efficient multiple gene editing. For the triple knockout cells, 80% of viable cells lacked expression of TCR, β2M, and CD70 (Table 9).

TABLE 9
% of viable cells lacking expression in 3KO
cell populations.

| | TRAC | β2M | CD70 | |
|---|---|---|---|---|
| | KO | KO | KO | 3KO |
| 3KO (CD70) | 99% | 79% | 99% | 80% |

To assess whether triple gene editing in T cells affects cell expansion, cell numbers were enumerated among double and triple gene edited T cells (unedited T cells were used as a control) over a two week period of post editing. 5×10⁶ cells were generated and plated for each genotype of T cells.

Cell proliferation (expansion) continued over the post-electroporation window test. Similar cell proliferation was observed among the double (β2M−/TRAC−) and triple β2M−/TRAC−/CD70−), knockout T cells, as indicated by the number of viable cells (data not shown). These data suggest that multiple gene editing does not impact T cell health as measured by T cell proliferation.

### Example 2: Generation of Anti-CD70 CAR T Cells with Multiple Knockouts

This example describes the production of allogeneic human T cells that lack expression of the TCR gene, β2M gene, and/or CD70 gene, and express a chimeric antigen receptor (CAR) targeting CD70. These cells are designated TCR⁻/β2M⁻/CD70⁻/anti-CD70 CAR⁺ or 3×KO (CD70) CD70 CAR⁺.

A recombinant adeno-associated adenoviral vector, serotype 6 (AAV6) (MOI 50, 000) comprising the nucleotide sequence of SEQ ID NO: 43 (comprising the donor template in SEQ ID NO: 44, encoding anti-CD70 CAR comprising the amino acid sequence of SEQ ID NO: 46 or SEQ ID NO: 81) was delivered with Cas9:sgRNA RNPs (1 μM Cas9, 5 μM gRNA) to activated allogeneic human T cells. The following sgRNAs were used: TRAC (SEQ ID NO: 6), β2M (SEQ ID NO: 10), and CD70 (SEQ ID NO: 2 or 66). The unmodified versions (or other modified versions) of the gRNAs may also be used (e.g., SEQ ID NOS: 3, 7, 11, and/or 67). About one (1) week post electroporation, cells were processed for flow cytometry to assess TRAC, β2M, and CD70, expression levels at the cell surface of the edited cell population. The following primary antibodies were used (Table 10):

TABLE 10
Antibodies.

| Antibody | Clone | Fluor | Catalogue # | Dilution |
|---|---|---|---|---|
| TCR | BW242/412 | PE | 130-091-236 (Miltenyi) | 1:100 |
| β2M | 2M2 | PE-Cy7 | 316318 (Biolegend) | 1:100 |
| CD70 | 113-16 | FITC | 355105 (Biolegend) | 1:100 |

T cell Proportion Assay. The proportions of CD4+ and CD8+ cells were then assessed in the edited T cell populations by flow cytometry using the following antibodies (Table 11):

TABLE 11
Antibodies.

| Antibody | Clone | Fluor | Catalogue # | Dilution |
|---|---|---|---|---|
| CD4 | RPA-T4 | BV510 | 300545 (Biolegend) | 1:100 |
| CD8 | SK1 | BV605 | 344741 (Biolegend) | 1:100 |

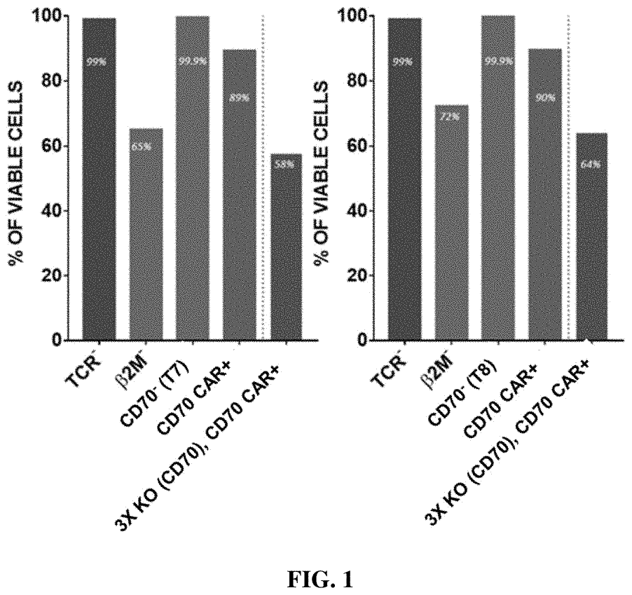

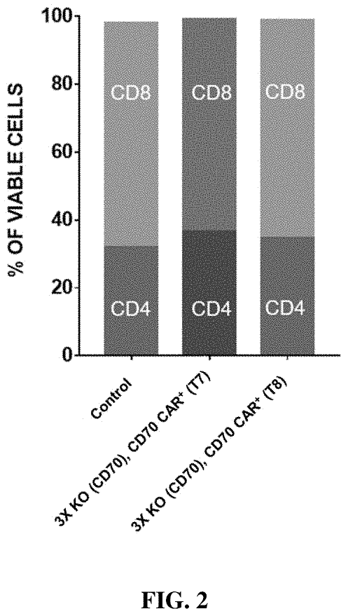

High efficiency gene editing and CAR expression was achieved in the edited anti-CD70 CAR T cell populations. In addition, editing did not adversely alter CD4/CD8 T cell populations. FIG. 1 shows highly efficient gene editing and anti-CD70 CAR expression in the triple knockout CART cell. More than 55% of viable cells lacked expression of TCR, β2M, and CD70, and also expressed the anti-CD70 CAR. FIG. 2 shows that normal proportions of CD4/CD8 T cell subsets were maintained in the TRAC−/β2M−/CD70−/anti-CD70 CAR+ cells, suggesting that these multiple gene edits do not affect T cell biology as measured by the proportion of CD4/CD8 T cell subsets.

### Example 3: Effect of CD70 KO on Cell Proliferation and Cytotoxicity of Anti-CD70 CAR T Cells In Vitro

(A) Cell Proliferation

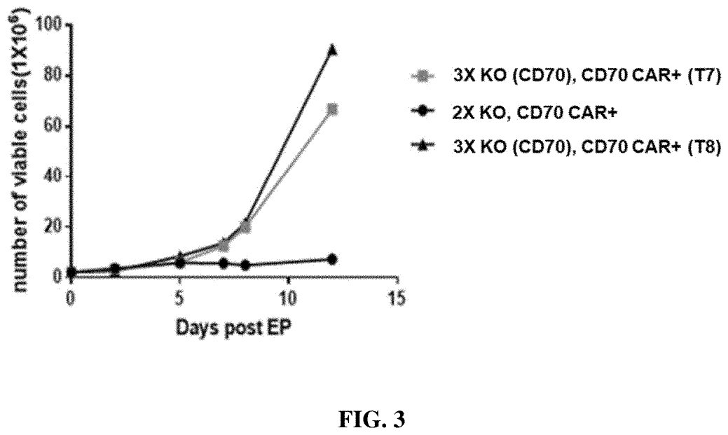

To further assess the impact of disrupting the CD70 gene in CART cells, anti-CD70 CAR T cells were generated as described in Example 2. Specifically, 3×KO (TRAC−/β2M−/CD70−) anti-CD70 CAR T cells were generated using two different gRNAs (T7 (SEQ ID NO: 2 and T8 (SEQ ID NO: 66)). After electroporation, cell expansion was assessed by enumerating double or triple gene edited T cells over a two week period of post editing. 5×10⁶ cells were generated and plated for each genotype of T cells. Proliferation was determined by counting number of viable cells. FIG. 3 shows that triple knockout TRAC⁻/β2M⁻/CD70⁻/anti-CD70 CAR⁺ T cells generated with either T7 or T8 gRNAs exhibited greater cell expansion relative to double knockout TRAC⁻/β2M⁻/anti-CD70 CAR⁺ T cells. These data suggest that knocking-out the CD70 gene gives a cell proliferation advantage to anti-CD70 CAR+ T cells.

(B) Cell Cytotoxicity

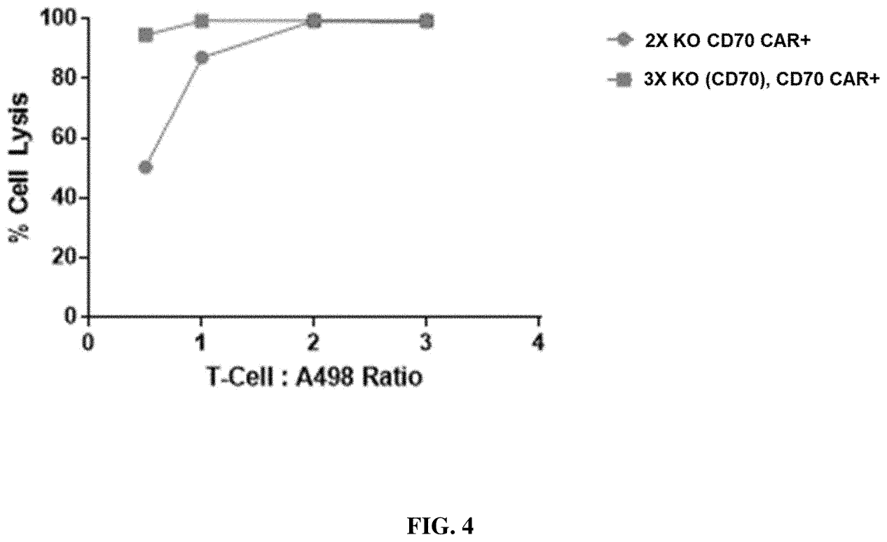

A cell killing assay was used to assess the ability of the TRAC⁻/β2M⁻/CD70⁻/anti-CD70 CAR⁺ T cells and TRAC⁻/β2M⁻/anti-CD70 CAR⁺ T cells to kill a CD70⁺ adherent renal cell carcinoma (RCC)-derived cell line (A498 cells). Adherent cells were seeded in 96-well plates at 50,000 cells per well and left overnight at 37° C. The next day edited anti-CD70 CAR T cells were added to the wells containing target cells at the indicated ratios. After the indicated incubation period, CAR T cells were removed from the culture by aspiration and 100 μL Cell titer-Glo (Promega) was added to each well of the plate to assess the number of remaining viable cells. The amount of light emitted per well was then quantified using a plate reader. The cells exhibited potent cell killing of RCC-derived cells following 24-hour co-incubation (FIG. 4). The anti-CD70 CAR T cells demonstrated higher potency when CD70 was knocked out, which is clearly visible at low T cell: A498 ratios (1:1 and 0.5:1) where cell lysis remains above 90% for TRAC⁻/β2M⁻/CD70⁻/anti-CD70 CAR⁺ T cells, while cells lysis drops below 90% for the TRAC⁻/β2M⁻/anti-CD70 CAR⁺ T cells. This suggests that knocking-out the CD70 gene gives a higher cell kill potency to anti-CD70 CAR+ T cells.

### Example 4: Knockout of CD70 Maintained Anti-CD70 CAR⁺ T Cell Killing Upon Serial Rechallenge

The anti-CD70 CAR⁺ T cells generated above were serially rechallenged with CD70+ kidney cancer cell line, A498, and evaluated for their ability to kill the CD70+ kidney cancer cell line A498.

A498 cells were plated in a T25 flask and mixed at a ratio of 2:1 (T-cell to A498) with 10×10⁶ anti-CD70 CAR⁺ T cells containing either two (TRAC⁻/β2M⁻) or three (TRAC⁻/β2M⁻/CD70⁻)) gRNA edits. Anti-CD70 CAR+ T cells with three edits are also referred to as CTX130.

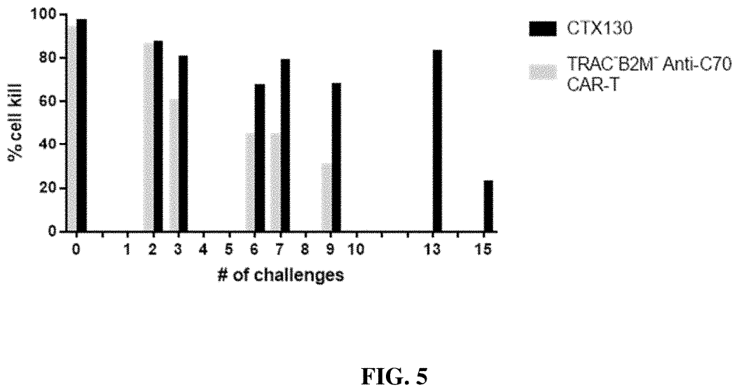

Two or three days after each challenge, cells were counted, washed, resuspended in fresh T cell media, and re-challenged the next day with the same ratio of two anti-CD70 CAR⁺ T cell per one A498 cell (2:1, CAR⁺ T:target). Challenging of anti-CD70 CAR⁺ T cells with CD70+A498 cells was repeated 13 times. Three to four days following each exposure to A498 cells (and prior to the next rechallenge), aliquots of the culture were taken and analyzed for the ability of the CART Cells to kill A498 target cells at a ratio of 2:1 (CART cell: Target cell). Cell kill was measured using Cell titer-glo (Promega). Prior to the first challenge with A498, anti-CD70 CAR+ T cells with 2×KO (TRAC⁻/β2M⁻) and 3×KO (TRAC⁻/β2M⁻/CD70⁻), each exhibited a target cell killing of A498 cells approaching 100%. By challenge nine however, the 2×KO (TRAC⁻/β2M⁻) anti-CD70 CAR⁺ T cells induced target cell killing of A498 cells below 40%, while 3×KO (TRAC⁻/β2M⁻/CD70⁻) anti-CD70 CAR⁺ T cells exhibited target cell killing above 60% (FIG. 5). The target cell killing for 3×KO (TRAC⁻/β2M⁻/CD70⁻) anti-CD70 CAR⁺ T cells remained above 60% even following 13 re-challenges with A498 cells, demonstrating that these CAR+ T cells were resistant to exhaustion.

### Example 5: Measurement of Cytokine Secretion by Anti-CD70 CAR+ T Cells (CTX130) in the Presence of CD70+ Cells

The objective of this study was to assess the ability of CTX130 to secrete effector cytokines in the presence of CD70 expressing cells.

Target cancer cell lines (A498, ACHN & MCF7) were obtained from ATCC (HTB-44, CRL-1611 & HTB-22). Expression of CD70 on target cell lines was evaluated. In brief, CTX130 or control T cells (unedited T cells) were co-cultured with target cell lines in U-bottom 96-well plates at varying ratios of T cells to target cells from 0.125:1 up to 4:1. The cells were cultured in total of 200 μL of target cell media for 24 hours, as described in each experiment. Assay was performed in media which did not contain addition of IL-2 and IL-7 to evaluate T cell activation in the absence of supplemental cytokines.

The ability of CTX130 or control T cells (unedited T cells with no anti-CD70 CAR expression) to specifically secrete the effector cytokines interferon-γ (INFγ) and interleukin-2 (IL-2) following co-culture with CD70 positive or CD70 negative target cells was assessed using a Luminex based MILLIPLEX assay as described herein. A498 and ACHN cell lines were used as CD70⁺ target lines, and the MCF7 cell line was used as a CD70− target line. Since the assay was performed in conjunction with the cytotoxicity assay, the protocol was as follows: Target cells were seeded (50,000 target cells per 96-well plate) overnight and then co-cultured with CTX130 or control T cells at varying ratios (0.125:1, 0.25:1, 0.5:1, 1:1, 2:1 and to 4:1 T cells to target cells). Twenty-four hours later, plates were centrifuged, supernatant was collected and stored at −80° C. until further processing. IL-2 and IFNγ were quantified as follows: the MILLIPLEX® kit (Millipore, catalog #HCYTOMAG-60K) was used to quantify IFN-γ and IL-2 secretion using magnetic microspheres, HCYIFNG-MAG (Millipore, catalog #HCYIFNG-MAG) and HIL2-MAG (Millipore, catalog #HIL2-MAG), respectively. The assay was conducted following manufacturer's protocol. In short, MILLIPLEX® standard and quality control (QC) samples were reconstituted, and serial dilutions of the working standards from 10,000 pg/mL to 3.2 pg/mL were prepared. MILLIPLEX® standards, QCs and cell supernatants were added to each plate, and assay media was used to dilute the supernatants. All samples were incubated with HCYIFNG-MAG and HIL2-MAG beads for 2 hours. After incubation, the plate was washed using an automated magnetic plate washer. Human cytokine/chemokine detection antibody solution was added to each well and incubated for 1 hour followed by incubation with Streptavidin-Phycoerythrin for 30 minutes. The plate was subsequently washed, samples were resuspended with 150 μL Sheath Fluid, and agitated on a plate shaker for 5 minutes. The samples were read using the Luminex® 100/200™ instrument with xPONENT® software and data acquisition and analysis was completed using MILLIPLEX® Analyst software. The Median Fluorescent Intensity (MFI) data is automatically analyzed using a 5-parameter logistic curve-fitting method for calculating the cytokine concentration measured in the unknown samples.

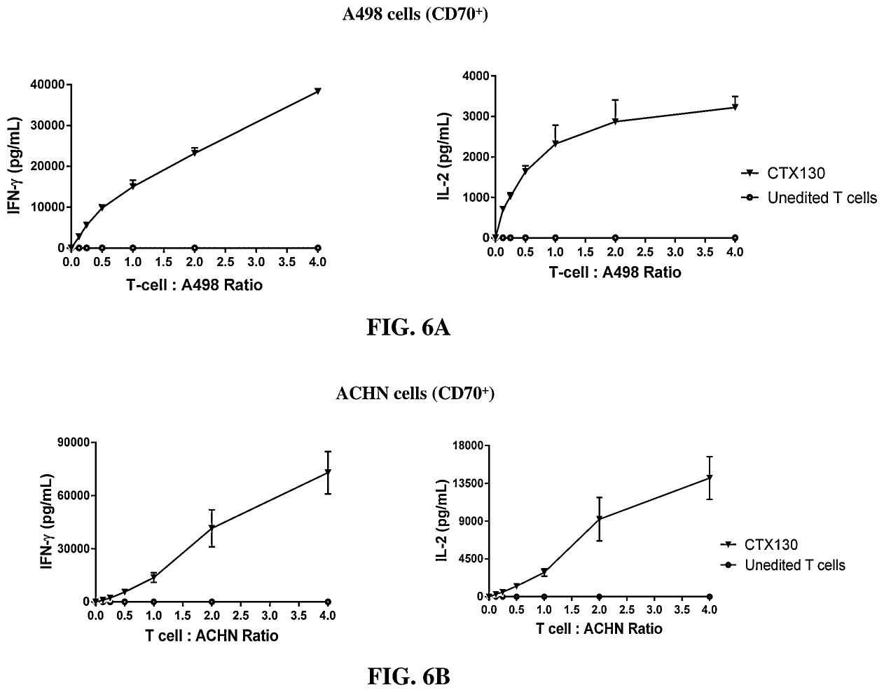

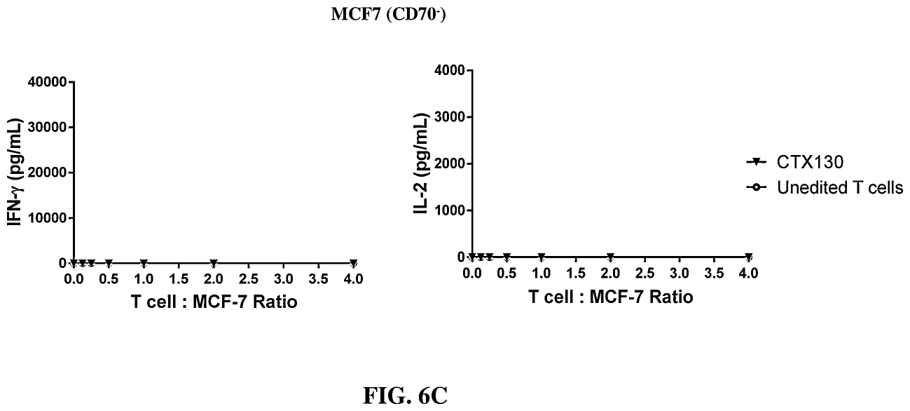

To determine if CTX130 secrete cytokines in the presence of CD70-positive and CD70-negative cells, the development lot 01 was co-cultured for 24 hours with A498, ACHN or MCF7 cells. CTX130 cells secreted both IFNγ and IL-2 following co-culture with CD70+ cells (A498 and ACHN), but not when co-cultured with CD70 negative cells (MCF7) (FIGS. 6A-6C, Tables 12-17). Unedited control T cells showed no specific effector cytokine secretion on the cell lines tested.

TABLE 12
Secretion of IFNγ by CTX130 cells in the presence of CD70+ cell line A498.

| | IFNγ (pg/mL) | | | | | |
|---|---|---|---|---|---|---|
| T cell: A498 ratio | CTX130 | | | Unedited T cells | | |
| 0 | 6.54* | 6.54* | 7.77 | 6.54* | 7.14 | 6.54* |
| 0.125 | 2592.57 | 2466.99 | 3213 | 6.54 | 6.54* | 6.54* |
| 0.25 | 5991 | 5592 | 5196 | 9.75 | 7.14 | 8.4 |
| 0.5 | 10713 | 9300 | 9354 | 7.14 | 6.54* | 9.75 |
| 1 | 16830 | 14514 | 13752 | 6.54* | 6.54 | 8.4 |
| 2 | 24645 | 22809 | 22053 | 8.4 | 14.01 | 15.54 |
| 4 | 38364 | 38364 | 38238 | 11.82 | 10.41 | 17.1 |

Samples marked with an asterisks (*) indicate the value was below the LoD (which was 6.54 pg/ml).

TABLE 13
Secretion of IL-2 by CTX130 cells in the presence of CD70+ cell line A498.

| | IL-2 (pg/mL) | | | | | |
|---|---|---|---|---|---|---|
| T cell: A498 ratio | CTX130 | | | Unedited T cells | | |
| 0 | 6.15* | 6.15* | 6.15* | 6.15* | 6.15* | 6.15* |
| 0.125 | 733.14 | 668.61 | 728.22 | 6.15* | 6.15* | 6.15* |
| 0.25 | 916.05 | 1056.24 | 1099.62 | 6.15* | 6.15* | 6.15* |
| 0.5 | 1753.2 | 1684.14 | 1473.69 | 6.15* | 6.15* | 6.15* |
| 1 | 2803.95 | 2277.39 | 1887.84 | 6.15* | 6.15* | 6.15* |
| 2 | 3375 | 2930.55 | 2294.85 | 6.15* | 6.15* | 6.15* |
| 4 | 3516 | 3162 | 2984.04 | 6.15* | 6.15* | 6.15* |

Samples marked with an asterisks (*) indicate the value was below the LoD (which was 6.15 pg/ml).

TABLE 14
Secretion of IFNγ by CTX130 cells in the presence of CD70+ cell line ACHN.

| | IFNγ (pg/mL) | | | | | |
|---|---|---|---|---|---|---|
| T cell: ACHN ratio | CTX130 | | | Unedited T cells | | |
| 0 | 2.92 | 5.4 | 7.12 | 4.36 | 4.88 | 2.36* |
| 0.125 | 757.56 | 1369.96 | 981 | 2.92 | 7.12 | 8.36 |
| 0.25 | 1776.44 | 2668.04 | 2507.68 | 4.36 | 3.4 | 7.12 |
| 0.5 | 4508 | 6904 | 5248 | 8.36 | 7.12 | 7.12 |
| 1 | 11148 | 16568 | 13624 | 9.64 | 3.88 | 9.64 |
| 2 | 32460 | 52872 | 39228 | 5.96 | 7.12 | 8.36 |
| 4 | 67268 | 86620 | 64944 | 9.64 | 12.4 | 16.88 |

Samples marked with an asterisks (*) indicate the value was below the LoD (which was 2.36 pg/ml).

TABLE 15
Secretion of IL-2 by CTX130 cells in the presence of CD70+ cell line ACHN.

| | IL-2 (pg/mL) | | | | | |
|---|---|---|---|---|---|---|
| T cell: ACHN ratio | CTX130 | | | Unedited T cells | | |
| 0 | 4.48* | 4.48* | 4.48* | 4.48* | 4.48* | 4.48* |
| 0.125 | 247.16 | 367.2 | 266.4 | 4.48* | 4.48* | 4.48* |
| 0.25 | 455.16 | 651.6 | 552.92 | 4.48* | 4.48* | 4.48* |
| 0.5 | 961.76 | 1466.04 | 1326.48 | 4.48* | 4.48* | 4.48* |
| 1 | 2437.04 | 3337.08 | 2891.04 | 4.48* | 4.48* | 4.48* |
| 2 | 7180 | 12148 | 8388 | 4.48* | 4.48* | 4.48* |
| 4 | 12324 | 17040 | 13028 | 4.48* | 4.48* | 4.48* |

Samples marked with an asterisks (*) indicate the value was below the LoD (which was 4.48 pg/ml).

TABLE 16
No secretion of IFNγ by CTX130 cells in the presence of CD70− cell line MCF7.

| | IFNγ (pg/mL) | | | | | |
|---|---|---|---|---|---|---|
| T cell: MCF7 ratio | CTX130 | | | Unedited T cells | | |
| 0 | 2.25* | 2.25* | 2.25* | 2.25* | 2.25* | 2.25* |
| 0.125 | 2.25* | 2.25* | 2.25* | 2.25* | 2.25* | 2.25* |
| 0.25 | 2.25* | 3.26 | 3.26 | 2.25* | 2.25* | 2.25* |
| 0.5 | 4.41 | 2.72 | 4.02 | 2.25* | 2.25* | 2.25* |
| 1 | 5.86 | 5.23 | 5.23 | 2.25* | 2.25* | 2.25* |
| 2 | 19.64 | 15.06 | 14.81 | 2.25* | 2.72 | 2.25 |
| 4 | 29.85 | 29.58 | 21.44 | 6.08 | 4.41 | 4.41 |

Samples marked with an asterisks (*) indicate the value was below the LoD (which was 2.25 pg/ml).

TABLE 17
No secretion of IL-2 by CTX130 cells in the presence of CD70− cell line MCF7.

| | IL-2 (pg/mL) | | | | | |
|---|---|---|---|---|---|---|
| T cell: MCF7 ratio | CTX130 | | | Unedited T cells | | |
| 0 | 2.74* | 2.74* | 2.74* | 2.74* | 2.74* | 2.74* |
| 0.125 | 2.74* | 2.74* | 2.74* | 2.74* | 2.74* | 2.74* |
| 0.25 | 2.74* | 2.74* | 2.74* | 2.74* | 2.74* | 2.74* |
| 0.5 | 2.74* | 2.74* | 2.74* | 2.74* | 2.74* | 2.74* |
| 1 | 2.74* | 2.74* | 2.74* | 2.74* | 2.74* | 2.74* |
| 2 | 2.74* | 2.74* | 2.74* | 2.74* | 2.74* | 2.74* |
| 4 | 2.74* | 2.74* | 2.74* | 2.74* | 2.74* | 2.74* |

Samples marked with an asterisks (*) indicate the value was below the LoD (which was 2.74 pg/ml).

These results demonstrate that CTX130 cells exhibit effector function by secreting IFNγ and IL-2 in the presence of renal cell carcinoma cells expressing CD70, but not in the presence of the CD70 negative cell line MCF7.

### Example 6: Selective Killing of CD70+ Cells by Anti-CD70 CAR+ T Cells (CTX130)

The objective of this study was to assess the ability of CTX130 to selectively lyse CD70 expressing cells in vitro.

The ability of CTX130 or control T cells (unedited T cells with no anti-CD70 CAR expression) to specifically kill CD70 positive or CD70 negative target cells was assessed using a CellTiter-Glo luminescent cell viability-based cytotoxicity assay. A498 and ACHN cell lines were used as CD70 positive target lines, and the MCF7 cell line was used as a CD70 negative target line (all obtained from ATCC). T cells from the development lot 01 were used in these experiments.

50,000 human target cells (CD70 positive A498 and ACHN, CD70 negative MCF7) per well of an opaque-walled 96-well plate (Corning, Tewksbury, Mass.) were plated overnight. The next day, the cells were co-cultured with T cells at varying ratios (0.125:1, 0.25:1, 0.5:1, 1:1, 2:1 and 4:1 T cells to target cells) for 24 hours. Target cells were incubated with unedited T cells (TCR+B2M+CAR−), or CTX130 cells. After manually washing off T cells with PBS, the remaining viable target cells were quantified using a CellTiter-Glo luminescent cell viability assay (CellTiterGlo® 2.0 Assay, Promega G9242). Fluorescence was measured using a Synergy H1 plate reader (Biotek Instruments, Winooski, Vt.). Prior to processing the cells for CellTiter-Glo analysis, supernatants were collected for quantification of cytokine secretion following co-culture.

The percent cell lysis was then calculated using the following equation using relative light units (RLU):

% Cell lysis=((RLU target cells with no effector−RLU target cells with effector))/(RLU target cell with no effector)×100

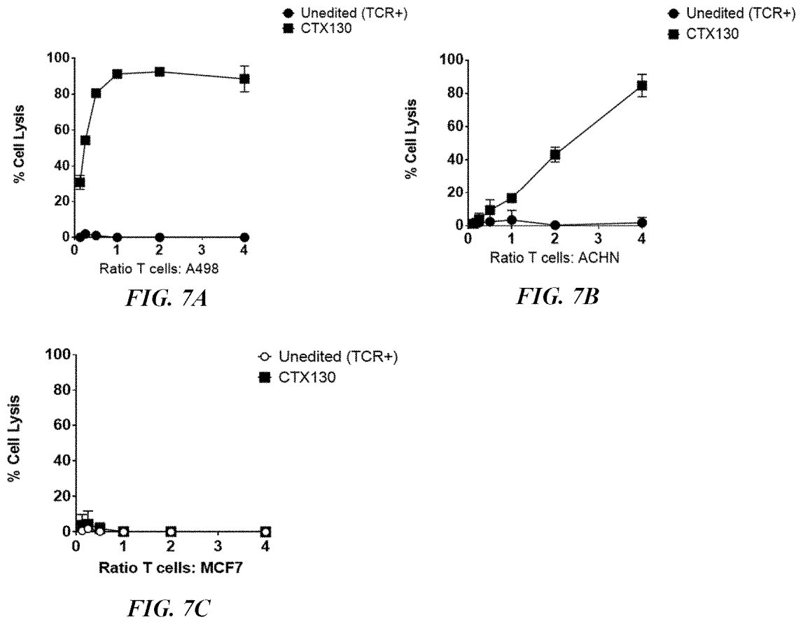

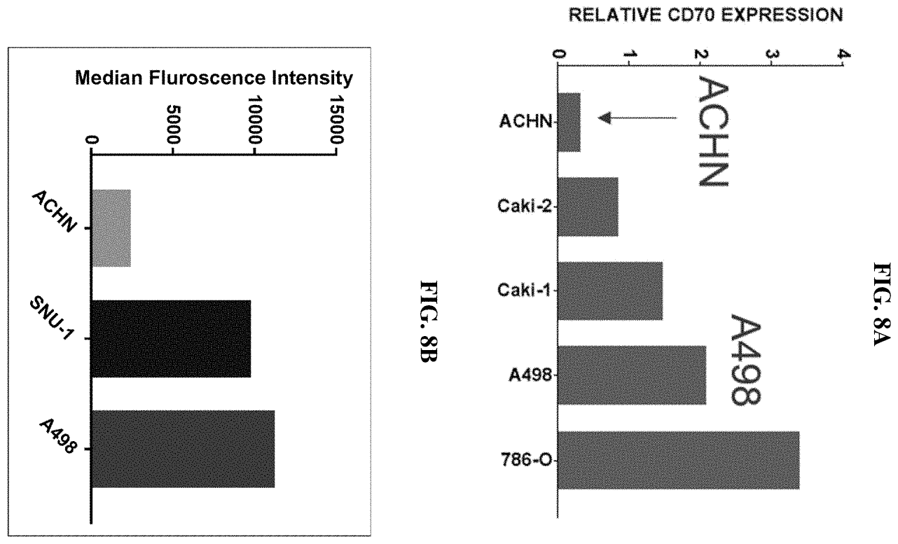

The development lot of CTX130 (lot 01) was tested for cell killing activity against the CD70+ cell lines A498 and ACHN. The CTX130 lot showed potent cell killing activity specifically against both high (A498; FIG. 7A) and low (ACHN; FIG. 7B) CD70 expressing cells, but not when co-cultured with CD70− MCF7 cells (FIG. 7C). In the absence of CAR expression, control unedited T cells were less effective at killing the CD70+ cells. See also data shown in Tables 18-20.

TABLE 18
Percent dead A498 cells in presence of CTX130 cells.

| T cell: A498 cell ratio | CTX130 | | | Unedited T cells | | |
|---|---|---|---|---|---|---|
| 0.125 | 33.6 | 32.8 | 26.5 | −3.1 | −0.8 | 0.3 |
| 0.25 | 55.6 | 53.1 | 54.3 | −1.2 | 2.7 | 3.1 |
| 0.5 | 82.4 | 80.7 | 78.5 | −3.5 | 1.8 | 1.4 |
| 1 | 92.0 | 90.3 | 91.4 | −6.5 | −1.5 | −2.6 |
| 2 | 94.5 | 91.3 | 91.6 | −6.0 | −1.1 | −1.0 |
| 4 | 87.7 | 81.8 | 96.0 | −7.4 | −5.9 | −6.7 |

TABLE 19
Percent dead ACHN cells in presence of CTX130 cells.

| T cell: ACHN cell ratio | CTX130 | | | Unedited T cells | | |
|---|---|---|---|---|---|---|
| 0.125 | 3.8 | −1.3 | −0.9 | 2.7 | −2.9 | 3.1 |
| 0.25 | 7.5 | 0.2 | 4.2 | 4.6 | −1.6 | 1.3 |
| 0.5 | 15.9 | 3.4 | 9.2 | 4.1 | 3.5 | −0.9 |
| 1 | 18.1 | 14.5 | 17.5 | 0.3 | 10.3 | −0.9 |
| 2 | 43.1 | 38.9 | 47.8 | −0.8 | −0.4 | 1.4 |
| 4 | 86.3 | 77.3 | 90.5 | −5.6 | 5.6 | −3.7 |

TABLE 20
Percent dead MCF7 cells in presence of CTX130 cells.

| T cell: MCF7 cell ratio | CTX130 | | | Unedited T cells | | |
|---|---|---|---|---|---|---|
| 0.125 | 10.8 | −4.4 | 0.2 | −0.7 | 1.9 | −1.0 |
| 0.25 | 13.0 | −10.2 | −0.3 | 2.6 | 2.8 | −0.1 |
| 0.5 | 5.6 | −12.3 | −7.1 | 0.8 | −1.4 | −9.5 |
| 1 | 0.6 | −15.3 | −10.3 | −1.0 | −3.7 | −12.5 |
| 2 | 0.7 | −22.6 | −10.6 | −3.5 | −8.1 | −13.7 |
| 4 | 0.1 | −26.2 | −16.2 | −12.8 | −10 | −20.5 |

These results demonstrated that CTX130 cells were able to lyse cancer cell lines in vitro in a CD70-specific manner.

### Example 7: CD70 KO Improves Cell Kill in Multiple Cell Types

(a) CD70 Expression in Various Cancer Cell Lines

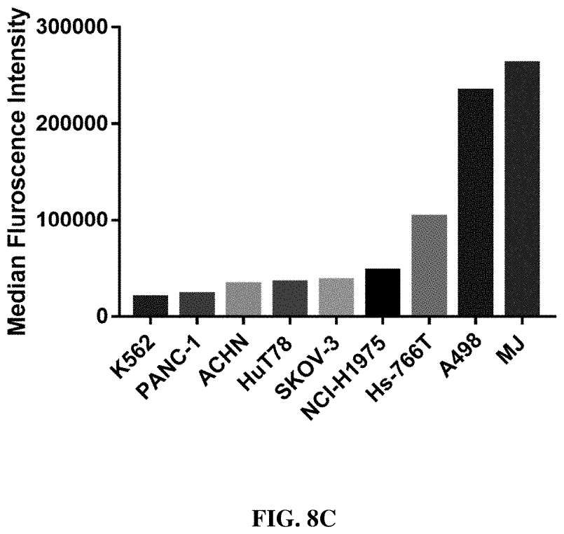

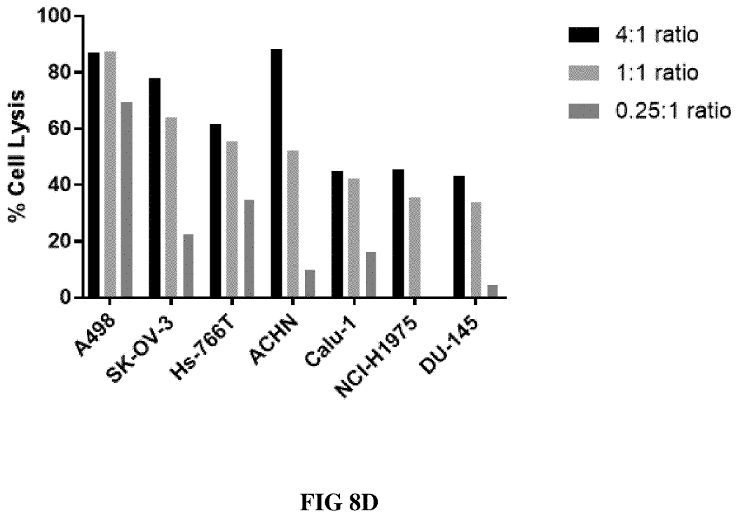

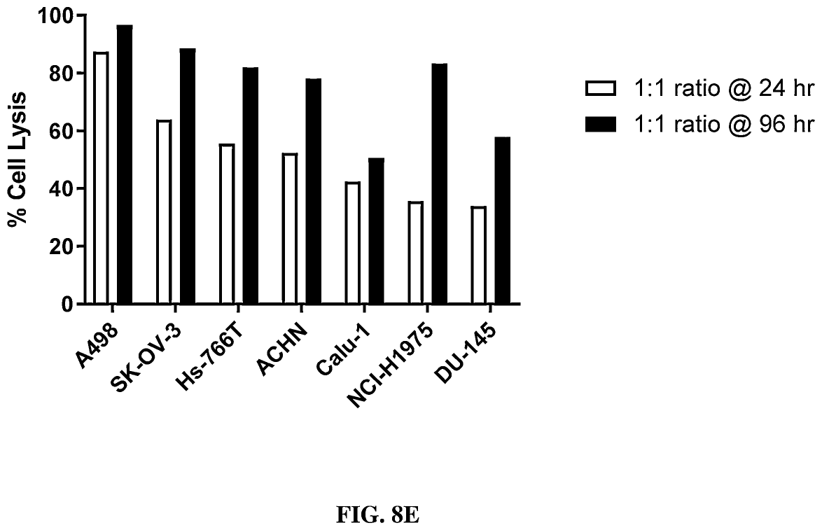

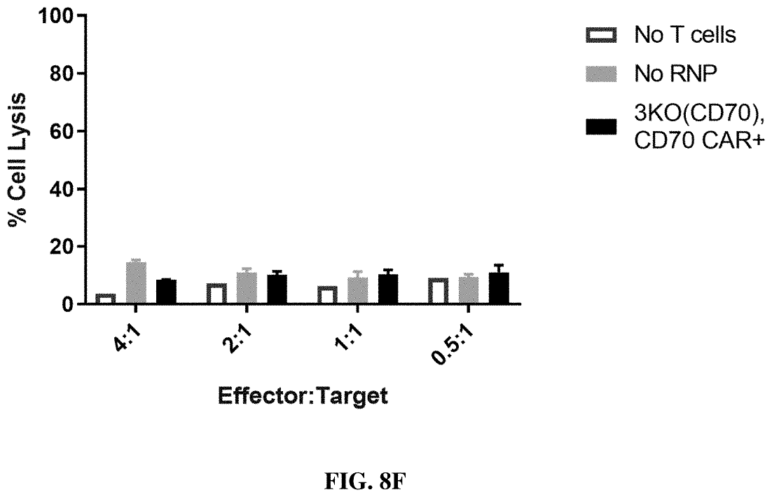

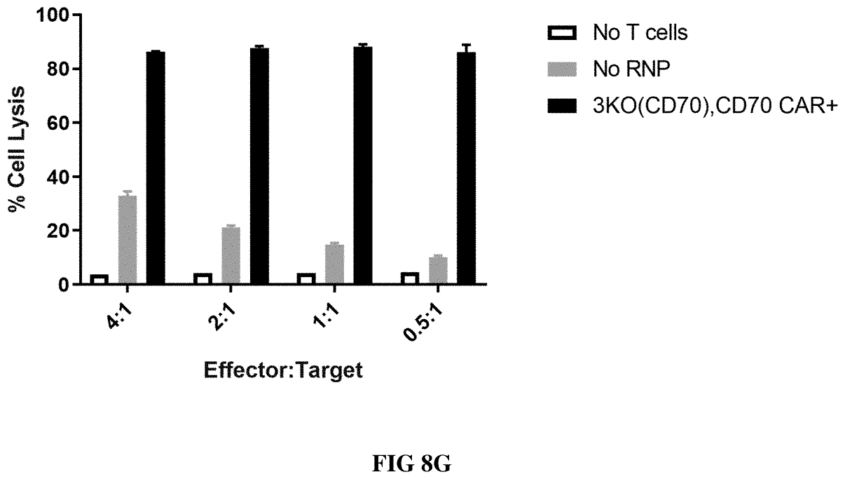

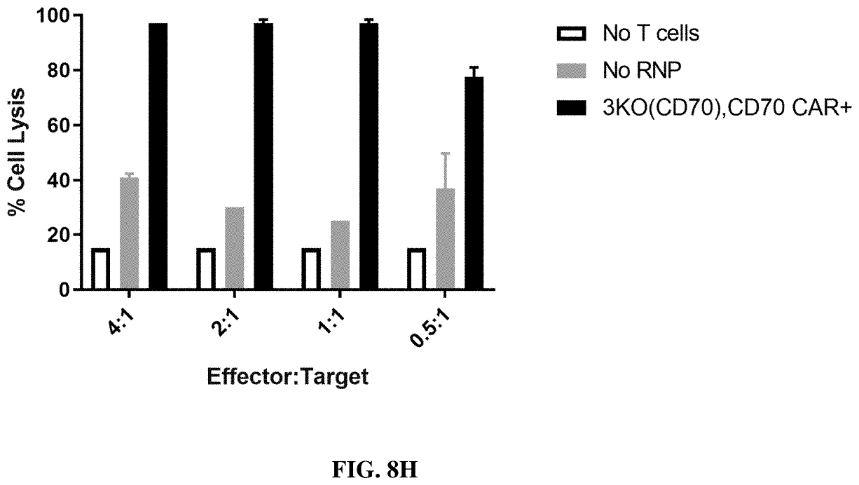

Relative CD70 expression was measured in various cancer cell lines to further evaluate the ability of anti-CD70 CAR⁺ T cells to kill various cancer types. CD70 expression was measured by FACS analysis using Alexa Fluor 647 anti-human CD70 antibody (BioLegend Cat. No. 355115). FIG. 8A shows the relative expression of CD70 in ACHN cells, as measured by FACS, compared to other kidney cancer cell lines A498, 786-O, cacki-1 and Caki-2. Additionally, non-kidney cancer cell lines were evaluated for CD70 expression by FACS analysis (Table 21, FIGS. 8A-8C) using either an Alexa Fluor 647 anti-human CD70 antibody (BioLegend Cat. No. 355115; FIG. 8B) or a FITC anti-human CD70 antibody (BioLegend Cat. No. 355105; in FIG. 8C). SNU-1 (intestinal cancer cells) exhibited high levels of CD70 expression that were similar to A498 (FIG. 8B). SKOV-3 (ovarian), HuT78 (lymphoma), NCI-H1975 (lung) and Hs-766T (pancreatic) cell lines exhibited levels of CD70 expression that were similar or higher than ACHN but lower than A498 (Table 21, FIG. 8C).

TABLE 21
Cell lines and relative CD70 expression.

| | | Relative CD70 |
|---|---|---|
| Cell Line | Cancer type | expression |
| A498 | Kidney Carcinoma | High |
| ACHN | Kidney (derived from metastasis) | Medium-Low |
| SK-OV-3 | Ovarian Adenocarcinoma | Medium |
| NCI-H1975 | Lung Adenocarcinoma (NSCLC) | Medium |
| Calu-1 | Lung Carcinoma | Low |
| DU 145 | Prostate Carcinoma | Low |
| SNU-1 | Gastric Carcinoma | High |
| Hs 766T | Pancreatic Carcinoma | Medium |
| MJ | T cell Lymphoma | High |
| HuT78 | T cell Lymphoma | Medium |
| HuT102 | T cell Lymphoma | Medium |
| PANC-1 | Pancreatic Carcinoma | Low |
| U937 | AML | No expression |
| K562 | chronic myelogenous leukemia | No expression |
| | | (Negative Control) |

Cell Kill Assay. The ability of multi-gene edited anti-CD70 CAR+ cells to kill various solid tumor cells was determined using a cell kill assay. To quantify cell killing, cells were washed, media was replaced with 200 mL of media containing a 1:500 dilution of 5 mg/mL DAPI (Molecular Probes) (to enumerate dead/dying cells). Finally, 25 mL of CountBright beads (Life Technologies) was added to each well. Cells were then processed by flow cytometry.
1) Cells/mL=((number of live target cell events)/(number of bead events))×((Assigned bead count of lot (beads/50 μL))/(volume of sample))
2) Total target cells were calculated by multiplying cells/mL×the total volume of cells.
3) The percent cell lysis was then calculated with the following equation:

% Cell lysis=(1−((Total Number of Target Cells in Test Sample)/(Total Number of Target Cells in Control Sample))×100

Indeed, it was found that TRAC⁻/β2M⁻/CD70⁻/anti-CD70 CAR⁺ (3×KO (CD70), CD70 CAR⁺) exhibited surprisingly potent cell killing of numerous solid tumor cell lines after only 24 hours of co-culture (FIG. 8D shows killing by 3×KO CAR+ T cells). 3×KO, CD70 CAR+ T cells killed >60% of kidney, pancreatic, and ovarian tumor cells (A498, ACHN, SK-OV-3, and Hs-766T) at a 4:1 effector:target cell ratio and >50% at a 1:1 effector:target cell ratio (FIG. 8D). Cell killing of cancer cell lines that had medium to low CD70 expression (NCI-H1975, Calu-1 and DU 145) was still effective with >30% killing at an effector:target cell ratio of 4:1 within 24 hours of co-culture (FIG. 8E). Longer exposure (i.e., 96 hours) to either 3×KO CD70 CAR+ T cells resulted in an increase in cancer cell killing across all cell types, particularly for SKOV-3, Hs-766T, and NIC-H1975 cells wherein killing was >80% at an effector:target cell ratio of 1:1 (FIG. 8E).

(b) Selective Killing of Additional CD70 Expressing Cell Lines

The ability of anti-CD70 CAR+ T cells to selectively kill CD70-expressing cells was determined. A flow cytometry assay was designed to test killing of cancer cell suspension lines (e.g., K562, MM.1S and HuT78 cancer cells that are referred to as “target cells”) by 3×KO (CD70) (TRAC⁻/B2M⁻/CD70⁻) anti-CD70 CAR+ T cells. Two of the target cell lines that were used were CD70-expressing cancer cells (e.g., MM.1S and HuT78), while a third that was used as negative control cancer cells lack CD70 expression (e.g., K562). The TRAC⁻/B2M⁻/CD70⁻/anti-CD70 CAR+ T cells were co-cultured with either the CD70-expressing MM.1S or HuT78 cell lines or the CD70-negative K562 cell line. The target cells were labeled with 5 μM efluor670 (eBiosciences), washed and seeded at a density of 50,000 target cells per well in a 96-well U-bottom plate. The target cells were co-cultured with TRAC⁻/B2M⁻/CD70⁻ anti-CD70 CAR+ T cells at varying ratios (0.5:1, 1:1, 2:1 and 4:1 CAR+ T cells to target cells) and incubated overnight. Target cell killing was determined following a 24 hour co-culture. The cells were washed and 200 μL of media containing a 1:500 dilution of 5 mg/mL DAPI (Molecular Probes) (to enumerate dead/dying cells) was added to each well. Cells were then analyzed by flow cytometry and the amount of remaining live target cells was quantified.

FIGS. 8F-8H demonstrate selective target cell killing by TRAC−/B2M−/CD70−anti-CD70 CAR+ T cells. A 24 hour co-culture with 3×KO (CD70) CAR+ T cells resulted in nearly complete killing of T cell lymphoma cells (HuT78), even at a low CAR+ T cell to CD70-expressing target cell ratio of 0.5:1 (FIG. 811). Likewise, a 24 hour co-culture resulted in nearly complete killing of multiple myeloma cells (MM.1S) at all CAR+ T cell to target cell ratios tested (FIG. 8G). Killing of target cells was found to be selective in that TRAC−/B2M−/anti-CD70 CAR+ T cells induced no killing of CD70-deficient K562 cells that was above the level of control samples (e.g., either cancer cells alone or co-culture with no RNP T cells) at any effector:target cell ratio tested (FIG. 8F).

SNU-1 cell kill by was assessed by visual assessment. Target cell killing following long exposure to CAR+ T cells was also assessed by microscopy for SNU-1 cancer cells. SNU-1 cells were plated at a density of 1 million cells per well in a 6 well plate and mixed at an effector:target ratio of 4:1 with 3×KO (CD70), anti-CD70 CAR⁺ T cells. The co-culture was incubated for six (6) days and the presence of viable cancer cells was assessed by microscope. All gastric carcinoma target cells (SNU-1) were eliminated in wells containing TRAC⁻/β2M⁻/CD70⁻/anti-CD70 CAR⁺ T cells, as compared to control wells, indicating cancer cells were completely eliminated by anti-CD70 CAR⁺ T cells with an extended co-culture.

### Example 8: Efficacy of Anti-CD70 CART Cells: Treatment in the Subcutaneous Renal Cell Carcinoma Tumor Xenograft Model in NOG Mice

The ability of T cells expressing a CD70 CAR to eliminate kidney carcinoma cells that express high levels of CD70 was evaluated in in vivo using subcutaneous renal cell carcinoma tumor xenograft models in mice. These models included a subcutaneous A498-NOG model, a subcutaneous 786-O-NSG model, a subcutaneous Caki-2-NSG model, and a subcutaneous Caki-1-NSG model. CTX130 cells were produced as described herein.

For each subcutaneous renal cell carcinoma tumor xenograft model, five million cells of the indicated cell type were injected subcutaneously into the right flank of NOG (NOD.Cg-Prkdc^{scid}Il2rg^{tm1Sug}/JicTac) mice. When mean tumor size reached an average size of approximately 150 mm³, mice were either left untreated or injected intravenously with 8×10⁶ CAR⁺ CTX130 (TRAC⁻/B2M⁻/CD70⁻/anti-CD70 CAR+ T cells) cells per mouse. In the subcutaneous A498-NOG model, an additional group of mice was injected with 7.5×10⁶ CAR+TRAC⁻B2M⁻anti-CD70 CAR-T cells per mouse.

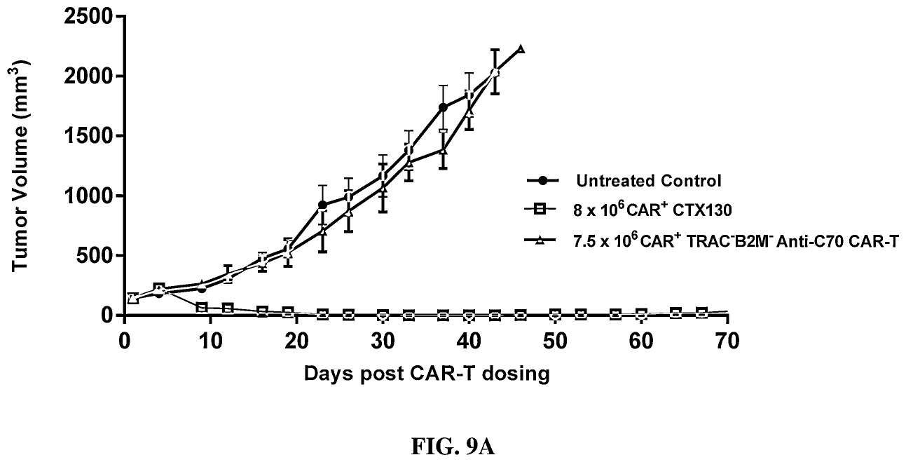

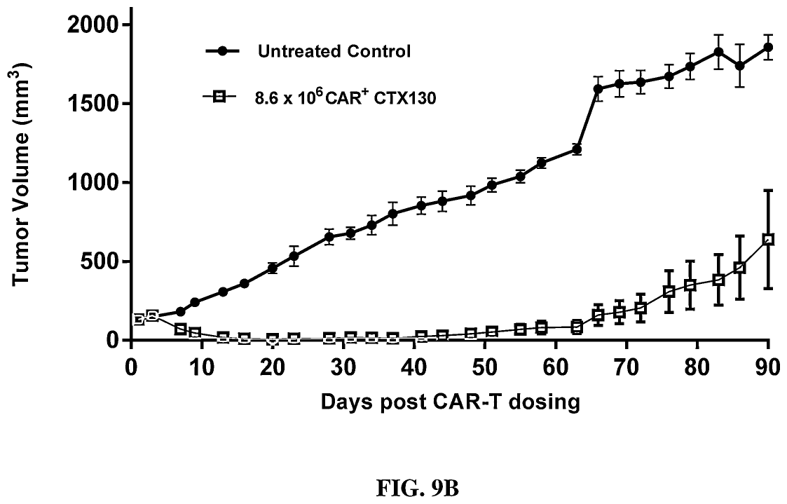

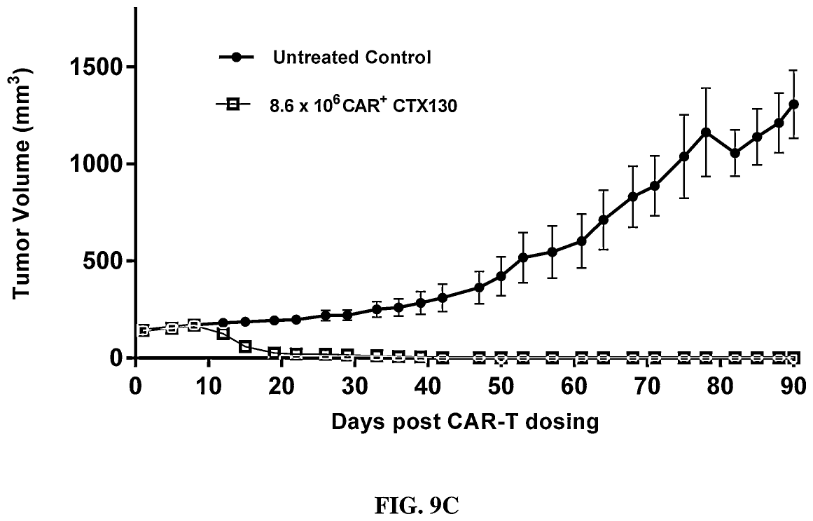

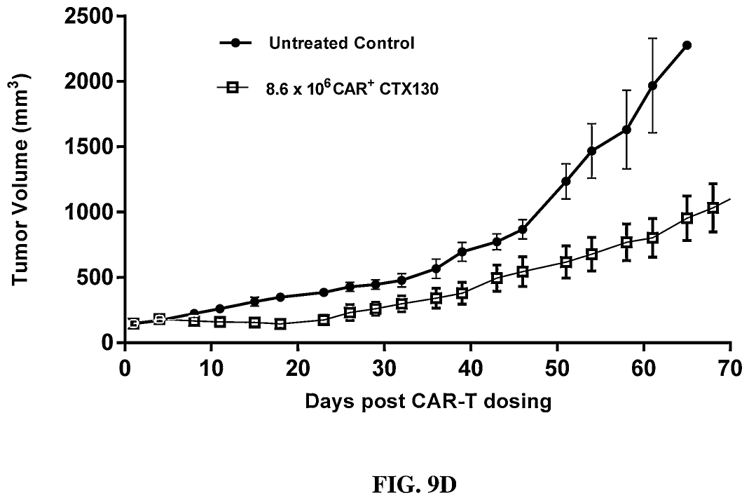

CTX130 cells completely eliminated tumor growth in the subcutaneous A498-NOG model (FIG. 9A) and the subcutaneous Caki-2-NSG model (FIG. 9C). Tumor growth in mice injected with TRAC⁻/B2M⁻/anti-CD70 CAR+ T cells was similar to that of the untreated control mice (FIG. 9A). CTX130 cells significantly reduced tumor growth in the subcutaneous 786-O-NSG model (FIG. 9B) and the subcutaneous Caki-1-NSG model (FIG. 9D).

Taken together, these results demonstrate that CTX130 cells reduced tumor growth in four types of subcutaneous renal cell carcinoma tumor xenograft models.

Tumor Re-Challenge Model Renal Cell Carcinoma Tumor Xenograft Model

The efficacy of CTX130 was also tested in a subcutaneous A498 xenograft model with re-challenge. In brief, five million A498 cells were injected subcutaneously in the right flank of NOD (NOD.Cg-Prkdc^{scid}Il2rg^{tm1Sug}/JicTac) mice. Tumors were allowed to grow to an average size of approximately 51 mm³ after which the tumor-bearing mice were randomized in two groups (N=5/group). Group 1 was left untreated while Group 2 received 7×10⁶ CAR+CTX130 cells and Group 3 received 8×10⁶ CAR+TRAC−B2M−anti-CD70 CAR T cells. On Day 25, a tumor re-challenge was initiated whereby 5×10⁶ A498 cells were injected into the left flank of treated mice and into a new control group (Group 4).

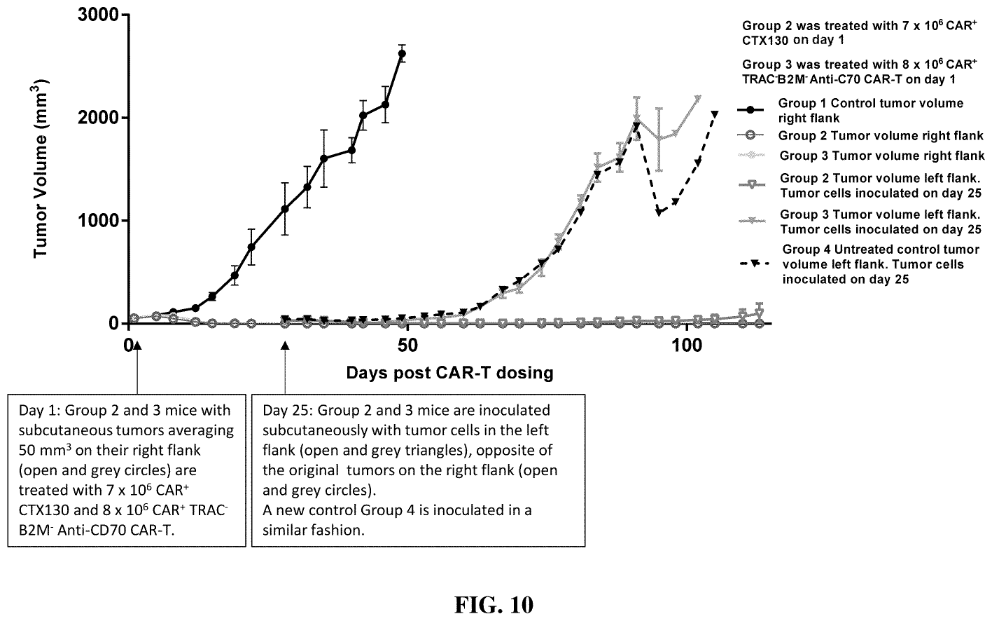

As shown in FIG. 10, mice treated with CTX130 cells exhibited no tumor growth post rechallenge by injection of A498 cells into the left flank while mice treated with anti-CD70 CAR T cells exhibited tumor growth of the A498 cells injected into the left flank. These results demonstrate that CTX130 cells retain higher in vivo efficacy after re-exposure to tumor cells than other anti-CD70 CAR+ T cells (CAR+TRAC−B2M−anti-CD70 CAR T cells).

Efficacy of CTX130 Redosing Renal Cell Carcinoma Tumor Xenograft Model

The efficacy of CTX130 was also tested in a subcutaneous A498 xenograft model with redosing. In brief, five million A498 cells were injected subcutaneously into the right flank of NOG (NOD.Cg-Prkdc^{scid}Il2rg^{tm1Sug}/JicTac) mice. When mean tumor size reached an average size of approximately 453 mm³, mice were either left untreated or injected intravenously (N=5) with 8.6×10⁶ CAR+CTX130 cells per mouse. Group 2 mice were treated with a second and third dose of 8.6×10⁶ CAR+CTX130 cells per mouse on day 17 and 36, respectively. Group 3 mice were treated with a second dose of 8.6×10⁶ CAR+CTX130 cells per mouse on day 36.

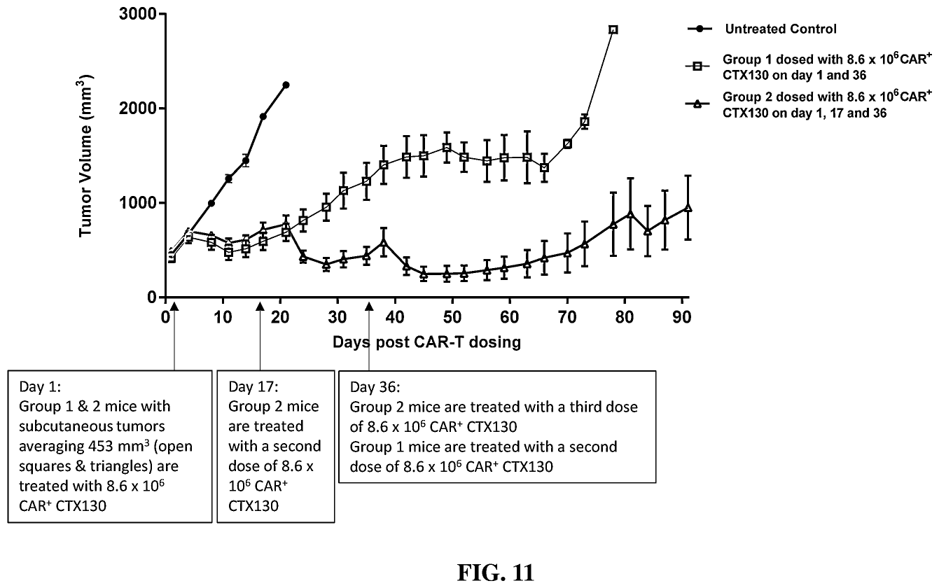

As shown in FIG. 11, mice dosed with CTX 130 cells on day 1 and then redosed on day 17 and 36 exhibited less tumor growth than mice administered only one redose on day 36. These results demonstrate that redosing of CTX130 cells provides enhanced suppression of tumor growth.

### Example 9: Efficacy of Anti-CD70 CART Cells: Treatment in CD70+Solid Tumor Xenograft Models in NOG Mice

The ability of T cells expressing an anti-CD70 CAR to eliminate tumor cells that express CD70 was evaluated in vivo using a murine subcutaneous tumor xenograft model.

CRISPR/Cas9 and AAV6 were used as above (see for example, Example 3) to generate human T cells that lack expression of the TCR, β2M, CD70 with concomitant expression from the TRAC locus using a CAR construct targeting CD70 (SEQ ID NO: 46 or SEQ ID NO: 81). In this example activated T cells were first electroporated with 3 distinct Cas9:sgRNA RNP complexes containing sgRNAs targeting TRAC (SEQ ID NO: 6), β2M (SEQ ID NO: 10), and CD70 (SEQ ID NO: 2). The DNA double stranded break at the TRAC locus was repaired by homology directed repair with an AAV6-delivered DNA template comprising a donor template (SEQ ID NO: 43; SEQ ID NO: 44) (encoding anti-CD70 CAR comprising the amino acid sequence of SEQ ID NO: 46 or SEQ ID NO: 81) containing right and left homology arms to the TRAC locus flanking a chimeric antigen receptor cassette (−/+ regulatory elements for gene expression).

The resulting modified T cells are 3×KO (TRAC−/132M−/CD70−) anti-CD70 CAR+ T cells. The ability of the anti-CD70 CAR+ T cells to ameliorate disease caused by a CD70+ tumor cell lines was evaluated in NOG mice using methods described herein.

### Treatment in the Ovarian Tumor Model

The ability of T cells expressing an anti-CD70 CAR to eliminate ovarian adenocarcinoma cells that express moderate levels of CD70 was evaluated in vivo using a subcutaneous ovarian carcinoma (SKOV-3) tumor xenograft model in mice.

The ability of the anti-CD70 CAR+ T cells to ameliorate disease caused by a CD70+ ovarian carcinoma cell line was evaluated in NOG mice using methods employed by Translational Drug Development, LLC (Scottsdale, Ariz.). In brief, twelve (12) 5-8 week-old female, CIEA NOG (NOD.Cg-Prkdc^{scid}Il2rg^{tm1Sug}/JicTac) mice were individually housed in ventilated microisolator cages, maintained under pathogen-free conditions, 5-7 days prior to the start of the study. Mice received a subcutaneous inoculation of 5×10⁶ SKOV-3 ovarian carcinoma cells/mouse in the right hind flank. When mean tumor size reached 25-75 mm³ (target of ˜50 mm³), the mice were further divided into two treatment groups as shown in Table 22. On Day 1, treatment group 2 received a single 200 μl intravenous dose of anti-CD70CAR+ T cells according to Table 22.

TABLE 22
Treatment groups

| | | | T cell | |
|---|---|---|---|---|
| | | SKOV-3 | treatment | |
| Group | CAR-T | cells | (i.v.) | N |
| 1 | None | 5 × 10⁶ | None | 5 |
| | | cells/mouse | | |
| 2 | 3× KO (CD70,) | 5 × 10⁶ | 1 × 10⁷ | 5 |
| | anti-CD70 CAR+ T cells | cells/mouse | cells/mouse | |

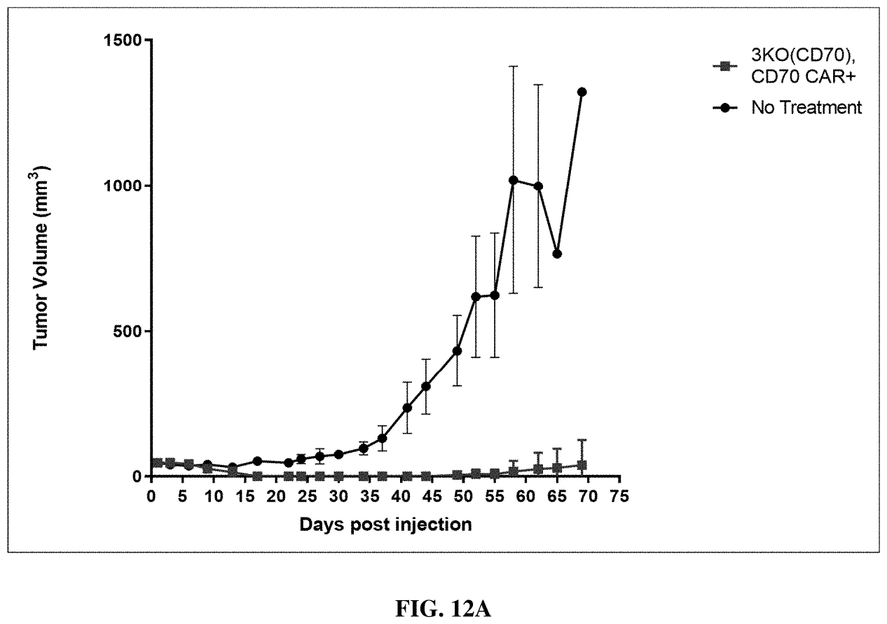

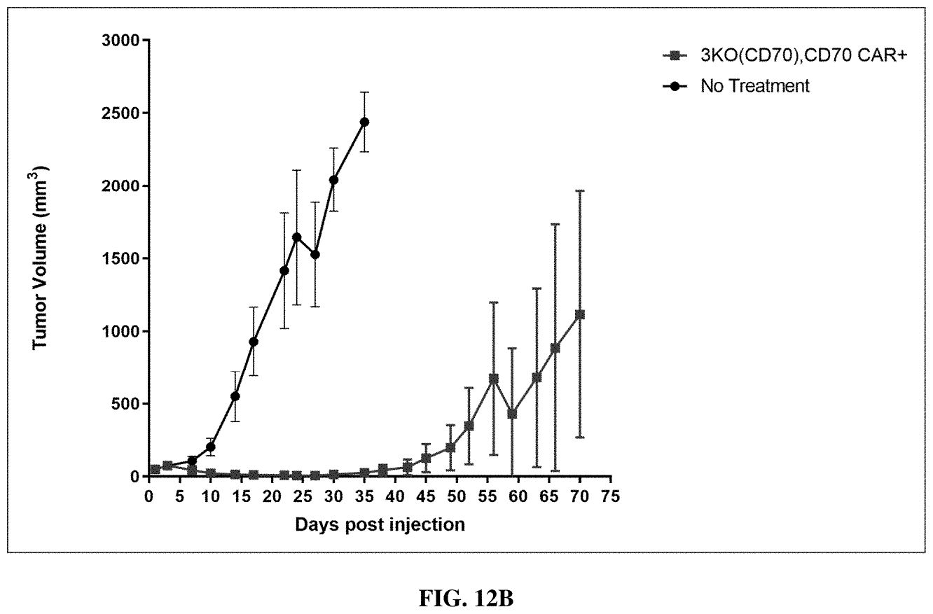

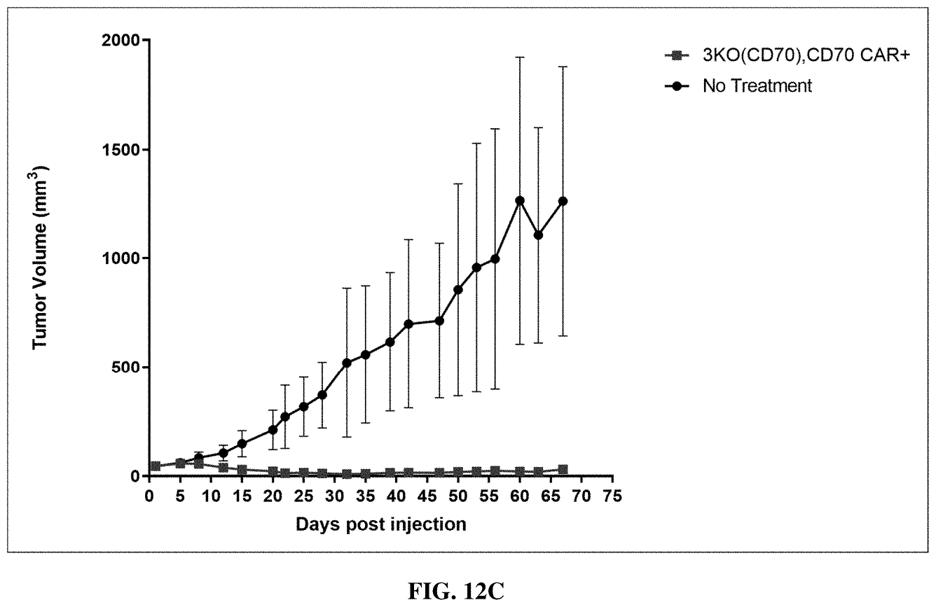

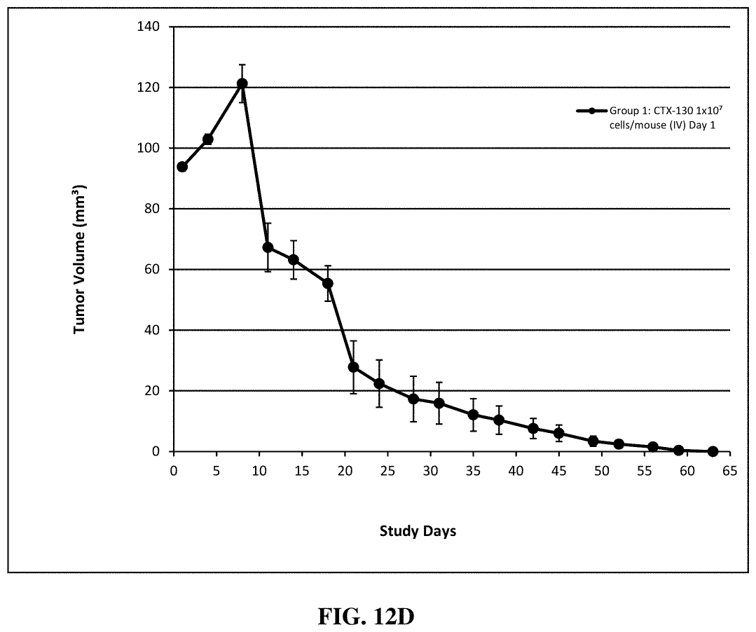

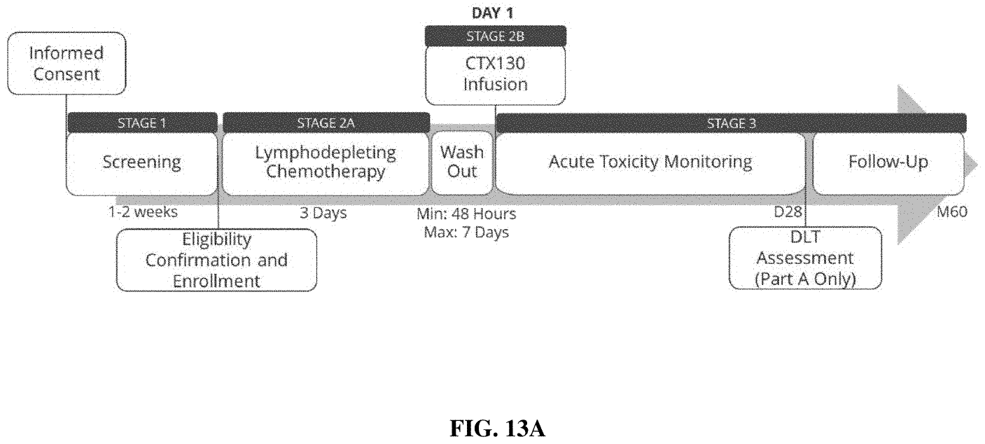

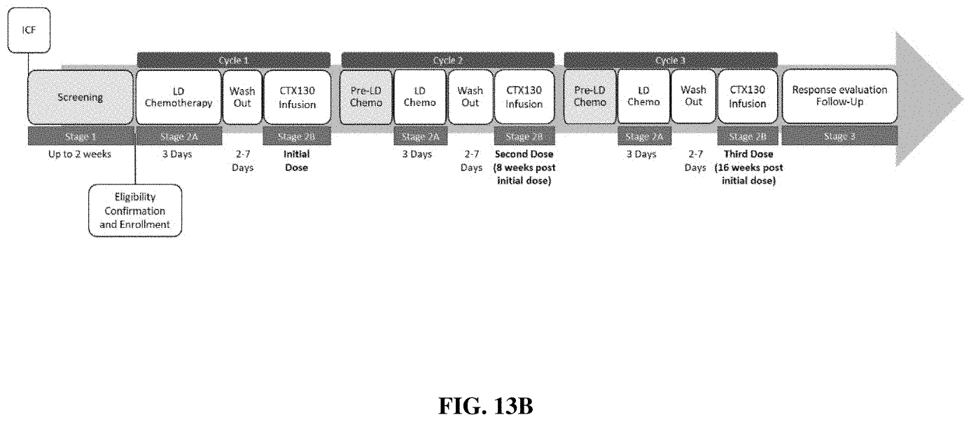

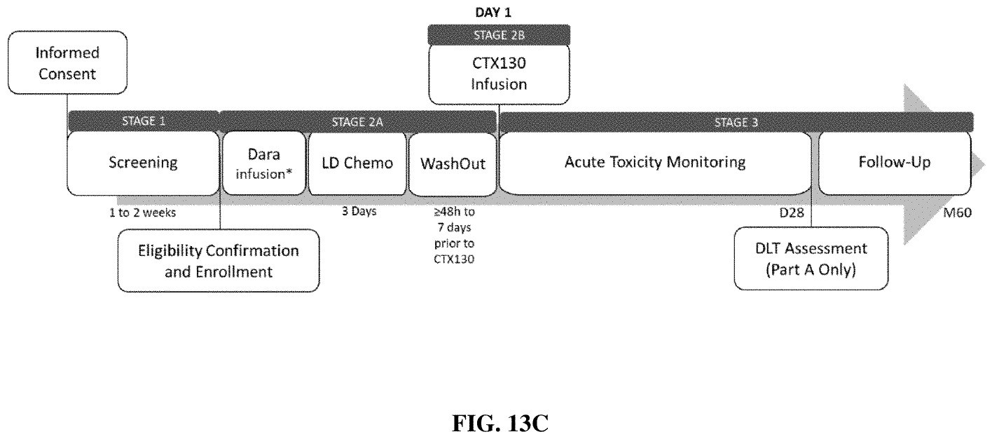

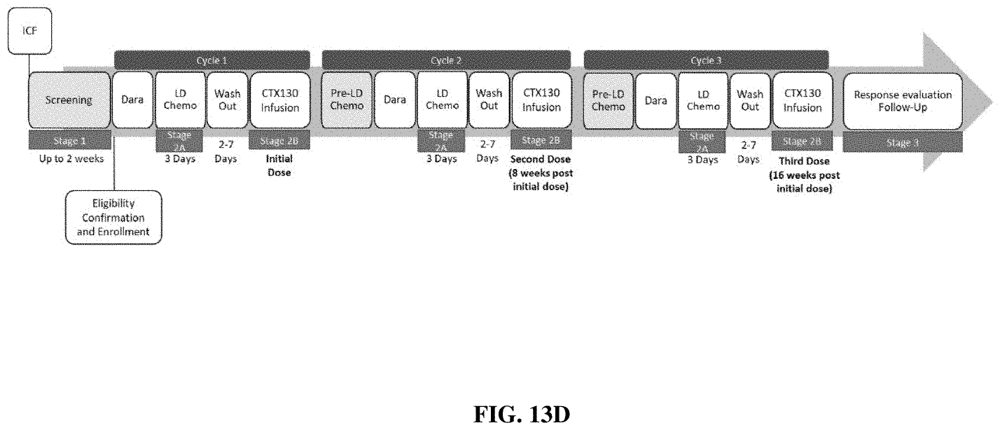

Tumor volume was measured 2 times weekly from day of treatment initiation. By day 9 post-injection, tumors treated with anti-CD70 CART cells began to show a decrease in tumor volume relative to tumors in untreated animals. By day 17 post-injection, CD70+ ovarian cancer tumors in mice treated with anti-CD70 CAR T cells were completely eliminated. This complete regression of tumor growth was sustained in treated animals through day 44 post-injection, whereupon 4 out of 5 mice treated with anti-CD70 CART cells remained tumor-free until the end-of-observation (day 69) (FIG. 12A). These data demonstrate that 3×KO (TRAC−/β2M−/CD70−) anti-CD70 CAR+ cells are highly potent in vivo for treating human ovarian tumors.

Treatment in the Non-Small Cell Lung Carcinoma (NSCLC) Tumor Model

The ability of T cells expressing a CD70 CAR to eliminate lung adenocarcionma cells that express moderate levels of CD70 was evaluated in in vivo using a subcutaneous lung carcinoma (NCI-H1975) tumor xenograft model in mice.

The ability of these anti-CD70 CAR+ T cells to ameliorate disease caused by a CD70+ lung carcinoma cell line was evaluated in NOG mice using methods employed by Translational Drug Development, LLC (Scottsdale, Ariz.). In brief, 12, 5-8 week-old female, CIEA NOG (NOD.Cg-Prkdc^{scid}Il2rg^{tm1Sug}/JicTac) mice were individually housed in ventilated microisolator cages, maintained under pathogen-free conditions, 5-7 days prior to the start of the study. Mice received a subcutaneous inoculation of 5×10⁶NCI-H1975 lung carcinoma cells/mouse in the right hind flank. When mean tumor size reached 25-75 mm³ (target of ˜50 mm³), the mice were further divided into 2 treatment groups as shown in Table 23. On Day 1, treatment group 2 received a single 200 μl intravenous dose of anti-CD70CAR+ T cells according to Table 23.

TABLE 23
Treatment groups

| | | | T cell | |
|---|---|---|---|---|
| | | NCI-H1975 | treatment | |
| Group | CAR-T | cells | (i.v.) | N |
| 1 | None | 5 × 10⁶ | None | 5 |
| | | cells/mouse | | |
| 2 | 3× KO (CD70,) | 5 × 10⁶ | 1 × 10⁷ | 5 |
| | anti-CD70 CAR+ T cells | cells/mouse | cells/mouse | |

Tumor volume was measured 2 times weekly from day of treatment initiation. By day 12 post-injection, tumors treated with anti-CD70 CAR T cells began to show a decrease in tumor volume relative to tumors in untreated animals. This complete regression of tumors in treated animals continue through day 33 post injection. Treatment with anti-CD70 CAR T cells resulted in potent activity against established H1975 lung cancer xenografts through 40 days post injection (tumor regrowth was suppressed in all mice up to day 40 with tumor size<100 mm³), whereupon tumors began to grow. (FIG. 12B). These data demonstrate that 3×KO (TRAC−/β2M−/CD70−) anti-CD70 CAR+ cells have potent activity against human CD70+ lung cancer tumors in vivo.

### Treatment in the Pancreatic Tumor Model

The ability of T cells expressing a CD70 CAR to eliminate pancreatic carcinoma cells that express moderate levels of CD70 was evaluated in in vivo using a subcutaneous pancreatic (Hs 766T) tumor xenograft model in mice.

The ability of these anti-CD70 CAR+ T cells to ameliorate disease caused by a CD70+ pancreatic carcinoma cell line was evaluated in NOG mice using methods employed by Translational Drug Development, LLC (Scottsdale, Ariz.). In brief, 12, 5-8 week-old female, CIEA NOG (NOD.Cg-Prkdc^{scid}Il2rg^{tm1Sug}/JicTac) mice were individually housed in ventilated microisolator cages, maintained under pathogen-free conditions, 5-7 days prior to the start of the study. Mice received a subcutaneous inoculation of 5×10⁶ Hs766T pancreatic carcinoma cells in the right hind flank. When mean tumor size reached 25-75 mm³ (target of ˜50 mm³), the mice were further divided into 2 treatment groups as shown in Table 24. On Day 1, treatment group 2 received a single 200 μl intravenous dose of anti-CD70 CAR+ T cells according to Table 24.

TABLE 24
Treatment groups

| | | | T cell | |
|---|---|---|---|---|
| | | Hs766T | treatment | |
| Group | CAR-T | cells | (i.v.) | N |
| 1 | None | 5 × 10⁶ | None | 5 |
| | | cells/mouse | | |
| 2 | 3× KO (CD70,) | 5 × 10⁶ | 1 × 10⁷ | 5 |
| | anti-CD70 CAR+ T cells | cells/mouse | cells/mouse | |

Tumor volume was measured 2 times weekly from day of treatment initiation. By Day 15 post-injection, tumors treated with anti-CD70 CAR T cells began to show a decrease in tumor volume in all treated mice. Treatment with anti-CD70 CAR+ T cells effectively reduced the size of the CD70+ pancreatic cancer tumors, in all mice tested (<37 mm³) with no evidence of further growth for the duration of the study (through Day 67) (FIG. 12C). These data demonstrate that 3×KO (TRAC−/132M−/CD70−) anti-CD70 CAR+ cells induce regression of human CD70+ pancreatic cancer tumors in vivo, with potent activity against established Hs766T pancreatic cancer xenografts and durable responses beyond 60 days following treatment initiation.

### Treatment in the Gastric Tumor Model

The ability of T cells expressing an anti-CD70 CAR to eliminate ovarian adenocarcinoma cells that express moderate levels of CD70 was evaluated in vivo using a subcutaneous gastric carcinoma (SNU-1) tumor xenograft model in mice.

The ability of these anti-CD70 CAR+ T cells to ameliorate disease caused by a CD70+ ovarian carcinoma cell line was evaluated in NOG mice using methods employed by Translational Drug Development, LLC (Scottsdale, Ariz.). In brief, twelve (12) 5-8 week old female, CIEA NOG (NOD.Cg-Prkdc^{scid}Il2rg^{tm1Sug}/JicTac) mice were individually housed in ventilated microisolator cages, maintained under pathogen-free conditions, 5-7 days prior to the start of the study. Mice received a subcutaneous inoculation of 5×10⁶ SNU-1 gastric carcinoma cells/mouse in the right hind flank. When mean tumor size reached 25-75 mm³ (target of ˜50 mm³), the mice were further divided into two treatment groups as shown in Table 25. On Day 1, treatment group 2 received a single 200 μl intravenous dose of anti-CD70CAR+ T cells according to Table 25.

TABLE 25
Treatment groups

| | | | T cell | |
|---|---|---|---|---|
| | | | treatment | |
| Group | CAR-T | SNU-1 cells | (i.v.) | N |
| 1 | None | 5 × 10⁶ | None | 5 |
| | | cells/mouse | | |
| 2 | 3× KO (CD70,) | 5 × 10⁶ | 1 × 10⁷ | 5 |
| | anti-CD70 CAR+ T cells | cells/mouse | cells/mouse | |

Tumor volume was measured 2 times weekly from day of treatment initiation. By day 10 post-injection, tumors treated with anti-CD70 CART cells began to show a decrease in tumor volume. By day 20 post-injection, CD70+ gastric cancer tumors in mice treated with anti-CD70 CAR T cells experienced another significant decline in tumor size. By day 60 post-injection, CD70+ gastric cancer tumors showed complete regression of tumor growth (FIG. 12D). These data demonstrate that 3×KO (TRAC−/β2M−/CD70−) anti-CD70 CAR+ cells are highly potent in vivo for treating human gastric tumors.

### Example 10. A Phase 1, Open-Label, Multicenter, Dose Escalation and Cohort Expansion Study of the Safety and Efficacy of Allogeneic CRISPR-Cas9-Engineered T Cells (CTX130) in Adult Subjects with Advanced, Relapsed or Refractory Renal Cell Carcinoma (RCC) with Clear Cell Differentiation

CTX130 is a CD70-directed allogeneic T cell immunotherapy comprised of T cells that are genetically modified using CRISPR-Cas9 gene editing components (sgRNA and Cas9 nuclease) to knock out the T cell receptor alpha constant (TRAC) and beta 2-microglobulin (β2M) genes, which contribute to graft versus host and host versus graft reactions, respectively.

Simultaneously, an anti-CD70 CAR is inserted at the TCR locus using an AAV vector. The CAR is comprised of a scFv specific for CD70, followed by a CD8 hinge and transmembrane region that is fused to the intracellular co-signaling domain of CD137 (i.e., 4-1BB) and the signaling domain of CD3ζ. The target for CTX130 (i.e., the CD70 protein) is also removed from the final CTX130 product using the CRISPR-Cas9 system by using an sgRNA that targets the CD70 loci. This creates a functional knockout of the CD70 gene and protein in cells in which both copies of the CD70 gene have been edited.

CTX130 cells can be prepared from healthy donor peripheral blood mononuclear cells obtained via a standard leukapheresis procedure. The product is stored onsite and thawed immediately prior to administration.

### 1. Study Overview

Study Population

Dose escalation and cohort expansion include adult subjects with advanced (unresectable or metastatic), relapsed or refractory renal cell carcinoma (RCC) with clear cell differentiation who have had prior exposure to both a checkpoint inhibitor (CPI) and a vascular endothelial growth factor (VEGF) inhibitor.

Duration of Subject Participation

Subjects participate in this study for up to 5 years after the last CTX130 infusion. After completion of this study, all subjects are asked to participate in a separate long-term follow-up study for an additional 10 years to assess safety and survival.

### 2. Study Purpose

The purpose of the Phase 1 dose escalation and cohort expansion study is to evaluate the safety and efficacy of anti-CD70 allogeneic CRISPR-Cas9 engineered T cells (CTX130) in subjects with advanced (unresectable or metastatic), relapsed or refractory RCC with clear cell differentiation. The study is divided into 2 parts: Part A (dose escalation), which includes Parts A1 through A4, followed by Part B (cohort expansion). Parts A1 and A3 evaluate the safety of a single escalating dose of CTX130 (with the option of additional doses of CTX130 following relapse, stable disease, or disease progression with clinical benefit); Parts A2 and A4 evaluate the safety of a multiple dose schedule for CTX130. Part B assesses the safety and efficacy of the recommended dosing regimen for CTX130 in cohort expansion.

CAR T cell therapies are adoptive T cell therapeutics (ACTs) used to treat human malignancies. Currently approved ACTs are autologous and require patient-specific cell collection and manufacturing, which has led to reintroduction of residual contaminating tumor cells. Also, low response rates in patients with chronic lymphocytic leukemia and lack of responses in patients with B cell acute lymphoblastic leukemia treated with autologous CAR T cell therapy have been partially attributed to the exhausted T cell phenotype. Finally, collection, shipment, manufacturing, and shipment back to the patient's treating physician is time-consuming and, as a result, some patients have experienced disease progression or death while awaiting treatment. An allogeneic off-the-shelf CAR T cell product could provide benefits such as immediate availability and chemotherapy-naïve T cells from healthy donors, thus a more consistent product relative to autologous CAR T cell therapies.

CRISPR-Cas9 engineering employs a recombinant AAV vector to insert an anti-CD70 CAR expression cassette into the TRAC locus, disrupting expression of the T cell receptor (TCR), which is intended to minimize the probability of graft vs host disease (GvHD). Expression of B2M, a component of major histocompatibility (MHC) class I molecules, is also targeted for disruption, which is intended to minimize the host's MHC-mediated immune rejection of the allogeneic T cell product, thus improving persistence of CTX130. This first-in-human trial in subjects with unresectable or metastatic clear cell renal cell carcinoma (ccRCC) evaluates the safety and efficacy of this CRISPR-Cas9 modified allogeneic CAR T cell approach.

CTX130, a CD70-directed genetically modified allogeneic T cell immunotherapy, is manufactured from the cells of healthy donors; therefore, the resultant manufactured cells are intended to provide each subject with a consistent final product of reliable quality. Furthermore, the manufacturing of CTX130, through precise delivery and insertion of the CAR at the TRAC site using AAV and homology-directed repair, does not present the risks associated with random insertion of lentiviral and retroviral vectors.

Finally, CD70 is the membrane-bound ligand of the CD27 receptor, which belongs to the tumor necrosis factor receptor superfamily. It is commonly expressed at elevated levels in multiple carcinomas and lymphomas, and it is a diagnostic biomarker for ccRCC. The tightly controlled normal tissue expression in humans is mostly limited to transient surface expression in blood and lymphoid tissues, specifically activated peripheral T and B lymphocytes, scattered T cells in tonsils, skin and intestine, germinal B cell centers, thymic epithelial cells, and natural killer cells. Based on studies in knockout animal models, CD70/CD27 does not seem to be essential for the development and function of the immune system in mice. Therefore, the above characteristics of CD70 render CTX130 a promising therapy for CD70− positive malignancies.

### 3. Study Objectives

Primary Objective, Part A: To assess the safety of a single escalating dose and multiple dose regimen of CTX130.

Primary Objective, Part B (Cohort Expansion): To assess the efficacy of CTX130 in subjects with unresectable or metastatic ccRCC, as measured by ORR according to Response Evaluation Criteria in Solid Tumors version 1.1 (RECIST v1.1).

Secondary Objectives (Parts A and B): To further characterize the efficacy of CTX130 over time; to further assess the safety of CTX130, and to describe and assess adverse events of special interest (AESIs), including CRS, tumor lysis syndrome (TLS), and GvHD; and to characterize PK (expansion and persistence) of CTX130 in blood.

Exploratory Objectives (Parts A and B): To identify genomic, metabolic, and/or proteomic biomarkers that are associated with disease, clinical response, resistance, safety, or pharmacodynamic activity; to further describe the kinetics of efficacy of CTX130; and to describe the effect of CTX130 on patient-reported outcomes (PRO).

### 4. Study Eligibility

Inclusion Criteria

To be considered eligible to participate in this study, a subject must meet all the inclusion criteria listed below:
1. ≥18 years of age and body weight≥42 kg.
2. Able to understand and comply with protocol-required study procedures and voluntarily sign a written informed consent document.
3. Diagnosed with unresectable or metastatic RCC with clear cell differentiation:
   Have previous exposure to both a CPI and a VEGF inhibitor and documented progression after adequate exposure for favorable risk by IMDC criteria (Heng et al., J Clin Oncol, 2009) or, at a minimum, a lack of response and/or progression after adequate exposure for intermediate and poor risk characteristics.
   Have a previously pathologically confirmed diagnosis of RCC with clear cell differentiation.
   Availability of tumor tissues.
   Have measurable disease, as assessed by radiologists per RECIST v1.1. Target lesions situated in a previously irradiated area are considered measurable if progression has been demonstrated in such lesions.
   Have at least 1 nontarget lesion that is suitable for biopsies.
4. Karnofsky performance status>80%, as assessed during the screening period.
5. Meets protocol-specified criteria to undergo daratumumab administration (Parts A3 and A4 only), LD chemotherapy, and CAR T cell infusion.
6. Adequate organ function:
   Renal: Creatinine clearance≥50 mL/min.
   Liver:
      Aspartate aminotransferase and alanine aminotransferase<3× upper limit of normal (ULN)
      Total bilirubin<2×ULN (for Gilbert's syndrome: total bilirubin<3 mg/dL and normal conjugated bilirubin)
   Albumin>90% of lower limit of normal
   Cardiac: Hemodynamically stable and left ventricular ejection fraction≥45% by echocardiogram.
   Pulmonary: Oxygen saturation level on room air>92%, per pulse oximetry.
   Hematologic: Platelet count>100,000/mm³, absolute neutrophil count>1500/mm³, and hemoglobin>9 g/dL without prior blood cell transfusion before screening.
   Coagulation: Activated partial thromboplastin time partial thromboplastin time≤1.5×ULN.
7. Female subjects of childbearing potential (postmenarcheal, has an intact uterus and at least 1 ovary, and is less than 1 year postmenopausal) must agree to use a highly effective method of contraception from enrollment through at least 12 months after last CTX130 infusion.
8. Male subjects must agree to use acceptable effective method(s) of contraception from enrollment through at least 12 months after last CTX130 infusion.

Exclusion Criteria

To be eligible for entry into the study, the subject must not meet any of the exclusion criteria listed below:
1. Prior treatment with any anti-CD70 targeting agents.
2. Prior treatment with any CAR T cells or any other modified T or NK cells.
3. Known contraindications to daratumumab (Parts A3 and A4 only), any LD chemotherapy agent(s), or any of the excipients of CTX130 product.
4. Subjects with central nervous system (CNS) manifestation of their malignancy as evidenced by positive screening magnetic resonance imaging (MRI) or past history.
5. History or presence of clinically relevant CNS pathology such as seizure, stroke, severe brain injury, cerebellar disease, history of posterior reversible encephalopathy syndrome with prior therapy, or another condition that may increase CAR T cell-related toxicities.
6. Ongoing, clinically significant pleural effusion or ascites or any pericardial effusion or a history of pleural effusion or ascites in the last 2 months.
7. Unstable angina, clinically significant arrhythmia, or myocardial infarction within 6 months prior to screening.
8. Diabetes mellitus with a current hemoglobin A1c level of >7.0%.
9. Ongoing bacterial, viral, or fungal infection requiring systemic anti-infectives.
10. Positive for presence of human immunodeficiency virus type 1 or 2, or active hepatitis B virus or hepatitis C virus infection. Subjects with prior history of hepatitis B or C infection who have documented undetectable viral load (by quantitative polymerase chain reaction [PCR] or nucleic acid testing) are permitted.
11. Previous or concurrent malignancy, except those treated with curative approach not requiring systemic therapy and have been in remission for >12 months, or any other localized malignancy that has a low risk of developing into metastatic disease.
12. Primary immunodeficiency disorder or active autoimmune disease requiring steroids and/or any other immunosuppressive therapy.
13. Prior solid organ transplantation or bone marrow transplant.
14. Use of systemic antitumor therapy or investigational agent within 14 days prior to enrollment. Use of physiological doses of steroids (e.g., ≤10 mg/day prednisone or equivalent) is permitted for subjects previously on steroids if clinically indicated.
15. Received live vaccines or herbal medicines as part of traditional Chinese medicine or non-over-the-counter herbal remedies within 28 days prior to enrollment.
16. Diagnosis of significant psychiatric disorder that could seriously impede the subject's ability to participate in the study.
17. Pregnant or breastfeeding females.

### 5. STUDY DESIGN

Investigational Plan

This is an open-label, multicenter, Phase 1 study evaluating the safety and efficacy of CTX130 in subjects with unresectable or metastatic RCC with clear cell differentiation. The study is divided into 2 parts: Part A (dose escalation), which includes Parts A1 through A4, followed by Part B (cohort expansion).

Parts A1 and A3 evaluate the safety of a single escalating dose of CTX130 with the option of additional doses of CTX130 following relapse, stable disease, or disease progression with clinical benefit; Parts A2 and A4 evaluate the safety of a multiple dose schedule for CTX130. Part B further assesses the safety and efficacy of the recommended dosing regimen for CTX130 in cohort expansion.

In Part A1, dose escalation begins in adult subjects diagnosed with unresectable or metastatic ccRCC who have progressed after both a CPI and a VEGF inhibitor. Dose escalation in Part A1 is performed according to the criteria described herein.

Enrollment into a Part A2 dose level may begin if that dose level has been deemed safe in Part A1. Each subject in Part A2 receives a total of up to 3 doses of CTX130: 1 dose is administered every 8 weeks (1 dose per cycle in Cycles 1-3). Prior to each CTX130 infusion, subjects receive the same LD chemotherapy dose regimen as administered prior to the initial CTX130 infusion.

Subjects in Part A3 receive daratumumab (Darzalex® or Darzalex Faspro™, Janssen; an anti-CD38 monoclonal antibody), a human IgG1 mAb that targets CD38 surface antigen, prior to LD chemotherapy to achieve depletion of CD38+ immune suppressor and effector cells (e.g., NK cells). CTX130 is an allogeneic CAR T cell with disruption of the β2M locus resulting in elimination of MHC class I expression on the cell surface, NK cells can potentially detect and clear “non-self” MHC class1 negative CAR T cells. Rapid NK cell recovery after LD chemotherapy was found to coincide with peak CTX130 expansion. Based on these observations, the suppression of specific NK cell subpopulations with daratumumab in addition to LD chemotherapy may reduce the potential host immune response to an allogeneic CAR T cell product, and therefore allow increased expansion and persistence of CTX130.

Dosing of CTX130 at any level in Part A3 does not begin until the dose level has been deemed safe in Part A1. Dose escalation/de-escalation is allowed according to the 3+3 design. Sentinel dosing is implemented for the starting dose level, i.e., the first subject completes the dose-limiting toxicity (DLT) evaluation period before the second and third subjects are dosed. The second and third subjects may be dosed concurrently. In subsequent dose levels or expansion of the same dose level, cohorts of up to 3 subjects may be enrolled and dosed concurrently. Daratumumab administration at 16 mg/kg IV or 1800 mg subcutaneous [SC] injection is repeated at Day 22. A third dose of daratumumab (16 mg/kg IV or 1800 mg SC) is administered on Day 42 in subjects who achieve SD or better. The Day 42 computed tomography (CT) scan must be read prior to repeat dosing with daratumumab.

Enrollment into a Part A4 dose level may begin if that dose level has been deemed safe in Part A3. Each subject receives a total of up to 3 doses of CTX130: 1 dose administered every 8 weeks (1 dose per cycle in Cycles 1-3). Dosing of subjects may occur concurrently. Prior to each CTX130 infusion, subjects receive 16 mg/kg IV or 1800 mg SC daratumumab, followed by the same LD chemotherapy dose regimen as administered prior to the initial CTX130 infusion. Daratumumab administration at 16 mg/kg IV or 1800 mg SC is repeated at Day 22 of each cycle.

In Part B, an expansion cohort is initiated to further assess the safety and efficacy of CTX130 using an optimal Simon 2-stage design. In the first stage of Part B, at least 23 subjects are treated with the recommended dose of CTX130 for Part B cohort expansion (using a dosing regimen determined in Part A). When 23 subjects are treated in Part B and have 3 months of evaluable response data, the DSMB reviews the data based on an interim analysis to make a decision on enrollment of 48 additional subjects, to bring the total number of subjects in Part B to approximately 71. Subjects in the expansion cohort are redosed with the RPBD.

Study Design

This is an open-label, multicenter, Phase 1 study evaluating the safety and efficacy of CTX130 in subjects with unresectable or metastatic RCC with clear cell differentiation. The study is divided into 2 parts: Part A (dose escalation), which includes Parts A1 through A4, followed by Part B (cohort expansion). Each part of the study consists of 3 main stages:
Stage 1: Screening to determine eligibility for treatment (up to 14 days)
Stage 2: Treatment (Stage 2A and Stage 2B); see Table 26 for treatment in each part of the study
Stage 3: Follow-up (5 years after last CTX130 infusion)

Subjects' clinical eligibility must be reconfirmed according to the protocol-specified criteria described herein prior to the initiation of daratumumab administration (subjects in Parts A3 and A4 only), prior to LD chemotherapy (all subjects), and prior to CTX130 infusion (all subjects). Lymphodepletion regimens and CTX130 dosing are summarized in Table 26.

For Parts A3 and A4, after at least 3 subjects are treated at a specific CTX130 dose with daratumumab, the total safety and efficacy data are to be reviewed and a specific dose level with a lower dose of daratumumab (e.g., 8 mg/kg IV) may be recommended.

During the post-CTX130 infusion period, subjects are monitored for acute toxicities (Days 1-28), including CRS, immune effector cell-associated neurotoxicity syndrome (ICANS), GvHD, and other AEs. Toxicity management guidelines are provided herein. During Part A, subjects are hospitalized for the first 7 days following each CTX130 infusion, or longer if required by local regulation or site practice. In Part A and Part B, subjects must remain within proximity of the investigative site (i.e., 1-hour transit time) for 28 days after each CTX130 infusion.

After the acute toxicity observation period, subjects are subsequently followed for up to 5 years after last CTX130 infusion with physical exams, regular laboratory and imaging assessments, and AE assessments. After completion of this study, subjects are asked to participate in a separate long-term follow-up study for an additional 10 years to assess long-term safety and survival.

TABLE 26
Lymphodepletion Regimens and CTX130 Dosing

| Study Part | Treatment by Stage |
|---|---|
| A1 | Stage 2A |
| (Single Dose | LD chemotherapy: Co-administration of fludarabine 30 mg/m² and |
| Escalation) | cyclophosphamide 500 mg/m² IV daily for 3 days. |
| | Stage 2B |
| | CTX130 starting at DL1, administered at least 48 hours (but no more than 7 |
| | days) after completion of LD chemotherapy. |
| | Option to receive up to 2 additional CTX130 doses with LD chemotherapy after 1) |
| | loss of response within the first 2 years after last dose of CTX130, or 2) stable |
| | disease or progressive disease with significant clinical benefit at Day 42. |
| A2 | Stage 2A |
| (Multiple Dose | LD chemotherapy: Co-administration of fludarabine 30 mg/m² and |
| Regimen) | cyclophosphamide 500 mg/m² IV daily for 3 days. |
| | Stage 2B |
| | CTX130 dosing starts at a dose level that has been deemed safe in Part A1. |
| | CTX130 is administered at least 48 hours (but no more than 7 days) after |
| | completion of LD chemotherapy. |
| | Each subject receives a total of up to 3 doses of CTX130 (1 dose every 8 weeks), |
| | regardless of disease response. |
| A3 | Stage 2A |
| (Single Dose | One dose of daratumumab 16 mg/kg IV or 1800 mg SC administered at least |
| Escalation with | 12h prior to starting LD chemotherapy and within 10 days of CTX130 |
| Daratumumab | infusion. Daratumumab administration at 16 mg/kg IV or 1800 mg SC is |
| added to the | repeated at Day 22 (and at Day 42 + 7 days in subjects who achieve stable |
| lymphodepletion | disease or better). |
| regimen) | LD chemotherapy: Co-administration of fludarabine 30 mg/m² + |
| | cyclophosphamide 500 mg/m² IV daily for 3 days. |
| | Stage 2B |
| | CTX130 dosing starts at a dose level deemed safe in Part Al, administered at |
| | least 48 hours (but no more than 7 days) after completion of LD |
| | chemotherapy. |
| | Option to receive up to 2 additional doses of CTX130 with daratumumab and LD |
| | chemotherapy after 1) loss of response within the first 2 years after last dose of |
| | CTX130, or 2) stable disease or progressive disease with significant clinical benefit |
| | at Day 42. |
| A4 | Stage 2A |
| (Multiple Dose | One dose of daratumumab 16 mg/kg IV or 1800 mg SC administered at least |
| Regimen with | 12h prior to starting LD chemotherapy and within 10 days of CTX130 |
| Daratumumab | infusion. Daratumumab administration at 16 mg/kg IV or 1800 mg SC is |
| added to the | repeated at Day 22. |
| lymphodepletion | LD chemotherapy: Co-administration of fludarabine 30 mg/m² and |
| regimen) | cyclophosphamide 500 mg/m² IV daily for 3 days. |
| | Stage 2B |
| | CTX130 dosing starts at a dose level that has been deemed safe in Part A3. |
| | CTX130 is administered at least 48 hours (but no more than 7 days) after |
| | completion of LD chemotherapy. |
| | Each subject to receive a total of up to 3 doses of CTX130 (1 dose every 8 weeks) |
| | with daratumumab if considered safe. |
| Part B (Cohort | Dosing regimen determined in Part A |
| Expansion) | |

DL: Dose Level; IV: intravenously; LD: lymphodepleting

Study Subjects

The total number of study subjects (Part A+Part B) is approximately 149.
Part A1: Up to 36 subjects.
Part A2: Up to 36 subjects.
Part A3: Up to 36 subjects.
Part A4: Up to 36 subjects.
Part B, Cohort Expansion: approximately 71 subjects are treated in Part B, contingent upon the outcome of an interim analysis.

Study Duration

Subjects participate in this study for up to 5 years after last CTX130 infusion. After completion of this study, subjects are asked to participate in a separate long-term follow-up study for an additional 10 years to assess long-term safety and survival.

CTX130 Dose Escalation

The following doses of CTX130, based on number of CAR⁺ T cells, may be evaluated in this study, starting with DL1 in Part A1 (Table 27). There is a dose limit of 7×10⁴ TCR⁺ cells/kg imposed for all dose levels.

TABLE 27
Dose Escalation of CTX130

| Dose Levels | Total CAR⁺ T Cell Dose |
|---|---|
| −1 (de-escalation) | 1 × 10⁶ |
| 1 | 3 × 10⁷ |
| 2 | 1 × 10⁸ |
| 3 | 3 × 10⁸ |
| 4 | 9 × 10⁸* |

CAR: chimeric antigen receptor.
*An intermediate dose level between DL3 and DL4, i.e., 4.5 × 10⁸, 6 × 10⁸, or 7.5 × 10⁸ CAR* T cells, may be administered based on review of Dose Level 4 safety data.

Dose escalation in Part A is performed using a standard 3+3 design in which 3 to 6 subjects are enrolled at each dose level depending on the occurrence of DLTs, as defined herein. The DLT evaluation period begins with the initial CTX130 infusion and lasts for 28 days.

Part A1: In DL1, subjects are treated in a staggered manner, such that a subject only receives CTX130 once the previous subject has completed the DLT evaluation period (i.e., staggered by 28 days). If occurrence of a DLT in >2 of 3 subjects at DL1 has resulted in dose de-escalation, dosing of all subjects at DL-1 is also staggered by 28 days. If no DLT occurs at DL1, dose escalation progresses to DL2, and dosing between each subject is staggered by 14 days. If no DLT occurs at the first 2 dose levels (DL1 and DL2), dosing is staggered by 7 days between each subject at subsequent dose levels (DL3 and DL4).

Part A2: Enrollment into a Part A2 dose level begins at a dose level that has been deemed safe in Part A1. Each subject receives a total of up to 3 doses of CTX130: 1 dose administered every 8 weeks (1 dose per cycle in Cycles 1-3).

Part A3: Dosing of CTX130 at any dose level in Part A3 does not begin unless the dose level has been deemed safe in Part A1. Dose escalation/de-escalation is allowed according to the 3+3 design. Sentinel dosing is implemented for the starting dose level, i.e., the first subject completes the DLT evaluation period before the second and third subjects are dosed. The second and third subjects may be dosed concurrently. In subsequent dose levels or expansion of the same dose level, cohorts of up to 3 subjects may be enrolled and dosed concurrently.

Part A4: Enrollment into a Part A4 dose level begins if that dose level has been deemed safe in Part A3. Each subject receives a total of up to 3 doses of CTX130: 1 dose administered every 8 weeks (1 dose per cycle in Cycles 1-3). Dosing of subjects may occur concurrently.

Subjects must receive CTX130 to be evaluated for DLT. If a subject discontinues the study any time prior to the initial CTX130 infusion, the subject is deemed nonevaluable for DLT and is replaced. If a DLT-evaluable subject (i.e., a subject that has been administered CTX130) has signs or symptoms of a potential DLT, the DLT evaluation period is extended according to the protocol-defined window to allow for improvement or resolution before a DLT is declared.

Dose escalation is performed according to the following rules:
If 0 of 3 subjects experience a DLT, escalate to the next dose level.
If 1 of 3 subjects experiences a DLT, expand the current dose level to 6 subjects.
   If 1 of 6 subjects experiences a DLT, escalate to the next dose level.
   If ≥2 of 6 subjects experience a DLT:
      If in Dose Level −1, evaluate alternative dosing schema or declare inability to determine recommended dose for Part B cohort expansion.
      If in Dose Level 1, de-escalate to Dose Level −1.
      If in Dose Level 2-4, declare previous dose level the maximum tolerated dose (MTD).
If ≥2 of 3 subjects experience a DLT:
   If in Dose Level −1, evaluate alternative dosing schema or declare inability to determine the recommended dose for Part B cohort expansion.
   If in Dose Level 1, decrease to Dose Level −1.
   If in Dose Level 2-4, declare previous dose level the MTD. If this is the starting dose level, de-escalate to a dose previously cleared in Part A1
No dose escalation beyond highest dose listed in Table 27.

Maximum Tolerated Dose Definition

The MTD is the highest dose for which DLTs are observed in fewer than 33% of subjects. An MTD may not be determined in this study. A decision to move to the Part B expansion cohort may be made in the absence of an MTD provided the dose is at or below the maximum dose studied (or MAD) in Part A of the study.

DLT Definitions

Toxicities are graded and documented according to NCI CTCAE version 5.0, except as provided for CRS (American Society for Transplantation and Cellular Therapy [ASTCT] criteria; Lee et al., Biol Blood Marrow Transplant, 2019), neurotoxicity (CTCAE v5.0 and ICANS criteria; Lee et al., Biol Blood Marrow Transplant, 2019), and GvHD (Mount Sinai Acute GvHD International Consortium [MAGIC] criteria; Harris et al., Biol Blood Marrow Transplant, 2016). AEs that have no plausible causal relationship with CTX130 are not considered DLTs.

DLTs are defined as:
Grade>2 GvHD if it does not respond to steroid treatment (e.g., 1 mg/kg/day) within 7 days (GvHD grading is provided in Table 42).
Any CTX130-related grade 3 to 5 toxicity occurring within 28 days immediately after infusion of CTX130, with the following exceptions (Table 28):

TABLE 28
Exception Criteria

| Exceptions | Criteria |
|---|---|
| #1 | Any grade 3 or 4 CRS, according to the CRS grading system (Table 37), |
| | that improves to grade ≤2 with appropriate medical intervention within 72 |
| | hours. |
| #2 | Grade 3 or 4 fever resolving within 72 hours with appropriate medical |
| | intervention. |
| #3 | Grade 3 fatigue lasting <7 days. |
| #4 | Any grade 3 or 4 abnormal liver function tests that improve to grade ≤2 |
| | within 14 days. |
| #5 | Any grade 3 toxicity involving vital organs other than cardiac |
| | (e.g., pulmonary, renal) that improves to grade ≤2 within 7 days. |
| #6 | Any grade 3 cardiac toxicity that improves to grade ≤2 within 72 hours. |
| #7 | Any grade 3 neurotoxicity that resolves to grade ≤2 within 72 hours. |
| #8 | Death due to disease progression. |
| #9 | GvHD that is not steroid-refractory and resolves to grade 1 within 14 days. |

If a subject has a potential DLT for which the protocol definition allows time for improvement or resolution, the DLT evaluation period is extended accordingly before a DLT is declared.

AEs occurring outside the DLT evaluation period that are assessed as related to CTX130 are when making dose escalation decisions.

CTX130 Repeat Dosing in Parts A1 and A3

In Parts A1 and A3 of this study, subjects are allowed to receive up to 2 additional doses of CTX130 after the conditioning regimen. To be considered for repeat dosing, subjects must have either: 1) achieved a PR or CR after initial or second CTX130 infusion, and within 2 years of last dose have an increase in tumor size (sum of target lesion diameters) of at least 10% or 2) achieved SD, or PD with significant clinical benefit, at the Day 42 study visit after the most recent CTX130 infusion. Repeat dosing decisions are based upon local CT scan/assessment.

The earliest time at which a subject could be redosed is 8 weeks after the initial or second CTX130 infusion.

To be redosed with CTX130, subjects in Part A1 and Part A3 must meet the following criteria:
Confirmation tumor is CD70⁺ (based on local or central assessment) if a lesion is available that is amenable to biopsy
No prior DLT during dose escalation
No prior grade≥3 CRS without resolution to grade≤2 within 72 hours following CTX130 infusion
No prior grade>1 GvHD following CTX130 infusion
No prior grade≥2 ICANS following CTX130 infusion
Meet initial study inclusion criteria (#1, #2, #4-8) and exclusion criteria (#2 [except prior treatment with CAR T cells]-17)
Meet criteria for daratumumab dosing (Part A3 only), LD chemotherapy and CTX130 infusion as described herein

Subjects who are redosed receive 3 days of LD chemotherapy and should be followed per the schedule of assessments (Tables 29-31) consistent with the initial dosing. All screening assessments must be repeated, including brain MM. In Part A3, daratumumab may be administered with repeat dosing of CTX130 following the same administration schedule.

Additional redosing considerations include the following:
If PD occurred prior to redosing, the most recent CT scan prior to redosing serves as the new baseline for tumor response evaluation. Redosing must occur within 28 days of that scan.
If SD is the response at Day 42 or if a subject achieved PR prior to redosing, the original baseline scan continues to be used for tumor response evaluation.
Subjects in the dose escalation cohorts who undergo redosing receive a CTX130 dose that is either (a) identical to that previously received, or (b) was subsequently deemed safe (minimum n=3)
Subjects in the expansion cohort are redosed with the RPBD.

CTX130 Multiple Dose Regimen in Parts A2 and A4

Given the potential clinical benefit that can be derived from redosing, subjects enrolled in Parts A2 and A4 receive a multiple dose regimen of up to 3 doses of CTX130: 1 dose is administered every 8 weeks (1 dose per cycle in Cycles 1-3; see schedule of assessments, Tables 29-31) to assess safety for subjects receiving multiple doses of CTX130.

Three subjects are treated per dose level, with the option to expand any dose level to 6 subjects. Enrollment into a Part A2 dose level may begin if that dose level has been deemed safe in Part A1, and enrollment into a Part A4 dose level may begin if that dose level has been deemed safe in Part A3. Up to 3 dose levels are evaluated in Part A2 and Part A4 if the corresponding single dose levels are deemed safe.

Prior to each CTX130 dose, subjects undergo pre-LD chemotherapy assessments followed by 3 days of LD chemotherapy (see Table 26). In Part A4, daratumumab is administered with each dosing of CTX130 following the same administration schedule as described herein.

To be considered for redosing in Cycle 2 and Cycle 3, each subject needs to meet the following criteria:
No DLT in prior cycle(s)
No prior grade≥3 CRS without resolution to grade≤2 within 72 hours following CTX130 infusion
No prior grade>1 GvHD following CTX130 infusion
No prior grade≥2 ICANS following CTX130 infusion
Meets initial study inclusion criteria (#1, #2, #4-8) and exclusion criteria (#2 [except prior treatment with CAR T cells]-17)
Meets criteria for LD chemotherapy and CTX130 infusion

In Part A4, daratumumab may be administered with CTX130 multiple dose regimen following the administration schedule.

### 6. Study Procedures

Both the dose escalation and expansion parts of the study consist of 3 distinct stages: (1) screening and eligibility confirmation, (2) daratumumab administration (Parts A3 and A4 only), LD chemotherapy, and CTX130 infusion, and (3) follow-up. During the screening period, subjects are assessed according to the eligibility criteria outlined previously. After enrollment, subjects in Part A1 and Part A2 receive LD chemotherapy, followed by infusion of CTX130; subjects in Part A3 and Part A4 receive daratumumab, followed by LD chemotherapy, then CTX130 infusion. After completing the treatment period, subjects are assessed for ccRCC response, disease progression, and survival. Throughout all study periods, subjects are regularly monitored for safety.

A complete schedule of assessments is provided in Tables 29-31. Subjects enrolled in Part A1 (single dose escalation) and Part A3 (single dose escalation with daratumumab added to the lymphodepletion regimen) follow the schedule of assessments shown in Tables 29-31, and subjects enrolled in Part A2 (multiple dose regimen) and Part A4 (multiple dose regimen with daratumumab added to the lymphodepletion regimen) follow the schedule of assessments shown in Tables 29-31. Subjects enrolled in Part B (cohort expansion) follow the schedule of assessments either in any one of Tables 29-31, depending on which dose level and dosing schedule is chosen for the RPBD. All subjects, whether enrolled in Part A or Part B, follow the schedule of assessments for Months 30-60 shown in Tables 29-31.

Descriptions of all required study procedures are provided in this section. In addition to protocol-mandated assessments, subjects should be followed per institutional guidelines, and unscheduled assessments should be performed when clinically indicated.

Missed evaluations should be rescheduled and performed as close to the originally scheduled date as possible. An exception is made when rescheduling becomes, medically unnecessary or unsafe because it is too close in time to the next scheduled evaluation. In that case, the missed evaluation should be abandoned.

For the purposes of this protocol, there is no Day 0. All visit dates and windows are calculated using Day 1 as the date of CTX130 infusion.

TABLE 29
Schedule of Assessments for Part A1 and Part A3 (Dose Escalation): Screening to Month 24

| | | Treatment | | | | | | | | | |
|---|---|---|---|---|---|---|---|---|---|---|---|
| | | D-10 to | | | | | | | | | |
| | | 12 hr | | | | | | | | | |
| | | prior to | | | | | | | | | |
| | | LD | | | | | | | | | |
| | | chemo | D-7 | | | | Follow-Up | | | | |
| Assessment | | Part A3 | to | | | | | D7 | D10 | D15 | D22 |
| Day | Screening¹ | only³³ | D-3 | D1 | D2 | D3 | D5 | +2 d | ±1 d | ±2 d | ±2 d |
| Eligibility | X | X | X | X | | | | | | | |
| confirmation² | | | | | | | | | | | |
| Informed consent | X | | | | | | | | | | |
| Medical history ³ | X | | | | | | | | | | |
| Physical exam⁴ | X | X | X | X | X | X | X | X | X | X | X |
| Vital signs⁵ | X | X | X | X | X | X | X | X | X | X | X |
| Height, weight⁶ | X | X | X | X | | | | | | | |
| Pregnancy test⁷ | X | X | X | | | | | | | | |
| Brain MRI⁸ | X | | | | | | | | | | |
| Karnofsky status | X | | | X | | X | | X | | X | X |
| Echocardiogram | X | | | | | | | | | | |
| 12-lead ECG⁹ | X | | X | X | | | | | | | |
| ICE assessment¹⁰ | X | | | X | X | X | X | X | X | X | X |
| PRO¹¹ | X | | | X | | | | | | X | |

| Concomitant | Continuous | |
|---|---|---|
| medications¹² | | |
| AEs¹³ | Continuous | |
| Hospital utilization | | Continuous |

Treatment

| | | | | |
|---|---|---|---|---|
| Daratumumab¹⁴ | X | | | X |
| Part A3 only | | | | |
| LD chemo¹⁶ | | X | | |
| CTX130¹⁷ | | | X | |

Metastatic ccRCC Disease/Response Assessments (Central)

| | | |
|---|---|---|
| CT scan¹⁸ | X | |
| Tumor biopsy^{19, 20} | X | X |

Laboratory Assessments (Local)

| | | | | | | | | | | | |
|---|---|---|---|---|---|---|---|---|---|---|---|
| CBC w/ differential | X | X | X | X | X | X | X | X | X | X | X |
| Serum chemistry²¹ | X | X | X | X²¹ | X²¹ | X²¹ | X²¹ | X²¹ | X²¹ | X²¹ | X²¹ |
| Coagulation | X | X | X | X | X | X | X | X | X | X | X |
| parameters | | | | | | | | | | | |
| Viral serology²² | X | | | | | | | | | | |
| SARS-CoV-2²³ | X | | | | | | | | | | |
| Lymphocyte | X | X | X | X | | | X | X | X | X | X |
| subsets²⁴ | | | | | | | | | | | |
| Ferritin, CRP, | X | | | X | X | X | X | X | X | X | X |
| Triglyceride | | | | | | | | | | | |
| sCD25³⁴ | | | | | | | | | | X | X |

Biomarkers (Blood, Central)

| CTX130 levels²⁵ | X | | | X²⁶ | X | X | X | X | X | X | X |
|---|---|---|---|---|---|---|---|---|---|---|---|
| | | | | pre/ | | | | | | | |
| | | | | post | | | | | | | |
| Cytokines²⁷ | X | | | X | X | X | X | X | X | X | X |
| BSAP, PINP²⁸ | X | | | X | | | | X | | X | X |
| Anti-CTX130 Ab | X | | | | | | | | | | |
| Daratumumab PK²⁹ | X | X²⁹ | X³⁰ | X²⁹ | | | | X | | X | X²⁹ |
| Part A3 only | | pre/ | | | | | | | | | pre/ |
| | | post | | | | | | | | | post |
| Cell-free DNA | X | | | | | | | | | | |
| Exploratory | X³¹ | X | X³² | X | X | X | X | X | X | X | X |
| biomarkers³⁰ | | | | | | | | | | | |
| | Follow-Up | | | | | | | | | | |
| | Part | | | | | | | | | | |
| | A3 | | | | | | | | | | |
| | only | M1/ | | M2/ | M3/ | M6/ | M9/ | M12/ | M15/ | M18/ | M24/ |
| Assessment | D25 | D28 | D42 | D56 | D84 | D168 | D252 | D336 | D420 | D504 | D672 |
| Day | ±2 d | ±2 d | ±2 d | ±7 d | ±7 d | ±14 d | ±14 d | ±14 d | ±14 d | ±14 d | ±21 d |
| Eligibility | | | | | | | | | | | |
| confirmation² | | | | | | | | | | | |
| Informed consent | | | | | | | | | | | |
| Medical history ³ | | | | | | | | | | | |
| Physical exam⁴ | X | X | X | X | X | X | X | X | X | X | X |
| Vital signs⁵ | X | X | X | X | X | X | X | X | X | X | X |
| Height, weight⁶ | | X | | | X | | | | | | |
| Pregnancy test⁷ | | X | | X | X | | | | | | |
| Brain MRI⁸ | | | | | | | | | | | |
| Karnofsky status | | X | X | X | X | X | X | X | X | X | X |
| Echocardiogram | | | | | | | | | | | |
| 12-lead ECG⁹ | | | X | | | | | | | | |
| ICE assessment¹⁰ | | X | X | X | | | | | | | |
| PRO¹¹ | | X | | X | X | X | X | X | X | X | X |

| Concomitant | Continuous |
|---|---|
| medications¹² | |
| AEs¹³ | Continuous |
| Hospital utilization | Continuous |

Treatment

| | |
|---|---|
| Daratumumab¹⁴ | X |
| Part A3 only | |
| LD chemo¹⁶ | |
| CTX130¹⁷ | |

Metastatic ccRCC Disease/Response Assessments (Central)

| | | | | | | | | |
|---|---|---|---|---|---|---|---|---|
| CT scan¹⁸ | X | X | X | X | X | X | X | X |
| Tumor biopsy^{19, 20} | X | | | | | | | |

Laboratory Assessments (Local)

| | | | | | | | | | | | |
|---|---|---|---|---|---|---|---|---|---|---|---|
| CBC w/ differential | X | X¹⁵ | X | X | X | X | X | X | X | X | X |
| Serum chemistry²¹ | X²¹ | X²¹ | X | X | X | X | X | X | X | X | X |
| Coagulation | | X | X | | | | | | | | |
| parameters | | | | | | | | | | | |
| Viral serology²² | | | | | | | | | | | |
| SARS-CoV-2²³ | | | | | | | | | | | |
| Lymphocyte | X | X | X | X | X | X | X | X | X | X | X |
| subsets²⁴ | | | | | | | | | | | |
| Ferritin, CRP, | X | X | X | | | | | | | | |
| Triglyceride | | | | | | | | | | | |
| sCD25³⁴ | X | X | | | | | | | | | |

Biomarkers (Blood, Central)

| | | | | | | | | | | | |
|---|---|---|---|---|---|---|---|---|---|---|---|
| CTX130 levels²⁵ | X | X | X | X | X | X | X | X | X | X | X |
| Cytokines²⁷ | X | X | X | X | X | X | | | | | |
| BSAP, PINP²⁸ | | X | | | X | X | | X | | | |
| Anti-CTX130 Ab | | X | | | X | X | | X | | | X |
| Daratumumab PK²⁹ | | | X²⁹ | | | | | | | | |
| Part A3 only | | | pre/ | | | | | | | | |
| | | | post | | | | | | | | |
| Cell-free DNA | | X | | X | X | X | X | X | | X | X |
| Exploratory | | X | X | X | X | X | X | X | X | X | X |
| biomarkers³⁰ | | | | | | | | | | | |

Ab: antibody; AE: adverse event; BSAP: bone-specific alkaline phosphatase; CBC: complete blood count; chemo: chemotherapy; ccRCC: clear cell renal cell carcinoma; CNS: central nervous system; COVID-19: coronavirus disease 2019; CRP: C-reactive protein; CRS: cytokine release syndrome; CT: computed tomography; D or d: day; EBV: Epstein-Barr virus; ECG: electrocardiogram; EORTC: European Organization for Research and Treatment of Cancer; EQ-5D-5L: EuroQol-5 Dimension-5 Level; FACT-G: Functional Assessment of Cancer Therapy-General; FKSI-19: Functional Assessment of Cancer Therapy-Kidney Symptom Index; GvHD: graft vs host disease; HBV: hepatitis B virus; HCV: hepatitis C virus; HHV-6: human herpesvirus 6; HIV: human immunodeficiency virus; ICE: immune effector cell-associated encephalopathy; LD: lymphodepleting; M: month; MRI: magnetic resonance imaging; PET: positron emission tomography; PINP: procollagen type I N propeptide; PK: pharmacokinetics; PRO: patient-reported outcome; SARS-CoV-2: severe acute respiratory syndrome coronavirus 2; sCD25: soluble CD25; SD: stable disease; TBNK: T, B, natural killer cells.
Note:
Assessments scheduled on CTX130 infusion days are to be performed pre-CTX130 infusion unless otherwise specified.
Note:
For Parts A1 and A3, this study allows for repeat dosing of subjects with CTX130 per redosing criteria described herein. Prior to repeat dosing, all screening assessments must be repeated, except for CT scan and specific central lab samples. Subjects who are redosed receive 3 days of LD chemotherapy prior to each CTX130 infusion, and should be followed per the schedule of assessments, consistent with the initial dosing except that tumor biopsy is not performed on Days 7 and 42. The earliest time at which a subject could be redosed is 12 weeks after the initial or second CTX130 infusion.
Note:
Certain assessments for visits after Day 8 may be performed as in-home or alternate-site visits. Assessments may include hospital utilization, changes in health and/or changes in medications, vital signs, weight, PRO questionnaire distribution, and blood sample collections for local and central laboratory assessments.
¹Screening assessments to be completed within 14 days after signing the informed consent form. The screening period may be extended beyond 14 days to allow for COVID-19 testing only. Subjects are allowed a one-time rescreening, which may take place within 3 months of the initial consent.
²Eligibility should be confirmed each time screening is completed. Eligibility should also be confirmed on day of daratumumab administration (Part A3 only), on first day of LD chemotherapy, and on day of CTX130 infusion. Eligibility should be reconfirmed after all assessments for that day are completed, and before dosing.
³Includes complete surgical and cardiac history.
⁴Includes assessment for signs and symptoms of GvHD: skin, oral mucosa, sclera, hands, and feet.
⁵Includes blood pressure, heart rate, respiratory rate, pulse oximetry, and temperature.
⁶Height at screening only.
⁷For female subjects of childbearing potential. Serum pregnancy tests are required at screening, within 72 hours of beginning LD chemotherapy (Part A1), initial daratumumab dose (Part A3), and at Day 28, Day 56, and Month 3 visits.
⁸Brain MRI to be performed at screening (i.e., within 28 days prior to CTX130 infusion).
⁹12-lead ECG test should be conducted at screening, prior to initial daratumumab administration (Part A3 only), LD chemotherapy, and CTX130 infusion, and on Day 42.
¹⁰On Day 1, prior to CTX130 administration. If CNS symptoms persist after Day 56, ICE assessment should continue to be performed approximately every 2 days until symptom resolution to grade 1 or baseline.
¹¹EORTC QLQ-C30, EQ-5D-5L, FKSI-19, and FACT-G questionnaires. PROs should be completed at screening, pre-dose on Day 1, then Day 15 and Day 28 post-CTX130 infusion, and thereafter as specified in the schedule of assessment.
¹²All concomitant medications are collected up to 3 months post-CTX130 infusion. Afterwards, only select concomitant medications are collected (i.e., immunomodulating agents, blood products, antitumor medications, hormones, and growth factors).
¹³AEs are collected for enrolled subjects according to the AE reporting requirements for each time period of the study as shown in Tables 29-31.
¹⁴Part A3 only: 1 dose of daratumumab (16 mg/kg IV or 1800 mg SC) administered at least 12 h prior to LD chemotherapy and within 10 days prior to CTX130 infusion. First 16 mg/kg IV dose may be split (to 8 mg/kg IV) over 2 consecutive days. A second dose of daratumumab (16 mg/kg IV or 1800 mg SC) is administered on Day 22. A third dose of daratumumab (16 mg/kg IV or 1800 mg SC) is administered on Day 42 in subjects who achieve SD or better. The Day 42 CT scan must be read prior to repeat dosing with daratumumab. If a subject experiences disease progression or unacceptable AEs related to daratumumab, redosing with daratumumab is not permitted. The third daratumumab dose can be administered up to 7 days after the Day 42 CT scan.
¹⁵For subjects experiencing grade ≥3 neutropenia, thrombocytopenia, or anemia that has not resolved within 28 days of CTX130 infusion, a CBC with differential must be performed weekly until resolution to grade ≤2.
¹⁶Subjects should start LD chemotherapy within 7 days of study enrollment. After completion of LD chemotherapy, ensure washout period of ≥48 hours (but ≤7 days) before CTX130 infusion. Physical exam, weight, and coagulation laboratories are performed prior to LD chemotherapy. Vital signs, CBC, clinical chemistry, and AEs/concomitant medications should be assessed and recorded daily (i.e., 3 times) during LD chemotherapy.
¹⁷CTX130 is administered 48 hours to 7 days after completion of LD chemotherapy.
¹⁸Baseline CT is performed within 14 days prior to first CTX130 infusion. CT for response assessment is performed 6 weeks after CTX130 infusion (Day 42) and at Months 3, 6, 9, 12, 15, 18, and 24 post-CTX130 infusion. Scans are assessed locally and centrally for determination of objectives. Whenever possible, the same CT equipment and test parameters should be used. MRI is performed where CT is contraindicated and after discussion with the medical monitor.
¹⁹Biopsy is performed at screening if post-progression biopsy tissue is not available/acceptable, and on Day 7 (+2 days) and Day 42 (±2 days) after CTX130 infusion. Subjects who are redosed do not have tumor biopsies performed on Days 7 and 42
²⁰If relapse occurs on study, every attempt should be made to obtain biopsy of relapsed tumor and send to a central laboratory.
²¹Creatinine is assessed more frequently between Days 1-28 to monitor for acute renal tubular damage: daily on Days 1-7, every other day between Days 8-15, and twice weekly until Day 28. If acute renal tubular damage is suspected, additional tests should be conducted, including urine sediment analysis and fractional excretion of sodium in urine, and consultation with a nephrologist should be initiated.
²²Includes HIV, HBV, HCV, EBV, and HHV-6 at screening; however, historical results obtained within 60 days of enrollment may be used to determine eligibility.
²³SARS-CoV-2 test is performed at screening. Screening test does not need to be repeated if within 3-4 days prior to starat of lymphodepleting regimen (LD chemotherapy with or without daratumumab)
²⁴Lymphocyte subset assessment at screening, before start of first day of LD chemo (Part A1) or pre initial daratumumab dose (Part A3), before CTX130 infusion, then all listed time points are assessed at local laboratory and include 6-color TBNK panel, or equivalent for T, B, and natural killer cells. Flow cytometry analysis to provide results for CD3⁺ and CD3⁻ T cell populations.
²⁵Samples for CTX130 levels should be collected from any lumbar puncture or tissue biopsy performed following CTX130 infusion. If CRS occurs, samples for assessment of CTX130 levels are collected every 48 hours (±5 hours) between scheduled visits until CRS resolves.
²⁶Two samples are collected on Day 1: one pre-CTX130 infusion and another 20 minutes (±5 min) after the end of CTX130 infusion.
²⁷Initial sample collection occurs at onset of symptoms. Additional cytokine samples should be collected every 12 hours (±5 h) for the duration of CRS.
²⁸Samples are collected at the same time of day (±2 hours) on the specified collection days.
²⁹Daratumumab-related assessments for Part A3 only. Day 1 sample collected prior to CTX130 infusion. Two samples are collected at each daratumumab dosing: 1 before administrations and another 30 (±15) min after the end of administration.
³⁰If CRS occurs, samples for assessment of exploratory biomarkers are collected every 48 hours (±5 hours) between scheduled visits until CRS resolves. Samples for exploratory biomarkers should be collected from any lumbar puncture performed following CTX130 infusion.
³¹In additional sample is collected at screening for germ-line DNA extraction.
³²Prior to first day of LD chemotherapy only.
³³All assessments occur the day of and prior to daratumumab dosing, except local laboratory assessments can be done within 24 hr prior to daratumumab
³⁴sCD25 also assessed for confirmation of suspected HLH

TABLE 30
Schedule of Assessments for Part A2 and Part A4 (Multiple Dose Regimen): Screening to Follow-up Visit 6

| | | | | | Cycles 1-3¹ | | | | | | |
|---|---|---|---|---|---|---|---|---|---|---|---|
| Assessment | | | | | CTX130 | | | | | | |
| Day of Cycle | | | | | Infusion⁶ | | | | | | |
| (Cycles 1, 2, | | Pre-LD | Daratumumab | LD | C1-3 | C1-3 | C1-3 | C1-3 | C1-3 | C1-3 | C1-3 |
| and 3) | Screening³ | Chemo⁴ | Part A4 only³⁷ | Chemo⁵ | D1 | D2 | D3 | D5 | D7 | D10 | D15 |
| Window | | | | | N/A | N/A | N/A | N/A | +2 d | ±1 d | ±2 d |
| Eligibility | X | | | X | X | | | | | | |
| confirmation⁸ | | | | | | | | | | | |
| Informed | X | | | | | | | | | | |
| consent | | | | | | | | | | | |
| Medical history⁹ | X | | | | | | | | | | |
| Daratumumab¹⁰ | | | | X | | | | | | | |
| Physical exam¹¹ | X | X | X | X | X | X | X | X | X | X | X |
| Vital signs¹² | X | X | X | X | X | X | X | X | X | X | X |
| Height, weight¹³ | X | X | X | X | X | | | | | | |
| Pregnancy test¹⁴ | X | X | X | X | | | | | | | |
| Brain MRI¹⁵ | X | X | | | | | | | | | |
| Karnofsky status | X | X | | | X | | X | | X | | X |
| Echocardiogram | X | X | | | | | | | | | |
| 12-lead ECG | X | X | X | X | X | | | | | | |
| ICE | X | X | | | X | X | X | X | X | X | X |
| assessment¹⁷ | | | | | | | | | | | |
| PRO¹⁸ | X | | | | X | | | | | | X |

| Concomitant | Continuous | |
|---|---|---|
| medications¹⁹ | | |
| AEs²⁰ | Continuous | |
| Hospital | | Continuous |
| utilization | | |

Metastatic ccRCC Disease/Response Assessments (Central)

| | | |
|---|---|---|
| CT scan²¹ | X | |
| Tumor | X | X²⁴ |
| biopsy^{22,23} | | |

Laboratory Assessments (Local)

| | | | | | | | | | | | |
|---|---|---|---|---|---|---|---|---|---|---|---|
| CBC w/ | X | X | X | X | X | X | X | X | X | X | X |
| differential | | | | | | | | | | | |
| Serum | X | X | X | X | X²⁵ | X²⁵ | X²⁵ | X²⁵ | X²⁵ | X²⁵ | X²⁵ |
| chemistry²⁵ | | | | | | | | | | | |
| Coagulation | X | X | X | X | X | X | X | X | X | X | X |
| parameters | | | | | | | | | | | |
| Viral serology²⁶ | X | X | | | | | | | | | |
| SARS-CoV-2 | X | | | | | | | | | | |
| Test²⁷ | | | | | | | | | | | |
| Lymphocyte | X | X | X | X | X | | | X | X | X | X |
| subsets²⁸ | | | | | | | | | | | |
| Ferritin, CRP, | X | X | | | X | X | X | X | X | X | X |
| triglyceride | | | | | | | | | | | |
| sCD25³⁸ | X | | X | | | | | | | | X |

Biomarkers (Blood, Central)

| CTX130 levels²⁹ | X | X | | | X³⁰ | X | X | X | X | X | X |
|---|---|---|---|---|---|---|---|---|---|---|---|
| | | | | | pre/ | | | | | | |
| | | | | | post | | | | | | |
| Cytokines³¹ | X | X | | | X | X | X | X | X | X | X |
| BSAP, PINP³² | X | X | | | X | | | | X | | X |
| Anti-CTX130 | X | X | | | | | | | | | |
| Ab | | | | | | | | | | | |
| Daratumumab | X | X | X³³ | X³⁶ | X³³ | | | | X | | X |
| PK³³ | | | Pre/ | | | | | | | | |
| Part A4 only | | | post | | | | | | | | |
| Cell-free DNA | X | X | | | | | | | | | |
| Exploratory | X³⁵ | X | | X³⁶ | X | X | X | X | X | X | X |
| biomarkers³⁴ | | | | | | | | | | | |
| | | Part | | | | | | | | | |
| Assessment | | A4 | | | | | | | | | |
| Day of Cycle | | only | | | | | | | | | |
| (Cycles 1, 2, | C1-3 | C1-3 | C1-3 | C1-3 | C1-3 | Follow-up Visits 1-6² | | | | | |
| and 3) | D22 | D25 | D28 | D42 | D56⁷ | FU-1 | FU-2 | FU-3 | FU-4 | FU-5 | FU-6 |
| Window | ±2 d | ±2 d | ±2 d | ±2 d | ±7 d | ±14 d | ±14 d | ±14 d | ±14 d | ±14 d | ±21 d |
| Eligibility | | | | | | | | | | | |
| confirmation⁸ | | | | | | | | | | | |
| Informed | | | | | | | | | | | |
| consent | | | | | | | | | | | |
| Medical history⁹ | | | | | | | | | | | |
| Daratumumab¹⁰ | X | | | | | | | | | | |
| Physical exam¹¹ | X | X | X | X | X | X | X | X | X | X | X |
| Vital signs¹² | X | X | X | X | X | X | X | X | X | X | X |
| Height, weight¹³ | | | X | | | | | | | | |
| Pregnancy test¹⁴ | | | X | | X | X | | | | | |
| Brain MRI¹⁵ | | | | | | | | | | | |
| Karnofsky status | X | | X | X | X | X | X | X | X | X | X |
| Echocardiogram | | | | | | | | | | | |
| 12-lead ECG | | | | X | | | | | | | |
| ICE | X | | X | X | X | | | | | | |
| assessment¹⁷ | | | | | | | | | | | |
| PRO¹⁸ | | | X | | X | X | X | X | X | X | X |
| Concomitant | Continuous | | | | | | | | | | |
| medications¹⁹ | | | | | | | | | | | |
| AEs²⁰ | Continuous | | | | | | | | | | |
| Hospital | Continuous | | | | | | | | | | |
| utilization | | | | | | | | | | | |

Metastatic ccRCC Disease/Response Assessments (Central)

| | | | | | | | |
|---|---|---|---|---|---|---|---|
| CT scan²¹ | X | X | X | X | X | X | X |
| Tumor | X²⁴ | | | | | | |
| biopsy^{22,23} | | | | | | | |

Laboratory Assessments (Local)

| | | | | | | | | | | | |
|---|---|---|---|---|---|---|---|---|---|---|---|
| CBC w/ | X | X | X¹⁶ | X | X | X | X | X | X | X | X |
| differential | | | | | | | | | | | |
| Serum | X²⁵ | X²⁵ | X²⁵ | X | X | X | X | X | X | X | X |
| chemistry²⁵ | | | | | | | | | | | |
| Coagulation | X | | X | X | | | | | | | |
| parameters | | | | | | | | | | | |
| Viral serology²⁶ | | | | | | | | | | | |
| SARS-CoV-2 | | | | | | | | | | | |
| Test²⁷ | | | | | | | | | | | |
| Lymphocyte | X | X | X | X | X | X | X | X | X | X | X |
| subsets²⁸ | | | | | | | | | | | |
| Ferritin, CRP, | X | X | X | X | | | | | | | |
| triglyceride | | | | | | | | | | | |
| sCD25³⁸ | X | X | X | | | | | | | | |

Biomarkers (Blood, Central)

| | | | | | | | | | | | |
|---|---|---|---|---|---|---|---|---|---|---|---|
| CTX130 levels²⁹ | X | X | X | X | X | X | X | X | X | X | X |
| Cytokines³¹ | X | X | X | X | X | X | | | | | |
| BSAP, PINP³² | X | | X | | | X | | X | | | |
| Anti-CTX130 | | | X | | | X | | X | | | X |
| Ab | | | | | | | | | | | |
| Daratumumab | X³³ | | | | | | | | | | |
| PK³³ | Pre/ | | | | | | | | | | |
| Part A4 only | post | | | | | | | | | | |
| Cell-free DNA | | | X | | X | X | X | X | | X | X |
| Exploratory | X | | X | X | X | X | X | X | X | X | X |
| biomarkers³⁴ | | | | | | | | | | | |

Ab: antibody; AE: adverse event; BSAP: bone-specific alkaline phosphatase; C1-3: Cycles 1, 2, and 3; CBC: complete blood count; ccRCC: clear cell renal cell carcinoma; chemo: chemotherapy; CNS: central nervous system; COVID-19: coronavirus disease 2019; CRP: C-reactive protein; CRS: cytokine release syndrome; CT: computed tomography; D or d: day; EBV: Epstein-Barr virus; ECG: electrocardiogram; EORTC: European Organization for Research and Treatment of Cancer; EQ-5D-5L: EuroQol-5 Dimension-5 Level; FACT-G: Functional Assessment of Cancer Therapy-General; FKSI-19: Functional Assessment of Cancer Therapy-Kidney Symptom Index; FU: follow-up; GvHD: graft vs host disease; HBV: hepatitis B virus; HCV: hepatitis C virus; HHV-6: human herpesvirus 6; HIV: human immunodeficiency virus; ICE: immune effector cell-associated encephalopathy; LD: lymphodepleting; M: month; MRI: magnetic resonance imaging; N/A: not applicable; PINP: procollagen type I N propeptide; PK: pharmacokinetics; PRO: patient-reported outcome; SARS-CoV-2: severe acute respiratory syndrome coronavirus 2; sCD25: soluble CD25; TBNK: T, B, natural killer cells.
Note:
Assessments scheduled on CTX130 infusion days are performed pre-CTX130 infusion unless otherwise specified.
¹Each cycle includes daratumumab administration (Part A4 only), LD chemo, 1 dose of CTX130, and follow-up through Day 56. Subjects complete 3 cycles, then proceed to follow-up visit 1 (FU-1).
²FU-1: 4 weeks after Cycle 3 Day 56 visit; subjects who may be unable to be redosed for 3 cycles may proceed to FU-1 visit after completion of Cycle 1 or Cycle 2 without additional dosing. FU-2 through FU-5: every 12 weeks after prior FU visit. FU-6: 24 weeks after FU-5.
³Screening assessments are completed within 14 days after signing informed consent form. The screening period may be extended beyond 14 days to allow for COVID-19 testing only. Subjects are allowed a one-time rescreening, which may take place within 3 months of the initial consent.
⁴Prior to Cycles 2 and 3 only. If Day 56 assessments are completed within 7 days of initiation of LD chemo, the assessments do not need to be repeated as part of pre-LD chemo visit. Part A4 only: all pre-LD chemo assessments must be completed prior to daratumumab administration.
⁵Subjects should start LD chemotherapy within 7 days of study enrollment. For subjects starting Cycle 2 or Cycle 3, LD chemotherapy should begin within 7 days of pre-LD chemo assessments. After completion of LD chemotherapy, ensure washout period of ≥48 hours (but ≤7 days) before CTX130 infusion. Physical exam, weight, and coagulation laboratories are performed prior to LD chemotherapy. Vital signs, CBC, clinical chemistry, and AEs/concomitant medications should be assessed and recorded daily (i.e., 3 times) during LD chemotherapy.
⁶CTX130 is administered 48 hours to 7 days after completion of LD chemotherapy.
⁷After completion of Day 56 assessments, subjects at the end of Cycle 1 or Cycle 2 proceeds to pre-LD chemo assessments and subsequently undergo daratumumab (Part A4 only), LD chemo followed by CTX130 infusion. After completion of Day 56 assessments in Cycle 3, subjects proceed to FU-1 Visit.
⁸Eligibility should be confirmed each time screening is completed. Eligibility should also be confirmed on day of daratumumab administration (Part A4 only), first day of LD chemotherapy, and day of CTX130 infusion. Eligibility should be reconfirmed after all assessments for that day are completed and before dosing.
⁹Includes complete surgical and cardiac history.
¹⁰Part A4 only: 1 dose of daratumumab 16 mg/kg IV or 1800 mg SC administered at least 12 h prior to LD chemotherapy and within 10 days of CTX130 infusion. The first 16 mg/kg dose may be split (to 8 mg/kg IV) over 2 consecutive days. An additional dose of daratumumab (16 mg/kg IV or 1800 mg SC) is administered on Day 22 (±2 days). If a subject experiences severe AEs related to daratumumab, redosing with daratumumab is not permitted.
¹¹Includes assessment for signs and symptoms of GvHD: skin, oral mucosa, sclera, hands, and feet.
¹²Includes blood pressure, heart rate, respiratory rate, pulse oximetry, and temperature.
¹³Height at screening only.
¹⁴For female subjects of childbearing potential. Assessed at local laboratory. Serum pregnancy tests required at screening, within 72 hours of start of either LD chemotherapy (Part A2) or initial daratumumab dose of cycle (Part A4), and at Day 28, Day 56, and FU-1.
¹⁵Brain MRI at screening is performed within 28 days prior to CTX130 infusion and during Cycles 2 and 3 as part of pre-LD chemo assessments (within 28-days prior to CTX130 infusion).
¹⁶For subjects experiencing grade ≥3 neutropenia, thrombocytopenia, or anemia that has not resolved within 28 days of CTX130 infusion, a CBC with differential is performed weekly until resolution to grade ≤2.
¹⁷On Day 1 prior to CTX130 administration. If CNS symptoms persist after Day 56, ICE assessment should continue to be performed approximately every 2 days until symptom resolution to grade 1 or baseline.
¹⁸EORTC QLQ-C30, EQ-5D-5L, FKSI-19, and FACT-G questionnaires. PROs should be completed at screening, predose on Day 1 and then Days 15, 28, and 56 post-CTX130 infusion, and thereafter as specified in the schedule of assessments.
¹⁹All concomitant medications are collected up to 3 months post-CTX130 infusion. Afterwards, only select concomitant medications are collected (i.e., immunomodulating agents, blood products, antitumor medications, hormones, and growth factors).
²⁰AEs are collected for enrolled subjects according to the AE reporting requirements for each time period of the study as shown in Tables 29-31.
²¹Baseline CT is performed within 14 days prior to first CTX130 infusion. CT for response assessment is performed 6 weeks after CTX130 infusion (Day 42) and at FU-1 through FU-6 visits post-CTX130 infusion. Scans are assessed locally and centrally for determination of objectives. Whenever possible, the same CT equipment and test parameters should be used. MRI is performed where CT is contraindicated and after discussion with the medical monitor.
²²Biopsy is performed at screening if postprogression biopsy tissue is not available/acceptable, Day 7 (+2 days), and Day 42 (±2 days) after CTX130 infusion.
²³If relapse occurs on study, every attempt should be made to obtain biopsy of relapsed tumor and send to a central laboratory.
²⁴Tumor biopsy for subjects in Cycle 1 only.
²⁵Creatinine is assessed more frequently between Days 1-28 to monitor for acute renal tubular damage: daily on Days 1-7, every other day between Days 8-15, and twice weekly until Day 28. If acute renal tubular damage is suspected, additional tests should be conducted, including urine sediment analysis and fractional excretion of sodium in urine, and consultation with a nephrologist should be initiated.
²⁶Includes HIV, HBV, HCV, EBV, and HHV-6 at screening; however, historical results obtained within 60 days of enrollment may be used to determine eligibility.
²⁷SARS-CoV-2 test is performed at screening. Screening test does not need to be repeated if within 3-4 days prior to start of lymphodepletion regimen.
²⁸Lymphocyte subset assessment at screening, during Cycles 2 and 3 as part of pre-LD chemo assessments, pre initial daratumumab dosing for each cycle (Part A4), or before start of first day of LD chemo (Part A2), before CTX130 infusion, then all listed time points is assessed at local laboratory and includes 6-color TBNK panel, or equivalent for T, B, and natural killer cells. Flow cytometry analysis to provide results for CD3+ and CD3− T cell populations.
²⁹Samples for CTX130 levels should be collected from any lumbar puncture or tissue biopsy performed following CTX130 infusion. If CRS occurs, samples for assessment of CTX130 levels is collected every 48 hours (±5 hours) between scheduled visits until CRS resolves.
³⁰Two samples are collected on Day 1: 1 pre-CTX130 infusion and another 20 minutes (±5 min) after end of CTX130 infusion.
³¹Initial sample collection to occur at onset of symptoms. Additional cytokine samples should be collected every 12 hours (±5 hours) for duration of CRS.
³²Samples are collected at the same time of day (±2 hours) on the specified collection days.
³³Daratumumab-related assessments for Part A4 only. Day 1 sample collected prior to CTX130 infusion; Two samples are to be collected at each daratumumab dosing: 1 before administration and another 30 (±15) min after the end of administrations.
³⁴If CRS occurs, samples for assessment of exploratory biomarkers are collected every 48 hours (±5 hours) between scheduled visits until CRS resolves. Samples for exploratory biomarkers should be collected from any lumbar puncture performed following CTX130 infusion.
³⁵An additional sample is collected at screening for germ-line DNA extraction.
³⁶Prior to first day of LD chemotherapy only.
³⁷All assessments must occur the day of and prior to the daratmumab dosing, except local laboratory assessments can be done within 24 hr prior to the daratumumab.,
³⁸sCD25 to be assessed at screening, pre initial daratumumab dosing for each cycle (Part A4 only), Day 15, Day 22, Day 25 (Part A4 only), Day 28, and for confirmation of suspected HLH.

TABLE 31
Schedule of Assessments: Months 30-60

| | M30 | M36 | M42 | M48 | M54 | M60 | M64¹ | Progressive | Secondary |
|---|---|---|---|---|---|---|---|---|---|
| Assessments | (±21 days) | (±21 days) | (±21 days) | (±21 days) | (±21 days) | (±21 days) | (±21 days) | Disease | Follow-up² |
| Vital signs³ | X | X | X | X | X | X | X | X | X |
| Physical exam | X | X | X | X | X | X | X | X | X |
| PRO⁴ | | X | | X | | X | X | X | |
| Concomitant | X | X | X | X | X | X | X | X | X |
| medications⁵ | | | | | | | | | |
| AEs⁶ | X | X | X | X | X | X | X | X | X |
| Disease assessment⁷ | X | X | X | X | X | X | X | X | |

Laboratory Assessments (Blood, Local)

| | | | | | | | | | |
|---|---|---|---|---|---|---|---|---|---|
| CBC with differential | X | X | X | X | X | X | X | X | X |
| Serum chemistry | X | X | X | X | X | X | X | X | X |
| Lymphocyte subsets⁸ | X | X | X | X | X | X | X | X | |

Biomarkers (Blood, Central)

| | | | | | | | | | |
|---|---|---|---|---|---|---|---|---|---|
| CTX130 persistence⁹ | X | X | X | X | X | X | X | X | X |
| Anti-CTX130 antibody | | X | | X | | X | X | X | |
| Exploratory | X | X | X | X | X | X | X | X | X |
| biomarkers | | | | | | | | | |

AE: adverse event; CBC: complete blood count; CT: computed tomography; EORTC: European Organization for Research and Treatment of Cancer; EQ-5D-5L: EuroQol-5 Dimension-5 Level; FACT-G: Functional Assessment of Cancer Therapy-General; FKSI-19: Functional Assessment of Cancer Therapy-Kidney Symptom Index; M: month; MRI: magnetic resonance imaging; PRO: patient-reported outcome; TBNK: T, B, natural killer cells.
¹The Month 64 visit is the last scheduled visit for subjects enrolled in Part A2 who complete 3 cycles of dosing.
²Subjects who are discontinued from the regular schedule of assessments and due to disease progression, investigator decision/start of new anti-cancer therapy, AEs, protocol violation, or pregnancy attend annual visits to collect safety information for up to 5 years.
³Includes sitting blood pressure, heart rate, respiratory rate, pulse oximetry, and temperature.
⁴EORTC QLQ-C30, EQ-5D-5L, FKSI-19, and FACT-G questionnaires.
⁵Only select concomitant medications are collected.
⁶AEs are collected for enrolled subjects according to the AE reporting requirements at each time period of the study, as shown in Tables 29-31.
⁷Disease assessment consists of review of physical exam, CBC, and clinical chemistry. Subjects with suspected malignancy undergo CT (or possible MRI) imaging and/or a tissue biopsy to confirm relapse. Every attempt should be made to obtain a biopsy of the relapsed tumor in subjects who progress.
⁸Assessed at local laboratory. Includes 6-color TBNK panel, or equivalent for T, B, and natural killer cells. Flow cytometry analysis to provide results for CD3⁺ and CD3⁻ T cell populations.
⁹Samples for CTX130 levels should be sent to a central laboratory from any lumbar puncture or tissue biopsy performed following CTX130 infusion.

### Subject Screening

Karnofsky Performance Status

Performance status is assessed at the time points outlined in the schedule of assessments using the Karnofsky scale to determine the subject's general well-being and ability to perform activities of daily life, with scores ranging from 0 to 100. A higher score means better ability to carry out daily activities.

The Karnofsky scale, shown in Table 32, is used to determine performance status in the current study (Péus et al., BMC Med Inform Decis Mak, 2013).

TABLE 32
Karnofsky Performance Status Scale

| | Karnofsky |
|---|---|
| Karnofsky Status | Grade |
| Normal, no complaints | 100 |
| Able to carry on normal activities, | 90 |
| minor signs or symptoms of disease | |
| Normal activity with effort | 80 |
| Cares for self, unable to carry on normal | 70 |
| activity or do active work | |
| Requires occasional assistance but able | 60 |
| to care for most of his/her needs | |
| Requires considerable assistance and | 50 |
| frequentmedical care | |
| Disabled, requires special care and | 40 |
| assistance | |
| Severely disabled, hospitalization | 30 |
| indicated though death nonimminent | |
| Very sick, hospitalization necessary, | 20 |
| active supportive treatment necessary | |
| Moribund | 10 |
| Dead | 0 |

Brain MRI

To rule out CNS metastasis, a brain MRI is performed at screening (i.e., within 28 days prior to CTX130 infusion). For subjects enrolled in Parts A2 and A4 (multiple dose regimens), brain MRI should also be performed during Cycles 2 and 3 as part of the pre-LD chemotherapy assessments (within 28 days prior to CTX130 infusion).

Echocardiogram

A transthoracic cardiac echocardiogram (for assessment of left ventricular ejection fraction) is performed and read by trained medical personnel at screening to confirm eligibility, and as part of pre-LD chemotherapy assessments during Cycles 2 and 3 for subjects enrolled in Parts A2 and A4. In case of cardiac symptoms during CRS, medically appropriate assessment should be initiated in accordance with institutional guidelines.

Electrocardiogram

Twelve (12)-lead electrocardiograms (ECGs) are obtained during screening (and as part of pre-LD chemotherapy assessments during Cycles 2 and 3 for subjects enrolled in Parts A2 and A4), prior to initial daratumumab administration (Part A3) or initial daratumumab administration for each cycle (Part A4), prior to LD chemotherapy on the first day of treatment, prior to CTX130 administration on Day 1, and on Day 42. QTc and QRS intervals are determined from ECGs. Additional ECGs may be obtained.

ccRCC Disease and Response Assessments

Disease evaluations are based on assessments in accordance with RECIST v1.1 (described herein) and are assessed. Determination of study eligibility and decisions regarding subject management and disease progression are made. For efficacy analyses, disease outcome is graded using RECIST v1.1. Where applicable, concordance rates between central and response assessments are determined. ccRCC disease and response evaluation should be conducted per the schedule of assessments.

Radiographic Disease Assessment (CT)

Whenever possible, the same CT equipment and test parameters should be used. MM is performed where CT is contraindicated and after discussion with the medical monitor.

Baseline CT is performed at screening (i.e., within 28 days prior to first CTX130 infusion). CT is also performed 6 weeks after each CTX130 infusion. For Part A1 and Part A3, scans is also completed at Months 3 (Day 84), 6, 9, 12, 15, 18, and 24 post-CTX130 infusion. For Part A2 and Part A4, scans is completed at follow-up visits 1-6. All scans are analyzed per RECIST v1.1, according to the schedule of assessments and as clinically indicated. Scans are assessed locally and centrally for determination of objectives.

To capture overall disease burden during local disease assessment, disease extent at baseline and in subsequent assessments should be described as completely as feasible on a per lesion basis. Five measurable target lesions should be selected if present, and measurable lesions in excess of 5 as well as nonmeasurable lesions should be captured as non-target lesions. At minimum the following information should be captured per lesion: description and anatomical location of lesion, whether lesion is nodal or extra-nodal, and lesion size as a bidimensional measurement. Bidimensional measurements should be collected for all measurable lesions.

CT scans should be acquired with >5-mm slices with no intervening gap (contiguous). Should a subject have a contraindication for CT IV contrast, a noncontrast CT of the chest and a contrast-enhanced MRI of the abdomen and pelvis may be obtained. MRIs should be acquired with slice thickness of 5 mm with no gap (contiguous). Every attempt should be made to image each subject using an identical acquisition protocol on the same scanner for all imaging time.

In addition, if a subject receives a fluorodeoxyglucose-positron emission tomography/CT scan for reasons outside of the study, it is possible that the CT component of the scan may be used to assess disease response.

For efficacy analyses, radiographic disease assessments are performed by the IRC in accordance with RECIST v1.1.

Tumor Biopsy

Subjects are required to undergo tumor biopsy at screening or, if a post-progression biopsy was performed within 3 months prior to enrollment and after the last systemic or targeted therapy, archival tissue may be provided. If archival tissue is of insufficient volume or quality to fulfill central laboratory requirements, a biopsy must be performed during screening.

Tumor biopsy is also performed on Day 7 (+2 days, or as soon as clinically feasible) and Day 42 (±2 days) after initial dosing only (i.e., Cycle 1). Subjects who are redosed in Parts A1 and A3 do not have tumor biopsies performed on Days 7 and 42. If a relapse occurs while a subject is on study, every attempt should be made to obtain biopsy of relapse tumor and send to a central laboratory.

Biopsies should come from measurable but nontarget lesions according to RECIST v1.1 analysis. When multiple biopsies are taken, efforts should be made to obtain them from similar tissues. Liver metastases are generally less desirable. Bone biopsies and other decalcified tissues are not acceptable due to interference with downstream assays. This sample is analyzed for presence of CTX130 as well as tumor-intrinsic and TME-specific biomarkers, including analysis of DNA, RNA, protein, and metabolites.

Patient-Reported Outcomes

Four PRO surveys are administered according to the schedule of assessments: the European Organization for Research and Treatment of Cancer (EORTC) QLQ-C30, the EuroQol-5 Dimension-5 Level (EQ-5D-5L), the NCCN Functional Assessment of Cancer Therapy (FACT) Kidney Symptom Index (FKSI-19), and FACT-General (FACT-G) questionnaires.

Questionnaires should be completed (self-administered in the language the subject is most familiar) before clinical assessments are performed.

The EORTC QLQ-C30 is a questionnaire designed to measure quality of life in cancer patients. It is composed of 5 multi-item functioning scales (physical, role, social, emotional, and cognitive function), 3 symptom scales (fatigue, nausea, pain), and additional single symptom items (financial impact, appetite loss, diarrhea, constipation, sleep disturbance, and quality of life). The EORTC QLQ-C30 is validated and has been widely used among cancer patients (Wisloff et al., Br J Haematol, 1996; Wisloff et al., Br J Haematol 1997). It is scored on a 4-point scale (1=not at all, 2=a little, 3=quite a bit, 4=very much). The EORTC QLQ-C30 instrument also contains 2 global scales that use 7-point scale scoring with anchors (1=very poor and 7=excellent).

The EQ-5D-5L is a generic measure of health status and contains a questionnaire that assesses 5 domains, including mobility, self-care, usual activities, pain/discomfort, and anxiety/depression, plus a visual analog scale.

The NCCN-FACT FKSI-19 is designed as a brief symptom index for patients with advanced kidney cancer and includes perspectives of both clinicians and patients. The index includes 19 items within 3 subscales: disease-related symptoms, treatment side effects, and general function and well-being (Cella et al., J Clin Oncol, 1993; Rothrock et al., Value Health, 2013).

The FACT-G questionnaire is designed to assess the health-related quality of life in patients undergoing cancer treatment. It is divided into physical, social/family, emotional, and functional domains (Cella et al., J Clin Oncol, 1993).

Immune Effector Cell-Associated Encephalopathy (ICE) Assessment

Neurocognitive assessment is performed using ICE assessment. The ICE assessment tool is a slightly modified version of the CARTOX-10 screening tool, which now includes a test for receptive aphasia (Neelapu et al., Nat Rev Clin Oncol, 2018). ICE assessment examines various areas of cognitive function: orientation, naming, following commands, writing, and attention (Table 33).

TABLE 33
Immune Effector Cell-associated Encephalopathy (ICE) Assessment

| | | Maximum |
|---|---|---|
| Domain | Assessment | Score |
| Orientation | Orientation to year, month, city, hospital | 4 points |
| Naming | Name 3 objects (e.g., point to clock, pen, | 3 points |
| | button) | |
| Following | Ability to follow commands (e.g., “Show | 1 point |
| command | me 2 fingers” or “Close your eyes and | |
| | stick out your tongue”) | |
| Writing | Ability to write a standard sentence | 1 point |
| | (includes a noun and verb) | |
| Attention | Ability to count backward from 100 by 10 | 1 point |

ICE score is reported as the total number of points (0-10) across all assessments.

ICE assessment is performed at screening (and as part of pre-LD chemotherapy assessments during Cycles 2 and 3 for subjects enrolled in Parts A2 and A4), before administration of CTX130 on Day 1, and on Days 2, 3, 5, 7, 10, 15, 22, 28, 42, and 56, and after each CTX130 dose. If CNS symptoms persist beyond Day 56, ICE assessment should continue to be performed approximately every 2 days until resolution of symptoms to grade 1 or baseline. To minimize variability, whenever possible the assessment should be performed by the same research staff member who is familiar with or trained in administration of the ICE assessment tool.

Laboratory Tests

Laboratory samples are collected and analyzed according to the schedule of assessments. Local laboratories meeting applicable local requirements (e.g., Clinical Laboratory Improvement Amendments) are utilized to analyze all tests listed in Table 34, according to standard institutional procedures.

TABLE 34
Local Laboratory Tests

| | |
|---|---|
| CBC with differential | Hematocrit, hemoglobin, red blood cell count, white blood cell |
| | count, neutrophils, lymphocytes, monocytes, basophils, |
| | eosinophils, platelet count, absolute neutrophil count |
| Serum chemistry | ALT (SGPT), AST (SGOT), bilirubin (total and direct), albumin, |
| | alkaline phosphatase, bicarbonate, BUN, calcium, chloride, |
| | creatinine, eGFR, glucose, hemoglobin Ale (only at screening), |
| | lactate dehydrogenase, magnesium, phosphorus, potassium, |
| | sodium, total protein, uric acid |
| Coagulation | PT, aPTT, international normalized ratio, fibrinogen |
| Viral serology¹ | HIV-1, HIV-2, hepatitis C virus antibody and RNA, hepatitis B |
| | surface antigen, hepatitis B surface antibody, hepatitis B core |
| | antibody, EBV, HHV-6 |
| Lymphocyte Subsets | 6-color TBNK panel or equivalent (T cells, B cells, and NK cells). |
| | Flow cytometry analysis to provide results for CD3+ and CD3− T |
| | cell populations. |
| CRS/HLH monitoring | Ferritin, CRP, triglycerides, sCD25² |
| Serum pregnancy³ | Human chorionic gonadotropin |
| COVID-19 | Rapid or PCR test performed 3-4 days prior to start of LD |
| | chemotherapy (or initial daratumumab dose in Parts A3 and A4 only) |

ALT: alanine aminotransferase; aPTT: activated partial thromboplastin time; AST: aspartate aminotransferase; BUN: blood urea nitrogen; CBC: complete blood count; COVID-19: coronavirus disease 2019; CRP: C-reactive protein; CRS: cytokine release syndrome; EBV: Epstein-Barr virus; eGFR: estimated glomerular fdtration rate; FU: follow- up; HHV-6: human herpesvirus 6; HIV-1/-2: human immunodeficiency virus type 1 or 2; HLH: hemophagocytic lymphohistiocytosis; NK: natural killer; LD: lymphodepleting; M: month; PCR: polymerase chain reaction; PT: prothrombin time; sCD25: soluble CD25; SGOT: serum glutamic oxaloacetic transaminase; SGPT: serum glutamic pyruvic transaminase; TBNK: T, B, and NK cells
¹Historical viral serology results obtained within 60 days of enrollment may be used to determine eligibility.
²sCD25 assessed at screening, pre initial daratumumab dosing (Part A3 only) or pre initial daratumumab dosing for each cycle (Part A4 only), Day 15, Day 22, Day 25 (Parts A3 and A4 only), Day 28, and for confirmation of suspected HLH.
³For females of childbearing potential only. See Tables 29-31 for schedule of pregnancy testing.

### Response Evaluation Criteria in Solid Tumors Version 1.1 (RECIST v1.1)

The following is adapted from RECIST v1.1 (Eisenhauer et al., Eur J Canc, 2009).

### Categorizing Lesions at Baseline

Measurable Lesions
Lesions that can be accurately measured in at least 1 dimension.
Lesions with longest diameter twice the slice thickness and at least 10 mm or greater when assessed by CT or MRI (slice thickness 5-8 mm)
Lesions with longest diameter at least 20 mm when assessed by chest X-ray
Superficial lesions with longest diameter 10 mm or greater when assessed by caliper
Malignant lymph nodes with the short axis 15 mm or greater when assessed by CT
NOTE: The shortest axis is used as the diameter for malignant lymph nodes, and the longest axis is used for all other measurable lesions.

Nonmeasurable Disease

Nonmeasurable disease includes lesions too small to be considered measurable (including nodes with short axis between 10 and 14.9 mm) and truly nonmeasurable disease such as pleural or pericardial effusions, ascites, inflammatory breast disease, leptomeningeal disease, lymphangitic involvement of skin or lung, clinical lesions that cannot be accurately measured with calipers, abdominal masses identified by physical exam that are not measurable by reproducible imaging techniques.
Bone disease: Bone disease is nonmeasurable with the exception of soft tissue components that can be evaluated by CT or MRI and meet the definition of measurability at baseline.
Previous local treatment: A previously irradiated lesion (or lesion subjected to other local treatment) is nonmeasurable unless it has progressed since completion of treatment.

Normal Sites
Cystic lesions: Simple cysts should not be considered as malignant lesions and should not be recorded either as target or nontarget disease. Cystic lesions thought to represent cystic metastases can be measurable lesions if they meet the specific definition above. If noncystic lesions are also present, these are preferred as target lesions.
Normal nodes: Nodes with short axis<10 mm are considered normal and should not be recorded or followed either as measurable or nonmeasurable disease.

### Recording Tumor Assessments

All sites of disease must be assessed at baseline. Baseline assessments should be done as close as possible prior to study start. For an adequate baseline assessment, all required scans must be done within 28 days prior to treatment and all disease must be documented appropriately. If baseline assessment is inadequate, subsequent statuses generally should be indeterminate.

Target Lesions

All measurable lesions up to a maximum of 2 lesions per organ, 5 lesions in total, representative of all involved organs, should be identified as target lesions at baseline. Target lesions should be selected on the basis of size (longest lesions) and suitability for accurate repeated measurements. Record the longest diameter for each lesion, except in the case of pathological lymph nodes, for which the short axis should be recorded. The sum of the diameters (longest for non-nodal lesions, short axis for nodal lesions) for all target lesions at baseline is the basis for comparison to assessments performed on study.
If 2 target lesions coalesce the measurement of the coalesced mass is used. If a large target lesion splits, the sum of the parts is used.
Measurements for target lesions that become small should continue to be recorded. If a target lesion becomes too small to measure, 0 mm should be recorded if the lesion is considered to have disappeared; otherwise a default value of 5 mm should be recorded.
NOTE: When nodal lesions decrease to <10 mm (normal), the actual measurement should still be recorded.

Nontarget Disease

All nonmeasurable disease is nontarget. All measurable lesions not identified as target lesions are also included as nontarget disease.

Multiple nontarget lesions in one organ may be recorded as a single item on the case report form (e.g., “multiple enlarged pelvic lymph nodes” or “multiple liver metastases”).

### Objective Response Status at Each Evaluation

Disease sites must be assessed using the same technique as baseline, including consistent administration of contrast and timing of scanning. If a change needs to be made, the case must be discussed with the radiologist to determine if substitution is possible. If not, subsequent objective statuses are indeterminate.

Target Disease
Complete response (CR): Complete disappearance of all target lesions with the exception of nodal disease. All target nodes must decrease to normal size (short axis<10 mm). All target lesions must be assessed.
Partial response (PR): Greater than or equal to 30% decrease under baseline of the sum of diameters of all target measurable lesions. The short diameter is used in the sum for target nodes, while the longest diameter is used in the sum for all other target lesions. All target lesions must be assessed.
Stable Disease: Does not qualify for CR, PR, or progression. All target lesions must be assessed. Stable can follow PR only in the rare case that the sum increases by less than 20% from the nadir, but enough that a previously documented 30% decrease no longer holds.
Objective progression (PD): 20% increase in the sum of diameters of target measurable lesions above the smallest sum observed (over baseline if no decrease in the sum is observed during therapy), with a minimum absolute increase of 5 mm.
Indeterminate: Progression has not been documented, and
   1 or more target measurable lesions have not been assessed
   or assessment methods used were inconsistent with those used at baseline
   or 1 or more target lesions cannot be measured accurately (e.g., poorly visible unless due to being too small to measure)
   or 1 or more target lesions were excised or irradiated and have not reappeared or increased.

Nontarget Disease
CR: Disappearance of all nontarget lesions and normalization of tumor marker levels. All lymph nodes must be ‘normal’ in size (<10 mm short axis).
Non-CR/non-PD: Persistence of any nontarget lesions and/or tumor marker level above the normal limits.
PD: Unequivocal progression of pre-existing lesions. Generally, the overall tumor burden must increase sufficiently to merit discontinuation of therapy. In the presence of SD or PR in target disease, progression due to unequivocal increase in nontarget disease should be rare.
Indeterminate: Progression has not been determined and 1 or more nontarget sites were not assessed or assessment methods were inconsistent with those used at baseline.

New Lesions

The appearance of any new unequivocal malignant lesion indicates PD. If a new lesion is equivocal, for example due to its small size, continued assessment clarifues the etiology. If repeat assessments confirm the lesion, then progression should be recorded on the date of the initial assessment. A lesion identified in an area not previously scanned is considered a new lesion.

Supplemental Investigations
If CR determination depends on a residual lesion that decreased in size but did not disappear completely, it is recommended the residual lesion be investigated with biopsy or fine needle aspirate. If no disease is identified, objective status is CR.
If progression determination depends on a lesion with an increase possibly due to necrosis, the lesion may be investigated with biopsy or fine needle aspirate to clarify status.

Subjective Progression

Subjects requiring discontinuation of treatment without objective evidence of disease progression should not be reported as PD on tumor assessment CRFs. Every effort should be made to document objective progression even after discontinuation of treatment.

TABLE 35
Objective Response Status at Each Evaluation

| | | New | Objective |
|---|---|---|---|
| Target Lesions | Nontarget Disease | Lesions | Status |
| CR | CR | No | CR |
| CR | Non-CR/non-PD | No | PR |
| CR | Indeterminate or missing | No | PR |
| PR | Non-CR/non-PD, | No | PR |
| | indeterminate, or missing | | |
| SD | Non-CR/non-PD, | No | Stable |
| | indeterminate, or missing | | |
| Indeterminate | Non-PD | No | Indeterminate |
| or missing | | | |
| PD | Any | Yes or no | PD |
| Any | PD | Yes or no | PD |
| Any | Any | Yes | PD |

CR: complete response; PD: progressive disease; PR: partial response.

If the protocol allows enrollment of subjects with only nontarget disease, the following table is used:

TABLE 36
Objective Response Status at Each Evaluation for
Subjects with Nontarget Disease Only

| | New | Objective |
|---|---|---|
| Nontarget Disease | Lesions | Status |
| CR | No | CR |
| Non-CR/non-PD | No | Non-CR/non-PD |
| Indeterminate | No | Indeterminate |
| Unequivocal progression | Yes or no | PD |
| Any | Yes | PD |

CR: complete response; PD: progressive disease.

### 7. Study Treatment

Daratumumab Administration

Subjects in Part A3 (single dose escalation with daratumumab added to the lymphodepletion regimen) and Part A4 (multiple dose regimen with daratumumab added to the lymphodepletion regimen) receive 1 dose of daratumumab (an anti-CD38 monoclonal antibody) 16 mg/kg by IV infusion or 1800 mg SC injection administered at least 12h prior to LD chemotherapy and within 10 days of CTX130 infusion. A second dose of daratumumab (16 mg/kg IV or 1800 mg SC) is administered on Day 22.

In Part A3 only, a third dose of daratumumab (16 mg/kg or 1800 mg SC) is administered on Day 42 in subjects who achieve SD or better. The Day 42 CT scan must be read prior to repeat dosing with daratumumab. The third daratumumab administration can be up to 7 days after the Day 42 CT scan.

Daratumumab administration (including pre- and postinfusion medications, preparation, infusion rates, and postinfusion monitoring) are performed according to the local prescribing information unless otherwise stated. To facilitate administration, the first 16 mg/kg IV dose may be split to 8 mg/kg IV over 2 consecutive days. After at least 3 subjects are treated at a specific CTX130 dose with daratumumab, the total safety and efficacy data are to be evaluated and a specific dose level with a lower dose of daratumumab (e.g., 8 mg/kg IV) may be recommended accordingly. To be considered for the additional doses of daratumumab, subjects in Part A3 and Part A4 must meet the following criteria at the time of daratumumab dosing:
No severe or unmanageable toxicity with prior daratumumab doses
No disease progression without significant clinical benefit
No ongoing uncontrolled infection
No ≥3 grade thrombocytopenia
No ≥3 grade 3 neutropenia
No CD4⁺ T cell count<100/4,

### Daratumumab Administration Reactions

To reduce the risk of infusion reactions with daratumumab IV or SC, 1 to 3 hours prior to infusion subjects are premedicated with corticosteroids (e.g., intravenous [IV] methylprednisolone 100 mg or equivalent; following the second infusion, the dose of corticosteroid may be reduced [oral or IV methylprednisolone 60 mg], antipyretics (e.g., oral acetaminophen [paracetamol] 650-1000 mg, or equivalent), and antihistamines (e.g., oral or IV diphenhydramine hydrochloride [or another H1-antihistamine] 25-50 mg, or equivalent).

Subjects are monitored frequently during the entire administration of daratumumab. For infusion reactions of any grade/severity, infusion is interrupted immediately, and symptoms managed. If an anaphylactic reaction or life-threatening (grade 4) reaction occurs, therapy is permanently discontinued and appropriate emergency care administered. For subjects with grade 1, 2, or 3 reactions, after symptom resolution, the infusion rate is reduced when restarting the infusion, as described in the approved prescribing information or per site practice.

To reduce the risk of delayed infusion reactions, oral corticosteroids (20 mg methylprednisolone or equivalent dose of an intermediate-acting or long-acting corticosteroid in accordance with local standards) are administered to subjects following the daratumumab administration, per local prescribing information.

For the second or third dose of daratumumab, only intermediate-acting corticosteroids (i.e., prednisone, methylprednisone) should be used to reduce the risk of interference with CTX130. If a subject has an unresolved event of infusion reaction after daratumumab treatment, the CTX130 infusion should be delayed and discussed with the medical monitor prior to proceeding.

### Additional Considerations

Daratumumab has been associated with herpes zoster (2%) and hepatitis B (1%) reactivation in patients with multiple myeloma. To prevent herpes zoster reactivation, initiate antiviral prophylaxis within 1 week after infusion and continue for 3 months following treatment as per local guidelines. For subjects with latent hepatitis B, consider hepatitis B prophylaxis prior to initiation of daratumumab and for 3 months following treatment (King et al., Asia Pac J Oncol Nurs, 2018).

Supportive care should be provided according to the approved local prescribing information.

Daratumumab binds to CD38 on red blood cells and results in a positive indirect antiglobulin test (indirect Coombs test). Typing and screening of blood occurs per the approved prescribing information to prevent interference with blood compatibility testing.

### Lymphodepleting Chemotherapy

All subjects receive LD chemotherapy prior to each infusion with CTX130. LD chemotherapy consists of:
Fludarabine 30 mg/m2 IV daily for 3 doses AND
Cyclophosphamide 500 mg/m2 IV daily for 3 doses.

Adult subjects with moderate impairment of renal function (creatinine clearance 50-70 ml/min/1.73 m²) should receive a reduced dose of fludarabine by at least 20% or in accordance with local prescribing information.

Both agents are started on the same day and administered for 3 consecutive days. Subjects should start LD chemotherapy within 7 days of study enrollment. LD chemotherapy must be completed at least 48 hours (but no more than 7 days) prior to CTX130 infusion.

Reference the current full prescribing information for fludarabine and cyclophosphamide for guidance regarding the storage, preparation, administration, supportive care instructions, and toxicity management associated with LD chemotherapy.

LD chemotherapy or the first daratumumab dose is delayed if any of the following signs or symptoms are present:
Significant worsening of clinical status that increases the potential risk of AEs associated with LD chemotherapy
Requirement for supplemental oxygen to maintain a saturation level of >92%
New uncontrolled cardiac arrhythmia
Hypotension requiring vasopressor support
Active infection: Positive blood cultures for bacteria or fungus, or active viral infection not responding to treatment, or negative culture but active infection
Platelet count≤100,000/mm³, absolute neutrophil count≤1500/mm³, and hemoglobin≤9 g/dL without prior blood cell transfusion
Grade≥2 acute neurological toxicity.

### Administration of CTX130

CTX130 consists of allogeneic T cells modified with CRISPR-Cas9, resuspended in cryopreservative solution (CryoStor CS5), and supplied in a 6-ml infusion vial. A flat dose of CTX130 (based on % CAR+ T cells) is administered as a single IV infusion. A dose limit of 7×10⁴ TCR⁺ cells/kg is imposed for all dose levels. The total dose may be contained in multiple vials. The infusion of each vial should occur within 20 minutes of thawing. Infusion should preferably occur through a central venous catheter. A leukocyte filter must not be used.

Prior to the start of CTX130 infusion, the site pharmacy must ensure that 2 doses of tocilizumab and emergency equipment are available for each specific subject treated. Subjects should be premedicated per the site standard of practice with oral acetaminophen (i.e., paracetamol or its equivalent per site formulary) and diphenhydramine hydrochloride IV or orally (or another H1-antihistamine per site formulary) approximately 30 to 60 minutes prior to CTX130 infusion.

CTX130 infusion is delayed if any of the following signs or symptoms are present:
New active uncontrolled infection
Worsening of clinical status compared to status prior to start of LD chemotherapy that places the subject at increased risk of toxicity
Grade≥2 acute neurological toxicity.

CTX130 is administered at least 48 hours (but no more than 7 days) after the completion of LD chemotherapy. For subjects who are considered for redosing but could not receive CTX130 within 14 days post-LD chemotherapy or LD chemotherapy could not be administered due to failure to meet the criteria described previously, a second attempt is admissible after discussion with the medical monitor.

### CTX130 Postinfusion Monitoring

Following CTX130 infusion, subjects' vitals should be monitored every 30 minutes for 2 hours after infusion or until resolution of any potential clinical symptoms.

Subjects in Part A are hospitalized for a minimum of 7 days after CTX130 infusion, or longer if required by clinical regulation or site practice. Postinfusion hospitalization in Part B is considered based on the safety information obtained during dose escalation and may be performed. In Parts A and B, the length of hospitalization may be extended where required by local regulation or site practice. In both Parts A and B, subjects must remain in proximity of the investigative site (i.e., 1-hour transit time) for at least 28 days after CTX130 infusion. Management of acute CTX130-related toxicities should occur ONLY at the study site.

Subjects are monitored for signs of CRS, TLS, neurotoxicity, GvHD, and other AEs according to the schedule of assessments (Tables 29-31). Guidelines for the management of CAR T cell-related toxicities are described herein. Subjects should remain hospitalized until CTX130-related nonhematologic toxicities (e.g., fever, hypotension, hypoxia, ongoing neurological toxicity) return to grade 1. Subjects may remain hospitalized for longer periods if considered necessary.

### Prior and Concomitant Medications

Allowed Medications and Procedures (Concomitant Treatments)

Necessary supportive measures for optimal medical care are given throughout the study, including IV antibiotics to treat infections, erythropoietin analogs, blood components, etc., except for prohibited medications listed herein.

All concurrent therapies, including prescription and nonprescription medication, and medical procedures must be recorded from the date of signed informed consent through 3 months after CTX130 infusion. Beginning 3 months post-CTX130 infusion, only the following selected concomitant medications will be collected: vaccinations, anticancer treatments (e.g., chemotherapy, radiation, immunotherapy), immunosuppressants (including steroids), and any investigational agents.

Prohibited/Restricted Medications and Procedures

The following medications are prohibited during certain periods of the study as specified below:

Prohibited Within 28 Days Prior to Enrollment to 3 Months After CTX130 Infusion
Live vaccines
Herbal medicine as part of traditional Chinese medicine or non-over-the-counter herbal remedies

Prohibited Throughout the Study Until the Start of New Anticancer Therapy
Any immunosuppressive therapy unless recommended to treat CRS or ICANS, or if previously discussed with and approved by the medical monitor.
Corticosteroid therapy at a pharmacologic dose (>10 mg/day of prednisone or equivalent doses of other corticosteroids) and other immunosuppressive drugs should be avoided after CTX130 administration unless medically indicated to treat new toxicity or as part of management of CRS or neurotoxicity associated with CTX130. Use of oral corticosteroids before and after daratumumab administration is permitted to prevent infusion reactions.
Any anticancer therapy (e.g., chemotherapy, immunotherapy, targeted therapy, radiation, or other investigational agents) other than daratumumab (Parts A3 and A4) or LD chemotherapy prior to disease progression. Palliative radiation therapy for symptom management is permitted depending on extent, dose, and site(s), which should be defined and reported to the medical monitor for determination.

Prohibited Within the First Month After CTX130 Infusion
Granulocyte-macrophage colony-stimulating factor (GM-CSF) due to the potential to worsen symptoms of CRS. Care should be taken with administration of granulocyte colony-stimulating factor (G-CSF) following CTX130 infusion, and the medical monitor must be consulted prior to administration. If after consultation with the medical monitor G-CSF administration is considered during LD chemotherapy, it should be stopped 18 hours prior to CTX130 infusion if given IV and stopped 24 hours prior to CTX130 infusion if given subcutaneously.

Prohibited within the First 28 Days after CTX130 Infusion
Self-medication by the subject with antipyretics (e.g., acetaminophen, aspirin).

### 8. Toxicity Management

### General Guidance

Prior to LD chemotherapy, infection prophylaxis (e.g., antiviral, antibacterial, antifungal agents) should be initiated according to institutional standard of care for ccRCC patients in an immunocompromised setting.

Subjects must be closely monitored for at least 28 days after CTX130 infusion. Significant toxicities have been reported with autologous CAR T cell therapies. Proactively monitor and treat all AEs in accordance with protocol guidance are required.

Although this is a first-in-human study and the clinical safety profile of CTX130 has not been described, the following general recommendations are provided based on prior experience with autologous CD19 CAR T cell therapies:
Fever is the most common early manifestation of CRS; however, subjects may also experience weakness, hypotension, or confusion as first presentation.
Diagnosis of CRS should be based on clinical symptoms and NOT laboratory values.
In subjects who do not respond to CRS-specific management, always consider sepsis and resistant infections. Subjects should be continually evaluated for resistant or emergent bacterial infections, as well as fungal or viral infections.
CRS, HLH, and TLS may occur at the same time following CAR T cell infusion. Subjects should be consistently monitored for signs and symptoms of all the conditions and managed appropriately.
Neurotoxicity may occur at the time of CRS, during CRS resolution, or following resolution of CRS. Grading and management of neurotoxicity are performed separately from CRS.
Tocilizumab must be administered within 2 hours from the time of order.

In addition to toxicities observed with autologous CAR T cells, signs of GvHD are monitored closely due to the allogeneic nature of CTX130.

The safety profile of CTX130 is continually assessed throughout the study, and are updated on a regular basis with new information regarding the identification and management of potential CTX130-related toxicity.

### Toxicity-Specific Guidance

### CTX130 Infusion-Related Reactions

Infusion-related reactions have been reported in autologous CAR T cell trials, including transient fever, chills, and/or nausea, most commonly occurring within 12 hours after administration. CTX130 is formulated with CryoStor CS5, a well-established cryopreservant medium that contains 5% dimethyl sulfoxide (DMSO). Histamine release associated with DMSO can result in adverse effects such as nausea, vomiting, diarrhea, flushing, fevers, chills, headache, dyspnea, or rashes. In the most severe cases, it can also cause bronchospasm, anaphylaxis, vasodilation and hypotension, and mental status changes.

If an infusion reaction occurs, acetaminophen (paracetamol) and diphenhydramine hydrochloride (or another H1-antihistamine) may be repeated every 6 hours after CTX130 infusion as needed.

Nonsteroidal anti-inflammatory drugs may be prescribed as needed if the subject continues to have fever not relieved by acetaminophen. Systemic steroids should NOT be administered except in cases of life-threatening emergency, as this intervention may have a deleterious effect on CAR T cells.

Infection Prophylaxis and Febrile Reaction

Infection prophylaxis should be managed according to the institutional standard of care for ccRCC patients in an immunocompromised setting.

In the event of febrile reaction, an evaluation for infection should be initiated and the subject managed appropriately with antibiotics, fluids, and other supportive care as medically indicated and determined by the treating physician. Viral and fungal infections should be considered throughout a subject's medical management if fever persists. If a subject develops sepsis or systemic bacteremia following CTX130 infusion, appropriate cultures and medical management should be initiated. Additionally, consideration of CRS should be given in any instances of fever following CTX130 infusion within 28 days postinfusion.

For Parts A3 and A4, prophylaxis for herpes zoster and hepatitis B reactivation in the setting of daratumumab treatment is strongly recommended, as per prescribing information.

Subjects undergoing CTX130 therapy have an increased risk of infections due to underlying malignancy, prior antitumor therapies, daratumumab treatment, lymphodepleting chemotherapy, the specific target of CAR T cells (e.g., CD70), and/or complications of the procedure (e.g., CRS, ICANS) as well as treatment of these complications. Infection prophylaxis is recommended.

Tumor Lysis Syndrome

Subjects receiving CAR T cell therapy may be at increased risk of TLS, which occurs when tumor cells release their contents into the bloodstream, either spontaneously or in response to therapy, leading to the characteristic findings of hyperuricemia, hyperkalemia, hyperphosphatemia, hypocalcemia, and elevated blood urea nitrogen. These electrolyte and metabolic disturbances can progress to clinical toxic effects, including renal insufficiency, cardiac arrhythmias, seizures, and death due to multiorgan failure (Howard et al., N Engl J Med, 2011). TLS has been reported in hematomalignancies as well as solid tumors. Most solid tumors pose a low risk for TLS. It has been most frequently observed in patients with hematomalignancies, in particular leukemic forms such as ALL, acute myeloid leukemia, and CLL, which have a high (>5%) risk for TLS, and noncutaneous T cell lymphomas, particularly adult T cell leukemia/lymphoma and DLBCL (Coiffier et al., J Clin Oncol, 2008). Additional risk factors include lactate dehydrogenase level higher than ULN, high tumor burden, and tumors with high replicative index. Patients with compromised renal function are also at elevated risk for developing TLS.

Subjects should be closely monitored for TLS via laboratory assessments and symptoms from the start of LD chemotherapy until 28 days following CTX130 infusion.

Subjects at increased risk of TLS should receive prophylactic allopurinol (or a non-allopurinol alternative such as febuxostat) and/or rasburicase (Cortes et al., J Clin Oncol, 2010) and increased oral/IV hydration during screening and before initiation of LD chemotherapy. Prophylaxis can be stopped after 28 days following CTX130 infusion or once the risk of TLS passes.

Sites should monitor and treat TLS as per their institutional standard of care, or according to published guidelines (Cairo et al., Br J Haematol, 2004). TLS management, including administration of rasburicase, should be instituted promptly when clinically indicated.

Cytokine Release Syndrome

CRS is a toxicity associated with immune therapies, including CAR T cells, resulting from a release of cytokines, in particular IL-6 and IL-1 (Norelli et al., Nat Med, 2018). CRS is due to hyperactivation of the immune system in response to CAR engagement of the target antigen, resulting in multicytokine elevation from rapid T cell stimulation and proliferation (Frey et al., Blood, 2014; Maude et al., Cancer J, 2014). CRS has been observed in clinical trials irrespective of the antigen-targeted agents, including CD19−, BCMA−, CD123−, and mesothelin-directed CAR T cells, and anti-NY-ESO 1 and MART 1-targeted TCR-modified T cells (Frey et al., Blood, 2014; Hattori et al., Biol Blood Marrow Transplant. 2019; Maude et al., N Engl J Med 2018; Neelapu et al., N Engl J Med, 2017; Raj e et al., N Engl J Med 2019; Tanyi et al., J Immunother, 2017). CRS is a major toxicity reported with autologous CAR T cell therapy that has also been observed in early phase studies with allogeneic CAR T cell therapy (Benjamin et al., Am Soc Hematol Ann Meeting, 2018).

CRS may be life-threatening. Clinically, CRS can be mistaken for a systemic infection or, in severe cases, septic shock. Frequently the earliest sign is elevated temperature, which should prompt an immediate differential diagnostic work-up and timely initiation of appropriate treatment.

The goal of CRS management is to prevent life-threatening states and sequelae while preserving the potential for the anticancer effects of CTX130. Symptoms usually occur 1 to 14 days after autologous CAR T cell therapy in hematologic malignancies.

CRS should be identified and treated based on clinical presentation and not laboratory measurements. If CRS is suspected, grading should be applied according to the American Society for Transplantation and Cellular Therapy (ASTCT; formerly known as American Society for Blood and Marrow Transplantation [ASBMT]) consensus recommendations (Lee et al., Biol Blood Marrow Transplant, 2019), and management should be performed according to the recommendations in Table 38, which are adapted from published guidelines (Lee et al., Biol Blood Marrow Transplant, 2019). Accordingly, grading of neurotoxicity is aligned with the ASTCT criteria for ICANS.

TABLE 37
Grading of CRS per ASTCT Consensus Criteria

| CRS | | | | |
|---|---|---|---|---|
| Parameter | Grade 1 | Grade 2 | Grade 3 | Grade 4 |
| Fever¹ | Temperature ≥38°C. | Temperature ≥38° C. | Temperature ≥38° C. | Temperature ≥38° C. |
| With | None | Not requiring | Requiring a vasopressor | Requiring multiple |
| hypotension | | vasopressors | with or without | vasopressors (excluding |
| | | | vasopressin² | vasopressin)² |
| And/or³ | None | Requiring | Requiring high-flow | Requiring positive pressure |
| Hypoxia | | low-flow | nasal cannula⁴, | (e.g., CPAP, BiPAP, |
| | | nasal cannula⁴ | facemask, nonrebreather | intubation, and mechanical |
| | | or blow-by | mask, or Venturi mask | ventilation) |

ASTCT: American Society for Transplantation and Cellular Therapy; BiPAP: bilevel positive airway pressure; C: Celsius; CPAP: continuous positive airway pressure; CRS: cytokine release syndrome.
Note:
CRS grading based on ASTCT consensus criteria (Lee et al., Biol Blood Marrow Transplant, 2019)
Note:
Organ toxicities associated with CRS may be graded according to CTCAE v5.0 but they do not influence CRS grading.
¹Fever is defined as temperature ≥38° C. not attributable to any other cause. In subjects who have CRS then receive antipyretics or anticytokine therapy such astocilizumab or steroids, fever is no longer required to grade subsequent CRS severity. In this case, CRS grading is driven by hypotension and/or hypoxia.
²See Table 39 for information on high-dose vasopressors.
³CRS grade is determined by the more severe event: hypotension or hypoxia not attributable to any other cause. For example, a subject with temperature of 39.5° C., hypotension requiring 1 vasopressor, and hypoxia requiring low-flow nasal cannula is classified as grade 3 CRS.
⁴Low-flow nasal cannula is defined as oxygen delivered at ≤6 L/minute. Low flow also includes blow-by oxygen delivery, sometimes used in pediatrics. High-flow nasal cannula is defined as oxygen delivered at >6 L/minute.

TABLE 38
CRS Grading and Management Guidance

| CRS | | | Hypotension |
|---|---|---|---|
| Severity¹ | Tocilizumab | Corticosteroids | Management |
| Grade 1 | Tocilizumab² may be considered | N/A | N/A |
| Grade 2 | Administer tocilizumab 8 mg/kg IV | Manage per | Manage per |
| | over 1 hour (not to exceed 800 mg)² | institutional | institutional |
| | Repeat tocilizumab every 8 hours as | guidelines if no | guidelines |
| | needed if not responsive to IV fluids | improvement after | |
| | or increasing supplemental oxygen. | initial tocilizumab | |
| | Limit to ≤3 doses in a 24-hour period; | therapy. | |
| | maximum total of 4 doses. | Continue | |
| | | corticosteroids use | |
| | | until the event is | |
| | | Grade ≤1, then taper | |
| | | appropriately. | |
| Grade 3 | Per grade 2 | Per grade 2 | Manage per |
| | | | institutional |
| | | | guidelines |
| Grade 4 | Per grade 2 | Per grade 2 | Manage per |
| | If no response to multiple doses of | | institutional |
| | tocilizumab and steroids, consider | | guidelines |
| | using other anticytokine therapies | | |
| | (e.g., anakinra). | | |

CRS: cytokine release syndrome; IV: intravenously; N/A: not applicable.
¹See (Lee et al., Biol Blood Marrow Transplant, 2019).
²Refer to tocilizumab prescribing information.

TABLE 39
High-dose Vasopressors

| Pressor | Dose^{a} |
|---|---|
| Norepinephrine monotherapy | ≥20 μg/min |
| Dopamine monotherapy | ≥10 μg/kg/min |
| Phenylephrine monotherapy | ≥200 μg/min |
| Epinephrine monotherapy | ≥10 μg/min |
| If on vasopressin | Vasopressin + norepinephrine equivalent |
| | of ≥10 μg/min^{b} |
| If on combination vasopressors | Norepinephrine equivalent of ≥20 ug/min^{b} |
| (not vasopressin) | |

^{a}All doses are required for ≥3 hours.
^{b}VASST Trial vasopressor equivalent equation: norepinephrine equivalent dose = [norepinephrine (μg/min)] + [dopamine (μg/min)/2] + [epinephrine (μg/min)] + [phenylephrine (μg/min)/10]

Throughout the duration of CRS, subjects should be provided with supportive care consisting of antipyretics, IV fluids, and oxygen. Subjects who experience grade≥2 CRS should be monitored with continuous cardiac telemetry and pulse oximetry. For subjects experiencing grade 3 CRS, consider performing an echocardiogram to assess cardiac function. For grade 3 or 4 CRS, consider intensive care supportive therapy. The potential of an underlying infection in cases of severe CRS should be considered, as the presentation (fever, hypotension, hypoxia) is similar. Resolution of CRS is defined as resolution of fever (fever: temperature≥38° C.), hypoxia, and hypotension (Lee et al., Biol Blood Marrow Transplant, 2019).

Immune Effector Cell-Associated Neurotoxicity Syndrome (ICANS)

Neurotoxicity has been documented in subjects with B cell malignancies treated with autologous CAR T cell therapies. Therefore, subjects are monitored for signs and symptoms of neurotoxicity associated with CAR T cell therapies in the current trial. Neurotoxicity may occur at the time of CRS, during the resolution of CRS, or following resolution of CRS, and its pathophysiology is unclear. The recent ASTCT (formerly known as ASBMT) consensus further defined ICANS as a disorder characterized by a pathologic process involving the CNS following any immune therapy that results in activation or engagement of endogenous or infused T cells and/or other immune effector cells (Lee et al., Biol Blood Marrow Transplant, 2019). The pathophysiology of neurotoxicity remains unclear; however, it is postulated that it may be due to a combination of cytokine release, trafficking of CAR T into CSF, and increased permeability of the blood-brain barrier (June et al., Science, 2018).

Signs and symptoms can be progressive and may include but are not limited to aphasia, altered level of consciousness, impairment of cognitive skills, motor weakness, seizures, and cerebral edema. ICANS grading (Table 40) was developed based on CAR T cell therapy-associated TOXicity (CARTOX) working group criteria used previously in autologous CAR T cell trials (Neelapu et al., Nat Rev Clin Oncol, 2018). ICANS incorporates assessment of level of consciousness, presence/absence of seizures, motor findings, presence/absence of cerebral edema, and overall assessment of neurologic domains by using a modified tool called the ICE (immune effector cell-associated encephalopathy) assessment tool (Table 33).

Evaluation of any new onset neurotoxicity should include a neurological examination (including ICE assessment tool, Table 33), brain MRI, and examination of the CSF, as clinically indicated. If a brain MRI is not possible, all subjects should receive a noncontrast CT scan to rule out intracerebral hemorrhage. Electroencephalogram should also be considered as clinically indicated. Endotracheal intubation may be needed for airway protection in severe cases.

Lumbar puncture is required for any grade 3 or higher neurocognitive toxicity and is strongly recommended for grade 1 and grade 2 events, if clinically feasible. Lumbar puncture must be performed within 48 hours of symptom onset. Infectious etiology should be ruled out by performing a lumbar puncture whenever possible (especially for subjects with grade 3 or 4 ICANS).

Viral encephalitis (e.g., human herpesvirus 6 [HHV-6] encephalitis) must be considered in the differential diagnosis for subjects who experience neurocognitive symptoms after receiving CTX130. Whenever lumbar puncture is performed, the following viral panel needs to be performed in addition to standard panel performed at site (which should include cell count, gram stain, Neisseria meningitidis): CSF PCR analysis for herpes simplex virus 1 and 2, enterovirus, varicella-zoster virus, cytomegalovirus, EBV, and HEW-6.

Results from the infectious disease panel must be available within 4 business days of the lumbar puncture to appropriately manage the subject.

In subjects diagnosed with HEW-6 encephalitis, treatment with ganciclovir or foscarnet should be initiated. Drug selection should be dictated by the drug's side effects, the subject's comorbidities and site clinical practice. The recommended duration of therapy is 3 weeks or as per site clinical practice (Hill et al., Curr Opin Virol, 2014; Ward et al., Haematologica, 2019). Once treatment is initiated, peripheral blood HEW-6 viral load should be checked weekly by PCR. An experienced bone marrow transplant physician and infectious disease expert in addition to the medical monitor need to be consulted.

CSF samples should be sent to a central laboratory for cytokine analysis and for presence of CTX130. Excess sample (if available) is stored for exploratory research.

Nonsedating, antiseizure prophylaxis (e.g., levetiracetam) should be considered, especially in subjects with a history of seizures, for at least 28 days following CTX130 infusion or upon resolution of neurological symptoms (unless the antiseizure medication is considered to be contributing to the detrimental symptoms). Subjects who experience grade≥2 ICANS should be monitored with continuous cardiac telemetry and pulse oximetry. For severe or life-threatening neurologic toxicities, intensive care supportive therapy should be provided. Neurology consultation should always be considered. Monitor platelets and for signs of coagulopathy and transfuse blood products appropriately to diminish risk of intracerebral hemorrhage. Table 40 provides neurotoxicity grading and Table 41 provides management guidance.

For subjects who receive active steroid management for more than 3 days, antifungal and antiviral prophylaxis is recommended to mitigate a risk of severe infection with prolonged steroid use. Consideration for antimicrobial prophylaxis should also be given.

TABLE 40
ICANS Grading

| Neurotoxicity | | | | |
|---|---|---|---|---|
| Domain | Grade 1 | Grade 2 | Grade 3 | Grade 4 |
| ICE score¹ | 7-9 | 3-6 | 0-2 | 0 (subject is |
| | | | | unarousable and |
| | | | | unable to undergo ICE |
| | | | | assessment) |
| Depressed level | Awakens | Awakens | Awakens only to | Subject is unarousable |
| of | spontaneously | to voice | tactile stimulus | or requires vigorous or |
| consciousness² | | | | repetitive tactile |
| | | | | stimuli to arise; stupor |
| | | | | or coma |
| Seizure | N/A | N/A | Any clinical | Life-threatening |
| | | | seizure, focal or | prolonged seizure |
| | | | generalized, that | (>5 min) or repetitive |
| | | | resolves rapidly, | clinical or electrical |
| | | | or nonconvulsive | seizures without return |
| | | | seizures on EEG | to baseline in between |
| | | | that resolve with | |
| | | | intervention | |
| Motor findings³ | N/A | N/A | N/A | Deep focal motor |
| | | | | weakness such as |
| | | | | hemiparesis or |
| | | | | paraparesis |
| Elevated ICP/ | N/A | N/A | Focal/local edema | Diffuse cerebral |
| cerebral edema | | | on neuroimaging⁴ | edema on |
| | | | | neuroimaging, |
| | | | | decerebrate or |
| | | | | decorticate posturing, |
| | | | | cranial nerve VI palsy, |
| | | | | papilladema, or |
| | | | | Cushing’s triad |

CTCAE: Common Terminology Criteria for Adverse Events; EEG: electroencephalogram; ICANS: immune effector cell-associated neurotoxicity syndrome; ICE: immune effector cell-associated encephalopathy (assessment tool); ICP: intracranial pressure; N/A: not applicable.
Note:
ICANS grade is determined by the most severe event (ICE score, level of consciousness, seizure, motor findings, raised ICP/cerebral edema) not attributable to any other cause.
¹A subject with an ICE score of 0 may be classified as grade 3 ICANS if awake with global aphasia, but a subject with an ICE score of 0 may be classified as grade 4 ICANS if unarousable (Table 33 for ICE assessment tool).
²Depressed level of consciousness should be attributable to no other cause (e.g., sedating medication).
³Tremors and myoclonus associated with immune effector therapies should be graded according to CTCAE v5.0 but do not influence ICANS grading.

TABLE 41
ICANS Management Guidance

| Severity | Management |
|---|---|
| Grade 1 | Provide supportive care per institutional practice. |
| Grade 2 | Consider administering dexamethasone 10 mg IV every 6 hours (or equivalent |
| | methylprednisolone) unless subject already on equivalent dose of steroids for CRS. |
| | Continue dexamethasone use until event is grade ≤1, then taper over 3 days. |
| Grade 3 | Administer dexamethasone 10 mg IV every 6 hours, unless subject already on |
| | equivalent dose of steroids for CRS. |
| | Continue dexamethasone use until event is grade ≤1, then taper over 3 days. |
| Grade 4 | Administer methylprednisolone 1000 mg IV per day for 3 days; if improves, then |
| | manage as above. |

CRS: cytokine release syndrome; ICANS: immune effector cell-associated neurotoxicity syndrome; IV: intravenously.

Headache, which may occur in a setting of fever or after chemotherapy, is a nonspecific symptom. Headache alone may not necessarily be a manifestation of ICANS and further evaluation should be performed. Weakness or balance problem resulting from deconditioning and muscle loss are excluded from definition of ICANS. Similarly, intracranial hemorrhage with or without associated edema may occur due to coagulopathies in these subjects and are also excluded from definition of ICANS. These and other neurotoxicities should be captured in accordance with CTCAE v5.0.

Hemophagocytic Lymphohistiocytosis

HLH has been reported after treatment with autologous CAR T cells (Barrett et al., Curr Opin Pediatr, 2014; Maude et al., N Engl J Med, 2014; Maude et al., Blood, 2015; Porter et al., Sci Transl Med, 2015; Teachey et al., Blood, 2013). HLH is a clinical syndrome that is a result of an inflammatory response following infusion of CAR T cells in which cytokine production from activated T cells leads to excessive macrophage activation. Signs and symptoms of HLH may include fevers, cytopenias, hepatosplenomegaly, hepatic dysfunction with hyperbilirubinemia, coagulopathy with significantly decreased fibrinogen, and marked elevations in ferritin and C-reactive protein (CRP). Neurologic findings have also been observed (Jordan et al., Blood, 2011; La Rosée, Hematology Am Soc Hematol Educ Program, 2015).

CRS and HLH may possess similar clinical syndromes with overlapping clinical features and pathophysiology. HLH will likely occur at the time of CRS or as CRS is resolving. HLH should be considered if there are unexplained elevated liver function tests or cytopenias with or without other evidence of CRS. Monitoring of CRP, ferritin, triglycerides, and fibrinogen may assist with diagnosis and define the clinical course. If these laboratory values further support a diagnosis of HLH, soluble CD25 blood levels should be determined in conjunction with a bone marrow biopsy and aspirate if safe to conduct for further confirmation. Where feasible, excess bone marrow samples should be sent to a central laboratory.

If HLH is suspected:
Frequently monitor coagulation parameters, including fibrinogen. These tests may be done more frequently than indicated in the schedule of assessments, and frequency should be driven based on laboratory findings.
Fibrinogen should be maintained >100 mg/dL to decrease risk of bleeding.
Coagulopathy should be corrected with blood products.
Given the overlap with CRS, manage according to grade 3 CRS with appropriate monitoring intensity (Table 37). Follow institutional guidelines for additional treatment of HLH.
The IL-1 inhibitor anakinra or INF-gamma inhibitor gamifant should be considered for management of HLH

Cytopenias

Grade 3 neutropenia and thrombocytopenia, at times lasting more than 28 days after CAR T cell infusion, have been reported in subjects treated with autologous CAR T cell products (Kymriah US prescribing information [USPI], 2018; Raje et al., N Engl J Med, 2019; Yescarta USPI, 2019). Therefore, subjects receiving CTX130 should be monitored for such toxicities and appropriately supported. Monitor platelets and for signs of coagulopathy and transfuse blood products appropriately to diminish risk of hemorrhage. Consideration should be given to antimicrobial and antifungal prophylaxis for any subject with prolonged neutropenia.

Due to the transient expression of CD70 on activated T and B lymphocytes, opportunistic infection such as viral reactivation may occur. Opportunistic infections may be considered when clinical symptoms arise.

During dose escalation, G-CSF may be considered in cases of grade 4 neutropenia post-CTX130 infusion. During cohort expansion, G-CSF may be administered cautiously.

For Parts A3 and A4, daratumumab may increase neutropenia and/or thrombocytopenia induced by background therapy. Monitor complete blood cell counts periodically during treatment according to the manufacturer's prescribing information for background therapies. Monitor subjects with neutropenia for signs of infection. Daratumumab dose delay may be required to allow recovery of neutrophils and/or platelets, as per prescribing information. Consider supportive care with growth factors for neutropenia or transfusions for thrombocytopenia.

Graft vs Host Disease

GvHD is seen in the setting of allogeneic SCT and is the result of immunocompetent donor T cells (the graft) recognizing the recipient (the host) as foreign. The subsequent immune response activates donor T cells to attack the recipient to eliminate foreign antigen-bearing cells. GvHD is divided into acute, chronic, and overlap syndromes based on both the time from allogeneic SCT and clinical manifestations. Signs of acute GvHD may include a maculopapular rash; hyperbilirubinemia with jaundice due to damage to the small bile ducts, leading to cholestasis; nausea, vomiting, and anorexia; and watery or bloody diarrhea and cramping abdominal pain (Zeiser et al., N Engl J Med, 2017).

To support the proposed clinical study, a nonclinical GLP-compliant GvHD and tolerability study was performed in immunocompromised mice treated at 2 IV doses: a high dose of 4×10⁷ CTX130 cells per mouse (approximately 1.6×10⁹ cells/kg) and a low dose of 2×10⁷ cells per mouse (approximately 0.8×10⁹ cells/kg). The high dose level exceeds the proposed highest clinical dose by more than 10-fold when normalized for body weight. No mice treated with CTX130 developed fatal GvHD during the course of the 12-week study. At necropsy, mononuclear cell infiltration was observed in some animals in the mesenteric lymph node and the thymus. Minimal to mild perivascular inflammation was also observed in the lungs of some animals. These findings are consistent with mild GvHD but did not manifest in clinical symptoms in these mice.

Further, due to the specificity of CAR insertion at the TRAC locus, it is highly unlikely for a T cell to be both CAR⁺ and TCR⁺. Remaining TCR⁺ cells are removed during the manufacturing process by immunoaffinity chromatography on an anti-TCR antibody column to achieve ≤0.14% TCR⁺ cells in the final product. A dose limit of 7×10⁴ TCR⁺ cells/kg is imposed for all dose levels. This limit is lower than the limit of 1×10⁵ TCR+ cells/kg based on published reports on the number of allogeneic cells capable of causing severe GvHD during SCT with haploidentical donors (Bertaina et al., Blood, 2014). Through this specific editing, purification, and strict product release criteria, the risk of GvHD following CTX130 should be low, although the true incidence is unknown. Subjects should be monitored closely for signs of acute GvHD following infusion of CTX130. The timing of potential symptoms is unknown. However, given that CAR T cell expansion is antigen-driven and likely occurs only in TCR⁻ cells, it is unlikely that the number of TCR⁺ cells appreciably increases above the number infused.

Diagnosis and grading of GvHD is performed according to MAGIC criteria (Harris et al., Biol Blood Marrow Transplant, 2016), as outlined in Table 42.

TABLE 42
Criteria for Grading Acute GvHD

| | Skin (active erythema | Liver | | | Lower GI |
|---|---|---|---|---|---|
| Stage | only) | (bilirubin) | | Upper GI | (stool output/day) |
| 0 | No active | <2 | mg/dL | No or intermittent | <500 mL/day or |
| | (erythematous) GvHD | | | nausea, vomiting, | <3 episodes/day |
| | rash | | | or anorexia | |
| 1 | Maculopapular rash | 2-3 | mg/dL | Persistent nausea, | 500-999 mL/day or |
| | <25% BSA | | | vomiting, or | 3-4 episodes/day |
| | | | | anorexia | |
| 2 | Maculopapular rash | 3.1-6 | mg/dL | — | 1000-1500 mL/day or |
| | 25-50% BSA | | | | 5-7 episodes/day |
| 3 | Maculopapular rash | 6.1-15 | mg/dL | — | >1500 mL/day or |
| | >50% BSA | | | | >7 episodes/day |
| 4 | Generalized | >15 | mg/dL | — | Severe abdominal pain with |

| | |
|---|---|
| erythroderma (>50% | or without ileus, or grossly |
| BSA) plus bullous | bloody stool (regardless of |
| formation and | stool volume) |
| desquamation | |
| >5% BSA | |

BSA: body surface area; GI: gastrointestinal; GvHD: graft versus host disease.

Overall GvHD grade is determined based on most severe target organ involvement.
Grade 0: No stage 1-4 of any organ
Grade 1: Stage 1-2 skin without liver, upper gastrointestinal (GI), or lower GI involvement
Grade 2: Stage 3 rash and/or stage 1 liver and/or stage 1 upper GI and/or stage 1 lower GI
Grade 3: Stage 2-3 liver and/or stage 2-3 lower GI, with stage 0-3 skin and/or stage 0-1 upper GI
Grade 4: Stage 4 skin, liver, or lower GI involvement, with stage 0-1 upper GI

Potential confounding factors that may mimic GvHD such as infections and reactions to medications should be ruled out. Skin and/or GI biopsy should be obtained for confirmation before or soon after treatment has been initiated. In instance of liver involvement, liver biopsy should be attempted if clinically feasible. Sample(s) of all biopsies are also sent to a central laboratory for pathology assessment.

Recommendations for management of acute GvHD are outlined in Table 43. To allow for intersubject comparability at the end of the trial, these recommendations shall follow except in specific clinical scenarios in which following them could put the subject at risk.

TABLE 43
Acute GvHD Management

| Grade | Management |
|---|---|
| 1 | Skin: Topical steroids or immunosuppressants; if Stage 2: Prednisone 1 mg/kg |
| | (or equivalent dose). |
| 2-4 | Initiate methylprednisone 2 mg/kg daily (or equivalent dose). |
| | IV form of steroid such as methylprednisolone should be considered if there are |
| | concerns with malabsorption. |
| | Steroid taper may begin after improvement is seen after ≥3 days of steroids. |
| | Taper should be 50% decrease of total daily steroid dose every 5 days. |
| | GI: in addition to steroids, start antidiarrheal agents as per standard practice. |

GI: gastrointestinal; IV: intravenous.

Decisions to initiate second-line GvHD therapy should be made sooner for subjects with more severe GvHD. For example, secondary therapy may be indicated after 3 days with progressive manifestations of GvHD, after 1 week with persistent grade 3 GvHD, or after 2 weeks with persistent grade 2 GvHD. Second-line systemic therapy may be indicated earlier in subjects who cannot tolerate high-dose glucocorticoid treatment (Martin et al., Biol Blood Marrow Transplant, 2012). Choice of secondary therapy and when to initiate is based on clinical judgment and local practice.

Management of refractory acute GvHD or chronic GvHD is per institutional guidelines. Anti-infective prophylaxis measures should be instituted per local guidelines when treating subjects with immunosuppressive agents (including steroids).

On Target Off-Tumor Toxicities

Activity of CTX130 Against Activated T and B Lymphocytes, Dendritic Cells

Activated T and B lymphocytes express CD70 transiently, and dendritic cells, as well as thymic epithelial cells express CD70 to a certain degree. Thus, these cells might become a target for activated CTX130. Management of infections and cytopenias is disclosed herein.

Activity of CTX130 Against Osteoblasts

Activity of CTX130 was detected in nonclinical studies in cell culture of human primary osteoblasts. Hence, bone turnover is monitored via calcium levels as well as 2 osteoblast-specific markers: amino-terminal propeptide of type I procollagen (PINP) and bone-specific alkaline phosphatase (B SAP), which are considered the most useful markers in the assessment of bone formation (Fink et al., Osteoporosis, 2000). Standardized assays for assessment of both markers in serum are available. The concentration of these peptide markers reflects the activity of osteoblasts and the formation of new bone collagen.

PINP and BSAP are measured through a central laboratory assessment at screening, baseline, Days 7, 15, 22, and 28, and Months 3, 6, and 12 of the study (Tables 29-31). Samples are collected at the same time of day (±2 hours) on the specified collection days because of the strong effect of circadian rhythm on bone turnover.

Activity of CTX130 Against Renal Tubular-Like Epithelium

Activity of CTX130 against renal tubular-like epithelial cells was detected in nonclinical studies of CTX130 in primary human kidney epithelium. Hence, subjects should be monitored for acute tubular damage by monitoring for an increase in serum creatinine of at least 0.3 mg/dL (26.5 μmol/L) over a 48-hour period and/or ≥1.5 times the baseline value within the previous 7 days. Serum creatinine is assessed daily for the first 7 days post-CTX130 infusion, every other day between Days 8 through 15 of treatment, and then twice weekly until Day 28 (Tables 29-31). If acute renal tubular damage is suspected, additional tests should be conducted, including urine sediment analysis and fractional excretion of sodium in urine, and consultation by a nephrologist should be initiated.

Uncontrolled T Cell Proliferation

Upon recognition of target tumor antigen, in vivo activation and expansion have been observed with CAR T cells (Grupp et al., N Engl J Med, 2013). Autologous CAR T cells have been detected in peripheral blood, bone marrow, cerebrospinal fluid, ascites and other compartments (Badbaran et al., Cancers (Basel), 2020). If a subject develops signs of uncontrolled T cell proliferation, a sample from the clinical investigation should be submitted to the central laboratory to determine the origin of the proliferating T cells.

Special Consideration During COVID-19 Pandemic

Subjects enrolled in this study undergo LD chemotherapy, are immunocompromised, and at increased risk of infections. Hence, the clinical study protocol requires exclusion of subjects in the case of any ongoing active infection during screening, prior to LD chemotherapy, and prior to CTX130 infusion, or delayed infusions. This measure includes subjects with active infection with severe acute respiratory syndrome coronavirus 2 (SARS-CoV-2), the causal agent of COVID-19 (coronavirus disease 2019).

Due to the rapidly changing evidence as well as locoregional differences, local regulations and institutional guidelines are deferred if the current situation allows a safe conduct of the study for an individual subject at a given time. Additionally, the minimal requirements regarding COVID-19 infection and vaccinations were defined in a memorandum to the study centers that is periodically updated as evidence and guidelines evolve.

### 9. Assessment of Safety

Definition of Adverse Event Parameters

Adverse Events

The International Conference on Harmonisation (ICH) Guideline for Good Clinical Practice (GCP) E6(R2) defines an AE as:

“Any untoward medical occurrence in a patient or clinical investigation subject administered a pharmaceutical product and which does not necessarily have a causal relationship with this treatment. An AE can therefore be any unfavorable and unintended sign (including an abnormal laboratory finding, for example), symptom or disease temporally associated with the use of a medicinal (investigational) product whether or not considered related to the medicinal (investigational) product.”

Additional criteria defining an AE are described below:
Aggravation of a preexisting disease or permanent disorder (any clinically significant worsening in the nature, severity, frequency, or duration of a preexisting condition.
Events resulting from protocol-mandated procedures (e.g., complications from invasive procedures)

The following are not considered to be adverse events:
Medical or surgical procedures, including elective or preplanned (scheduled prior to the subject being enrolled into the study), e.g., surgery, endoscopy, tooth extraction, transfusion. These should be recorded in the relevant eCRF. (Note: an untoward medical event occurring during the prescheduled elective procedure or routinely scheduled treatment should be recorded as an AE or SAE).
Pre-existing diseases or conditions that do not worsen during or after administration of the investigational medicinal product
Hospitalization planned for study treatment infusion or observation
The malignancy under study or signs and symptoms associated with the disease, as well as progression or relapse of the underlying malignancy.

Only abnormal laboratory results considered to be clinically significant should be reported as AEs (e.g., an abnormal laboratory finding associated with clinical symptoms, of prolonged duration, or that requires additional monitoring and/or medical intervention). Whenever possible, these should be reported as a clinical diagnosis rather than the abnormal parameter itself (i.e., neutropenia versus neutrophil count decreased). Abnormal laboratory results without clinical significance should not be recorded as AEs.

Adverse events can occur before, during, or after treatment, and can be either treatment-emergent (i.e., occurring post-CTX130 infusion) or non-treatment-emergent. A non-treatment-emergent AE is any new sign or symptom, disease, or other untoward medical event that occurs after written informed consent has been obtained but before the subject has received CTX130.

Abnormal Laboratory Findings

Abnormal laboratory findings considered to be clinically significant should be reported as adverse events (e.g., an abnormal laboratory finding associated with clinical symptoms, prolonged duration that requires additional monitoring and/or medical intervention). Whenever possible, these should be reported as a clinical diagnosis rather than the abnormal parameter itself (i.e., neutropenia versus neutrophil count decreased). Abnormal laboratory results without clinical significance should not be recorded as AEs.

Disease Progression

Disease progression and sign and symptom of disease progression should not be reported as an AE with the following exception:
Atypical or accelerated progression of malignancy under study that in its nature, presentation, or severity differs from the normal course of the disease, with symptoms meeting serious criteria. In this case, worsening of the underlying condition should be reported as the SAE.
Disease progression with outcome of death within 30 days of study dose regardless of relationship to CTX130 should be recorded as an SAE and reported.

### Serious Adverse Events

An SAE is any untoward medical occurrence that at any dose:
Results in death
Is life-threatening. This definition implies that the subject is at immediate risk of death from the event as it occurred. It does not include an event that, had it occurred in a more severe form, might have caused death.
Requires inpatient hospitalization or prolongation of existing hospitalization. In general, hospitalization signifies that the subject has been at the hospital or emergency ward (usually involving at least an overnight stay) for observation and/or treatment that would not have been appropriate in an outpatient setting.
Results in persistent or significant disability/incapacity
Results in a congenital anomaly/birth defect
Other important/significant medical events

Medical and scientific judgment should be exercised in deciding whether expedited reporting is appropriate in other situations, such as important medical events that may not be immediately life-threatening or result in death or hospitalization but may jeopardize the subject or may require intervention to prevent one of the other outcomes listed in the definition above.

Hospitalization for study treatment infusions, or planned hospitalizations following CTX130 infusion, are not considered SAEs. Furthermore, hospitalizations for observation or prolongation of hospitalization for observation alone should not be reported as an SAE unless they are associated with a medically significant event that meets other SAE criteria as assessed.

Adverse Events of Special Interest

Based on the reported clinical experience of autologous CAR T cells considered to be in the same pharmacological class, the following AESIs are identified:
CTX130 infusion-related reactions
Grade≥3 infections and infestations
Tumor lysis syndrome
Cytokine release syndrome
Immune effector cell-associated neurotoxicity syndrome
Hemophagocytic lymphohistiocytosis
Graft vs host disease
Uncontrolled T cell proliferation

In addition to the AESIs listed above, any new autoimmune disorder that is determined to be possibly related or related to CTX130 should be reported any time after CTX130 infusion (Fraietta et al., Nature, 2018).

### Assessment of Adverse Events

Assessment of Causality

The relationship between each AE and CTX130, LD chemotherapy, daratumumab administration, and any protocol-mandated study procedure (all assessed individually) should be assessed. The following should be considered: (1) the temporal association between the timing of the event and administration of the treatment or procedure, (2) a plausible biological mechanism, and (3) other potential causes of the event (e.g., concomitant therapy, underlying disease) when making their assessment of causality.

The assessment of relationship is made based on the following definitions:
Related: There is a clear causal relationship between the study treatment or procedure and the AE.
Possibly related: There is some evidence to suggest a causal relationship between the study treatment or procedure and the AE, but alternative potential causes also exist.
Not related: There is no evidence to suggest a causal relationship between the study treatment or procedure and the AE.

If the relationship between the AE/SAE and CTX130 is determined to be “possible,” the event is considered related to CTX130 for the purposes of regulatory reporting.

An event is considered “not related” to use of CTX130 if any of the following tests are met:
An unreasonable temporal relationship between administration of CTX130 and the onset of the event (e.g., the event occurred either before or too long after administration of CTX130 for the AE to be considered product-related).
A causal relationship between CTX130 and the event is biologically implausible.
A clearly more likely alternative explanation for the event is present (e.g., typical adverse reaction to a concomitant drug and/or typical disease-related event).

An individual AE/SAE is considered “related” to use of CTX130 if the “not related” criteria are not met. If an SAE is assessed to be not related to any study intervention, an alternative etiology must be provided in the case report form (CRF).

Relationship to Protocol Procedures and/or Other Etiologies

If an SAE is determined to be not related to treatment with CTX130, LD chemotherapy, or daratumumab, the relationship of the SAE should be considered and an alternate etiology on the SAE Report Form based on criteria defined below should be provided:
Protocol-related procedure/intervention: The event occurred as a result of a procedure or intervention required during the study (e.g., blood collection, washout of an existing medication) for which there is no alternative etiology present in the subject's medical record. This is applicable to non-treatment-emergent SAEs (i.e., SAEs that occur prior to the administration of CTX130) as well as treatment-emergent SAEs.
Medical history: The event is related to pre-existing conditions other than the disease under study.
Underlying disease progression: Worsening of underlying malignancy being treated.
Non-study treatment concomitant or prior therapies (e.g., prior or new anticancer therapy, treatments for other chronic conditions, etc.).

Assessment of Severity

Assessment of severity is one of the responsibilities in the evaluation of AEs and SAEs. Severity is graded according to NCI CTCAE v5.0 (except for CRS, ICANS, and GvHD, which are graded according to the criteria in Tables 37, 40, and 42, respectively). The determination of severity for events for which CTCAE grade or protocol-specified criteria are not available should be made based on medical judgment (and documented in the CRF) using the severity categories of grades 1 to 5 described in Table 44.

TABLE 44
Adverse Event Severity

| | |
|---|---|
| Grade 1 | Mild; asymptomatic or mild symptoms; clinical or diagnostic observations |
| | only; intervention not indicated |
| Grade 2 | Moderate; minimal, local, or noninvasive intervention indicated; limiting |
| | age-appropriate instrumental ADL 1 |
| Grade 3 | Severe or medically significant but not immediately life-threatening; |
| | hospitalization or prolongation of hospitalization indicated; disabling; |
| | limiting self-care ADL 2 |
| Grade 4 | Life-threatening consequences; urgent intervention indicated |
| Grade 5 | Death related to AE |

ADL: Activities of Daily Living; AE: adverse event.
¹Instrumental ADL refer to preparing meals, shopping for groceries or clothes, using the telephone, managing money, etc.
²Self-care ADL refer to bathing, dressing and undressing, feeding self, using the toilet, taking medications, and not bedridden.

Adverse Event Outcome

The outcome of an AE or SAE classified and reported as follows:
Fatal
Not recovered/not resolved
Recovered/resolved
Recovered/resolved with sequelae
Recovering/resolving
Unknown

When recording and reporting death and fatal/grade 5 events, note that “death” is a subject outcome, and “fatal” is an event outcome and should describe the SAE that was the cause of death. Subjects withdrawn from the study because of AEs are followed until the outcome is determined.

### 10. Stopping Rules and Study Termination

### Stopping Rules for Trial

The study is paused if 1 or more of the following events occur:
Life-threatening (grade 4) toxicity attributable to CTX130 that is unmanageable, unexpected, and unrelated to LD chemotherapy
Death related to CTX130 within 30 days of infusion
After at least 15 subjects in Part A1, in Part A2, or in Part B have received CTX130, occurrence of grade>2 GvHD that is steroid-refractory in >20% of subjects
After at least 15 subjects in Part A1, in Part A2, or in Part B are enrolled, determination of unexpected, clinically significant, or unacceptable risk to subjects that occurred in >35% of the subjects (e.g., grade 3 neurotoxicity not resolving within 7 days to grade≤2)
New malignancy (distinct from recurrence/progression of previously treated malignancy)

In the event enrollment is permanently suspended, subjects who are already enrolled in the study do not proceed with daratumumab administration (Parts A3 and A4 only), LD chemotherapy, and CTX130 infusion. Subjects who have already been treated with CTX130 remain in the study and are continued to be followed per the study protocol or are asked to transition to a long-term safety follow-up study.

### Stopping Rules for Individual Subjects

Stopping rules for individual subjects are as follows:
Any medical condition that would put the subject at risk during continuing study-related treatments or follow-up.
If a subject is found not to have met eligibility criteria or has a major protocol deviation before the start of LD chemotherapy or before the start of daratumumab (Parts A3 and A4).
If a subject has unresolved infusion reaction due to daratumumab treatment that does not resolve within 72h (Parts A3 and A4).

### End of Study Definition

The end of the study is defined as the time at which the last subject completes the Month 60 visit (the last protocol-defined assessment), or is considered lost to follow-up, or withdraws consent, or dies.

### 11. Statistical Analyses

### General Methods

Study data is summarized for disposition, demographic and baseline characteristics, safety, and clinical antitumor activity.

Categorical data is summarized by frequency distributions (number and percentages of subjects) and continuous data will be summarized by descriptive statistics (mean, standard deviation, median, minimum, and maximum).

Subjects treated at the RPBD of CTX130 during Part A are pooled with those receiving the same dosing regimen of CTX130 during the expansion phase, unless otherwise specified. All summaries, listings, figures, and analyses are performed by dosing regimen (dose level and frequency).

Primary analysis time is defined as when 71 subjects in Part B have completed the 3-month disease response assessment, or are lost to follow-up, withdraw from the study, or die, whichever occurs first (defined in full analysis set [FAS]). The study data is analyzed and reported in the primary clinical study report based on primary analysis time. Additional data cumulated from primary analysis time to end of study is reported. Full details of statistical analyses are specified in the statistical analysis plan.

### Study Objectives

The primary objective of Part A is to assess the safety of a single escalating dose and multiple dose regimen of CTX130 in subjects with unresectable or metastatic ccRCC.

The primary objective of Part B is to assess the efficacy of CTX130 in subjects with unresectable or metastatic ccRCC, as measured by ORR according to RECIST v1.1.

### Study Endpoints

Primary Endpoints
Part A1 and Part A3 (single dose escalation): Incidence of AEs, defined as DLTs.
Part A2 and Part A4 (multiple dose regimen): Incidence of AEs after multiple doses of CTX130.
Part B (Cohort Expansion): ORR, defined as the proportion of subjects who have achieved a best overall response of CR or PR according to RECIST v1.1, as assessed by an IRC.

Secondary Endpoints
Efficacy: Efficacy is assessed per RECIST v1.1.
   ORR: Proportion of subjects who have achieved a best overall response of CR or PR according to RECIST v1.1.
   Best overall response: CR, PR, SD, PD, or not evaluable.
   Time to response: Time between the date of CTX130 infusion until first radiographically documented response (PR/CR).
   Duration of response (DoR): Time between first objective response of PR/CR and date of disease progression or death due to any cause. This is reported only for subjects who have had PR/CR events.
   Progression-free survival (PFS): The difference between the date of CTX130 infusion and the date of disease progression or death due to any cause. Subjects who have not progressed and are still on study at the data cutoff date are censored at their last RECIST assessment date.
   Overall survival: Time between the date of CTX130 infusion and death due to any cause. Subjects who are alive at the data cutoff date are censored at the last date the subject was known alive.

Safety

The incidence and severity of AEs and clinically significant laboratory abnormalities is summarized and reported according to CTCAE v5.0, except for CRS, which is graded according to ASTCT criteria (Lee et al., Biol Blood Marrow Transplant, 2019); neurotoxicity, which is graded according to ICANS (Lee et al., Biol Blood Marrow Transplant, 2019) and CTCAE v5.0; and GvHD, which are graded according to MAGIC criteria (Harris et al., Biol Blood Marrow Transplant, 2016).

Pharmacokinetics

The levels of CTX130 in blood over time are assessed using PCR. Complementary analyses using more sensitive genomic techniques or flow cytometry to confirm the presence of CAR protein on the cellular surface may also be performed. Such analyses may be used to confirm the presence of CTX130 in blood and to further characterize other cellular immunophenotypes.

Exploratory Endpoints
Levels of CTX130 in tissues. The expansion and persistence of CTX130 in tumor biopsy or CSF may be evaluated in any samples collected per protocol-specific sampling.
Incidence of anti-CTX130 antibodies
Immunoprofiling of lymphocyte populations
Cytokine profile following administration of CTX130
Impact of anticytokine therapy on effectiveness of CRS treatments, CTX130 proliferation, and the clinical response
Incidence and type of subsequent (post-CTX130) anticancer therapy
Time to CR: Time between date of the CTX130 infusion until first confirmed CR
Time to disease progression: Time between the date of CTX130 infusion until first evidence of disease progression
First or second subsequent therapy-free survival: Time between date of CTX130 infusion and date of first subsequent therapy or death due to any cause, or PFS
Change from baseline in PROs, as measured by EORTC QLQ-C30, EQ-5D-5L, FKSI-19, and FACT-G questionnaires
Change from baseline in cognitive outcomes, as assessed by ICE
Other genomic, proteomic, metabolic, or pharmacodynamic endpoints

### Analysis Sets

The following analysis sets are evaluated and used for presentation of the data:

Part A

DLT-evaluable set: All subjects who receive CTX130 and either have completed the DLT evaluation period following the initial infusion or have discontinued earlier after experiencing a DLT.

Parts A and B

Safety analysis set (SAS): All subjects who were enrolled and received at least 1 dose of CTX130. Subjects are classified according to the treatment received, where treatment received is defined as the assigned dose level/schedule if it was received at least once, or the first dose level/schedule received if assigned treatment was never received. The SAS is the primary set for the analysis of safety data.

Full analysis set (FAS): All subjects who were enrolled and received CTX130 infusion and have at least 1 baseline and 1 postbaseline scan assessment. The FAS is the primary analysis set for clinical activity assessment.

### Sample Size and Power Consideration

Part A
Part A1 (single dose escalation) sample size is up to 36 DLT-evaluable subjects, depending on the number of dose levels evaluated and the occurrence of DLTs.
Part A2 (multiple dose regimen) sample size is up to 36 subjects treated with CTX130 in 3 dose levels. Three subjects are treated at each dose level, with the option to expand any dose level to 12 subjects.
Part A3 (single dose escalation with daratumumab added to the lymphodepletion regimen) sample size is up to 36 DLT-evaluable subjects in 3 dose levels. Three subjects are treated at each dose level, with the option to expand any level to 12 subjects.
Part A4 (multiple dose regimen with daratumumab added to the lymphodepletion regimen) sample size is up to 36 subjects treated with CTX130 infusion in 3 dose levels. Three subjects are treated at each dose level, with the option to expand any dose level to 12 subjects.

Part B

Part B (cohort expansion) is conducted using an optimal Simon 2-stage design. In the first stage of Part B, if ≥5 out of 23 subjects treated with CTX130 achieve an objective response (CR or PR) as assessed by the IRC, the DSMB may decide to expand enrollment to include an additional 48 treated subjects (71 total) in the second stage; otherwise, the enrollment is paused. A sample size of 71 subjects has 80% power (α=0.025, 1-sided test) to reject the null hypothesis that the Part B ORR is less than or equal to the historical response rate of 15% for standard of care (Barata et al., Br J Cancer, 2018; Nadal et al., Ann Oncol, 2016; Powles et al., Br J Cancer, 2018), if assuming the true ORR with CTX130 is 30%.

### Statistical Analyses

Part A1 and Part A3

DLTs are listed and their incidence summarized by Medical Dictionary for Regulatory Activities (MedDRA) primary system organ class (SOC) and/or preferred term (PT), worst grade based on CTCAE v5.0, type of AE, and dose level. The DLT-evaluable set is the primary analysis set for evaluating DLTs in Parts A1 and A3.

Part A2 and Part A4

AEs are listed and their incidence summarized by MedDRA primary SOC and/or PT, worst grade based on CTCAE v5.0, type of AE, and dose level.

Part B

The primary endpoint of ORR is evaluated for subjects who have received CTX130 at the RPBD in both Parts A and B based on IRC assessment. The FAS is the primary analysis set for efficacy. ORR as determined by the IRC is summarized, and 95% confidence intervals (CIs) are calculated.

Sensitivity analyses of ORR is also performed.

General Efficacy Analysis

Time-to-event endpoints are analyzed using Kaplan-Meier methods where appropriate. Estimates of the median and other quantiles (including 25th percentile and 75th percentile) based on the Kaplan-Meier method are calculated and the associated 95% CIs are provided. The survival rate at specific time points, based on the Kaplan-Meier method, is produced. The time-to-event endpoints that are analyzed include:
Duration of response: Among responders only, DoR is calculated as the date of the first occurrence of response to the date of documented disease progression or death, whichever occurs first. Subjects without disease progression or death are censored at the last evaluable response assessment date.
Progression-free survival: Defined as duration from first date of study treatment until documented objective tumor progression or death. Subjects without disease progression or death are censored at the last evaluable response assessment date.
Overall survival: Defined as the time between date of CTX130 infusion and death due to any cause. Subjects who are alive at the data cutoff date are censored at the last date the subject was known alive.

General Safety Analysis

The SAS is used for all listings and summaries of safety data. Safety data is summarized by dose level.

Adverse Events

AEs are graded according to CTCAE v5.0, except for CRS (ASTCT criteria), neurotoxicity (ICANS and CTCAE v5.0), and GvHD (MAGIC criteria).

Treatment-emergent adverse events are defined as AEs that start or worsen on or after the initial CTX130 infusion. The incidence of TEAEs is summarized according to MedDRA by SOC and/or PT, severity (based on CTCAE v5.0), and relation to study treatment.

Summaries of all TEAEs are produced.
All AEs, regardless of start and end time, are listed, and a flag indicating TEAE or not is presented in the listing.

Laboratory Abnormalities

For laboratory tests covered by the CTCAE v5.0, laboratory data is graded accordingly. For laboratory tests covered by CTCAE, grade 0 is assigned for all non-missing values not graded as 1 or higher.

The following summaries are generated separately for hematology and chemistry laboratory tests:
Descriptive statistics for the actual values (and/or change from baseline) or frequencies of clinical laboratory parameters over time
Tables of the worst on-treatment CTCAE grades
Listing of all laboratory data with values flagged to show the corresponding CTCAE grades and the classifications relative to the laboratory normal ranges

In addition to the above-mentioned tables and listings, graphical displays of key safety parameters, such as scatter plots of actual or change in laboratory tests over time or box plots may be specified in the statistical analysis plan.

Efficacy Interim Analysis

One interim analysis for futility is performed by independent biometrics team members (biostatistician and statistical programmer) and reviewed by the DSMB. The interim analysis occurs when 23 subjects in FAS have been treated in Part B and have 3 months of evaluable response data or discontinued from the study earlier.

Biomarker Analysis

Incidence of anti-CTX130 antibodies, levels of CTX130 CAR+ T cells in blood, and levels of cytokines in blood is summarized.

Tumor, blood, possibly bone marrow and aspirate (only in subjects with treatment-emergent HLH), and possibly CSF samples (only in subjects with treatment-emergent neurotoxicity) will be collected to identify genomic, metabolic, and/or proteomic biomarkers that may be indicative of clinical response, resistance, safety, disease, pharmacodynamic activity, or the mechanism of action of CTX130. Samples will be collected and shipped for testing at a central laboratory.

Analysis of CTX130 Levels (Pharmacokinetic Analysis)

Analysis of levels of transduced CD70-directed CAR⁺ T cells is performed on blood samples collected according to the schedule described in Tables 29-31. In subjects experiencing signs or symptoms of CRS, additional blood samples should be drawn every 48 hours (±5 hours) between scheduled collections. The time course of the expansion and persistence of CTX130 in blood is described using a PCR assay that measures copies of CAR construct. Complementary analyses using more sensitive genomic techniques or flow cytometry to confirm the presence of CAR protein on the cellular surface may also be performed.

Samples for analysis of CTX130 levels are measured from blood, CSF (only in subject with treatment-emergent neurotoxicity), bone marrow (only in subjects with treatment-emergent HLH) or tumor biopsy performed following CTX130 infusion. The expansion and persistence of CTX130 in blood, CSF, bone marrow or tumor tissue may be evaluated in any of these samples collected as per protocol-specified sampling.

Cytokines

Cytokines including, but not limited to, CRP, IL-1β, sIL-1Rα, IL-2, sIL-2Rα, IL-4, IL-6, IL-8, IL-10, IL-12p70, IL-13, IL-15, IL-17a, interferon γ, TNFα, and GM-CSF, are analyzed in a central laboratory. Correlational analysis performed in multiple prior CAR T cell clinical studies have identified these cytokines, and others, as potential predictive markers for severe CRS, as summarized in a recent review (Wang et al., Clin Cnacer Res 2018). Blood for cytokines is collected at specified times as described in Tables 29-31. In subjects experiencing signs or symptoms of CRS, initial sample collection occurs at onset of symptoms, and additional samples should be drawn every 12 hours (±5 hours) until resolution.

Anti-CTX130 Antibody

The CAR construct is composed of humanized scFv. Blood is collected throughout the study to assess for potential immunogenicity, per the schedule of assessments.

PK analysis of daratumumab is performed on blood samples collected according to the schedule described in Tables 29-31.

The trafficking of daratumumab in CSF, bone marrow, or tumor tissues may be evaluated in any of these samples collected as per protocol-specific sampling.

Exploratory Research Biomarkers

Exploratory research may be conducted to identify molecular (genomic, metabolic, and/or proteomic) biomarkers and immunophenotypes that may be indicative or predictive of clinical response, resistance, safety, disease, pharmacodynamic activity, and/or the mechanism of action of treatment. Samples are collected per the schedule of assessments.

### Results

To date, all subjects that participated in this study have completed Stage 1 (eligibility screening) within 14 days. After having met the eligibility criteria, three subjects started lymphodepleting therapy within 24 hours of completing Stage 1. All eligible subjects have completed the screening period (stage 1) and received LD chemotherapy in less than 8 days, with two subject completing screening and starting an LD chemo dose within 72 hrs. All subjects receiving LD chemotherapy have progressed to receiving the DL1 dose of CTX130 within 2-3 days following completion of the LD chemotherapy. Table 45 below summarizes patients subject to the treatment disclosed herein.

TABLE 45
Summary of CTX130 Exposure in Instant Study

| | | | Number of | Number of |
|---|---|---|---|---|
| | | | Subjects | |
| | | | who | Subjects |
| | | CTX130 Dose | Received a | who |
| | | Level | Single | Received 2 |
| | Treatment | (Total CAR+ | Infusion of | Infusions of |
| Population | Cohort | T Cells) | CTX 130 | CTX130 |
| Unresectable | Part A1 | DL1 (3 × 10⁷) | 2 (66.7) | 1 (33.3) |
| or Metastatic | (LD | N = 3 | | |
| Clear Cell | chemotherapy + | DL2(1 × 10⁸) | 3 (100.0) | 0 |
| Renal Cell | CTX130) | N = 3 | | |
| Carcinoma | | DL3 (3 × 10⁸) | 3 (100.0) | 0 |
| | | N = 3 | | |
| | | DL4 (9 × 10⁸) | 2 (66.7) | 1 (33.3) |
| | | N = 3 | | |
| | | Total Part A1 | 10 (83.3) | 2 (16.7) |
| | | N = 12 | | |
| | Part A3 | DL2 (1 × 10⁸) | 1 (100) | 0 |
| | (daratumumab + | N = 1 | | |
| | LD | | | |
| | chemotherapy + | | | |
| | CTX130) | | | |

| | | |
|---|---|---|
| Total Number of subjects in all cohorts (N = 13) | 11 (84.6) | 2 (15.4) |

None of the treated subjects in this study exhibited any DLTs so far. Similarly, no DTLs were observed in a parallel study using CTX130 to treat subjects with a T or B cell malignancy. See, e.g., International Patent Application Nos. PCT/IB2020/060719, filed Nov. 13, 2020 and PCT/IB2020/060720, filed Nov. 13, 2020, the relevant disclosures of each of which are incorporated by reference for the subject matter and purpose referenced herein. Further, the allogeneic CAR-T cell therapy exhibited desired pharmacokinetic features in the treated human subjects, including CAR-T cell expansion and persistence after infusion. Significant CAR T cell distribution, expansion and persistence has been observed as early as DL1. Up to 87-fold expansion of CTX130 in peripheral blood over T₀ has been observed in the one RCC subject evaluated to date and persistence of CTX130 cells can be detected in DL1 subjects at least 28 days following infusion. Similar patterns of CAR T cell distribution, expansion and persistence are observed in the corresponding T or B cell malignancy study, where 20-fold expansion of CTX130 has been observed and CTX130 cells have been detected up to 14 days post-infusion.

Efficacy was evaluated using Response Evaluation Criteria in Solid Tumors (RECIST) v 1.1. Response to CTX130 as of the data cut-off in this study is summarized in Table 46 below. Based on the available response assessments for the 13 subjects treated as indicated in Table 45 above, overall responsees were PD in 4 subjects, SD in 8 subjects, and CR (ongoing at Month 9 assessment) in 1 subject.

TABLE 46
Summary of Overall Responses

| | Part A1 | | | | Part A3 |
|---|---|---|---|---|---|
| | DL1 | DL2 | DL3 | DL4 | DL2 |
| | N = 3 | N = 3 | N = 3 | N = 3 | N = 1 |
| Complete Response | 1 (33.3) | 0 | 0 | 0 | 0 |
| n (%) | | | | | |
| Partial Response | 0 | 0 | 0 | 0 | 0 |
| n (%) | | | | | |
| Stable Disease | 2 (66.7) | 2 (66.7) | 1 (33.3) | 3 (100) | 0 |
| n (%) | | | | | |
| Progressive Disease | 2 | 1 (33.3) | 2 (66.7) | 0 | 1 (100) |
| n (%) | | | | | |

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same FAShion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

The term “about” or “approximately” means within an acceptable error range for the particular value as determined by one of ordinary skill in the art, which will depend in part on how the value is measured or determined, i.e., the limitations of the measurement system. For example, “about” can mean within an acceptable standard deviation, per the practice in the art. Alternatively, “about” can mean a range of up to ±20%, preferably up to ±10%, more preferably up to ±5%, and more preferably still up to ±1% of a given value. Alternatively, particularly with respect to biological systems or processes, the term can mean within an order of magnitude, preferably within 2-fold, of a value. Where particular values are described in the application and claims, unless otherwise stated, the term “about” is implicit and in this context means within an acceptable error range for the particular value.

It should also be understood that, unless clearly indicated to the contrary, in any methods claimed herein that include more than one step or act, the order of the steps or acts of the method is not necessarily limited to the order in which the steps or acts of the method are recited.

1. A method for treating a solid tumor, the method comprising multiple cycles of treatment, wherein each cycle of treatment comprises:
(i) performing a lymphodepletion treatment to a human patient having a solid tumor, which optionally is a CD70+ solid tumor; and
(ii) administering to the human patient an effective amount of a population of genetically engineered T cells after step (i), wherein the population of genetically engineered T cells comprises T cells expressing a chimeric antigen receptor (CAR) that binds CD70.

2. The method of claim 1, wherein the human patient presents a feature of:
(a) loss of response within 2 years after administration of the genetically engineered T cells; or
(b) stable disease or progressive disease with significant clinical benefit at about 6 weeks after administration of the genetically engineered T cells.

3. The method of claim 1, wherein the administration of the genetically engineered T cells in two consecutive cycles of treatment is about 8 weeks apart.

4. The method of claim 1, wherein the human patient does not show one or more of the following prior to a subsequent cycle of the treatment:
(a) dose-limiting toxicity (DLT),
(b) Grade≥3 CRS that does not resolve to ≤Grade 2 within 72 hours after the last dose of the genetically engineered T cells,
(c) Grade>1 GvHD, and
(d) Grade≥2 ICANS.

5. The method of claim 1, further comprising, between two consecutive cycles of the treatment, confirming presence of CD70+ tumor cells in the human patient.

6. The method of claim 1, wherein each cycle of the treatment further comprises: (iii) administering to the human patient a first dose of an anti-CD38 antibody; and optionally (iv) administering to the human patient a second dose of the anti-CD38 antibody.

7. The method of claim 6, wherein the anti-CD38 antibody is daratumumab.

8. The method of claim 6, wherein the first dose of the anti-CD38 antibody is administered to the human patient at least 12 hours prior to the lymphodepletion treatment in step (i) and within 10 days of administration of the genetically engineered T cells in step (ii).

9. The method of claim 6, wherein the second dose of the anti-CD38 antibody in step (iv) is administered to the human patient about three weeks after the administration of the genetically engineered T cells in step (ii).

10. The method of claim 6, wherein each cycle of the treatment further comprises (v) administering to the human patient a third dose of the anti-CD38 antibody.

11. The method of claim 10, wherein the human patient achieves stable disease or a better response.

12. The method of claim 10, wherein the third dose of the anti-CD38 antibody is performed about 6-7 weeks after the administration of the genetically engineered T cells in step (ii).

13. The method of claim 1, which comprises two or three cycles of the treatment.

14. A method for treating a solid tumor, the method comprising:
(i) administering to a human patient having a solid tumor, which optionally is a CD70+ solid tumor, one or more doses of an anti-CD38 antibody,
(ii) performing a lymphodepletion treatment to the human patient after the first dose of the anti-CD38 antibody; and
(iii) administering to the human patient an effective amount of a population of genetically engineered T cells, which expresses a chimeric antigen receptor (CAR) that binds CD70 and is deficient in MHC Class I expression.

15-19. (canceled)

20. The method of claim 1, wherein the lymphodepletion treatment comprises co-administering to the human patient fludarabine at 30 mg/m² and cyclophosphamide at 500 mg/m² intravenously per day for three days.

21. The method of claim 1, wherein the lymphodepletion is performed about 2-7 days prior to the administration of the genetically engineered T cells.

22. The method of claim 1, wherein the effective amount of the genetically engineered T cells range from about 1×10⁶ CAR+ cells to about 9×10⁸ CAR+ cells, optionally about 3×10⁷ CAR+ cells to about 1×10⁸ CAR+ cells, about 1×10⁸ CAR+ cells to about 3×10⁸ CAR+ cells, about 3×10⁸ CAR+ cells to about 4.5×10⁸ CAR+ cells, about 4.5×10⁸ CAR+ cells to about 6×10⁸ CAR+ cells, about 6×10⁸ CAR+ cells to about 7.5×10⁸ CAR+ cells, or about 7.5×10⁸ CAR+ cells to about 9×10⁸ CAR+ cells.

23. The method of claim 22, wherein the effective amount of the genetically engineered T cells is about 3×10⁷, 1×10⁸, 3×10⁸, 4.5×10⁸, 6×10⁸, 7.5×10⁸, or 9×10⁸ CAR+ T cells.

24. The method of claim 1, wherein prior to the administration of the genetically engineered T cells and after the lymphodepletion treatment, the human patient does not show one or more of the following features:
(a) active uncontrolled infection,
(b) worsening of clinical status compared to the clinical status prior to the lymphodepletion treatment, and
(c) Grade≥2 acute neurological toxicity.

25. The method of claim 10, wherein the first dose, the second dose, and/or the third dose of the anti-CD38 antibody is 16 mg/kg via intravenous infusion.

26. The method of claim 25, wherein the first dose, the second dose, and/or the third dose of the anti-CD38 antibody are split evenly into two portions, which are administered to the human patient in two consecutive days.

27. The method of claim 10, wherein the first dose, the second dose, and/or the third dose of the anti-CD38 antibody is 8 mg/kg via intravenous infusion.

28. The method of claim 10, wherein the first dose, the second dose, and/or the third dose of the anti-CD38 antibody are 1800 mg via subcutaneous injection.

29. The method of claim 10, wherein the human patient is free of one or more of the following prior to administration of a subsequent dose of the anti-CD38 antibody:
(a) severe or unmanageable toxicity with prior doses of the anti-CD38 antibody,
(b) disease progression without significant clinical benefit,
(c) ongoing uncontrolled infection,
(d) ≥grade 3 thrombocytopenia;
(e) ≥3 neutropenia; and
(f) CD4+ T cell count<100/μl.

30. The method of claim 1, wherein prior to the lymphodepletion treatment, the human patient does not show one or more of the following features:
(a) significant worsening of clinical status,
(b) requirement for supplemental oxygen to maintain a saturation level of greater than 92%,
(c) uncontrolled cardiac arrhythmia,
(d) hypotension requiring vasopressor support,
(e) active infection,
(f) platelet count≤100,000/mm³, absolute neutrophil count≤1500/mm³, and/or hemoglobin≤9 g/dL without prior blood cell transfusion; and
(g) Grade≥2 acute neurological toxicity.

31. The method of claim 1, further comprising monitoring the human patient for development of acute toxicity after administration of the genetically engineered T cells.

32. The method of claim 31, wherein acute toxicity comprises cytokine release syndrome (CRS), neurotoxicity, tumor lysis syndrome, GvHD, viral encephalitis, on target off-tumor toxicity, and uncontrolled T cell proliferation, optionally wherein the neurotoxicity is immune effector cell-associated neurotoxicity (ICANS).

33. The method of claim 32, wherein the on target off-tumor toxicity comprises activity of the population of genetically engineered T cells against activated T lymphocytes, B lymphocytes, dendritic cells, osteoblasts and/or renal tubular-like epithelium.

34. The method of claim 1, wherein the solid tumor is renal cell carcinoma (RCC).

35. The method of claim 34, wherein the human patient has unresectable or metastatic RCC.

36. The method of claim 35, wherein the human patient has relapsed or refractory RCC.

37. The method of claim 1, wherein the human patient has clear cell differentiation.

38. The method of claim 1, wherein the human patient has undergone at least one line of prior anti-cancer therapy.

39. The method of claim 38, wherein the prior anti-cancer therapy comprises a checkpoint inhibitor, a tyrosine kinase inhibitor, a vascular growth factor inhibitor, or a combination thereof.

40. The method of claim 1, wherein the human patient is subject to an additional anti-cancer therapy after treatment with the population of genetically engineered T cells.

41. The method of claim 1, wherein the human patient has one or more of the following features:
(a) Karnofsky performance status (KPS)≥80%, and
(b) adequate organ function,
(c) free of treatment with prior anti-CD70 or adoptive T cell or NK cell therapy,
(d) free of contraindications to lymphodepletion therapy,
(e) free of central nervous system (CNS) manifestation of malignancy,
(f) free of prior central nervous system disorders,
(g) free of pleural effusion or ascites or pericardial infusion,
(h) free of unstable angina, arrhythmia, and/or myocardial infarction,
(i) free of diabetes mellitus,
(j) free of uncontrolled infections,
(k) free of immunodeficiency disorders or autoimmune disorders that require immunosuppressive therapy,
(l) free of liver vaccine or herbal medicines, and
(m) free of solid organ transplantation or bone marrow transplant.

42. The method of claim 1, wherein the human patient is an adult.

43. The method of claim 1, wherein the genetically engineered T cells comprise a disrupted TRAC gene, a disrupted β2M gene, a disrupted CD70 gene, or a combination thereof.

44. The method of claim 43, wherein the genetically engineered T cells comprise a disrupted β2M gene.

45. The method of claim 43, wherein the genetically engineered T cells comprise a disrupted TRAC gene, a disrupted β2M gene, and a disrupted CD70 gene.

46. The method of claim 43, wherein the genetically engineered T cells comprise a nucleotide sequence encoding the CAR, which is inserted into a genetic site of the T cells, optionally wherein the genetic site is the disrupted TRAC gene.

47. The method of claim 46, wherein the population of genetically engineered T cells comprises T cells having a disrupted TRAC gene, a disrupted β2M gene, and a disrupted CD70 gene, and wherein a nucleotide sequence encoding the CAR that binds CD70 is inserted into the disrupted TRAC gene.

48. The method of claim 43, wherein the disrupted TRAC gene is produced by a CRISPR/Cas9 gene editing system, which comprises a guide RNA comprising a spacer sequence of SEQ ID NO: 8 or 9.

49. The method of claim 48, wherein the disrupted TRAC gene has a deletion of the region targeted by the spacer sequence of SEQ ID NO: 8 or 9, or a portion thereof.

50. The method of claim 43, wherein the disrupted β2M gene is produced by a CRISPR/Cas9 gene editing system, which comprises a guide RNA comprising a spacer sequence of SEQ ID NO: 12 or 13.

51. The method of claim 43, wherein the disrupted CD70 gene is produced by a CRISPR/Cas9 gene editing system, which comprises a guide RNA comprising a spacer sequence of SEQ ID NO: 4 or 5.

52. The method of claim 1, wherein the CAR that binds CD70 comprises an extracellular domain, a CD8 transmembrane domain, a 4-1BB co-stimulatory domain, and a CD3ζ cytoplasmic signaling domain, and wherein the extracellular domain is a single-chain antibody fragment (scFv) that binds CD70.

53. The method of claim 52, wherein the scFv comprises a heavy chain variable domain (V_{H}) comprising SEQ ID NO: 49, and a light chain variable domain (V_{L}) comprising SEQ ID NO: 50.

54. The method of claim 53, wherein the scFv comprises SEQ ID NO: 48.

55. The method of claim 52, wherein the CAR comprises SEQ ID NO: 46 or SEQ ID NO: 81.

56. The method of claim 1, wherein the population of genetically engineered T cells comprise ≥30% CAR+ T cells, ≤0.5% TCR+ T cells, ≤30% B2M+ T cells, and ≤20% CD70+ T cells.

57. (canceled)


SEQUENCE LISTING (81 sequences)

SEQ ID NO: 1 - protein, 1368 aa, Artificial Sequence [sequence omitted]

SEQ ID NO: 2 - RNA, 100 nt, Artificial Sequence
  Synthetic
  misc_feature (1)..(4): modified with 2′-O-methyl phosphorothioate
  misc_feature (97)..(100): modified with 2′-O-methyl phosphorothioate
gcuuuggucccauuggucgcguuuuagagcuagaaauagcaaguuaaaauaaggcuaguccguuaucaacuugaaaaaguggcaccgagucggugcuuuu

SEQ ID NO: 3 - RNA, 100 nt, Artificial Sequence; identical to SEQ ID NO: 2
  Synthetic

SEQ ID NO: 4 - RNA, 20 nt, Artificial Sequence
  Synthetic
  misc_feature (1)..(4): modified with 2′-O-methyl phosphorothioate
gcuuuggucccauuggucgc

SEQ ID NO: 5 - RNA, 20 nt, Artificial Sequence
  Synthetic
gcuuuggucccauuggucgc

SEQ ID NO: 6 - RNA, 100 nt, Artificial Sequence
  Synthetic
  misc_feature (1)..(4): modified with 2′-O-methyl phosphorothioate
  misc_feature (97)..(100): modified with 2′-O-methyl phosphorothioate
agagcaacagugcuguggccguuuuagagcuagaaauagcaaguuaaaauaaggcuaguccguuaucaacuugaaaaaguggcaccgagucggugcuuuu

SEQ ID NO: 7 - RNA, 100 nt, Artificial Sequence; identical to SEQ ID NO: 6
  Synthetic

SEQ ID NO: 8 - RNA, 20 nt, Artificial Sequence
  Synthetic
  misc_feature (1)..(4): modified with 2′-O-methyl phosphorothioate
agagcaacagugcuguggcc

SEQ ID NO: 9 - RNA, 20 nt, Artificial Sequence
  Synthetic
agagcaacagugcuguggcc

SEQ ID NO: 10 - RNA, 100 nt, Artificial Sequence
  Synthetic
  misc_feature (1)..(4): modified with 2′-O-methyl phosphorothioate
  misc_feature (97)..(100): modified with 2′-O-methyl phosphorothioate
gcuacucucucuuucuggccguuuuagagcuagaaauagcaaguuaaaauaaggcuaguccguuaucaacuugaaaaaguggcaccgagucggugcuuuu

SEQ ID NO: 11 - RNA, 100 nt, Artificial Sequence; identical to SEQ ID NO: 10
  Synthetic

SEQ ID NO: 12 - RNA, 20 nt, Artificial Sequence
  Synthetic
  misc_feature (1)..(4): modified with 2′-O-methyl phosphorothioate
gcuacucucucuuucuggcc

SEQ ID NO: 13 - RNA, 20 nt, Artificial Sequence
  Synthetic
gcuacucucucuuucuggcc

SEQ ID NO: 14 - DNA, 23 bp, Artificial Sequence
  Synthetic
gctttggtcccattggtcgcggg

SEQ ID NO: 15 - DNA, 20 bp, Artificial Sequence
  Synthetic
gctttggtcccattggtcgc

SEQ ID NO: 16 - DNA, 23 bp, Artificial Sequence
  Synthetic
agagcaacagtgctgtggcctgg

SEQ ID NO: 17 - DNA, 20 bp, Artificial Sequence
  Synthetic
agagcaacagtgctgtggcc

SEQ ID NO: 18 - DNA, 23 bp, Artificial Sequence
  Synthetic
gctactctctctttctggcctgg

SEQ ID NO: 19 - DNA, 20 bp, Artificial Sequence
  Synthetic
gctactctctctttctggcc

SEQ ID NO: 20 - RNA, 100 nt, Artificial Sequence
  Synthetic
  misc_feature (1)..(20): n is a, c, g, or u
nnnnnnnnnnnnnnnnnnnnguuuuagagcuagaaauagcaaguuaaaauaaggcuaguccguuaucaacuugaaaaaguggcaccgagucggugcuuuu

SEQ ID NO: 21 - RNA, 96 nt, Artificial Sequence
  Synthetic
  misc_feature (1)..(20): n is a, c, g, or u
nnnnnnnnnnnnnnnnnnnnguuuuagagcuagaaauagcaaguuaaaauaaggcuaguccguuaucaacuugaaaaaguggcaccgagucggugc

SEQ ID NO: 22 - RNA, 111 nt, Artificial Sequence
  Synthetic
  misc_feature (1)..(17): n is a, c, g, or u
  misc_feature (18)..(30): may be absent
  misc_feature (105)..(111): may be absent
nnnnnnnnnnnnnnnnnnnnnnnnnnnguuuuagagcuagaaauagcaaguuaaaauaaggcuaguccguuaucaacuugaaaaaguggcaccgagucggugcuuuuuuuu

SEQ ID NO: 23 - DNA, 19 bp, Artificial Sequence
  Synthetic
aagagcaacaaatctgact

SEQ ID NO: 24 - DNA, 39 bp, Artificial Sequence
  Synthetic
aagagcaacagtgctgtgcctggagcaacaaatctgact

SEQ ID NO: 25 - DNA, 33 bp, Artificial Sequence
  Synthetic
aagagcaacagtgctggagcaacaaatctgact

SEQ ID NO: 26 - DNA, 34 bp, Artificial Sequence
  Synthetic
aagagcaacagtgcctggagcaacaaatctgact

SEQ ID NO: 27 - DNA, 19 bp, Artificial Sequence
  Synthetic
aagagcaacagtgctgact

SEQ ID NO: 28 - DNA, 41 bp, Artificial Sequence
  Synthetic
aagagcaacagtgctgtgggcctggagcaacaaatctgact

SEQ ID NO: 29 - DNA, 38 bp, Artificial Sequence
  Synthetic
aagagcaacagtgctggcctggagcaacaaatctgact

SEQ ID NO: 30 - DNA, 41 bp, Artificial Sequence
  Synthetic
aagagcaacagtgctgtgtgcctggagcaacaaatctgact

SEQ ID NO: 31 - DNA, 79 bp, Artificial Sequence
  Synthetic
cgtggccttagctgtgctcgcgctactctctctttctgcctggaggctatccagcgtgagtctctcctaccctcccgct

SEQ ID NO: 32 - DNA, 78 bp, Artificial Sequence
  Synthetic
cgtggccttagctgtgctcgcgctactctctctttcgcctggaggctatccagcgtgagtctctcctaccctcccgct

SEQ ID NO: 33 - DNA, 75 bp, Artificial Sequence
  Synthetic
cgtggccttagctgtgctcgcgctactctctctttctggaggctatccagcgtgagtctctcctaccctcccgct

SEQ ID NO: 34 - DNA, 84 bp, Artificial Sequence
  Synthetic
cgtggccttagctgtgctcgcgctactctctctttctggatagcctggaggctatccagcgtgagtctctcctaccctcccgct

SEQ ID NO: 35 - DNA, 55 bp, Artificial Sequence
  Synthetic
cgtggccttagctgtgctcgcgctatccagcgtgagtctctcctaccctcccgct

SEQ ID NO: 36 - DNA, 82 bp, Artificial Sequence
  Synthetic
cgtggccttagctgtgctcgcgctactctctctttctgtggcctggaggctatccagcgtgagtctctcctaccctcccgct

SEQ ID NO: 37 - DNA, 58 bp, Artificial Sequence
  Synthetic
cacaccacgaggcagatcaccaagcccgcgcaatgggaccaaagcagcccgcaggacg

SEQ ID NO: 38 - DNA, 61 bp, Artificial Sequence
  Synthetic
cacaccacgaggcagatcaccaagcccgcgaaccaatgggaccaaagcagcccgcaggacg

SEQ ID NO: 39 - DNA, 48 bp, Artificial Sequence
  Synthetic
cacaccacgaggcagatcaccaatgggaccaaagcagcccgcaggacg

SEQ ID NO: 40 - DNA, 59 bp, Artificial Sequence
  Synthetic
cacaccacgaggcagatcaccaagcccgcgccaatgggaccaaagcagcccgcaggacg

SEQ ID NO: 41 - DNA, 59 bp, Artificial Sequence
  Synthetic
cacaccacgaggcagatcaccaagcccgcaccaatgggaccaaagcagcccgcaggacg

SEQ ID NO: 42 - DNA, 35 bp, Artificial Sequence
  Synthetic
cacaccacgaggcagatcaccaagcccgcaggacg

SEQ ID NO: 43 - DNA, 4688 bp, Artificial Sequence
  Synthetic
cctgcaggcagctgcgcgctcgctcgctcactgaggccgcccgggcgtcgggcgacctttggtcgcccggcctcagtgagcgagcgagcgcgcagagagggagtggccaactccatcactaggggttcctgcggccgcacgcgtgagatgtaaggagctgctgtgacttgctcaaggccttatatcgagtaaacggtagtgctggggcttagacgcaggtgttctgatttatagttcaaaacctctatcaatgagagagcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaccaccatggcgcttccggtgacagcactgctcctccccttggcgctgttgctccacgcagcaaggccgcaggtccagttggtgcaaagcggggcggaggtgaaaaaacccggcgcttccgtgaaggtgtcctgtaaggcgtccggttatacgttcacgaactacgggatgaattgggttcgccaagcgccggggcagggactgaaatggatggggtggataaatacctacaccggcgaacctacatacgccgacgcttttaaagggcgagtcactatgacgcgcgataccagcatatccaccgcatacatggagctgtcccgactccggtcagacgacacggctgtctactattgtgctcgggactatggcgattatggcatggactactggggtcagggtacgactgtaacagttagtagtggtggaggcggcagtggcggggggggaagcggaggagggggttctggtgacatagttatgacccaatccccagatagtttggcggtttctctgggcgagagggcaacgattaattgtcgcgcatcaaagagcgtttcaacgagcggatattcttttatgcattggtaccagcaaaaacccggacaaccgccgaagctgctgatctacttggcttcaaatcttgagtctggggtgccggaccgattttctggtagtggaagcggaactgactttacgctcacgatcagttcactgcaggctgaggatgtagcggtctattattgccagcacagtagagaagtcccctggaccttcggtcaaggcacgaaagtagaaattaaaagtgctgctgcctttgtcccggtatttctcccagccaaaccgaccacgactcccgccccgcgccctccgacacccgctcccaccatcgcctctcaacctcttagtcttcgccccgaggcatgccgacccgccgccgggggtgctgttcatacgaggggcttggacttcgcttgtgatatttacatttgggctccgttggcgggtacgtgcggcgtccttttgttgtcactcgttattactttgtattgtaatcacaggaatcgcaaacggggcagaaagaaactcctgtatatattcaaacaaccatttatgagaccagtacaaactactcaagaggaagatggctgtagctgccgatttccagaagaagaagaaggaggatgtgaactgcgagtgaagttttcccgaagcgcagacgctccggcatatcagcaaggacagaatcagctgtataacgaactgaatttgggacgccgcgaggagtatgacgtgcttgataaacgccgggggagagacccggaaatggggggtaaaccccgaagaaagaatccccaagaaggactctacaatgaactccagaaggataagatggcggaggcctactcagaaataggtatgaagggcgaacgacgacggggaaaaggtcacgatggcctctaccaagggttgagtacggcaaccaaagatacgtacgatgcactgcatatgcaggccctgcctcccagataataataaaatcgctatccatcgaagatggatgtgtgttggttttttgtgtgtggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagctaccttcaggacaaaagtcagggaagggctctctgaagaaatgctacttgaagataccagccctaccaagggcagggagaggaccctatagaggcctgggacaggagctcaatgagaaaggtaaccacgtgcggaccgaggctgcagcgtcgtcctccctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 44 - DNA, 4364 bp, Artificial Sequence
  Synthetic
gagatgtaaggagctgctgtgacttgctcaaggccttatatcgagtaaacggtagtgctggggcttagacgcaggtgttctgatttatagttcaaaacctctatcaatgagagagcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaccaccatggcgcttccggtgacagcactgctcctccccttggcgctgttgctccacgcagcaaggccgcaggtccagttggtgcaaagcggggcggaggtgaaaaaacccggcgcttccgtgaaggtgtcctgtaaggcgtccggttatacgttcacgaactacgggatgaattgggttcgccaagcgccggggcagggactgaaatggatggggtggataaatacctacaccggcgaacctacatacgccgacgcttttaaagggcgagtcactatgacgcgcgataccagcatatccaccgcatacatggagctgtcccgactccggtcagacgacacggctgtctactattgtgctcgggactatggcgattatggcatggactactggggtcagggtacgactgtaacagttagtagtggtggaggcggcagtggcggggggggaagcggaggagggggttctggtgacatagttatgacccaatccccagatagtttggcggtttctctgggcgagagggcaacgattaattgtcgcgcatcaaagagcgtttcaacgagcggatattcttttatgcattggtaccagcaaaaacccggacaaccgccgaagctgctgatctacttggcttcaaatcttgagtctggggtgccggaccgattttctggtagtggaagcggaactgactttacgctcacgatcagttcactgcaggctgaggatgtagcggtctattattgccagcacagtagagaagtcccctggaccttcggtcaaggcacgaaagtagaaattaaaagtgctgctgcctttgtcccggtatttctcccagccaaaccgaccacgactcccgccccgcgccctccgacacccgctcccaccatcgcctctcaacctcttagtcttcgccccgaggcatgccgacccgccgccgggggtgctgttcatacgaggggcttggacttcgcttgtgatatttacatttgggctccgttggcgggtacgtgcggcgtccttttgttgtcactcgttattactttgtattgtaatcacaggaatcgcaaacggggcagaaagaaactcctgtatatattcaaacaaccatttatgagaccagtacaaactactcaagaggaagatggctgtagctgccgatttccagaagaagaagaaggaggatgtgaactgcgagtgaagttttcccgaagcgcagacgctccggcatatcagcaaggacagaatcagctgtataacgaactgaatttgggacgccgcgaggagtatgacgtgcttgataaacgccgggggagagacccggaaatggggggtaaaccccgaagaaagaatccccaagaaggactctacaatgaactccagaaggataagatggcggaggcctactcagaaataggtatgaagggcgaacgacgacggggaaaaggtcacgatggcctctaccaagggttgagtacggcaaccaaagatacgtacgatgcactgcatatgcaggccctgcctcccagataataataaaatcgctatccatcgaagatggatgtgtgttggttttttgtgtgtggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagctaccttcaggacaaaagtcagggaagggctctctgaagaaatgctacttgaagataccagccctaccaagggcagggagaggaccctatagaggcctgggacaggagctcaatgagaaagg

SEQ ID NO: 45 - DNA, 1527 bp, Artificial Sequence
  Synthetic
atggcgcttccggtgacagcactgctcctccccttggcgctgttgctccacgcagcaaggccgcaggtccagttggtgcaaagcggggcggaggtgaaaaaacccggcgcttccgtgaaggtgtcctgtaaggcgtccggttatacgttcacgaactacgggatgaattgggttcgccaagcgccggggcagggactgaaatggatggggtggataaatacctacaccggcgaacctacatacgccgacgcttttaaagggcgagtcactatgacgcgcgataccagcatatccaccgcatacatggagctgtcccgactccggtcagacgacacggctgtctactattgtgctcgggactatggcgattatggcatggactactggggtcagggtacgactgtaacagttagtagtggtggaggcggcagtggcggggggggaagcggaggagggggttctggtgacatagttatgacccaatccccagatagtttggcggtttctctgggcgagagggcaacgattaattgtcgcgcatcaaagagcgtttcaacgagcggatattcttttatgcattggtaccagcaaaaacccggacaaccgccgaagctgctgatctacttggcttcaaatcttgagtctggggtgccggaccgattttctggtagtggaagcggaactgactttacgctcacgatcagttcactgcaggctgaggatgtagcggtctattattgccagcacagtagagaagtcccctggaccttcggtcaaggcacgaaagtagaaattaaaagtgctgctgcctttgtcccggtatttctcccagccaaaccgaccacgactcccgccccgcgccctccgacacccgctcccaccatcgcctctcaacctcttagtcttcgccccgaggcatgccgacccgccgccgggggtgctgttcatacgaggggcttggacttcgcttgtgatatttacatttgggctccgttggcgggtacgtgcggcgtccttttgttgtcactcgttattactttgtattgtaatcacaggaatcgcaaacggggcagaaagaaactcctgtatatattcaaacaaccatttatgagaccagtacaaactactcaagaggaagatggctgtagctgccgatttccagaagaagaagaaggaggatgtgaactgcgagtgaagttttcccgaagcgcagacgctccggcatatcagcaaggacagaatcagctgtataacgaactgaatttgggacgccgcgaggagtatgacgtgcttgataaacgccgggggagagacccggaaatggggggtaaaccccgaagaaagaatccccaagaaggactctacaatgaactccagaaggataagatggcggaggcctactcagaaataggtatgaagggcgaacgacgacggggaaaaggtcacgatggcctctaccaagggttgagtacggcaaccaaagatacgtacgatgcactgcatatgcaggccctgcctcccagataa

SEQ ID NO: 46 - protein, 508 aa, Artificial Sequence
  Synthetic
MALPVTALLLPLALLLHAARPQVQLVQSGAEVKKPGASVKVSCKASGYTFTNYGMNWVRQAPGQGLKWMGWINTYTGEPTYADAFKGRVTMTRDTSISTAYMELSRLRSDDTAVYYCARDYGDYGMDYWGQGTTVTVSSGGGGSGGGGSGGGGSGDIVMTQSPDSLAVSLGERATINCRASKSVSTSGYSFMHWYQQKPGQPPKLLIYLASNLESGVPDRFSGSGSGTDFTLTISSLQAEDVAVYYCQHSREVPWTFGQGTKVEIKSAAAFVPVFLPAKPTTTPAPRPPTPAPTIASQPLSLRPEACRPAAGGAVHTRGLDFACDIYIWAPLAGTCGVLLLSLVITLYCNHRNRKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 47 - DNA, 735 bp, Artificial Sequence
  Synthetic
caggtccagttggtgcaaagcggggcggaggtgaaaaaacccggcgcttccgtgaaggtgtcctgtaaggcgtccggttatacgttcacgaactacgggatgaattgggttcgccaagcgccggggcagggactgaaatggatggggtggataaatacctacaccggcgaacctacatacgccgacgcttttaaagggcgagtcactatgacgcgcgataccagcatatccaccgcatacatggagctgtcccgactccggtcagacgacacggctgtctactattgtgctcgggactatggcgattatggcatggactactggggtcagggtacgactgtaacagttagtagtggtggaggcggcagtggcggggggggaagcggaggagggggttctggtgacatagttatgacccaatccccagatagtttggcggtttctctgggcgagagggcaacgattaattgtcgcgcatcaaagagcgtttcaacgagcggatattcttttatgcattggtaccagcaaaaacccggacaaccgccgaagctgctgatctacttggcttcaaatcttgagtctggggtgccggaccgattttctggtagtggaagcggaactgactttacgctcacgatcagttcactgcaggctgaggatgtagcggtctattattgccagcacagtagagaagtcccctggaccttcggtcaaggcacgaaagtagaaattaaa

SEQ ID NO: 48 - protein, 245 aa, Artificial Sequence
  Synthetic
QVQLVQSGAEVKKPGASVKVSCKASGYTFTNYGMNWVRQAPGQGLKWMGWINTYTGEPTYADAFKGRVTMTRDTSISTAYMELSRLRSDDTAVYYCARDYGDYGMDYWGQGTTVTVSSGGGGSGGGGSGGGGSGDIVMTQSPDSLAVSLGERATINCRASKSVSTSGYSFMHWYQQKPGQPPKLLIYLASNLESGVPDRFSGSGSGTDFTLTISSLQAEDVAVYYCQHSREVPWTFGQGTKVEIK

SEQ ID NO: 49 - protein, 118 aa, Artificial Sequence
  Synthetic
QVQLVQSGAEVKKPGASVKVSCKASGYTFTNYGMNWVRQAPGQGLKWMGWINTYTGEPTYADAFKGRVTMTRDTSISTAYMELSRLRSDDTAVYYCARDYGDYGMDYWGQGTTVTVSS

SEQ ID NO: 50 - protein, 111 aa, Artificial Sequence
  Synthetic
DIVMTQSPDSLAVSLGERATINCRASKSVSTSGYSFMHWYQQKPGQPPKLLIYLASNLESGVPDRFSGSGSGTDFTLTISSLQAEDVAVYYCQHSREVPWTFGQGTKVEIK

SEQ ID NO: 51 - protein, 16 aa, Artificial Sequence
  Synthetic
GGGGSGGGGSGGGGSG

SEQ ID NO: 52 - protein, 22 aa, Artificial Sequence
  Synthetic
MLLLVTSLLLCELPHPAFLLIP

SEQ ID NO: 53 - protein, 21 aa, Artificial Sequence
  Synthetic
MALPVTALLLPLALLLHAARP

SEQ ID NO: 54 - protein, 84 aa, Artificial Sequence
  Synthetic
FVPVFLPAKPTTTPAPRPPTPAPTIASQPLSLRPEACRPAAGGAVHTRGLDFACDIYIWAPLAGTCGVLLLSLVITLYCNHRNR

SEQ ID NO: 55 - protein, 23 aa, Artificial Sequence
  Synthetic
IYIWAPLAGTCGVLLLSLVITLY

SEQ ID NO: 56 - DNA, 126 bp, Artificial Sequence
  Synthetic
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 57 - protein, 42 aa, Artificial Sequence
  Synthetic
KRGRKKLLYIFKQPFMRPVQTTQEEDGCSCRFPEEEEGGCEL

SEQ ID NO: 58 - DNA, 120 bp, Artificial Sequence
  Synthetic
tcaaagcggagtaggttgttgcattccgattacatgaatatgactcctcgccggcctgggccgacaagaaaacattaccaaccctatgcccccccacgagacttcgctgcgtacaggtcc

SEQ ID NO: 59 - protein, 40 aa, Artificial Sequence
  Synthetic
SKRSRLLHSDYMNMTPRRPGPTRKHYQPYAPPRDFAAYRS

SEQ ID NO: 60 - DNA, 336 bp, Artificial Sequence
  Synthetic
cgagtgaagttttcccgaagcgcagacgctccggcatatcagcaaggacagaatcagctgtataacgaactgaatttgggacgccgcgaggagtatgacgtgcttgataaacgccgggggagagacccggaaatggggggtaaaccccgaagaaagaatccccaagaaggactctacaatgaactccagaaggataagatggcggaggcctactcagaaataggtatgaagggcgaacgacgacggggaaaaggtcacgatggcctctaccaagggttgagtacggcaaccaaagatacgtacgatgcactgcatatgcaggccctgcctcccaga

SEQ ID NO: 61 - protein, 112 aa, Artificial Sequence
  Synthetic
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 62 - DNA, 800 bp, Artificial Sequence
  Synthetic
gagatgtaaggagctgctgtgacttgctcaaggccttatatcgagtaaacggtagtgctggggcttagacgcaggtgttctgatttatagttcaaaacctctatcaatgagagagcaatctcctggtaatgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttca

SEQ ID NO: 63 - DNA, 1178 bp, Artificial Sequence
  Synthetic
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccactggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 64 - DNA, 49 bp, Artificial Sequence
  Synthetic
aataaaatcgctatccatcgaagatggatgtgtgttggttttttgtgtg

SEQ ID NO: 65 - DNA, 804 bp, Artificial Sequence
  Synthetic
tggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagctaccttcaggacaaaagtcagggaagggctctctgaagaaatgctacttgaagataccagccctaccaagggcagggagaggaccctatagaggcctgggacaggagctcaatgagaaagg

SEQ ID NO: 66 - RNA, 100 nt, Artificial Sequence
  Synthetic
  misc_feature (1)..(4): modified with 2′-O-methyl phosphorothioate
  misc_feature (97)..(100): modified with 2′-O-methyl phosphorothioate
gcccgcaggacgcacccauaguuuuagagcuagaaauagcaaguuaaaauaaggcuaguccguuaucaacuugaaaaaguggcaccgagucggugcuuuu

SEQ ID NO: 67 - RNA, 100 nt, Artificial Sequence; identical to SEQ ID NO: 66
  Synthetic

SEQ ID NO: 68 - RNA, 20 nt, Artificial Sequence
  Synthetic
  misc_feature (1)..(4): modified with 2′-O-methyl phosphorothioate
gcccgcaggacgcacccaua

SEQ ID NO: 69 - RNA, 20 nt, Artificial Sequence
  Synthetic
gcccgcaggacgcacccaua

SEQ ID NO: 70 - protein, 300 aa, Artificial Sequence
  Synthetic
MANCEFSPVSGDKPCCRLSRRAQLCLGVSILVLILVVVLAVVVPRWRQQWSGPGTTKRFPETVLARCVKYTEIHPEMRHVDCQSVWDAFKGAFISKHPCNITEEDYQPLMKLGTQTVPCNKILLWSRIKDLAHQFTQVQRDMFTLEDTLLGYLADDLTWCGEFNTSKINYQSCPDWRKDCSNNPVSVFWKTVSRRFAEAACDVVHVMLNGSRSKIFDKNSTFGSVEVHNLQPEKVQTLEAWVIHGGREDSRDLCQDPTIKELESIISKRNIQFSCKNIYRPDKFLQCVKNPEDSSCTSEI

SEQ ID NO: 71 - protein, 452 aa, Artificial Sequence
  Synthetic
EVQLLESGGGLVQPGGSLRLSCAVSGFTFNSFAMSWVRQAPGKGLEWVSAISGSGGGTYYADSVKGRFTISRDNSKNTLYLQMNSLRAEDTAVYFCAKDKILWFGEPVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 72 - protein, 124 aa, Artificial Sequence
  Synthetic
EVQLLESGGGLVQPGGSLRLSCAVSGFTFNSFAMSWVRQAPGKGLEWVSAISGSGGGTYYADSVKGRFTISRDNSKNTLYLQMNSLRAEDTAVYFCAKDKILWFGEPVFDYWGQGTLVTVSSAS

SEQ ID NO: 73 - protein, 213 aa, Artificial Sequence
  Synthetic
EIVLTQSPATLSLSPGERATLSCRASQSVSSYLAWYQQKPQAPRLLIYDASNRATGIPARFSGSGSGTDFTLTISSLEPEDFAVYYCQQRSNWPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 74 - protein, 107 aa, Artificial Sequence [sequence omitted]

SEQ ID NO: 75 - protein, 5 aa, Artificial Sequence
  Synthetic
SFAMS

SEQ ID NO: 76 - protein, 17 aa, Artificial Sequence
  Synthetic
AISGSGGGTYYADSVKG

SEQ ID NO: 77 - protein, 13 aa, Artificial Sequence
  Synthetic
DKILWFGEPVFDY

SEQ ID NO: 78 - protein, 11 aa, Artificial Sequence
  Synthetic
RASQSVSSYLA

SEQ ID NO: 79 - protein, 7 aa, Artificial Sequence
  Synthetic
DASNRAT

SEQ ID NO: 80 - protein, 9 aa, Artificial Sequence
  Synthetic
QQRSNWPPT

SEQ ID NO: 81 - protein, 487 aa, Artificial Sequence
  Synthetic
QVQLVQSGAEVKKPGASVKVSCKASGYTFTNYGMNWVRQAPGQGLKWMGWINTYTGEPTYADAFKGRVTMTRDTSISTAYMELSRLRSDDTAVYYCARDYGDYGMDYWGQGTTVTVSSGGGGSGGGGSGGGGSGDIVMTQSPDSLAVSLGERATINCRASKSVSTSGYSFMHWYQQKPGQPPKLLIYLASNLESGVPDRFSGSGSGTDFTLTISSLQAEDVAVYYCQHSREVPWTFGQGTKVEIKSAAAFVPVFLPAKPTTTPAPRPPTPAPTIASQPLSLRPEACRPAAGGAVHTRGLDFACDIYIWAPLAGTCGVLLLSLVITLYCNHRNRKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR